(19) United States
(12) Patent Application Publication
(10) Pub. No.: US 2024/0115607 A1
(43) Pub. Date: Apr. 11, 2024

(54) CHIMERIC ANTIGEN RECEPTOR (CAR) CONSTRUCTS AND NK CELLS EXPRESSING CAR CONSTRUCTS

(71) Applicants: LI CHEN, Shanghai (CN); Huaxing ZHU, Shanghai (CN); CYTOCARES (SHANGHAI) INC., Shanghai (CN)

(72) Inventors: Qiming XU, Shanghai (CN); Li CHEN, Shanghai (CN); Huaxing ZHU, Shanghai (CN)

(73) Assignee: CYTOCARES (SHANGHAI) INC., Shanghai (CN)

(21) Appl. No.: 18/273,661

(22) PCT Filed: Jan. 25, 2022
(86) PCT No.: PCT/CN2022/073808

§ 371 Date: Jul. 21, 2023

Related U.S. Application Data
(60) Provisional application No. 63/141,621, filed on Jan. 26, 2021.

(51) Int. Cl. A61K 35/17; A61K 39/00; A61P 35/00; C07K 14/705

(52) U.S. Cl. CPC A61K 35/17; A61K 39/4631; A61K 39/464411; A61P 35/00; C07K 14/70503; A61K 2239/15; A61K 2239/17; A61K 2239/21; A61K 2239/22

(57) ABSTRACT

The technology described herein is directed to Natural Killer (NK) cell CAR polypeptides comprising intracellular signaling domains, intracellular costimulatory domains, and/or transmembrane domains from NK-associated polypeptides. In various aspects, described herein are polynucleotides, vectors, or cells expressing said NK CAR polypeptides, and pharmaceutical compositions comprising said NK CAR polypeptides, polynucleotides, vectors, or cells. Also described herein are methods of using said NK CAR polypeptides, for example to treat various diseases and disorders, such as cancer or infectious diseases.

### CROSS-REFERENCE TO RELATED APPLICATIONS

This application claims benefit under 35 U.S.C. § 119(e) of U.S. Provisional Application No. 63/141,621 filed Jan. 26, 2021, the contents of which are incorporated herein by reference in their entirety.

### SEQUENCE LISTING

The instant application contains a Sequence Listing which has been submitted in ASCII format via CE-PCT and is hereby incorporated by reference in its entirety. Said ASCII copy, created on Dec. 29, 2021, is named 087825-000003WOPT_SL.txt and is 307,704 bytes in size.

### TECHNICAL FIELD

The technology described herein relates to natural killer (NK) cell chimeric antigen receptor (CAR) constructs.

### BACKGROUND

Adoptive T cell therapies using chimeric antigen receptor (CAR) constructs have been used as a treatment for cancers. CAR-T cell therapies can result in T cell overactivity and cytokine release syndrome (CRS). Such cytokine storms can lead to organ failure or death in severe cases. As such, there is an urgent need for CAR technology that can be expressed in immune cell types other than T cells. NK cells are functionally similar to CD8+ cytotoxic T cells and considered an alternative for T cells in cytotoxic killing of tumor cells. Compared to CAR-T cells, CAR-NK technology offers significant advantages, e.g., lower occurrence of CRS or neurotoxicity, and high feasibility for “off-the-shelf” manufacturing. At present, most CAR-NK cell studies use CAR constructs designed for CAR-T cells, not optimized for NK cell activation. As such, there is an urgent need for development of CAR construct for specific activation of NK cell to achieve enhanced anti-tumor activities.

### SUMMARY

This technology relates to Chimeric Antigen Receptor (CAR) constructs that can be expressed in Natural Killer (NK) cells, and their use in the treatment of cancer and infectious disease. Described herein are CAR constructs that can be used for NK cell engineering in methods of modifying NK cells to express these constructs and in methods using CAR modified NK cells as therapeutic agents. Generally, an NK CAR construct comprises an extracellular domain comprising an extracellular binding domain (i.e., an antigen recognition region), a transmembrane domain (TD), and an intracellular domain comprising 1 or 2 signaling domain(s), and/or a co-stimulatory domain. This technology provides CAR constructs with transmembrane domains and/or intracellular domains different from those used in CAR-T therapy, and adapted for use in modifying NK cells, e.g., to target them to cells expressing a given marker or tumor antigen. NK cells modified with the CAR constructs described herein have more potent NK cell activation and cytotoxic activity than those currently used for CAR-T therapy. This technology thus provides CAR constructs that induce high levels of NK cell proliferation, activation, cytokine secretion, and cytolytic activity.

In one aspect, described herein is a chimeric antigen receptor (CAR) polypeptide for expression in natural killer (NK) cells comprising: (a) an extracellular binding domain; (b) a transmembrane domain; and (c) an intracellular domain comprising at least one of the following: (i) an intracellular signaling domain from an NK cell receptor; (ii) an intracellular signaling domain from an NK cell membrane-bound signaling adaptor protein; and/or (iii) an intracellular co-stimulatory domain from a co-stimulatory receptor.

In some embodiments of any of the aspects, the polypeptide comprises from N-terminus to C-terminus: (a) the extracellular binding domain; (b) the transmembrane domain; and (c) the intracellular domain.

In some embodiments of any of the aspects, the polypeptide comprises from C-terminus to N-terminus: (a) the extracellular binding domain; (b) the transmembrane domain; and (c) the intracellular domain.

In some embodiments of any of the aspects, the NK cell receptor or NK cell membrane-bound signaling adaptor protein is selected from the group consisting of: (a) Natural Killer Cell Receptor 2B4; (b) Natural Killer-, T- And B-Cell Antigen (NTB-A); (c) CD2-Like Receptor Activating Cytotoxic Cells (CRACC); (d) cluster of differentiation 2 (CD2); (e) high-affinity IgE receptor (FcεR1); and (f) CD3-zeta (CD3ζ).

In some embodiments of any of the aspects, the NK cell receptor is selected from the group consisting of: 2B4, NTB-A, CRACC, and CD2.

In some embodiments of any of the aspects, the NK cell receptor is 2B4.

In some embodiments of any of the aspects, the NK cell receptor is NTB-A.

In some embodiments of any of the aspects, the NK cell receptor is CRACC.

In some embodiments of any of the aspects, the NK cell receptor is CD2.

In some embodiments of any of the aspects, the NK cell membrane-bound signaling adaptor protein is CD3ζ or FcεR1.

In some embodiments of any of the aspects, the intracellular signaling domain from the NK cell receptor or the intracellular signaling domain from the NK cell membrane-bound signaling adaptor protein comprises the amino acid sequence of one of SEQ ID NOs: 7-12 or an amino acid sequence that is at least 80% identical to one of SEQ ID NOs: 7-12.

In some embodiments of any of the aspects, the co-stimulatory receptor is 4-1BB and/or IL2 receptor beta (IL2RB).

In some embodiments of any of the aspects, the intracellular co-stimulatory domain from the co-stimulatory receptor comprises the amino acid sequence of one of SEQ ID NOs: 15-16 or an amino acid sequence that is at least 80% identical to one of SEQ ID NOs: 15-16.

In some embodiments of any of the aspects, the intracellular domain further comprises at least one self-cleaving peptide.

In some embodiments of any of the aspects, the self-cleaving peptide is T2A, P2A, E2A, or F2A.

In some embodiments of any of the aspects, the self-cleaving peptide comprises the amino acid sequence of one of SEQ ID NOs: 19-20 or an amino acid sequence that is at least 80% identical to one of SEQ ID NOs: 19-20.

In some embodiments of any of the aspects, the intracellular domain further comprises a cytokine.

In some embodiments of any of the aspects, the cytokine is IL-15 or IL-21.

In some embodiments of any of the aspects, the cytokine comprises the amino acid sequence of one of SEQ ID NOs: 23-24 or an amino acid sequence that is at least 80% identical to one of SEQ ID NOs: 23-24.

In some embodiments of any of the aspects, the intracellular domain further comprises at least one self-cleaving peptide and at least one cytokine.

In some embodiments of any of the aspects, the cytokine is adjacent and distal to the self-cleaving peptide, such that the cytokine is separated from the polypeptide by the self-cleaving peptide.

In some embodiments of any of the aspects, the transmembrane domain comprises a transmembrane domain of a natural NK cell receptor.

In some embodiments of any of the aspects, the transmembrane domain of the natural NK cell receptor is selected from the group consisting of Natural Killer Group 2D (NKG2D); Natural Killer Cell P46-Related Protein (NKp46); and DNAX Accessory Molecule-1 (DNAM1).

In some embodiments of any of the aspects, the transmembrane domain comprises a transmembrane domain of CD8.

In some embodiments of any of the aspects, the transmembrane domain comprises the amino acid sequence of one of SEQ ID NOs: 29-32 or 117-118 or an amino acid sequence that is at least 80% identical to one of SEQ ID NOs: 29-32 or 117-118.

In some embodiments of any of the aspects, the extracellular binding domain is an antibody, an antigen-binding fragment thereof, a F(ab) fragment, a F(ab′) fragment, a single chain variable fragment (scFv), or a single-domain antibody (sdAb).

In some embodiments of any of the aspects, the extracellular binding domain comprises a scFv.

In some embodiments of any of the aspects, the extracellular binding domain specifically binds to a tumor-associated antigen.

In some embodiments of any of the aspects, the tumor-associated antigen is CD19.

In some embodiments of any of the aspects, the tumor-associated antigen is CD33.

In some embodiments of any of the aspects, the extracellular binding domain comprises the amino acid sequence of one of SEQ ID NOs: 35-36 or an amino acid sequence that is at least 80% identical to one of SEQ ID NOs: 35-36.

In some embodiments of any of the aspects, the polypeptide further comprises a signal peptide located N-terminal to the extracellular binding domain.

In some embodiments of any of the aspects, the signal peptide is a CD8 signal peptide.

In some embodiments of any of the aspects, the signal peptide comprises the amino acid sequence of SEQ ID NO: 38 or an amino acid sequence that is at least 80% identical to SEQ ID NO: 38.

In some embodiments of any of the aspects, the polypeptide further comprises a detectable marker distal to the extracellular binding domain.

In some embodiments of any of the aspects, the detectable marker is 3×FLAG.

In some embodiments of any of the aspects, the detectable marker comprises the amino acid sequence of SEQ ID NO: 40 or an amino acid sequence that is at least 80% identical to SEQ ID NO: 40.

In some embodiments of any of the aspects, the polypeptide further comprises a linker domain distal to the extracellular binding domain and/or proximal to the signal peptide and/or detectable marker.

In some embodiments of any of the aspects, the linker comprises the amino acid sequence of SEQ ID NO: 42 or an amino acid sequence that is at least 80% identical to SEQ ID NO: 42.

In some embodiments of any of the aspects, the polypeptide further comprises a spacer domain located between the extracellular binding domain and the transmembrane domain.

In some embodiments of any of the aspects, the spacer domain comprises a CD8 hinge domain.

In some embodiments of any of the aspects, the spacer comprises the amino acid sequence of SEQ ID NO: 44 or an amino acid sequence that is at least 80% identical to SEQ ID NO: 44.

In some embodiments of any of the aspects, the polypeptide comprises the amino acid sequence of one of SEQ ID NOs: 80-114 or an amino acid sequence that is at least 80% identical to one of SEQ ID NOs: 80-114.

In one aspect described herein is a chimeric antigen receptor (CAR) polypeptide for expression in natural killer (NK) cells comprising: (a) an extracellular binding domain; (b) a transmembrane domain; and (c) intracellular domain comprising at least one intracellular signaling domain from a NK cell receptor.

In some embodiments of any of the aspects, the polypeptide comprises from N-terminus to C-terminus: (a) the extracellular binding domain; (b) the transmembrane domain; and (c) the intracellular domain.

In some embodiments of any of the aspects, the polypeptide comprises from C-terminus to N-terminus: (a) the extracellular binding domain; (b) the transmembrane domain; and (c) the intracellular domain.

In some embodiments of any of the aspects, the NK cell receptor is selected from the group consisting of: 2B4, NTB-A, CRACC, and CD2.

In some embodiments of any of the aspects, the NK cell receptor is 2B4.

In some embodiments of any of the aspects, the NK cell receptor is NTB-A.

In some embodiments of any of the aspects, the NK cell receptor is CRACC.

In some embodiments of any of the aspects, the NK cell receptor is CD2.

In some embodiments of any of the aspects, the intracellular signaling domain from the NK cell receptor comprises the amino acid sequence of one of SEQ ID NOs: 7-10 or an amino acid sequence that is at least 80% identical to one of SEQ ID NOs: 7-10.

In some embodiments of any of the aspects, the transmembrane domain comprises a transmembrane domain of a natural NK cell receptor.

In some embodiments of any of the aspects, the transmembrane domain of the natural NK cell receptor is selected from the group consisting of Natural Killer Group 2D (NKG2D); Natural Killer Cell P46-Related Protein (NKp46); and DNAX Accessory Molecule-1 (DNAM1).

In some embodiments of any of the aspects, the transmembrane domain comprises the amino acid sequence of one of SEQ ID NOs: 29-31 or 117-118 or an amino acid sequence that is at least 80% identical to one of SEQ ID NOs: 29-31 or 117-118.

In some embodiments of any of the aspects, the extracellular binding domain is an antibody, an antigen-binding fragment thereof, a F(ab) fragment, a F(ab′) fragment, a single chain variable fragment (scFv), or a single-domain antibody (sdAb).

In some embodiments of any of the aspects, the extracellular binding domain comprises an scFv.

In some embodiments of any of the aspects, the extracellular binding domain specifically binds to a tumor-associated antigen.

In some embodiments of any of the aspects, the tumor-associated antigen is CD19.

In some embodiments of any of the aspects, the tumor-associated antigen is CD33.

In some embodiments of any of the aspects, the extracellular binding domain comprises the amino acid sequence of one of SEQ ID NOs: 35-36 or an amino acid sequence that is at least 80% identical to one of SEQ ID NOs: 35-36.

In some embodiments of any of the aspects, the polypeptide further comprises a signal peptide located N-terminal to the extracellular binding domain.

In some embodiments of any of the aspects, the signal peptide is a CD8 signal peptide.

In some embodiments of any of the aspects, the signal peptide comprises the amino acid sequence of SEQ ID NO: 38 or an amino acid sequence that is at least 80% identical to SEQ ID NO: 38.

In some embodiments of any of the aspects, the polypeptide further comprises a detectable marker distal to the extracellular binding domain.

In some embodiments of any of the aspects, the detectable marker is 3×FLAG.

In some embodiments of any of the aspects, the detectable marker comprises the amino acid sequence of SEQ ID NO: 40 or an amino acid sequence that is at least 80% identical to SEQ ID NO: 40.

In some embodiments of any of the aspects, the polypeptide further comprises a linker domain distal to the extracellular binding domain and/or proximal to the signal peptide and/or detectable marker.

In some embodiments of any of the aspects, the linker comprises the amino acid sequence of SEQ ID NO: 42 or an amino acid sequence that is at least 80% identical to SEQ ID NO: 42.

In some embodiments of any of the aspects, the polypeptide further comprises a spacer domain located between the extracellular binding domain and the transmembrane domain.

In some embodiments of any of the aspects, the spacer domain comprises a CD8 hinge domain.

In some embodiments of any of the aspects, the spacer comprises the amino acid sequence of SEQ ID NO: 44 or an amino acid sequence that is at least 80% identical to SEQ ID NO: 44.

In some embodiments of any of the aspects, the polypeptide comprises the amino acid sequence of one of SEQ ID NOs: 90-105 or SEQ ID NOs: 110-114 or an amino acid sequence that is at least 80% identical to one of SEQ ID NOs: 90-105 or SEQ ID NOs: 110-114.

In one aspect described herein is a chimeric antigen receptor (CAR) polypeptide for expression in natural killer (NK) cells comprising: (a) an extracellular binding domain; (b) a transmembrane domain; and (c) an intracellular domain comprising at least one of the following: (i) an intracellular signaling domain from a NK cell membrane-bound signaling adaptor protein; and/or (ii) an intracellular co-stimulatory domain from a co-stimulatory receptor.

In some embodiments of any of the aspects, the polypeptide comprises from N-terminus to C-terminus: (a) the extracellular binding domain; (b) the transmembrane domain; and (c) the intracellular domain.

In some embodiments of any of the aspects, the polypeptide comprises from C-terminus to N-terminus: (a) the extracellular binding domain; (b) the transmembrane domain; and (c) the intracellular domain.

In some embodiments of any of the aspects, the NK cell membrane-bound signaling adaptor protein is CD3ζ or FcεR1.

In some embodiments of any of the aspects, the intracellular signaling domain from the NK cell membrane-bound signaling adaptor protein comprises the amino acid sequence of one of SEQ ID NOs: 11-12 or an amino acid sequence that is at least 80% identical to one of SEQ ID NOs: 11-12.

In some embodiments of any of the aspects, the co-stimulatory receptor is 4-1BB and/or IL2 receptor beta (IL2RB).

In some embodiments of any of the aspects, the intracellular co-stimulatory domain from the co-stimulatory receptor comprises the amino acid sequence of one of SEQ ID NOs: 15-16 or an amino acid sequence that is at least 80% identical to one of SEQ ID NOs: 15-16.

In some embodiments of any of the aspects, the intracellular domain further comprises at least one self-cleaving peptide.

In some embodiments of any of the aspects, the self-cleaving peptide is T2A, P2A, E2A, or F2A.

In some embodiments of any of the aspects, the self-cleaving peptide comprises the amino acid sequence of one of SEQ ID NOs: 19-20 or an amino acid sequence that is at least 80% identical to one of SEQ ID NOs: 19-20.

In some embodiments of any of the aspects, wherein the intracellular domain further comprises a cytokine.

In some embodiments of any of the aspects, the cytokine is IL-15 or IL-21.

In some embodiments of any of the aspects, the cytokine comprises the amino acid sequence of one of SEQ ID NOs: 23-24 or an amino acid sequence that is at least 80% identical to one of SEQ ID NOs: 23-24.

In some embodiments of any of the aspects, the intracellular domain further comprises at least one self-cleaving peptide and at least one cytokine.

In some embodiments of any of the aspects, the cytokine is adjacent and distal to the self-cleaving peptide, such that the cytokine is separated from the polypeptide by the self-cleaving peptide.

In some embodiments of any of the aspects, the transmembrane domain comprises a transmembrane domain of CD8.

In some embodiments of any of the aspects, the transmembrane domain comprises the amino acid sequence of SEQ ID NO: 32 or an amino acid sequence that is at least 80% identical to SEQ ID NO: 32.

In some embodiments of any of the aspects, the extracellular binding domain is an antibody, an antigen-binding fragment thereof, a F(ab) fragment, a F(ab′) fragment, a single chain variable fragment (scFv), or a single-domain antibody (sdAb).

In some embodiments of any of the aspects, the extracellular binding domain comprises a scFv.

In some embodiments of any of the aspects, the extracellular binding domain specifically binds to a tumor-associated antigen.

In some embodiments of any of the aspects, the tumor-associated antigen is CD19.

In some embodiments of any of the aspects, the tumor-associated antigen is CD33.

In some embodiments of any of the aspects, the extracellular binding domain comprises the amino acid sequence of one of SEQ ID NOs: 35-36 or an amino acid sequence that is at least 80% identical to one of SEQ ID NOs: 35-36.

In some embodiments of any of the aspects, the polypeptide further comprises a signal peptide located N-terminal to the extracellular binding domain.

In some embodiments of any of the aspects, the signal peptide is a CD8 signal peptide.

In some embodiments of any of the aspects, the signal peptide comprises the amino acid sequence of SEQ ID NO: 38 or an amino acid sequence that is at least 80% identical to SEQ ID NO: 38.

In some embodiments of any of the aspects, the polypeptide further comprises a detectable marker distal to the extracellular binding domain.

In some embodiments of any of the aspects, the detectable marker is 3×FLAG.

In some embodiments of any of the aspects, the detectable marker comprises the amino acid sequence of SEQ ID NO: 40 or an amino acid sequence that is at least 80% identical to SEQ ID NO: 40.

In some embodiments of any of the aspects, the polypeptide further comprises a linker domain distal to the extracellular binding domain and/or proximal to the signal peptide and/or detectable marker.

In some embodiments of any of the aspects, the linker comprises the amino acid sequence of SEQ ID NO: 42 or an amino acid sequence that is at least 80% identical to SEQ ID NO: 42.

In some embodiments of any of the aspects, the polypeptide further comprises a spacer domain located between the extracellular binding domain and the transmembrane domain.

In some embodiments of any of the aspects, the spacer domain comprises a CD8 hinge domain.

In some embodiments of any of the aspects, the spacer comprises the amino acid sequence of SEQ ID NO: 44 or an amino acid sequence that is at least 80% identical to SEQ ID NO: 44.

In some embodiments of any of the aspects, the polypeptide comprises the amino acid sequence of one of SEQ ID NOs: 80-89 or SEQ ID NOs: 106-109 or an amino acid sequence that is at least 80% identical to one of SEQ ID NOs: 80-89 or SEQ ID NOs: 106-109.

In one aspect described herein is a polynucleotide encoding a polypeptide as described herein.

In some embodiments of any of the aspects, the polynucleotide comprises one of SEQ ID NOs: 45-79, or a nucleic acid sequence that is at least 80% identical to one of SEQ ID NOs: 45-79.

In one aspect described herein is a vector comprising a polynucleotide as described herein.

In some embodiments of any of the aspects, the vector comprises a lentiviral vector.

In one aspect described herein is a lentivirus comprising a polypeptide as described herein, a polynucleotide as described herein, or a vector as described herein.

In one aspect described herein is a cell or population thereof comprising a polypeptide as described herein, a polynucleotide as described herein, a vector as described herein, or a lentivirus as described herein.

In some embodiments of any of the aspects, the cell comprises an immune cell.

In some embodiments of any of the aspects, the immune cell comprises a natural killer (NK) cell.

In one aspect described herein is a pharmaceutical composition comprising a polypeptide as described herein, a polynucleotide as described herein, a vector as described herein, a lentivirus as described herein, or a cell as described herein, and a pharmaceutically acceptable carrier.

In one aspect described herein is a method of increasing the activation of an NK cell or population thereof comprising contacting the cell or population thereof with a polypeptide as described herein, a polynucleotide as described herein, a vector as described herein, or a lentivirus as described herein.

In some embodiments of any of the aspects, contacting the NK cell or population thereof with the polypeptide, polynucleotide, vector, or lentivirus increases the activity of the NK cell by at least 10%, at least 20%, at least 30%, at least 40%, at least 50%, at least 60%, at least 70%, at least 80%, at least 90%, at least 100%, at least 200%, at least 300%, at least 400%, or at least 500% compared to prior to contacting with the polypeptide, polynucleotide, vector, or lentivirus.

In some embodiments of any of the aspects, the increased activation of the NK cell or population thereof comprises increased expression of a cytokine or granzyme selected from the group consisting of TNFα, IFNγ, GM-CSF, and Granzyme B.

In some embodiments of any of the aspects, the increased activation of the NK cell or population thereof results in an increased specific lysis of a target cell.

In some embodiments of any of the aspects, the target cell expresses a surface antigen that binds specifically to the extracellular binding domain of the polypeptide.

In some embodiments of any of the aspects, the target cell is a cancer cell.

In some embodiments of any of the aspects, the target cell is a cell infected by a virus or bacteria.

In one aspect described herein is a method of treating a subject in need of a CAR-based therapy comprising administering to the subject a therapeutically effective amount of the CAR-based therapy selected from the group consisting of: a polypeptide as described herein, a polynucleotide as described herein, a vector as described herein, a lentivirus as described herein, a cell or population thereof as described herein, and a pharmaceutical composition as described herein.

In some embodiments of any of the aspects, the subject has cancer or has been diagnosed with cancer.

In some embodiments of any of the aspects, the subject has or has been diagnosed with adrenal cancer, anal cancer, appendix cancer, bile duct cancer, bladder cancer, bone cancer, brain cancer, breast cancer, cervical cancer, colorectal cancer, gallbladder cancer, gestational trophoblastic disease, head and neck cancer, Hodgkin lymphoma, intestinal cancer, kidney cancer, leukemia, liver cancer, lung cancer, melanoma, Merkel cell carcinoma, mesothelioma, multiple myeloma, neuroendocrine tumors, Non-Hodgkin lymphoma, oral cancer, ovarian cancer, pancreatic cancer, prostate cancer, sinus cancer, skin cancer, a sarcoma, a soft tissue sarcoma, spinal cancer, stomach cancer, testicular cancer, throat cancer, a tumor, thyroid cancer, uterine cancer, vaginal cancer or vulvar cancer.

In some embodiments of any of the aspects, the administration of the CAR-based therapy results in increased specific lysis of cancer cells targeted by the CAR.

In some embodiments of any of the aspects, the subject has an infectious disease or has been diagnosed with an infectious disease.

In some embodiments of any of the aspects, the infectious disease is a viral or bacterial disease.

In some embodiments of any of the aspects, the administration of the CAR-based therapy results in increased specific lysis of infected cells targeted by the CAR.

In one aspect described herein is a method of making a therapeutic composition, the method comprising introducing a nucleic acid encoding a polypeptide as described herein, a polynucleotide as described herein, a vector as described herein, or a lentivirus as described herein to an NK cell under conditions permitting expression of the polypeptide in the cell.

In some embodiments of any of the aspects, prior to introducing the nucleic acid, polynucleotide, vector, or lentivirus, the NK cell is removed from a subject in need of the therapeutic composition.

In some embodiments of any of the aspects, after introducing the nucleic acid, polynucleotide, vector, or lentivirus, the NK cell is returned to the subject.

### BRIEF DESCRIPTION OF THE DRAWINGS

FIG. 1 is a line graph showing cytotoxicity of CD19 NK CARs against a CD19 expressing A549 cell line.

FIG. 2 is a bar graph showing cytotoxicity of CD19 NK CARs against Raji cell line (Calcein-AM assay).

FIG. 3 is a bar graph showing cytotoxicity of CD19 NK CARs against Raji cell line (Calcein-AM assay).

FIG. 4A is a line graph showing cytotoxicity of CD19 NK CARs against Nalm6-GFP-luciferase cell line (Bioluminescence assay). FIG. 4B is a line graph showing cytotoxicity of NK CARs against Nalm6-GFP-luciferase cell line (Bioluminescence assay).

FIG. 5 is a bar graph showing cytotoxicity of CD19 NK CARs against Raji cell line (Calcein-AM assay).

FIG. 6A is a line graph showing cytotoxicity of CD19 NK CARs against Nalm6-GFP-luciferase cell line (Bioluminescence assay). FIG. 6B is a line graph showing cytotoxicity of NK CARs against Nalm6-GFP-luciferase cell line (Bioluminescence assay).

FIG. 7 is a bar graph showing cytotoxicity of CD19 NK CARs against Raji cell line (Calcein-AM assay).

FIG. 8 is a line graph showing cytotoxicity of CD19 NK CARs against Nalm6-GFP-luciferase cell line (Bioluminescence assay).

FIG. 9 is a bar graph showing cytotoxicity of CD19 NK CARs against Raji cell line (Calcein-AM assay).

FIG. 10 is a line graph showing cytotoxicity of CD19 NK CARs against Nalm6-GFP-luciferase cell line (Bioluminescence assay).

FIG. 11 is a bar graph showing release of cytokine TNF-alpha by different CD19 NK CAR cells.

FIG. 12 is a bar graph showing release of cytokine IFN-gamma by different CD19 NK CAR cells.

FIG. 13 is a bar graph showing release of cytokine GM-CSF by different CD19 NK CAR cells.

FIG. 14 is a bar graph showing release of Granzyme B by different CD19 NK CAR cells.

FIG. 15 is a schematic of Group I and Group II CD19 NK CAR constructs.

FIG. 16 is a schematic of Group III and Group IV CD19 NK CAR constructs.

FIG. 17 is a schematic of NK CAR constructs.

FIG. 18 is a schematic of CD33 NK CAR constructs.

FIG. 19 is a bar graph showing cytotoxicity of CD33 NK CARs against a U937 cell line.

FIG. 20A-20E is a series of 24 h FACS plots of CD33 NK CAR cells at an effector to target ratio of 1:1.

FIG. 21A-21D is a series of 24 h FACS plots of CD33 NK CAR cells at an effector to target ratio of 1:4.

FIG. 22A-22E is a series of 48 h FACS plots of CD33 NK CAR cells at an effector to target ratio of 1:1.

FIG. 23A-23D is a series of 48 h FACS plots of CD33 NK CAR cells at an effector to target ratio of 1:4.

FIG. 24A is a bar graph summarizing 24 h and 48 h FACS data of CD33 NK CAR cells at an effector to target ratio of 1:1. FIG. 24B is a bar graph summarizing 24 h and 48 h FACS data of CD33 NK CAR cells at an effector to target ratio of 1:4.

### DETAILED DESCRIPTION

This technology relates to Chimeric Antigen Receptor (CAR) constructs that can be expressed in Natural Killer (NK) cells, and their use to engineer or modify NK cells to target cancer cells expressing a given cell surface marker or tumor antigen, or cells infected with a pathogen. Some CAR constructs designed, for example, to target T cells to cells expressing a given marker or tumor antigen can function to target NK cells to the same target cells, but the efficiency of such activity is poor given the differences in intracellular signaling in NK cells versus T cells. As such, CAR constructs designed for function in NK cells can provide superior results. Thus, described herein are CAR constructs that can be used for engineering NK cells to target and kill cells expressing a given cell surface marker or tumor antigen. The following describes the compositions, methods and considerations involved in engineering, preparing and using CAR-NK cells for the treatment of cancer and infectious disease.

Accordingly, the technology described herein is directed to Natural Killer (NK) cell CAR polypeptides comprising intracellular signaling domains, intracellular costimulatory domains, and/or transmembrane domains from NK-associated polypeptides. In various aspects, described herein are polynucleotides, vectors, or cells expressing such NK CAR polypeptides, and pharmaceutical compositions comprising such NK CAR polypeptides, polynucleotides, vectors, or cells. Also described herein are methods of using such NK CAR polypeptides, for example to treat various diseases and disorders, such as cancer or infectious diseases.

### Polypeptides

In various aspects, described herein are NK CAR polypeptides that comprise at least one of the following domains: an extracellular binding domain, a transmembrane domain, an intracellular signaling domain, an intracellular co-stimulatory domain, a detectable marker, a self-cleaving peptide, and/or a cytokine, or any combination thereof. Specific CARs described herein are not to be construed as limitations, but rather exemplifications applying the principles and technology described. In various aspects, described herein are NK CAR polypeptides that comprise an extracellular binding domain, a transmembrane domain, and at least one intracellular signaling domain. For example, the following combinations are contemplated herein (see e.g., Table 7):

TABLE 7
Exemplary Combinations of Domains in an NK-CAR Polypeptide.

| ED | TM | IS | IC | DM | SP | Cy | ED | TM | IS | IC | DM | SP | Cy |
|---|---|---|---|---|---|---|---|---|---|---|---|---|---|
| | | | | | | | | | X | | | X | |
| X | | | | | | | X | | X | | | X | |
| | X | | | | | | | X | X | | | X | |
| X | X | | | | | | X | X | X | | | X | |
| | | X | | | | | | | | X | | X | |
| X | | X | | | | | X | | | X | | X | |
| | X | X | | | | | | X | | X | | X | |
| X | X | X | | | | | X | X | | | | X | |
| | | | X | | | | | | X | X | | X | |
| X | | | X | | | | X | | X | X | | X | |
| | X | | X | | | | | X | X | X | | X | |
| X | X | | X | | | | X | X | X | X | | X | |
| | | X | X | | | | | | | | X | X | |
| X | | X | X | | | | X | | | | X | X | |
| | X | X | | | | | | X | | | X | X | |
| X | X | X | X | | | | X | X | | | X | X | |
| | | | | X | | | | | X | | X | X | |
| X | | | | X | | | X | | X | | X | X | |
| | X | | | X | | | | X | | | X | X | |
| X | X | | | X | | | X | X | X | | X | X | |
| | | X | | X | | | | | | X | X | X | |
| X | | X | | X | | | X | | | X | X | X | |
| | X | X | | X | | | | X | | X | X | X | |
| X | X | X | | X | | | X | X | | X | X | X | |
| | | | X | X | | | | | X | X | X | X | |
| | | | X | X | | | X | | X | X | X | X | |
| | X | | X | X | | | | X | X | X | X | X | |
| X | X | | X | X | | | X | X | X | X | X | X | |
| | | X | X | X | | | | | | | | | X |
| X | | X | X | X | | | X | | | | | | X |
| | X | X | X | X | | | | X | | | | | X |
| X | X | X | X | X | | | X | X | | | | | X |
| | | | | | X | | | | X | | | | X |
| X | | | | | X | | X | | X | | | | X |
| | X | | | | X | | | X | X | | | | X |
| X | X | | | | X | | X | X | X | | | | X |
| | | | X | | | X | | | X | | | X | X |
| X | | | X | | | X | X | | X | | | X | X |
| | X | | X | | | X | | X | X | | | X | X |
| X | X | | X | | | X | X | X | X | | | X | X |
| | | X | X | | | X | | | | X | | X | X |
| X | | X | X | | | X | X | | | X | | X | X |
| | X | X | X | | | X | | X | | X | | X | X |
| X | X | X | X | | | X | X | X | | X | | X | X |
| | | | | X | | X | | | X | X | | X | X |
| X | | | | X | | X | X | | X | X | | X | X |
| | X | | | X | | X | | X | X | X | | X | X |
| X | X | | | X | | X | X | X | X | X | | X | X |
| | | X | | X | | X | | | | | X | X | X |
| X | | X | | X | | X | X | | | | X | X | X |
| | X | X | | X | | X | | X | | | X | X | X |
| X | X | X | | X | | X | X | X | | | X | X | X |
| | | | X | X | | X | | | X | | X | X | X |
| X | | | X | X | | X | X | | X | | X | X | X |
| | X | | X | X | | X | | X | X | | X | X | X |
| X | X | | X | X | | X | X | X | X | | X | X | X |
| | | X | X | X | | X | | | | X | X | X | X |
| X | | X | X | X | | X | X | | | X | X | X | X |
| | X | X | X | X | | X | | X | | X | X | X | X |
| X | X | X | X | X | | X | X | X | | X | X | X | X |
| | | | | | X | X | | | X | X | X | X | X |
| X | | | | | X | X | X | | X | X | X | X | X |
| | X | | | | X | X | | X | X | X | X | X | X |
| X | X | | | | X | X | X | X | X | X | X | X | X |

“ED” indicates extracellular domain (which can itself comprise an extracellular binding domain, a signal peptide, a linker domain, a detectable marker, and/or a spacer domain).
“TM indicates transmembrane domain.
“IS” indicates intracellular signaling domain.
“IC” indicates intracellular co-stimulatory domain.
“DM” indicates detectable marker.
“SP” indicates self-cleaving peptide.
“Cy” indicates cytokine.

In some embodiments of any of the aspects, an NK-CAR polypeptide can comprise an extracellular binding domain, a signal peptide, a linker domain, a spacer domain, a transmembrane domain, an intracellular signaling domain, an intracellular co-stimulatory domain, a detectable marker, a self-cleaving peptide, and/or a cytokine, as described further herein. For discussions of various polypeptide domains described herein, see e.g., U.S. patent Ser. No. 10/640,570 B2; Li et al., 2018, Cell Stem Cell 23: 1-12; Bryceson et al., 2006; Blood 107: 159-166; Claus et al., 2008, Frontiers in Bioscience 13: 956-965; the contents of each of which are incorporated herein by reference in their entireties. Exemplary NK CAR polypeptides are described further herein.

### Intracellular Domains

In various aspects, described herein are NK CAR polypeptides comprising at least one intracellular domain. As described further herein, such intracellular domains can include, but are not limited to: intracellular signaling domains, intracellular co-stimulatory domains, self-cleaving peptides, cytokines, and detectable markers. An NK CAR polypeptide can comprise any combination of intracellular signaling domains, intracellular co-stimulatory domains, self-cleaving peptides, cytokines, and detectable markers, as exemplified in Table 7.

In some embodiments of any of the aspects, the CAR polypeptide comprises 1, 2, 3, 4, 5, or more intracellular domains, e.g., selected from at least one intracellular signaling domain, at least one intracellular co-stimulatory domain, at least one self-cleaving peptide, at least one cytokine, and/or at least one detectable marker. In some embodiments of any of the aspects, the CAR polypeptide comprises one intracellular domain. In some embodiments of any of the aspects, the CAR polypeptide comprises two intracellular domains. In some embodiments of any of the aspects, the CAR polypeptide comprises three intracellular domains. In some embodiments of any of the aspects, the CAR polypeptide comprises four intracellular domains. In some embodiments of any of the aspects, the CAR polypeptide comprises five intracellular domains. In some embodiments of any of the aspects, the CAR polypeptide comprises six intracellular domains. In embodiments comprising multiple intracellular domains, the multiple intracellular domains can be different individual intracellular domains or multiple copies of the same intracellular domain, or a combination of the foregoing.

Non-limiting examples of intracellular domains for use in the NK CAR polypeptides described herein include: (a) an intracellular signaling domain from an NK cell receptor; (b) an intracellular signaling domain from an NK cell membrane-bound signaling adaptor protein; and/or (c) an intracellular co-stimulatory domain from a co-stimulatory receptor.

In some embodiments of any of the aspects, the CAR polypeptide comprises: (a) an intracellular signaling domain from an NK cell receptor; (b) an intracellular signaling domain from an NK cell membrane-bound signaling adaptor protein; and (c) an intracellular co-stimulatory domain from a co-stimulatory receptor. In some embodiments of any of the aspects, the CAR polypeptide comprises: (a) an intracellular signaling domain from an NK cell receptor. In some embodiments of any of the aspects, the CAR polypeptide comprises: (b) an intracellular signaling domain from an NK cell membrane-bound signaling adaptor protein. In some embodiments of any of the aspects, the CAR polypeptide comprises: (c) an intracellular co-stimulatory domain from a co-stimulatory receptor.

In some embodiments of any of the aspects, the CAR polypeptide comprises: (a) an intracellular signaling domain from an NK cell receptor; and (b) an intracellular signaling domain from an NK cell membrane-bound signaling adaptor protein. In some embodiments of any of the aspects, the CAR polypeptide comprises: (a) an intracellular signaling domain from an NK cell receptor; and (c) an intracellular co-stimulatory domain from a co-stimulatory receptor. In some embodiments of any of the aspects, the CAR polypeptide comprises: (b) an intracellular signaling domain from an NK cell membrane-bound signaling adaptor protein; and (c) an intracellular co-stimulatory domain from a co-stimulatory receptor.

Intracellular Signaling Domains

The intracellular signaling domain(s) of the NK CAR polypeptides described herein are responsible for activation of at least one of the normal effector functions of the immune cell in which the CAR has been placed. The term “effector function” refers to a specialized function of a cell. Effector function of a natural killer (NK) cell, for example, can be cytolytic activity. Thus the term “intracellular signaling domain” refers to the portion of a polypeptide which transduces the effector function signal (e.g., from the extracellular biding domain specifically binding its cognate antigen) and directs the cell to perform a specialized function. While usually the entire intracellular signaling domain can be employed, in many cases it is not necessary to use the entire chain. To the extent that a truncated portion of the intracellular signaling domain is used, such truncated portion can be used in place of the intact chain as long as it transduces the effector function signal. The term “intracellular signaling domain” is thus meant to include any truncated portion of the intracellular signaling domain sufficient to transduce the effector function signal.

In some embodiments of any of the aspects, the CAR polypeptide comprises 1, 2, 3, 4, 5, or more intracellular signaling domains. In some embodiments of any of the aspects, the CAR polypeptide comprises three intracellular signaling domains. In embodiments comprising multiple intracellular signaling domains, the multiple intracellular signaling domains can be different individual intracellular signaling domains or multiple copies of the same intracellular signaling domain, or a combination of the foregoing.

In some embodiments of any of the aspects, the CAR polypeptide comprises an intracellular signaling domain from an NK cell receptor and/or an intracellular signaling domain from an NK cell membrane-bound signaling adaptor protein. In some embodiments of any of the aspects, the NK cell receptor or NK cell membrane-bound signaling adaptor protein (e.g., from which the at least one intracellular signaling domain is derived) is selected from the group consisting of. Natural Killer Cell Receptor 2B4; Natural Killer-, T- And B-Cell Antigen (NTB-A); CD2-Like Receptor Activating Cytotoxic Cells (CRACC); cluster of differentiation 2 (CD2); high-affinity IgE receptor (FcεR1); and CD3-zeta (CD3ζ). In some embodiments of any of the aspects, the intracellular signaling domain from the NK cell receptor or from the NK cell membrane-bound signaling adaptor protein comprises an immunoreceptor tyrosine-based activation motif (ITAM), a conserved sequence of four amino acids repeated twice, which can initiate a variety of signaling pathways and subsequently NK cell activation. In some embodiments of any of the aspects, the intracellular signaling domain from the NK cell receptor or from the NK cell membrane-bound signaling adaptor protein comprises an immunoreceptor tyrosine-based switch motif (ITSMs), which can lead to activation or inhibition depending on the type of adaptor protein bound.

In some embodiments of any of the aspects, the intracellular signaling domain of a CAR polypeptide as described herein comprises one of SEQ ID NOs: 7-12, or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of SEQ ID NOs: 7-12, that maintains the same functions as one of SEQ ID NOs: 7-12 (e.g., intracellular signaling upon activation of the CAR polypeptide by binding its cognate antigen). In some embodiments of any of the aspects, the intracellular signaling domain of a CAR polypeptide as described herein comprises one of SEQ ID NOs: 7-12, or an amino acid sequence that is at least 95% identical to the sequence of one of SEQ ID NOs: 7-12 that maintains the same function.

In some embodiments of any of the aspects, the intracellular signaling domain of a CAR polypeptide as described herein is encoded by a nucleic acid sequence comprising one of SEQ ID NOs: 1-6 or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 1-6 that maintains the same function or a codon-optimized version of one of SEQ ID NOs: 1-6. In some embodiments of any of the aspects, the intracellular signaling domain of a CAR polypeptide as described herein is encoded by a nucleic acid sequence comprising one of SEQ ID NOs: 1-6 or a sequence that is at least 95% identical to one of SEQ ID NOs: 1-6 that maintains the same function.

The cytoplasmic signaling sequences within the cytoplasmic signaling portion of the NK CAR polypeptides described herein may be linked to each other in a random or specified order. Optionally, a short oligo- or polypeptide linker, preferably between 2 and 10 amino acids in length may form the linkage. A glycine-serine doublet provides a particularly suitable linker.

NK Cell Receptors

Chimeric Antigen Receptors for modification of NK cells can include variants and/or domains of naturally occurring NK cell receptors. As described herein, the NK CAR polypeptide can comprise an intracellular signaling domain from an NK cell receptor. As used herein, the term “NK cell receptor” refers to a polypeptide, expressed by an NK cell, comprising an extracellular domain that interacts with ligands outside of the cell; an NK cell receptor further comprises a transmembrane domain and at least one intracellular signaling domain, examples of which are described further herein. The NK cell receptor can be an activating receptor, an inhibitory receptor, a chemotactic receptor, an adhesion receptor, or a cytokine receptor, depending on the type of ligand and the type of intracellular signal transduced by the intracellular signaling domain upon binding of the ligand. In some embodiments of any of the aspects, the NK CAR polypeptide described herein comprises an NK activating cell receptor. Non-limiting examples of NK activating cell receptors or domain(s) thereof (e.g., an intracellular signaling domain) that can be adapted or modified for use in a NK CAR polypeptide as described herein include: NKp46, CD16, hNKp30, hNKp44, hNKp80, mNKR-P1C, NKG2D, mNKG2D-S, hKIR-S, mAct Ly49, CD94/NKG2C, CRACC, Ly9, CD84, NTBA, and 2B4; see e.g., Vivier et al. Science 331(6013):44-9 (2011), the content of which is incorporated herein by reference in its entirety. Non-limiting examples of NK activating receptors and their corresponding adaptors are provided in Table 10. In some embodiments, an NK CAR polypeptide comprises an intracellular signaling domain of an activating receptor or adaptor protein listed in Table 10.

TABLE 10
Activating Receptors and Their Corresponding Adaptor Proteins

| Activating Receptor | Adaptor(s) |
|---|---|
| NKp46 | CD3ζ, FcRγ |
| CD16 | CD3ζ, FcRγ |
| h NKp30 | CD3ζ, FcRγ |
| h NKp44 | DAP12 |
| h NKp80 | — |
| m NKR-P1C | FcRγ |
| NKG2D | DAP10 |
| m NKG2D-S | DAP12 |
| h KIR-S | DAP12 |
| m Act. Ly49 | DAP12, DAP10 |
| CD94/NKG2C | DAP12 |
| CRACC | SAP, EAT2 |
| Ly9 | SAP |
| CD84 | SAP, EAT2 |
| NTBA | SAP |
| 2B4 | SAP, EAT2, ERT |

In some embodiments of any of the aspects, the NK cell receptor or domain(s) thereof (e.g., an intracellular signaling domain) that can be adapted or modified for use in a NK CAR polypeptide as described herein is a member of the signaling lymphocyte activation molecule (SLAM) family of immune cell receptors. The SLAM family is closely related to the CD2 family of the immunoglobulin (Ig) superfamily of molecules. Non-limiting examples of SLAM family members include SLAM, CD48, CD229, 2B4, CD84, NTB-A, CRACC, BLAME, and CD2F-10. In some embodiments of any of the aspects, the NK cell receptor is a member of the CD2 family. In some embodiments of any of the aspects, the NK cell receptor is selected from the group consisting of: 2B4, NTB-A, CRACC, and CD2.

1. 2B4

In some embodiments of any of the aspects, the NK cell receptor or domain(s) thereof (e.g., an intracellular signaling domain) that can be adapted or modified for use in a NK CAR polypeptide as described herein is Natural Killer Cell Receptor 2B4. The NK Cell Type I Receptor Protein 2B4 is also referred to interchangeably as CD244, Natural Killer Cell Receptor 2B4 (NKR2B4), Signaling Lymphocytic Activation Molecule Family Member 4 (SLAMF4), NK Cell Activation-Inducing Ligand (NAIL), or Nmrk. 2B4 is a cell surface receptor expressed on natural killer (NK) cells (and some T cells) that mediates non-major histocompatibility complex (MHC) restricted killing. The natural ligand of 2B4 is CD48, which is expressed on hematopoietic cells. The interaction between NK-cell and target cells via the 2B4 receptor modulates NK-cell cytolytic activity. Alternatively spliced transcript variants encoding different isoforms have been described for the 2B4 gene. The intracellular signaling domain of 2B4 stimulates NK cell cytotoxicity, production of IFN-gamma, and granule exocytosis. SLAM family receptors (such as 2B4, CRACC, or NTB-A), can comprise immunoreceptor tyrosine-based switch motifs (ITSMs) with the consensus sequence T-X-Y-X-X-[VI], which have overlapping specificity for activating and inhibitory SH2 domain-containing binding partners. The ITSMs can mediate the interaction with the SH2 domain of the adaptor proteins SH2D1A (SAP) and SH2D1B (EAT-2).

In some embodiments of any of the aspects, 2B4 comprises SEQ ID NO: 123 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 123, that maintains the same function as SEQ ID NO: 123 (e.g., intracellular signaling in response to extracellular ligand binding; ITSM functionality). In some embodiments of any of the aspects, the intracellular signaling domain of an NK-CAR as described herein comprises an intracellular portion of 2B4 (e.g., SEQ ID NO: 123). In some embodiments of any of the aspects, the intracellular signaling domain comprises SEQ ID NO: 7 (i.e., residues 251-370 of SEQ ID NO: 123) or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 7 that maintains the same function as SEQ ID NO: 7 (e.g., intracellular signaling in response to extracellular ligand binding; ITSM functionality).

The ITSMs of 2B4 include residues 269-274 (ITSM 1), 295-300, (ITSM 2), 315-320 (ITSM 3), and 340-345 (ITSM 4) of SEQ ID NO: 123, with tyrosine phosphorylation sites at residues 271, 297, 317, and 342 of SEQ ID NO: 123. Accordingly, in some embodiments of any of the aspects, an intracellular signaling domain, which comprises a sequence substantially similar (e.g., at least 70% identical) to the intracellular signaling domain of 2B4 (e.g., residues 251-370 of SEQ ID NO: 123; SEQ ID NO: 7), does not comprise mutations in residues 269-274, 295-300, 315-320, or 340-345 of SEQ ID NO: 123, does not comprise mutations in residues 271, 297, 317, or 342 of SEQ ID NO: 123, or comprises tyrosines at residues 271, 297, 317, or 342 of SEQ ID NO: 123.

natural killer cell receptor 2B4 isoform 2 precursor [Homo sapiens], NCBI
Reference Sequence: NP_001160135.1, 370 amino acids (aa); bold text
indicates ITSMs and double-underlined text indicates phosphorylation sites.
SEQ ID NO: 123
MLGQVVTLILLLLLKVYQGKGCQGSADHVVSISGVPLQLQPNSIQTKVDSIAWKKLLPSQNGFH
HILKWENGSLPSNTSNDRFSFIVKNLSLLIKAAQQQDSGLYCLEVTSISGKVQTATFQVFVFESLLP
DKVEKPRLQGQGKILDRGRCQVALSCLVSRDGNVSYAWYRGSKLIQTAGNLTYLDEEVDINGT
HTYTCNVSNPVSWESHTLNLTQDCQNAHQEFRFWPFLVIIVILSALFLGTLACFCVWRRKRKEKQ
SETSPKEFLTI [symbol] EDVKDLKTRRNHEQEQTFPGGGSTI [symbol] SMIQSQSSAPTSQEPAYTL [symbol] SLIQPSRK
SGSRKRNHSPSFNSTI [symbol] EVIGKSQPKAQNPARLSRKELENFDVYS

SEQ ID NOs: 1 (polynucleotide) and 7 (amino acid) provide an exemplary 2B4 intracellular signaling domain (e.g., residues 251-370 of 2B4; see e.g., SEQ ID NO: 123 for an exemplary full-length 2B4 polypeptide sequence).

SEQ ID NO: 1
TGGAGGAGGAAAAGGAAGGAGAAGCAGTCCGAGACATCCCCCAAAGAGT
TTTTAACCATTTATGAGGACGTGAAGGACCTCAAGACCAGAAGGAACCA
CGAGCAAGAACAGACCTTTCCCGGCGGCGGATCCACCATCTACAGCATG
ATCCAGAGCCAGAGCTCCGCCCCTACAAGCCAAGAACCCGCTTACACTT
TATATTCTTTAATCCAGCCTTCTCGTAAGAGCGGCTCCCGTAAGAGGAA
CCACAGCCCCAGCTTTAATAGCACCATTTACGAGGTCATCGGCAAGTCC
CAGCCTAAGGCCCAAAACCCCGCTCGTCTCTCTCGTAAGGAGCTGGAGA
ATTTCGACGTGTACAGC
SEQ ID NO: 7
WRRKRKEKQSETSPKEFLTIYEDVKDLKTRRNHEQEQTFPGGGSTIYSM
IQSQSSAPTSQEPAYTLYSLIQPSRKSGSRKRNHSPSFNSTIYEVIGKS
QPKAQNPARLSRKELENFDVYS

2. CRACC

In some embodiments of any of the aspects, the NK cell receptor or domain(s) thereof (e.g., an intracellular signaling domain) that can be adapted or modified for use in a NK CAR polypeptide as described herein is CD2-Like Receptor Activating Cytotoxic Cells (CRACC). CRACC is also referred to interchangeably as SLAM family member 7 (SLAMF7), CD319, CS1, Membrane Protein FOAP-12, CD2 Subset 1, Protein 19A, Novel LY9 (Lymphocyte Antigen 9) Like Protein, or 19A24 Protein. CRACC is a cell surface receptor of the CD2 family that triggers NK cell-mediated cytotoxicity. CRACC can associate with the small cytoplasmic adapter proteins, SH2D1A/SAP and/or SH2D1B/EAT-2. CRACC comprises cytoplasmic tyrosine-based motifs, immunoreceptor tyrosine-based switch motifs (ITSMs), which resemble those found in the NK cell receptor 2B4.

In some embodiments of any of the aspects, CRACC comprises SEQ ID NO: 124 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 124, that maintains the same function as SEQ ID NO: 124 (e.g., intracellular signaling in response to extracellular ligand binding; ITSM functionality). In some embodiments of any of the aspects, the intracellular signaling domain of an NK-CAR as described herein comprises an intracellular portion of CRACC (e.g., SEQ ID NO: 124). In some embodiments of any of the aspects, the intracellular signaling domain comprises SEQ ID NO: 8 (i.e., residues 248-335 of SEQ ID NO: 124) or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 8, that maintains the same function as SEQ ID NO: 8 (e.g., intracellular signaling in response to extracellular ligand binding; ITSM functionality).

CRACC comprises an ITSM-like motif (e.g., residues 282-287 of SEQ ID NO: 124) and an ITSM (e.g., residues 302-307 of SEQ ID NO: 124), with tyrosine phosphorylation sites at residues 284 and 304 of SEQ ID NO: 124. Accordingly, in some embodiments of any of the aspects, an intracellular signaling domain, which comprises a sequence substantially similar (e.g., at least 70% identical) to the intracellular signaling domain of CRACC (e.g., residues 248-335 of SEQ ID NO: 124; SEQ ID NO: 8), does not comprise mutations in residues 282-287 or 302-307 of SEQ ID NO: 124, does not comprise mutations in residues 284 or 304 of SEQ ID NO: 124, or comprises tyrosines at residues 284 or 304 of SEQ ID NO: 124.

CRACC (SLAM family member 7) isoform a precursor [Homo sapiens], NCBI
Reference Sequence: NP_067004.3, 335 aa; bold text indicates the ITSM-like
motif and ITSM, respectively, and double-underlined text indicates
phosphorylation sites.
SEQ ID NO: 124
MAGSPTCLTLIYILWQLTGSAASGPVKELVGSVGGAVTFPLKSKVKQVDSIVWTFNTTPLVTIQP
EGGTIIVTQNRNRERVDFPDGGYSLKLSKLKKNDSGIYYVGIYSSSLQQPSTQEYVLHVYEHLSKP
KVTMGLQSNKNGTCVTNLTCCMEHGEEDVIYTWKALGQAANESHNGSILPISWRWGESDMTFI
CVARNPVSRNFSSPILARKLCEGAADDPDSSMVLLCLLLVPLLLSLFVLGLFLWFLKRERQEEYIE
EKKRVDICRETPNICPHSGENTE [symbol] DTIPHTNRTILKEDPANTV [symbol] STVEIPKKMENPHSLLTMPDTP
RLFAYENVI

SEQ ID NOs: 2 (polynucleotide) and 8 (amino acid) provide an exemplary CRACC intracellular signaling domain (e.g., residues 248-335 of CRACC; see e.g., SEQ ID NO: 124 for an exemplary full-length 2B4 polypeptide sequence).

SEQ ID NO: 2
TGGTTTCTGAAGAGAGAGAGGCAAGAGGAGTACATCGAGGAGAAGAAGA
GGGTGGACATCTGCAGAGAGACCCCCAACATCTGCCCCCATTCCGGCGA
GAACACCGAGTACGACACCATCCCCCACACCAACAGAACCATTCTGAAG
GAGGACCCCGCCAACACCGTGTACAGCACCGTGGAGATCCCCAAGAAGA
TGGAGAATCCCCACTCTCTGCTGACCATGCCCGACACCCCCAGACTGTT
CGCCTATGAGAATGTTATC
SEQ ID NO: 8
WFLKRERQEEYIEEKKRVDICRETPNICPHSGENTEYDTIPHTNRTILK
EDPANTVYSTVEIPKKMENPHSLLTMPDTPRLFAYENVI

3. NTB-A

In some embodiments of any of the aspects, the NK cell receptor or domain(s) thereof (e.g., an intracellular signaling domain) that can be adapted or modified for use in a NK CAR polypeptide as described herein is Natural Killer-, T- And B-Cell Antigen (NTB-A). NTB-A is also referred to interchangeably as NK-T-B-Ag, SLAM Family Member 6 (SLAMF6), CD352, Ly108, NTBA, KALI, KALIb, or SF2000. NTB-A is expressed on NK cells, as well as T and B lymphocytes. NTB-A undergoes tyrosine phosphorylation and associates with the Src homology 2 domain-containing protein (SH2D1A) as well as with SH2 domain-containing phosphatases (SHPs). NTB-A can function as a coreceptor in the process of NK cell activation.

In some embodiments of any of the aspects, NTB-A comprises SEQ ID NO: 126 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 126, that maintains the same function as SEQ ID NO: 126 (e.g., intracellular signaling in response to extracellular ligand binding; ITSM functionality). In some embodiments of any of the aspects, the intracellular signaling domain of an NK-CAR as described herein comprises an intracellular portion of NTB-A (e.g., SEQ ID NO: 126). In some embodiments of any of the aspects, the intracellular signaling domain comprises SEQ ID NO: 10 (i.e., residues 248-331 of SEQ ID NO: 126) or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 10, that maintains the same function as SEQ ID NO: 10 (e.g., intracellular signaling in response to extracellular ligand binding; ITSM functionality).

NTB-A comprises two ITSMs at residues 282-287 and 306-311 of SEQ ID NO: 126, with tyrosine phosphorylation sites at residues 284 and 308 of SEQ ID NO: 126. Accordingly, in some embodiments of any of the aspects, an intracellular signaling domain, which comprises a sequence substantially similar (e.g., at least 70% identical) to the intracellular signaling domain of NTB-A (e.g., residues 248-331 of SEQ ID NO: 126; SEQ ID NO: 10), does not comprise mutations in residues 282-287 or 306-311 of SEQ ID NO: 126, or does not comprise mutations in residues 284 or 308 of SEQ ID NO: 126, or comprises tyrosines at residues 284 or 308 of SEQ ID NO: 126.

NTB-A (SLAM family member 6) isoform 2 precursor [Homo sapiens], NCBI
Reference Sequence: NP_443163.1, 331 aa; bold text indicates the ITSMs,
and double-underlined text indicates phosphorylation sites.
SEQ ID NO: 126
MLWLFQSLLFVFCFGPGNVVSQSSLTPLMVNGILGESVTLPLEFPAGEKVNFITWLFNETSLAFIV
PHETKSPEIHVTNPKQGKRLNFTQSYSLQLSNLKMEDTGSYRAQISTKTSAKLSSYTLRILRQLRN
IQVTNHSQLFQNMTCELHLTCSVEDADDNVSFRWEALGNTLSSQPNLTVSWDPRISSEQDYTCIA
ENAVSNLSFSVSAQKLCEDVKIQYTDTKMILFMVSGICIVFGFIILLLLVLRKRRDSLSLSTQRTQG
PESARNLEYVSVSPTNNTV [symbol] ASVTHSNRETEIWTPRENDTITI [symbol] STINHSKESKPTFSRATALDNV

SEQ ID NOs: 4 (polynucleotide) and 10 (amino acid) provide an exemplary NTB-A intracellular signaling domain (e.g., residues 248-331 of NTB-A; see e.g., SEQ ID NO: 126 for an exemplary full-length NTB-A polypeptide sequence)

SEQ ID NO: 4
CTGAGAAAGAGAAGAGACTCTCTGTCTCTGAGCACCCAAAGAACACAAG
GCCCCGAGAGCGCCAGAAATCTGGAGTACGTGAGCGTGAGCCCCACCAA
CAACACCGTGTACGCCAGCGTGACCCACAGCAACAGAGAGACCGAGATC
TGGACCCCTAGAGAGAACGACACCATCACCATCTACAGCACCATCAACC
ACAGCAAGGAGAGCAAGCCCACATTCTCTAGAGCCACCGCTCTGGATAA
CGTGGTG
SEQ ID NO: 10
LRKRRDSLSLSTQRTQGPESARNLEYVSVSPTNNTVYASVTHSNRETEI
WTPRENDTITIYSTINHSKESKPTFSRATALDNVV

4. CD2

In some embodiments of any of the aspects, the NK cell receptor or domain(s) thereof (e.g., an intracellular signaling domain) that can be adapted or modified for use in a NK CAR polypeptide as described herein is cluster of differentiation 2 (CD2). CD2 is also referred to interchangeably as Lymphocyte-Function Antigen-2 (LFA-2), P50, Sheep Red Blood Cell Receptor (SRBC), T11, Leu-5, Erythrocyte Receptor, Rosette Receptor, or LFA-3 Receptor. CD2 is expressed by thymocytes, peripheral T cells, NK cells, and a subset of thymic B cells. CD2 interacts with LFA3 (CD58) on antigen presenting cells. CD2 itself has no enzymatic activity but transmits signals in part via interactions with CD3zeta. CD2 recruits CD16 to the NK cell immunological synapse in spontaneous (e.g., antibody-independent) NK cell cytotoxicity; see e.g., Christian B et al., CD2 Immunobiology. Front Immunol. 2020; 11: 1090, the content of which is incorporated herein by reference in its entirety.

In some embodiments of any of the aspects, CD2 comprises SEQ ID NO: 125 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 125, that maintains the same function as SEQ ID NO: 125 (e.g., intracellular signaling in response to extracellular ligand binding). In some embodiments of any of the aspects, the intracellular signaling domain of an NK-CAR as described herein comprises an intracellular portion of CD2 (e.g., SEQ ID NO: 125). In some embodiments of any of the aspects, the intracellular signaling domain comprises SEQ ID NO: 9 (i.e., residues 236-251 of SEQ ID NO: 125) or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 9, that maintains the same function as SEQ ID NO: 9 (e.g., intracellular signaling in response to extracellular ligand binding).

T-cell surface antigen CD2 isoform 2 precursor [Homo sapiens], NCBI
Reference Sequence: NP_001758.2, 351 aa
SEQ ID NO: 125
MSFPCKFVASFLLIFNVSSKGAVSKEITNALETWGALGQDINLDIPSFQMSDDIDDIKWEKTSDKK
KIAQFRKEKETFKEKDTYKLFKNGTLKIKHLKTDDQDIYKVSIYDTKGKNVLEKIFDLKIQERVS
KPKISWTCINTTLTCEVMNGTDPELNLYQDGKHLKLSQRVITHKWTTSLSAKFKCTAGNKVSKE
SSVEPVSCPEKGLDIYLIIGICGGGSLLMVFVALLVFYITKRKKQRSRRNDEELETRAHRVATEER
GRKPHQIPASTPQNPATSQHPPPPPGHRSQAPSHRPPPPGHRVQHQPQKRPPAPSGTQVHQQKGPP
LPRPRVQPKPPHGAAENSLSPSSN

SEQ ID NOs: 3 (polynucleotide) and 9 (amino acid) provide an exemplary CD2 intracellular signaling domain (e.g., residues 236-351 of CD2; see e.g., SEQ ID NO: 125 for an exemplary full-length CD2 polypeptide sequence).

SEQ ID NO: 3
AAGAGGAAGAAGCAGAGGTCTAGAAGAAACGACGAGGAGCTCGAGACAA
GAGCCCACAGAGTGGCTACCGAAGAGAGAGGCAGAAAGCCCCACCAGAT
CCCCGCCAGCACACCCCAGAACCCCGCCACAAGCCAGCATCCCCCCCCT
CCTCCCGGCCACAGATCCCAAGCTCCCAGCCACAGACCTCCTCCCCCCG
GCCATAGGGTGCAGCATCAGCCCCAGAAAAGACCTCCCGCCCCCAGCGG
AACCCAAGTGCATCAGCAGAAGGGCCCTCCTCTGCCCAGACCTAGGGTG
CAACCCAAGCCCCCTCATGGCGCTGCCGAGAACTCTCTGAGCCCTAGCA
GCAAC
SEQ ID NO: 9
KRKKQRSRRNDEELETRAHRVATEERGRKPHQIPASTPQNPATSQHPPP
PPGHRSQAPSHRPPPPGHRVQHQPQKRPPAPSGTQVHQQKGPPLPRPRV
QPKPPHGAAENSLSPSSN

Adaptor Proteins

In some embodiments of any of the aspects, the CAR polypeptide comprises an intracellular signaling domain from an NK cell membrane-bound signaling adaptor protein. As used herein, the term “NK cell membrane-bound signaling adaptor protein,” also referred to interchangeably as an “adaptor protein,” refers to a polypeptide, expressed by an NK cell and associated with the cell membrane, that specifically interacts with an NK cell receptor and transduces the receptor's signal through the adaptor's intracellular signaling domain. In some embodiments of any of the aspects, the NK CAR polypeptides described herein comprise an intracellular region of an adaptor protein that associates with an NK activating cell receptor, which often does not comprise its own intracellular signaling domain. Non-limiting examples of NK adaptor proteins or domain(s) thereof (e.g., an intracellular signaling domain) that can be adapted or modified for use in a NK CAR polypeptide as described herein include CD3zeta, FcRγ (which can comprise FcεRIγ), DAP12, DAP10, SAP, EAT2, and ERT. In some embodiments of any of the aspects, the NK cell membrane-bound signaling adaptor protein is FcεRI (e.g., FcεRIγ) or CD3zeta.

1. FcεRI

In some embodiments of any of the aspects, the NK cell membrane-bound signaling adaptor protein or domain(s) thereof (e.g., an intracellular signaling domain) that can be adapted or modified for use in a NK CAR polypeptide as described herein is high-affinity IgE receptor (FcεRI). FcεRI is also referred to as Fc epsilon RI. FcεRI is the high-affinity receptor for the Fc region of immunoglobulin E (IgE), an antibody isotype involved in allergies and parasite immunity. FcεRI is a tetrameric receptor complex, consisting of one alpha (FcεRIα—antibody binding site), one beta (FcεRIβ—which amplifies the downstream signal), and two gamma chains (FcεRIγ—the site where the downstream signal initiates) connected by two disulfide bridges on mast cells and basophils. In some embodiments of any of the aspects, the NK cell adaptor protein is FcεRIγ, which is also referred to interchangeably as Fc-Epsilon RI-Gamma or FCER1G. On human natural killer (NK) cells, FcεRIγ can also associate with FcγRIIIa (CD16a) to form a low affinity receptor for IgG.

In some embodiments of any of the aspects, FcεRIγ comprises SEQ ID NO: 127 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 127, that maintains the same function as SEQ ID NO: 127 (e.g., intracellular signaling; ITAM functionality). In some embodiments of any of the aspects, the intracellular signaling domain of an NK-CAR as described herein comprises an intracellular portion of FcεRIγ (e.g., SEQ ID NO: 127). In some embodiments of any of the aspects, the intracellular signaling domain comprises SEQ ID NO: 12 (i.e., residues 45-86 of SEQ ID NO: 127) or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 12, that maintains the same function as SEQ ID NO: 12 (e.g., intracellular signaling; ITAM functionality).

FcεRIγ comprises an ITAM at residues 62 to 82 of SEQ ID NO: 127, with tyrosine phosphorylation sites at residues 65 and 76 of SEQ ID NO: 127. Accordingly, in some embodiments of any of the aspects, an intracellular signaling domain, which comprises a sequence substantially similar (e.g., at least 70% identical) to the intracellular signaling domain of FcεRIγ (e.g., residues 45-86 of SEQ ID NO: 127; SEQ ID NO: 12), does not comprise mutations in residues 62-82 of SEQ ID NO: 127, does not comprise mutations in residues 65 or 76 of SEQ ID NO: 127, or comprises tyrosines at residues 65 or 76 of SEQ ID NO: 127.

high affinity immunoglobulin epsilon receptor subunit gamma precursor
[Homo sapiens], NCBI Reference Sequence: NP_004097.1, 86 aa; bold text
indicates the ITAM, and double-underlined text indicates phosphorylation sites.
SEQ ID NO: 127
MIPAVVLLLLLLVEQAAALGEPQLCYILDAILFLYGIVLTLLYCRLKIQVRKAAITSYEKSDGV [symbol] T
GLSTRNQET [symbol] ETLKHEKPPQ

SEQ ID NOs: 6 (polynucleotide) and 12 (amino acid) provide an exemplary FceR intracellular signaling domain (e.g., residues 45-86 of FceRI; see e.g., SEQ ID NO: 127 for an exemplary full-length FceRI polypeptide sequence).

SEQ ID NO: 6
CGTCTGAAGATCCAAGTTAGGAAGGCCGCCATCACCTCCTACGAGAAGA
GCGACGGCGTGTACACCGGTTTAAGCACAAGGAACCAAGAAACCTACGA
GACTTTAAAGCACGAGAAGCCCCCCCAG
SEQ ID NO: 12:
RLKIQVRKAAITSYEKSDGVYTGLSTRNQETYETLKHEKPPQ

2. CD3-Zeta

In some embodiments of any of the aspects, the NK cell membrane-bound signaling adaptor protein or domain(s) thereof (e.g., an intracellular signaling domain) that can be adapted or modified for use in a NK CAR polypeptide as described herein is CD3-zeta (CD3ζ). CD3-zeta is also referred to interchangeably as CD247, T-Cell Surface Glycoprotein CD3 Zeta Chain, TCRZ, or IMD25. CD3-zeta comprises three ITAMs, which when phosphorylated creates multiple docking sites for the protein kinase ZAP70 leading to ZAP70 phosphorylation and its conversion into a catalytically active enzyme. In NK cells, CD3-zeta can act as an adaptor protein to transduce intracellular signaling for NK activating receptors NKp46, CD16, or hNKp30.

In some embodiments of any of the aspects, CD3-zeta comprises SEQ ID NO: 128 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 128, that maintains the same function as SEQ ID NO: 128 (e.g., intracellular signaling; ITAM functionality). In some embodiments of any of the aspects, the intracellular signaling domain of an NK-CAR as described herein comprises an intracellular portion of CD3-zeta (e.g., SEQ ID NO: 128). In some embodiments of any of the aspects, the intracellular signaling domain comprises SEQ ID NO: 11 (i.e., residues 52-163 of SEQ ID NO: 128) or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 11, that maintains the same function as SEQ ID NO: 11 (e.g., intracellular signaling; ITAM functionality).

CD3-zeta comprises ITAMs at residues 69-89, 107-127, and 138-158 of SEQ ID NO: 128, with tyrosine or serine/threonine phosphorylation sites at residues 72, 83, 110, 122, 141, and 152 of SEQ ID NO: 128. Accordingly, in some embodiments of any of the aspects, an intracellular signaling domain, which comprises a sequence substantially similar (e.g., at least 70% identical) to the intracellular signaling domain of CD3-zeta (e.g., residues 52-163 of SEQ ID NO: 128; SEQ ID NO: 11), does not comprise mutations in residues 69-89, 107-127, or 138-158 of SEQ ID NO: 128, or does not comprise mutations in residues 72, 83, 110, 122, 141, or 152 of SEQ ID NO: 128, or comprises tyrosines at residues 72, 83, 110, 122, 141, or 152 of SEQ ID NO: 128.

SEQ ID NO: 128, T-cell surface glycoprotein CD3 zeta chain isoform 2 precursor [Homo sapiens], NCBI Reference Sequence: NP_000725.1, 163 aa; bold text indicates the ITAMs, and double-underlined text indicates phosphorylation sites. In some embodiments of any of the aspects, glutamine-65 (bold-italicized) in SEQ ID NO: 128 can be changed to lysine-65.

MKWKALFTAAILQAQLPITEAQSFGLLDPKLCYLLDGILFIYGVILTALFLRVKFSRSADAPAYQQ
GQNQL [symbol] NELNLGRREE [symbol] DVLDKRRGRDPEMGGKPRRKNPQEGL [symbol] NELQKDKMAEA [symbol] SEIG
MKGERRRGKGHDGL [symbol] QGLSTATKDT [symbol] DALHMQALPPR

SEQ ID NOs: 5 (polynucleotide) and 11 (amino acid) provide an exemplary CD3z intracellular signaling domain (e.g., residues 52-163 of CD3z; see e.g., SEQ ID NO: 128 for an exemplary full-length CD3z polypeptide sequence).

SEQ ID NO: 5
AGAGTCAAGTTCTCCAGGTCCGCCGACGCCCCCGCGTACAAGCAGGGCC
AGAACCAGCTCTATAACGAGCTCAATCTAGGACGAAGAGAGGAGTACGA
TGTTTTGGACAAGAGACGTGGCCGGGACCCTGAGATGGGGGGAAAGCCG
AGAAGGAAGAACCCTCAGGAAGGCCTGTACAATGAACTGCAGAAAGATA
AGATGGCGGAGGCCTACAGTGAGATTGGGATGAAAGGCGAGCGCCGGAG
GGGCAAGGGGCACGATGGCCTTTACCAGGGTCTCAGTACAGCCACCAAG
GACACCTACGACGCCCTTCACATGCAGGCCCTGCCCCCTCGC
SEQ ID NO: 11
RVKFSRSADAPAYKQGQNQLYNELNLGRREEYDVLDKRRGRDPEMGGKP
RRKNPQEGLYNELQKDKMAEAYSEIGMKGERRRGKGHDGLYQGLSTATK
DTYDALHMQALPPR

Co-Stimulatory Domains

Signals generated through the NK activating receptor and/or NK adaptor protein can be insufficient for full activation of the NK cell such that a secondary or co-stimulatory signal is also required. As used herein, the term “co-stimulatory receptor” refers to a polypeptide, expressed by an NK cell, comprising an extracellular domain that interacts with a co-stimulatory ligand outside of the cell; the co-stimulatory receptor typically further comprises a transmembrane domain and intracellular signaling domains, examples of which are described further herein. The NK cell can be fully activated once signals are transduced by both the activating receptor and the co-stimulatory receptor, each of which optionally transduces its signal through an adaptor protein. In some embodiments of any of the aspects, the co-stimulatory receptor or domain(s) thereof (e.g., co-stimulatory domain) that can be adapted or modified for use in a NK CAR polypeptide as described herein is 4-1BB and/or IL2 receptor beta (IL2RB). In some embodiments of any of the aspects, the CAR polypeptide comprises the co-stimulatory domain of 4-1BB. In some embodiments of any of the aspects, the CAR polypeptide comprises the co-stimulatory domain of IL-2RB. In some embodiments of any of the aspects, the CAR polypeptide comprises the co-stimulatory domains of 4-1BB and IL-2RB.

In some embodiments of any of the aspects, the CAR polypeptide comprises 1, 2, 3, 4, 5, or more co-stimulatory domains. In some embodiments of any of the aspects, the CAR polypeptide comprises three co-stimulatory domains. In embodiments comprising multiple co-stimulatory domains, the multiple co-stimulatory domains can be different individual co-stimulatory domains or multiple copies of the same co-stimulatory domain, or a combination of the foregoing.

In some embodiments of any of the aspects, the co-stimulatory domain of a CAR polypeptide as described herein comprises one of SEQ ID NOs: 15-16, or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of SEQ ID NO: 15-16, that maintains the same functions as SEQ ID NOs: 15-16 (e.g., co-stimulation).

In some embodiments of any of the aspects, the co-stimulatory domain of a CAR polypeptide as described herein is encoded by a nucleic acid sequence comprising one of SEQ ID NOs: 13-14, or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 13-14 that maintains the same function or a codon-optimized version of one of SEQ ID NOs: 13-14.

1. 4-1BB

In some embodiments of any of the aspects, the co-stimulatory receptor or domain(s) thereof (e.g., co-stimulatory domain) that can be adapted or modified for use in a NK CAR polypeptide as described herein is 4-1BB. 4-1BB is also referred to interchangeably as CD137, tumor necrosis factor receptor superfamily member 9 (TNFRSF9), or Induced by Lymphocyte Activation (ILA). TRAF adaptor proteins have been shown to bind to 4-1BB and transduce the signals leading to activation of NF-kappaB. 4-1BB expression on human NK cells associates with phenotypic markers of activation and proinflammatory cytokine secretion. The cytoplasmic domain of 4-1BB has been shown to interact with a leucine-rich repeat (LRR)-containing protein, (LRR-1), with LRR-1 acting as a negative regulator of 4-1BB.

In some embodiments of any of the aspects, 4-1BB comprises SEQ ID NO: 129 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 129, that maintains the same function as SEQ ID NO: 129 (e.g., co-stimulation). In some embodiments of any of the aspects, the co-stimulatory domain of an NK-CAR as described herein comprises an intracellular portion of 4-1BB (e.g., SEQ ID NO: 129). In some embodiments of any of the aspects, the co-stimulatory domain comprises SEQ ID NO: 15 (i.e., residues 214-255 of SEQ ID NO: 129) or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 15 that maintains the same function as SEQ ID NO: 15 (e.g., co-stimulation).

4-1BB (tumor necrosis factor receptor
superfamily member 9 precursor)
[Homo sapiens], NCBI Reference
Sequence: NP_001552.2, 255 aa.
SEQ ID NO: 129
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCEL,

SEQ ID NOs: 13 (polynucleotide) and 15 (amino acid) provide an exemplary 4-1BB intracellular co-stimulatory domain (e.g., residues 214-255 of 4-1BB; see e.g., SEQ ID NO: 129 for an exemplary full-length 4-1BB polypeptide sequence).

SEQ ID NO: 13
AAAAGGGGCAGAAAGAAACTGCTCTACATCTTCAAGCAGCCCTTCATGAG
GCCCGTCCAAACCACCCAGGAGGAGGACGGATGCAGCTGTAGATTCCCCG
AAGAGGAGGAAGGCGGCTGCGAGCTG
SEQ ID NO: 15
KRGRKKLLYIFKQPFMRPVQTTQEEDGCSCRFPEEEEGGCEL

2. IL2RB

In some embodiments of any of the aspects, the co-stimulatory receptor or domain(s) thereof (e.g., co-stimulatory domain) that can be adapted or modified for use in a NK CAR polypeptide as described herein is Interleukin 2 Receptor Subunit Beta (IL-2RB). IL-2RB is also referred to interchangeably as CD122, Interleukin-15 Receptor Subunit Beta, P75, or IMD63. The interleukin 2 receptor is present in three forms with respect to ability to bind interleukin 2. The low affinity form is a monomer of the alpha subunit and is not involved in signal transduction. The intermediate affinity form consists of an alpha/beta subunit heterodimer, while the high affinity form consists of an alpha/beta/gamma subunit heterotrimer. Both the intermediate and high affinity forms of the receptor are involved in receptor-mediated endocytosis and transduction of mitogenic signals from interleukin 2. IL-2 can enhance NK cell responses toward infection in vivo and can activate NK cells in vitro.

In some embodiments of any of the aspects, IL-2RB comprises SEQ ID NO: 130 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 130, that maintains the same function as SEQ ID NO: 130 (e.g., co-stimulation). In some embodiments of any of the aspects, the co-stimulatory domain of an NK-CAR as described herein comprises an intracellular portion of IL-2RB (e.g., SEQ ID NO: 130). In some embodiments of any of the aspects, the intracellular signaling domain comprises SEQ ID NO: 16 (i.e., residues 266-337 and 530-551 of SEQ ID NO: 130) or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 16 that maintains the same function as SEQ ID NO: 16 (e.g., co-stimulation).

interleukin-2 receptor subunit beta precursor
[Homo sapiens], NCBI Reference Sequence:
NP_000869.1, 551 aa.
SEQ ID NO: 130
MAAPALSWRLPLLILLLPLATSWASAAVNGTSQFTCFYNSRANISCVWSQ
DGALQDTSCQVHAWPDRRRWNQTCELLPVSQASWACNLILGAPDSQKLTT
VDIVTLRVLCREGVRWRVMAIQDFKPFENLRLMAPISLQVVHVETHRCNI
SWEISQASHYFERHLEFEARTLSPGHTWEEAPLLTLKQKQEWICLETLTP
DTQYEFQVRVKPLQGEFTTWSPWSQPLAFRTKPAALGKDTIPWLGHLLVG
LSGAFGFIILVYLLINCRNTGPWLKKVLKCNTPDPSKFFSQLSSEHGGDV
QKWLSSPFPSSSFSPGGLAPEISPLEVLERDKVTQLLLQQDKVPEPASLS
SNHSLTSCFTNQGYFFFHLPDALEIEACQVYFTYDPYSEEDPDEGVAGAP
TGSSPQPLQPLSGEDDAYCTFPSRDDLLLFSPSLLGGPSPPSTAPGGSGA
GEERMPPSLQERVPRDWDPQPLGPPTPGVPDLVDFQPPPELVLREAGEEV
PDAGPREGVSFPWSRPPGQGEFRALNARLPLNTDAYLSLQELQGQDPTHL
V,

SEQ ID NOs: 14 (polynucleotide) and 16 (amino acid) provide an exemplary IL-2RB intracellular co-stimulatory domain (e.g., residues 266-337 and 530-551 of IL-2RB; see e.g., SEQ ID NO: 130 for an exemplary full-length IL-2B polypeptide sequence).

SEQ ID NO: 14
AACTGCAGGAACACCGGGCCATGGCTGAAGAAGGTCCTGAAGTGTAACAC
CCCAGACCCCTCGAAGTTCTTTTCCCAGCTGAGCTCAGAGCATGGAGGAG
ACGTCCAGAAGTGGCTCTCTTCGCCCTTCCCCTCATCGTCCTTCAGCCCT
GGCGGCCTGGCACCTGAGATCTCGCCACTAGAAGTGCTGGAGAGGGACAA
GGTGACGCAGCTGCTCCCCCTGAACACTGATGCCTACTTGTCCCTCCAAG
AACTCCAGGGTCAGGACCCAACTCACTTGGTG
SEQ ID NO: 16
NCRNTGPWLKKVLKCNTPDPSKFFSQLSSEHGGDVQKWLSSPFPSSSFSP
GGLAPEISPLEVLERDKVTQLLPLNTDAYLSLQELQGQDPTHLV

Self-Cleaving Peptides

In several aspects, described herein are NK CAR polypeptides comprising a self-cleaving peptide. As used herein, the term “self-cleaving peptide” refers to a short amino acid sequence (e.g., approximately 18-22 aa) that can catalyze its own cleavage. In some embodiments of any of the aspects, a CAR polypeptide as described herein comprises intracellular signaling domain(s) and/or co-stimulatory domain(s) that are physically linked to another polypeptide (e.g., a cytokine) through a self-cleaving peptide domain. The self-cleaving peptide allows the nucleic acids of a first polypeptide (e.g., an NK CAR) and a second polypeptide (and/or third polypeptide, etc.; e.g., at least one cytokine) to be present in the same vector, but after translation the self-cleaving peptide cleaves the translated polypeptide into the multiple separate polypeptides.

In some embodiments of any of the aspects, a CAR polypeptide as described herein comprises 1, 2, 3, 4, 5, or more self-cleaving peptides. In some embodiments of any of the aspects, the CAR polypeptide or system comprises one self-cleaving peptide, e.g., in between a first polypeptide and a second polypeptide. In some embodiments of any of the aspects, the CAR polypeptide or system comprises two self-cleaving peptides, e.g., in between a first polypeptide and a second polypeptide, and in between a second polypeptide and a third polypeptide. In embodiments comprising multiple self-cleaving peptides, the multiple self-cleaving peptides can be different individual self-cleaving peptides or multiple copies of the same self-cleaving peptide, or a combination of the foregoing.

In some embodiments of any of the aspects, the self-cleaving peptide belongs to the 2A peptide family. Non-limiting examples of 2A peptides include P2A, E2A, F2A and T2A (see e.g., Table 8). F2A is derived from foot-and-mouth disease virus 18; E2A is derived from equine rhinitis A virus; P2A is derived from porcine teschovirus-1 2A; T2A is derived from thosea asigna virus 2A. In some embodiments of any of the aspects, the N-terminal end of the 2A peptide comprises the sequence “GSG” (Gly-Ser-Gly). In some embodiments of any of the aspects, the N-terminal end of the 2A peptide does not comprise the sequence “GSG” (Gly-Ser-Gly).

TABLE 8
Exemplary Self-Cleaving Peptides

| SEQ ID NO: | Name | Sequence |
|---|---|---|
| 19 | P2A | (GSG)ATNFSLLKQAGDVEENPGP |
| 20 | T2A | (GSG)RAEGRGSLLTCGDVEENPGP |
| 132 | T2A | (GSG)EGRGSLLTCGDVEENPGP |
| 133 | E2A | (GSG)QCTNYALLKLAGDVESNPGP |
| 134 | F2A | (GSG)VKQTLNFDLLKLAGDVESNPGP |

The 2A-peptide-mediated cleavage commences after protein translation. The cleavage is triggered by breaking of peptide bond between the Proline (P) and Glycine (G) in the C-terminal region of the 2A peptide. The molecular mechanism of 2A-peptide-mediated cleavage involves ribosomal “skipping” of glycyl-prolyl peptide bond formation rather than true proteolytic cleavage. Different 2A peptides have different efficiencies of self-cleaving, with P2A being the most efficient and F2A the least efficient. Therefore, up to 50% of F2A-linked proteins can remain in the cell as a fusion protein.

In some embodiments of any of the aspects, the self-cleaving peptide of a CAR polypeptide as described herein comprises one of SEQ ID NOs: 19-20, 132-134, or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of SEQ ID NOs: 19-20, 132-134, that maintains the same functions as one of SEQ ID NOs: 19-20, 132-134 (e.g., self-cleavage).

In some embodiments of any of the aspects, the self-cleaving peptide of a CAR polypeptide as described herein is encoded by a nucleic acid sequence comprising one of SEQ ID NOs: 17, 18, or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 17 or 18 that maintains the same function or a codon-optimized version of one of SEQ ID NOs: 17 or 18.

P2A
SEQ ID NO: 17
GGCAGCGGCGCCACCAACTTCTCTTTACTGAAGCAAGCTGGCGACGTGGA
GGAGAATCCCGGTCCC,
T2A
SEQ ID NO: 18
GGCAGCGGAAGAGCCGAGGGAAGAGGCTCTTTACTGACTTGTGGCGACGT
GGAAGAGAATCCCGGCCCC,

Cytokines

In several aspects, described herein are NK CAR polypeptides comprising at least one cytokine. As used herein, the term “cytokine” refers to a small protein that mediates and/or regulates a biological or cellular function or process (e.g. immunity, inflammation, and hematopoiesis). The term “cytokine” as used herein includes “lymphokines,” “chemokines,” “monokines,” and “interleukins”. Examples of cytokines that modulate NK cell functions include, but are not limited to, IL-10, IL-12, IL-15, IL-18, IL-21, IFN-γ, GM-CSF, TGF-β, TNF-α, and IFN-α. The term “cytokine” as used herein is meant to also include cytokine variants comprising one or more amino acid mutations in the amino acid sequences of the corresponding wild-type cytokine. In some embodiments of any of the aspects, the at least one cytokine is linked to the NK CAR polypeptide using at least one self-cleaving peptide as described herein. Any combination of self-cleaving peptide and cytokine can be used. Use of a self-cleaving peptide allows the cytokine to be cleaved from the end of the NK CAR polypeptide. If the cytokine comprises a signal peptide, then the cytokine can be secreted from the cell, to act on the cell that secreted it or on nearby cells.

In some embodiments of any of the aspects, a CAR polypeptide as described herein comprises 1, 2, 3, 4, 5, or more cytokines. In some embodiments of any of the aspects, the CAR polypeptide or system comprises one cytokine. In some embodiments of any of the aspects, the CAR polypeptide or system comprises two cytokines. In embodiments comprising multiple cytokines, the multiple cytokines can be different individual cytokines or multiple copies of the same cytokine, or a combination of the foregoing.

In some embodiments of any of the aspects, the intracellular domain of the NK CAR comprises at least one self-cleaving peptide and at least one cytokine. In some embodiments of any of the aspects, the intracellular domain of the NK CAR comprises one self-cleaving peptide and one cytokine. In some embodiments of any of the aspects, the cytokine is adjacent and distal to (i.e., farther from the transmembrane domain compared to) the self-cleaving peptide, such that the cytokine is separated from the polypeptide by the self-cleaving peptide. In some embodiments of any of the aspects, the intracellular domain of the NK CAR comprises two self-cleaving peptides and two cytokines. In some embodiments of any of the aspects, the first self-cleaving peptide is adjacent and proximal to (i.e., closer to the transmembrane domain compared to) the first cytokine, and the second self-cleaving peptide is adjacent and in between the first and second cytokines, such that both cytokines are separated from the polypeptide by the self-cleaving peptides.

In some embodiments of any of the aspects, the NK CAR polypeptide comprises IL-15 or IL-21. In some embodiments of any of the aspects, the NK CAR polypeptide comprises IL-15. In some embodiments of any of the aspects, the NK CAR polypeptide comprises IL-21. In some embodiments of any of the aspects, the NK CAR polypeptide comprises IL-15 and IL-21. IL-15 is constitutively expressed by a large number of cell types and tissues, including monocytes, macrophages, dendritic cells (DC), keratinocytes, fibroblasts, myocytes, and nerve cells. IL-15 binds to and signals through a complex composed of IL-2/IL-15 receptor beta chain (CD122) and the common gamma chain (gamma-C, CD132). IL-15 induces the proliferation of natural killer cells. Interleukin-21 (IL-21) is naturally expressed by activated human CD4+ T cells, T follicular cells, and NK T cells. The IL-21 receptor (IL-21R) is expressed on the surface of T, B and NK cells. IL-21 induces cell division/proliferation in NK cells and cytotoxic T cells. As such, inclusion of IL-15 and/or IL-21 can increase the proliferation of an NK cell expressing an NK CAR comprising a cytokine, or a nearby NK cell.

In some embodiments of any of the aspects, the cytokine of a CAR polypeptide as described herein comprises one of SEQ ID NOs: 23, 24, or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of SEQ ID NOs: 23 or 24, that maintains the same functions as one of SEQ ID NOs: 23 or 24 (e.g., promoting NK cell proliferation).

In some embodiments of any of the aspects, the cytokine of a CAR polypeptide as described herein is encoded by a nucleic acid sequence comprising one of SEQ ID NOs: 21, 22, or a nucleic acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 21 or 22 that maintains the same function or a codon-optimized version of one of SEQ ID NOs: 21 or 22.

SEQ ID NOs: 21 and 23 provide polynucleotide and amino acid sequences for an exemplary IL-15 cytokine (e.g., isoform 1; see e.g., interleukin-15 isoform 1 preproprotein [Homo sapiens], NCBI Reference Sequence: NP_000576.1, 162 aa).

SEQ ID NO: 21
ATGAGGATCAGCAAGCCCCATTTAAGGAGCATCTCCATCCAGTGCTATTT
ATGTTTACTGCTGAACAGCCATTTTTTAACCGAGGCCGGCATCCACGTGT
TCATTTTAGGCTGCTTTTCCGCCGGTTTACCCAAGACCGAGGCTAACTGG
GTGAACGTGATCAGCGATTTAAAGAAGATCGAGGATTTAATCCAGAGCAT
GCACATCGATGCCACTTTATATACCGAGAGCGACGTGCACCCCAGCTGCA
AGGTGACCGCCATGAAGTGTTTTTTACTGGAGCTGCAAGTTATCTCTTTA
GAGAGCGGCGACGCCAGCATCCACGACACCGTGGAGAATTTAATCATTTT
AGCCAACAACAGCCTCTCCAGCAACGGCAATGTGACAGAGAGCGGCTGCA
AGGAGTGCGAGGAGCTGGAGGAGAAGAACATCAAGGAGTTTTTACAGAGC
TTCGTGCACATCGTGCAGATGTTCATCAACACCAGC
SEQ ID NO: 23
MRISKPHLRSISIQCYLCLLLNSHFLTEAGIHVFILGCFSAGLPKTEANW
VNVISDLKKIEDLIQSMHIDATLYTESDVHPSCKVTAMKCFLLELQVISL
ESGDASIHDTVENLIILANNSLSSNGNVTESGCKECEELEEKNIKEFLQS
FVHIVQMFINTS

SEQ ID NOs: 22 and 24 provide polynucleotide and amino acid sequences for an exemplary IL-21 cytokine (e.g., isoform 1; e.g., residues 8-162 of IL-21 (155 aa); see e.g., interleukin-21 isoform 1 precursor [Homo sapiens], NCBI Reference Sequence: NP_068575.1, 162 aa).

SEQ ID NO: 22
ATGGAGAGAATCGTGATCTGTTTAATGGTGATCTTTTTAGGCACTTTAGT
GCACAAGTCCAGCTCCCAAGGTCAAGATAGACACATGATTCGTATGAGGC
AGCTGATCGACATCGTGGACCAGCTGAAGAACTACGTGAACGATTTAGTG
CCCGAATTTTTACCCGCTCCCGAGGACGTGGAGACCAACTGCGAGTGGAG
CGCCTTCTCTTGTTTCCAGAAGGCTCAGCTGAAGAGCGCCAACACCGGCA
ACAACGAGAGGATTATCAACGTGTCCATCAAGAAGCTGAAGAGGAAGCCT
CCCTCCACCAACGCCGGCAGAAGGCAGAAGCACAGACTGACTTGTCCCTC
TTGTGACAGCTACGAGAAGAAGCCCCCTAAGGAGTTTTTAGAGAGATTCA
AGTCTTTACTGCAGAAGATGATCCACCAGCATTTAAGCTCTCGTACCCAC
GGCAGCGAGGATTCC
SEQ ID NO: 24
MERIVICLMVIFLGTLVHKSSSQGQDRHMIRMRQLIDIVDQLKNYVNDLV
PEFLPAPEDVETNCEWSAFSCFQKAQLKSANTGNNERIINVSIKKLKRKP
PSTNAGRRQKHRLTCPSCDSYEKKPPKEFLERFKSLLQKMIHQHLSSRTH
GSEDS

### Transmembrane Domains

In various aspects, described herein are NK CAR polypeptides comprising a transmembrane domain. With respect to the transmembrane domain, the CAR can be designed to comprise a transmembrane domain that is fused to the extracellular binding domain and/or the intracellular domain of the CAR. In some embodiments of any of the aspects, the CAR polypeptide comprises 1, 2, 3, 4, 5, or more transmembrane domains. In some embodiments of any of the aspects, each CAR polypeptide comprises one transmembrane domain. In embodiments comprising multiple transmembrane domains, the multiple transmembrane domains can be different individual transmembrane domains or multiple copies of the same transmembrane domain, or a combination of the foregoing.

In some embodiments of any of the aspects, the transmembrane domain can be derived either from a natural or from a synthetic source. Where the source is natural, the domain can be derived from any membrane-bound or transmembrane protein. In some embodiments of any of the aspects, the CAR polypeptide described herein comprises at least the transmembrane region(s) of a transmembrane protein selected from the group consisting of: NKG2D, NKp46, DNAM, CD8, the alpha or beta or zeta chain of the T-cell receptor, CD28, CD23zeta, CD28, 4-1BB, CD3 epsilon, CD45, CD4, CD5, CD8, CD9, CD16, CD22, CD33, CD37, CD64, CD80, CD86, CD134, CD137, and CD154. In some embodiments, an NK CAR polypeptide comprises a transmembrane domain of an activating receptor listed in Table 10. Alternatively, the transmembrane domain can be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the intracellular signaling domain of the CAR. A glycine-serine doublet provides a particularly suitable linker.

In some embodiments of any of the aspects, the NK CAR comprises the transmembrane domain from the group consisting of Natural Killer Group 2D (NKG2D); Natural Killer Cell P46-Related Protein (NKp46); DNAX Accessory Molecule-1 (DNAM1); and CD8. In some embodiments of any of the aspects, the transmembrane domain comprises a transmembrane domain of a natural NK cell receptor. In some embodiments of any of the aspects, the transmembrane domain of the natural NK cell receptor is selected from the group consisting of Natural Killer Group 2D (NKG2D); Natural Killer Cell P46-Related Protein (NKp46); and DNAX Accessory Molecule-1 (DNAM1). In some embodiments of any of the aspects, the transmembrane domain comprises a transmembrane domain of CD8. In some embodiments of any of the aspects, the transmembrane domain further comprises a portion of the adjacent intracellular domain from the same NK cell receptor. As a non-limiting example, the NKp46 or DNAM1 transmembrane domains further comprise a portion of the adjacent intracellular domain from NKP46 or DNAM1, respectively. Inclusion of the intracellular domain can increase the signaling of the NK CAR since such receptors can activate NK cells by signaling through their own cytoplasmic tail. See e.g., Bryceson et al., 2006, Blood 107: 159-166. In some embodiments of any of the aspects, the transmembrane domain can be selected or modified by amino acid substitution to avoid binding of such domains to the transmembrane domains of the same or different surface membrane proteins to minimize interactions with other members of the receptor complex.

In some embodiments of any of the aspects, the transmembrane domain of a CAR polypeptide as described herein comprises one of SEQ ID NOs: 29-32 or 117-118, or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of SEQ ID NO: 29-32 or 117-118, that maintains the same functions as SEQ ID NOs: 29-32 or 117-118 (e.g., localizes to the cell membrane).

In some embodiments of any of the aspects, the transmembrane domain of a CAR polypeptide as described herein is encoded by a nucleic acid sequence comprising one of SEQ ID NOs: 25-28 or 115-116, or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 25-28 or 115-116 that maintains the same function, or a codon-optimized version of one of SEQ ID NOs: 25-28 or 115-116.

In some embodiments of any of the aspects, the transmembrane domain of a CAR polypeptide as described herein further comprises an intracellular signaling domain from the same protein as the transmembrane domain, and comprises one of SEQ ID NOs: 30-31, or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of SEQ ID NO: 30-31, that maintains the same functions as SEQ ID NOs: 30-31 (e.g., localizes to the cell membrane and/or intracellular signaling).

In some embodiments of any of the aspects, the transmembrane domain of a CAR polypeptide as described herein is encoded by a nucleic acid sequence further comprising an intracellular signaling domain from the same protein as the transmembrane domain, and comprising one of SEQ ID NOs: 26-27, or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 26-27 that maintains the same function, or a codon-optimized version of one of SEQ ID NOs: 26-27.

In some embodiments of any of the aspects, the transmembrane domain of a CAR polypeptide as described herein does not further comprise an intracellular signaling domain from the same protein as the transmembrane domain, and comprises one of SEQ ID NOs: 29, 32, 117, 118, or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of SEQ ID NO: 29 or 32 or 117-118, that maintains the same functions as SEQ ID NOs: 29, 32, 117, or 118 (e.g., localizes to the cell membrane).

In some embodiments of any of the aspects, the transmembrane domain of a CAR polypeptide as described herein is encoded by a nucleic acid sequence that does not further comprise an intracellular signaling domain from the same protein as the transmembrane domain, and comprises one of SEQ ID NOs: 25, 28, 115, 116, or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 25, 28, 115, or 116 that maintains the same function, or a codon-optimized version of one of SEQ ID NOs: 25, 28, 115, or 116.

1. NKG2D

In some embodiments of any of the aspects, the NK CAR polypeptide comprises at least the transmembrane domain of the NK cell receptor NKG2D. NKG2D is also referred to interchangeably as CD314, Killer Cell Lectin-Like Receptor Subfamily K Member 1 (KLRK1), NK Cell Receptor D, or D12S2489E. NKG2D is a transmembrane protein belonging to the NKG2 family of C-type lectin-like receptors. NKG2D is characterized by a type II membrane orientation (has an extracellular C terminus) and the presence of a C-type lectin domain. It binds to a diverse family of ligands that include MHC class I chain-related A and B proteins and UL-16 binding proteins, where ligand-receptor interactions can result in the activation of NK and T cells. NKG2D is an activating NK receptor that signals through the adaptor protein DAP10.

In some embodiments of any of the aspects, NKG2D comprises SEQ ID NO: 135 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 135, that maintains the same function as SEQ ID NO: 135 (e.g., localizes to the cell membrane and/or intracellular signaling). In some embodiments of any of the aspects, an NK-CAR as described herein comprises a transmembrane domain of NKG2D (e.g., SEQ ID NO: 135). In some embodiments of any of the aspects, the transmembrane domain comprises SEQ ID NO: 29 (i.e., residues 52-72 of SEQ ID NO: 135) or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 29, that maintains the same function as SEQ ID NO: 29 (e.g., localizes to the cell membrane).

NKG2-D type II integral membrane protein,
UniProtKB - P26718 (NKG2D_HUMAN), 216 aa
SEQ ID NO: 135
MGWIRGRRSRHSWEMSEFHNYNLDLKKSDFSTRWQKQRCPVVKSKCRENA
SPFFFCCFIAVAMGIRFIIMVAIWSAVFLNSLFNQEVQIPLTESYCGPCP
KNWICYKNNCYQFFDESKNWYESQASCMSQNASLLKVYSKEDQDLLKLVK
SYHWMGLVHIPTNGSWQWEDGSILSPNLLTIIEMQKGDCALYASSFKGYI
ENCSTPNTYICMQRTV,

SEQ ID NOs: 25 and 29 provide polynucleotide and amino acid sequences for an exemplary NKG2D transmembrane domain (e.g., residues 52-72 of NKG2D; see e.g., SEQ ID NO: 135 for an exemplary full-length NKG2D polypeptide sequence).

SEQ ID NO: 25
CCTTTCTTCTTCTGCTGCTTCATCGCCGTGGCCATGGGCATTCGTTTCAT
CATCATGGTGGCC
SEQ ID NO: 29
PFFFCCFIAVAMGIRFIIMVA

2. NKp46

In some embodiments of any of the aspects, the NK CAR polypeptide comprises at least the transmembrane domain of the NK cell receptor NKp46. NKp46 is also referred to interchangeably as Natural Cytotoxicity Triggering Receptor 1 (NCR1), CD335, and Lymphocyte Antigen 94 Homolog (LY94). NKp46 is a 46 kDa type I membrane glycoprotein that is expressed on resting and activated NK cells. Its extracellular region contains two C2-type, Ig-like domains. The transmembrane domain contains a positively charged amino acid (Arg) which could be involved in stabilizing its association with CD3ζ. Its intracellular region does not contain immunoreceptor tyrosine-based activating motifs (ITAM), but it is linked to intracytoplasmic transduction machinery by its association with CD3ζ and FcεRIγ adaptor proteins. Nkp46, along with NKp30 and NKp44, are referred to as natural cytotoxicity receptors (NCR). These receptors play very important roles in cells that kill virus-infected target cells, tumor cells and MHC-class I-unprotected cells.

In some embodiments of any of the aspects, Nkp46 comprises SEQ ID NO: 136 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 136, that maintains the same function as SEQ ID NO: 136 (e.g., localizes to the cell membrane and/or intracellular signaling). In some embodiments of any of the aspects, an NK-CAR as described herein comprises a transmembrane domain of NKp46 (e.g., SEQ ID NO: 136). In some embodiments of any of the aspects, the transmembrane domain comprises SEQ ID NO: 117 (i.e., residues 259-279 of SEQ ID NO: 136) or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 117, that maintains the same function as SEQ ID NO: 117 (e.g., localizes to the cell membrane).

In some embodiments of any of the aspects, an NK-CAR as described herein comprises a transmembrane domain of NKp46 and an intracellular signaling domain of NKp46 (e.g., SEQ ID NO: 136). In some embodiments of any of the aspects, the transmembrane and intracellular signaling domains comprises SEQ ID NO: 30 (i.e., residues 259-304 of SEQ ID NO: 136) or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 30, that maintains the same function as SEQ ID NO: 30 (e.g., localizes to the cell membrane and/or intracellular signaling).

In some embodiments of any of the aspects, an NK-CAR as described herein comprises an intracellular signaling domain of NKp46 (e.g., SEQ ID NO: 136). In some embodiments of any of the aspects, the intracellular signaling domain comprises residues 280-304 of SEQ ID NO: 136 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to a sequence comprising residues 280-304 of SEQ ID NO: 136, that maintains the same function as residues 280-304 of SEQ ID NO: 136 (e.g., intracellular signaling).

natural cytotoxicity triggering receptor 1
isoform a precursor [Homo sapiens], NCBI
Reference Sequence: NP_004820.2, 304 aa
SEQ ID NO: 136
MSSTLPALLCVGLCLSQRISAQQQTLPKPFIWAEPHFMVPKEKQVTICCQ
GNYGAVEYQLHFEGSLFAVDRPKPPERINKVQFYIPDMNSRMAGQYSCIY
RVGELWSEPSNLLDLVVTEMYDTPTLSVHPGPEVISGEKVTFYCRLDTAT
SMFLLLKEGRSSHVQRGYGKVQAEFPLGPVTTAHRGTYRCFGSYNNHAWS
FPSEPVKLLVTGDIENTSLAPEDPTFPADTWGTYLLTTETGLQKDHALWD
HTAQNLLRMGLAFLVLVALVWFLVEDWLSRKRTRERASRASTWEGRRRLN
TQTL,

SEQ ID NOs: 26 and 30 provide polynucleotide and amino acid sequences for an exemplary NKp46 transmembrane and intracellular domain (e.g., residues 259-304 of NKp46; see e.g., SEQ ID NO: 136 for an exemplary full-length NKp46 polypeptide sequence).

bolded text indicates sequence encoding an
exemplary transmembrane region of NKp46
(e.g., residues 259-279 of SEQ ID NO: 136;
nucleotides (nt) 1-63 of SEQ ID NO: 26;
SEQ ID NO: 115); unformatted text indicates
sequence encoding the intracellular
signaling domain of NKp46 (see e.g.,
residues 280-304 of SEQ ID NO: 136).
SEQ ID NO: 26
ATGGGACTGGCCTTTCTGGTGCTGGTGGCTCTGGTGTGGTTTCTGGTGGA
GGACTGGCTGAGCAGAAAGAGGACCAGAGAGAGAGCCAGCAGAGCCAGCA
CATGGGAGGGCAGAAGGAGACTGAACACCCAGACCCTG;

SEQ ID NO: 30; bolded text indicates an exemplary transmembrane region of NKp46 (see e.g., residues 259-279 of SEQ ID NO: 136; amino acids (aa) 1-21 of SEQ ID NO: 30; SEQ ID NO: 117); unformatted text indicates the intracellular signaling domain of NKp46 (see e.g., residues 280-304 of SEQ ID NO: 136). MGLAFLVLVALVWFLVEDWLSRKRTRERASRASTWEGRRRLNTQTL

SEQ ID NOs: 115 and 117 provide polynucleotide and amino acid sequences for an exemplary NKp46 transmembrane domain (e.g., residues 259-279 of NKp46).

SEQ ID NO: 115
ATGGGACTGGCCTTTCTGGTGCTGGTGGCTCTGGTGTGGTTTCTGGTGGA
GGACTGGCTGAGC
SEQ ID NO: 117
MGLAFLVLVALVWFLVEDWLS,

3. DNAM1

In some embodiments of any of the aspects, the NK CAR polypeptide comprises at least the transmembrane domain of the NK cell receptor DNAM1. DNAM1 is also referred to interchangeably as DNAX Accessory Molecule-1, CD226, T Lineage-Specific Activation Antigen 1 Antigen (TLiSA1), or Platelet And T Cell Activation Antigen 1 (PTA1). DNAM1 is a glycoprotein expressed on the surface of NK cells, platelets, monocytes and a subset of T cells. It is a member of the Ig-superfamily containing 2 Ig-like domains of the V-set. DNAM1 is involved in intercellular adhesion, lymphocyte signaling, cytotoxicity and lymphokine secretion mediated by cytotoxic T-lymphocyte (CTL) and NK cells. DNAM-1 is associated with LFA-1 in NK cells, is phosphorylated by a PKC, and binds to CD155 and CD112. Antibodies to DNAM-1 inhibit NK-cell cytotoxicity toward tumor cells.

In some embodiments of any of the aspects, DNAM1 comprises SEQ ID NO: 137 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 137, that maintains the same function as SEQ ID NO: 137 (e.g., localizes to the cell membrane and/or intracellular signaling). In some embodiments of any of the aspects, an NK-CAR as described herein comprises a transmembrane domain of DNAM1 (e.g., SEQ ID NO: 137). In some embodiments of any of the aspects, the transmembrane domain comprises SEQ ID NO: 118 (i.e., residues 255-275 of SEQ ID NO: 137) or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 118, that maintains the same function as SEQ ID NO: 118 (e.g., localizes to the cell membrane).

In some embodiments of any of the aspects, an NK-CAR as described herein comprises a transmembrane domain of DNAM1 and an intracellular signaling domain of DNAM1 (e.g., SEQ ID NO: 137). In some embodiments of any of the aspects, the transmembrane and intracellular signaling domains comprise SEQ ID NO: 31 (i.e., residues 255-336 of SEQ ID NO: 137) or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 31, that maintains the same function as SEQ ID NO: 31 (e.g., localizes to the cell membrane and/or intracellular signaling).

In some embodiments of any of the aspects, an NK-CAR as described herein comprises an intracellular signaling domain of DNAM1 (e.g., SEQ ID NO: 136). In some embodiments of any of the aspects, the intracellular signaling domain comprises residues 276-336 of SEQ ID NO: 137 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to a sequence comprising residues 276-336 of SEQ ID NO: 137, that maintains the same function as residues 276-336 of SEQ ID NO: 137 (e.g., intracellular signaling).

CD226 antigen isoform a precursor [Homo
sapiens], NCBI Reference Sequence:
NP_001290547.1, 336 aa
SEQ ID NO: 137
MDYPTLLLALLHVYRALCEEVLWHTSVPFAENMSLECVYPSMGILTQVEW
FKIGTQQDSIAIFSPTHGMVIRKPYAERVYFLNSTMASNNMTLFFRNASE
DDVGYYSCSLYTYPQGTWQKVIQVVQSDSFEAAVPSNSHIVSEPGKNVTL
TCQPQMTWPVQAVRWEKIQPRQIDLLTYCNLVHGRNFTSKFPRQIVSNCS
HGRWSVIVIPDVTVSDSGLYRCYLQASAGENETFVMRLTVAEGKTDNQYT
LFVAGGTVLLLLFVISITTIIVIFLNRRRRRERRDLFTESWDTQKAPNNY
RSPISTSQPTNQSMDDTREDIYVNYPTFSRRPKTRV,

SEQ ID NOs: 27 and 31 provide polynucleotide and amino acid sequences for an exemplary DNAM1 transmembrane and intracellular domain (e.g., residues 255-336 of DNAM1; see e.g., SEQ ID NO: 137 for an exemplary full-length DNAM1 polypeptide sequence).

bolded text indicates sequence encoding an
exemplary transmembrane region of DNAMI
(e.g., residues 255-275 of SEQ ID NO: 137;
nucleotides (nt) 1-63 of SEQ ID NO: 27;
SEQ ID NO: 116); unformatted text indicates
sequence encoding the intracellular
signaling domain of DNAMI (see e.g.,
residues 276-336 of SEQ ID NO: 137).
SEQ ID NO: 27
GGCGGCACCGTGCTGCTGCTGCTGTTCGTGATCTCCATCACCACCATCAT
CGTCATCTTTCTGAACAGAAGGAGGAGAAGGGAGAGGAGGGATCTGTTCA
CCGAGAGCTGGGACACCCAGAAGGCCCCCAACAACTATAGAAGCCCCATC
AGCACCAGCCAGCCCACCAACCAGAGCATGGACGACACCAGAGAGGACAT
CTACGTGAACTACCCCACCTTCAGCAGAAGACCCAAGACCAGAGTG;
bolded text indicates an exemplary
transmembrane region of DNAMI (e.g.,
residues 255-275 of SEQ ID NO: 137;
amino acids (aa) 1-21 of SEQ ID NO:
31; SEQ ID NO: 118).
SEQ ID NO: 31
GGTVLLLLFVISITTIIVIFLNRRRRRERRDLFTESWDTQKAPNNYRSPI
STSQPTNQSMDDTREDIYVNYPTFSRRPKTRV;

SEQ ID NOs: 116 and 118 provide polynucleotide and amino acid sequences for an exemplary DNAM1 transmembrane domain (e.g., residues 255-275 of SEQ ID NO: 137); unformatted text indicates the intracellular signaling domain of DNAM1 (see e.g., residues 276-336 of SEQ ID NO: 137).

SEQ ID NO: 116
GGCGGCACCGTGCTGCTGCTGCTGTTCGTGATCTCCATCACCACCATCAT
CGTCATCTTTCTG
SEQ ID NO: 118,
GGTVLLLLFVISITTIIVIFL

4. CD8

In some embodiments of any of the aspects, the NK CAR polypeptide comprises at least the transmembrane domain of CD8. CD8 forms a dimer, consisting of a pair of CD8 chains. The most common form of CD8 is composed of a CD8-α and CD8-β chain. Accordingly, in some embodiments of any of the aspects, the NK CAR polypeptide comprises at least the transmembrane domain of CD8alpha. CD8alpha is also referred to interchangeably as CD8a, T-Lymphocyte Differentiation Antigen T8/Leu-2, or MAL. CD8 plays a role in T cell signaling and aiding with cytotoxic T cell antigen interactions. CD8 is predominantly expressed on the surface of cytotoxic T cells, but can also be found on natural killer cells, cortical thymocytes, and dendritic cells.

In some embodiments of any of the aspects, CD8 comprises SEQ ID NO: 138 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 138, that maintains the same function as SEQ ID NO: 138 (e.g., localizes to the cell membrane and/or intracellular signaling). In some embodiments of any of the aspects, an NK-CAR as described herein comprises a transmembrane domain of CD8 (e.g., SEQ ID NO: 138). In some embodiments of any of the aspects, the transmembrane domain comprises SEQ ID NO: 32 (i.e., residues 183-206 of SEQ ID NO: 138) or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 32, that maintains the same function as SEQ ID NO: 32 (e.g., localizes to the cell membrane).

SEQ ID NO: 138, T-cell surface glycoprotein CD8
alpha chain, UniProtKB-P01732 (CD8A_HUMAN), 235 aa
MALPVTALLLPLALLLHAARPSQFRVSPLDRTWNLGETVELKCQVLLSNP
TSGCSWLFQPRGAAASPTFLLYLSQNKPKAAEGLDTQRFSGKRLGDTFVL
TLSDFRRENEGYYFCSALSNSIMYFSHFVPVFLPAKPTTTPAPRPPTPAP
TIASQPLSLRPEACRPAAGGAVHTRGLDFACDIYIWAPLAGTCGVLLLSL
VITLYCNHRNRRRVCKCPRPVVKSGDKPSLSARYV

SEQ ID NOs: 28 and 32 provide polynucleotide and amino acid sequences for an exemplary CD8 transmembrane domain (e.g., residues 183-206 of CD8; see e.g., SEQ ID NO: 138 for an exemplary full-length CD8a polypeptide sequence).

SEQ ID NO: 28
ATCTACATCTGGGCCCCTCTGGCTGGCACATGTGGCGTGCTGCTGCTCTC
CCTGGTGATCACCCTGTACTGC
SEQ ID NO: 32,
IYIWAPLAGTCGVLLLSLVITLYC

### Extracellular Domains

In several aspects, described herein are NK CAR polypeptides comprising at least one extracellular domain (also referred to herein as an ectodomain). As described further herein, such extracellular domains can include, but are not limited to: extracellular binding domains, signal peptides, linker domains, detectable markers, and spacer domains. An NK CAR polypeptide can comprise any combination of extracellular binding domains, signal peptides, linker domains, detectable markers, and spacer domains. For example, the following combinations are contemplated herein (see e.g., Table 8):

TABLE 8
Exemplary Combinations of Extracellular Domains in an NK-CAR Polypeptide.

| EB | SI | LD | DM | SD | EB | SI | LD | DM | SD | EB | SI | LD | DM | SD |
|---|---|---|---|---|---|---|---|---|---|---|---|---|---|---|
| | | | | | X | X | | X | | | X | X | | X |
| X | | | | | | | X | X | | X | X | X | | X |
| | X | | | | X | | X | X | | | | | X | X |
| X | X | | | | | X | X | X | | X | | | X | X |
| | | X | | | X | X | X | X | | | X | | X | X |
| X | | X | | | | | | | X | X | X | | X | X |
| | X | X | | | X | | | | X | | | X | X | X |
| X | X | X | | | | X | | | X | X | | X | X | X |
| | | | X | | X | X | | | X | | X | X | X | X |
| X | | | X | | | | X | | X | X | X | X | X | X |
| | X | | X | | X | | X | | X | | | | | |

“EB” indicates extracellular binding domain.
“SI” indicates signal peptide.
“LD” indicates linker domain.
“DM” indicates detectable marker.
“SD” indicates spacer domain.

In some embodiments of any of the aspects, the CAR polypeptide comprises 1, 2, 3, 4, 5, or more extracellular domains. In some embodiments of any of the aspects, the CAR polypeptide comprises five extracellular domains. In some embodiments of any of the aspects, the CAR polypeptide comprises four extracellular domains. In embodiments comprising multiple extracellular domains, the multiple extracellular domains can be different individual extracellular domains or multiple copies of the same extracellular domain, or a combination of the foregoing.

Extracellular Binding Domains

In some embodiments of any of the aspects, a NK CAR polypeptide as described herein comprises an extracellular binding domain. In some embodiments of any of the aspects, the extracellular binding domain recognizes and binds to a tumor antigen or to a cell surface marker expressed on a tumor cell. In some embodiments of any of the aspects, a CAR polypeptide as described herein comprises at least 1, at least 2, at least 3, at least 4, at least 5, at least 6, at least 7, at least 8, at least 9, or at least 10 extracellular binding domains. In some embodiments of any of the aspects, the CAR polypeptide or system comprises one extracellular binding domain. In some embodiments of any of the aspects, the CAR polypeptide comprises two extracellular binding domains. In embodiments comprising multiple extracellular binding domains, the multiple extracellular binding domains can be different individual extracellular binding domains or multiple copies of the same extracellular binding domains, or a combination of the foregoing.

In some embodiments of any of the aspects, the extracellular binding domain is an antibody, an antigen-binding fragment thereof, a F(ab) fragment, a F(ab′) fragment, a single chain variable fragment (scFv), or a single-domain antibody (sdAb). In some embodiments of any of the aspects, the extracellular binding domain is monovalent, bivalent, or multivalent. In some embodiments of any of the aspects, the extracellular binding domain comprises a human, humanized, or chimeric antibody construct. Additionally, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to a target.

Antibody reagents specific for the targets and/or markers described herein, e.g., tumor antigens are known in the art. For example, such reagents are readily commercially available. In some embodiments of any of the aspects, the extracellular binding domain can be an antibody reagent comprising one or more (e.g., one, two, three, four, five, or six) CDRs of any one of the antibodies described herein or known in the art. In some embodiments of any of the aspects, an antibody reagent specific for a target and/or marker described herein (e.g., that binds specifically to a tumor antigen as described herein) can be an antibody reagent comprising the six CDRs of any one of the antibodies described herein or known in the art. In some embodiments of any of the aspects, an antibody reagent specific for a target and/or marker described herein (e.g., that binds specifically to a tumor antigen as described herein) can be an antibody reagent comprising the three heavy chain CDRs of any one of the antibodies described herein or known in the art. In some embodiments of any of the aspects, an antibody reagent specific for a target and/or marker described herein (e.g., that binds specifically to a tumor antigen as described herein) can be an antibody reagent comprising the three light chain CDRs of any one of the antibodies described herein or known in the art. In some embodiments of any of the aspects, an antibody reagent specific for a target and/or marker described herein (e.g., that binds specifically to a tumor antigen as described herein) can be an antibody reagent comprising the VH and/or VL domains of any one of the antibodies described herein or known in the art. In some embodiments of any of the aspects, an antibody reagent specific for a target and/or marker described herein (e.g., that binds specifically to a tumor antigen as described herein) can be an antibody reagent comprising the VH and VL domains of any one of the antibodies described herein or known in the art. Such antibody reagents are specifically contemplated for use in the methods and/or compositions described herein.

As used herein, “antibody variable domain” refers to the portions of the light and heavy chains of antibody molecules that include amino acid sequences of Complementarity Determining Regions (CDRs; i.e., CDR1, CDR2, and CDR3), and Framework Regions (FRs). VH refers to the variable domain of the heavy chain. VL refers to the variable domain of the light chain. For the methods and compositions described herein, the amino acid positions assigned to CDRs and FRs may be defined according to Kabat (Sequences of Proteins of Immunological Interest (National Institutes of Health, Bethesda, Md., 1987 and 1991)). Amino acid numbering of antibodies or antigen binding fragments is also according to that of Kabat.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality. The light chain and heavy chain-derived sequences can be provided in N- to C-terminal order respectively, or in the opposite order.

As used herein, the term “linker” refers to a chemical or peptide structure that covalently joins two polypeptide moieties. For example, a VH domain and a VL domain of an antibody can be joined by a peptide linker to form a VH/VL single chain antigen binding domain (e.g., as an scFv). Lengths of linkers can be varied to modify the ability of linked domains to form, e.g., intramolecular or intermolecular dimers. For example, a diabody includes a short linker peptide between VH and VL domains, usually 5 amino acids, that will not permit the VH and VL domains to pair to form an antigen-binding domain; expression of two different VH-VL constructs with this short linker arrangement in a cell permits the VH domain of a first VH-VL polypeptide chain to dimerize with the VL domain of the second VH-VL polypeptide chain, and the corresponding VL domain of the first VH-VL polypeptide chain to dimerize with the VH domain of the second VH-VL polypeptide chain, thereby generating a bispecific construct. In contrast, when the VH and VL domains are separated by a longer peptide linker, most often 15-20 amino acids, the VH domain and the VL domain on the same polypeptide chain can dimerize to form an scFv. Non-limiting examples of such linkers are described further herein.

In some embodiments of any of the aspects, the extracellular binding domain comprises an scFv. In some embodiments of any of the aspects, the extracellular binding domain comprises an scFv that specifically binds to a tumor antigen. In some embodiments of any of the aspects, the extracellular binding domain of a CAR polypeptide as described herein specifically binds to any tumor antigen, including those described herein. In some embodiments of any of the aspects, the extracellular binding domain comprises any known antibody, an antigen-binding fragment thereof, a F(ab) fragment, a F(ab′) fragment, a single chain variable fragment (scFv), or a single-domain antibody (sdAb) that binds to a tumor antigen or epitope as described herein.

Non-limiting examples of tumor antigens that can be targeted include EphA2, HER2, AXL, GD2, Glypican-3, 5T4, 8H9, αvβ6 integrin, BCMA, B7-H3, B7-H6, CAIX, CA9, CD19, CD20, CD22, kappa light chain, CD30, CD33, CD38, CD44, CD44v6, CD44v7/8, CD70, CD123, CD138, CD171, CEA, CSPG4, EGFR, EGFRvIII, EGP2, EGP40, EPCAM, ERBB3, ERBB4, ErbB3/4, FAP, FAR, FBP, fetal AchR, Folate Receptor a, GD2, GD3, HLA-AI MAGE A1, HLA-A2, IL1 1Ra, IL13Ra2, KDR, Lambda, Lewis-Y, MCSP, Mesothelin, Muc1, Muc16, NCAM, NKG2D ligands, NY-ESO-1, PRAME, PSCA, PSC1, PSMA, RORI, SURVIVIN, TAG72, TEM1, TEM8, VEGRR2, carcinoembryonic antigen, HMW-MAA, and VEGF receptors. Other exemplary antigens that can be targeted are antigens that are present within the extracellular matrix of tumors, such as oncofetal variants of fibronectin, tenascin, or necrotic regions of tumors.

Additional tumor-selective molecules that can be targeted include any membrane protein or biomarker that is expressed or overexpressed in tumor cells including, but not limited to, integrins (e.g., integrin αvβ, α5β1), EGF Receptor Family (e.g., EGFR2, Erbb2/HER2/neu, Erbb3, Erbb4), proteoglycans (e.g., heparan sulfate proteoglycans), disialogangliosides (e.g., GD2, GD3), B7-H3 (aka CD276), cancer antigen 125 (CA-125), epithelial cell adhesion molecule (EpCAM), vascular endothelial growth factor receptors 1 and 2 (VEGFR-1, VEGFR-2), CD52, carcinoembryonic antigen (CEA), tumor associated glycoproteins (e.g., TAG-72), cluster of differentiation 19 (CD19), CD20, CD22, CD30, CD33, CD40, CD44, CD74, CD152, mucin 1 (MUC1), tumor necrosis factor receptors (e.g., TRAIL-R2), insulin-like growth factor receptors, folate receptor a, transmembrane glycoprotein NMB (GPNMB), C-C chemokine receptors (e.g., CCR4), prostate specific membrane antigen (PSMA), recepteur d'origine nantais (RON) receptor, cytotoxic T-lymphocyte antigen 4 (CTLA4), and other tumor specific receptors or antigens.

Non-limiting examples of tumor antigens include the following: Differentiation antigens such as MART-1/MelanA (MART-I), gp100 (Pmel 17), tyrosinase, TRP-1, TRP-2 and tumor-specific multilineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In some embodiments of any of the aspects, the tumor antigen is a tumor antigen described in International Application PCT/US2015/020606 or US Patent Applications US20170209492 or US20170335281, the contents of each of which are herein incorporated by reference in their entireties. In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (RORI); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abi) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCRI); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MARTI); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAXS); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIRI); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLECi2A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1). In some embodiments, the tumor antigen is GFRa4 (see e.g., Spinasanta, “The Endocrine Society's 97th Annual Meeting & Expo: Targeted Therapies in Medullary Thyroid Cancer” Mar. 13, 2015).

In some embodiments of any of the aspects, the extracellular binding domain comprises an antibody or extracellular binding domain (e.g., antigen-binding domain) thereof selected from the group consisting of: 20-(74)-(74) (milatuzumab; veltuzumab), 20-2b-2b, 3F8, 74-(20)-(20) (milatuzumab; veltuzumab), 8H9, A33, AB-16B5, abagovomab, abciximab, abituzumab, ABP 494 (cetuximab biosimilar), abrilumab, ABT-700, ABT-806, Actimab-A (actinium Ac-225 lintuzumab), actoxumab, adalimumab, ADC-1013, ADCT-301, ADCT-402, adecatumumab, aducanumab, afelimomab, AFM13, afutuzumab, AGEN1884, AGS15E, AGS-16C3F, AGS67E, alacizumab pegol, ALD518, alemtuzumab, alirocumab, altumomab pentetate, amatuximab, AMG 228, AMG 820, anatumomab mafenatox, anetumab ravtansine, anifrolumab, anrukinzumab, APN301, APN311, apolizumab, APX003/SIM-BD0801 (sevacizumab), APX005M, arcitumomab, ARX788, ascrinvacumab, aselizutnab, ASG-15ME, atezolizumab, atinumab, ATL101, atlizumab (also referred to as tocilizumab), atorolimumab, Avelumab, B-701, bapineuzumab, basiliximab, bavituximab, BAY1129980, BAY1187982, bectumomab, begelomab, belimumab, benralizumab, bertilimumab, besilesomab, BETALUTIN (177Lu-tetraxetan-tetulomab), bevaciztunab, BEVZ92 (bevacizumab biosimilar), bezlotoxumab, BGB-A317, BHQ880, BI 836880, BI-505, biciromab, bimagnunab, bimekizumab, bivatuzumab mertansine, BM-8962, blinatumomab, blosozumab, BMS-936559, BMS-986012, BMS-986016, BMS-986148, BMS-986178, BNC101, bococizumab, brentuximab vedotin, BREVAREX, briakinumab, brodalumab, brolucizumab, brontictuzumab, C2-2b-2b, canakinumab, cantuzumab mertansine, carttuzumab ravtansine, caplacizumab, capromab pendetide, carlumab, catumaxomab, CBR96-doxorubicin immunoconjugate, CBT124 (bevacizumab), CC-90002, CDX-014, CDX-1401, cedelizumab, certolizumab pegol, cetuximab, CGEN-15001T, CGEN-15022, CGEN-15029, CGEN-15049, CGEN-15052, CGEN-15092, Ch.14.18, citatuzumab bogatox, cixutumumab, clazakizumab, clenoliximab, clivatuzumab tetraxetan, CM-24, codrituzumab, coltuximab ravtansine, conatutnumab, concizumab, COTARA (iodine 1-131 derlotuximab biotin), cR6261, crenezumab, DA-3111 (trastuzumab biosimilar), dacetuzumab, daclizumab, dalotuzumab, dapirolizumab pegol, darattunumab, Daratumumab ENHANZE (daratumumab), DARLEUKIN, dectrekumab, demcizumab, denintuzumab mafodotin, denosumab, Depatuxizumab, Depatuxiztunab mafodotin, derlotuximab biotin, detumomab, DI-B4, dinutuximab, diridavtunab, DKN-01, DMOT4039A, dorlimomab aritox, drozittunab, DS-1123, DS-8895, duligotumab, dupilumab, dmvalumab, dusigitumab, ecromeximab, eculizumab, edobacomab, edrecolomab, efalizumab, efinumab, eldelumab, elgemtumab, elotuzumab, elsilimomab, emactuzumab, emibetuzumab, enavatuzumab, enfortumab vedotin, enlimomab pegol, enoblituzumab, enokizumab, enoticumab, ensituximab, epitumomab cituxetan, epratuzumab, erliztunab, ertumaxomab, etaracizumab, etrolizumab, evinacumab, evolocumab, exbivinunab, fanolesomab, faralimomab, farletuzumab, fasinumab, FBTA05, felviztunab, fezakinumab, FF-21101, FGFR2 Antibody-Drug Conjugate, FIBROMUN, ficlatuzumab, figitumumab, firivumab, flanvotumab, fletikumab, fontolizumab, foralumab, foravirumab, FPAI44, fresolimumab, FS102, fulranumab, futuximab, galiximab, ganitumab, gantenerumab, gavilimomab, gemtuzumab ozogamicin, Gerilimzumab, gevokizumab, girentuximab, glembatumumab vedotin, GNR-006, GNR-011, golimumab, gomiliximab, GSK2849330, GSK2857916, GSK3174998, GSK3359609, guselkumab, Hul4.18K322A MAb, hu3S193, Hu8F4, HuL2G7, HuMab-5B1, ibalizumab, ibritumomab tiuxetan, icrucumab, idarucizumab, IGN002, IGN523, igovomab, IMAB362, IMAB362 (claudiximab), imalumab, IMC-CS4, IMC-D II, imciromab, imgatuzumab, IMGN529, IMMU-IO2 (yttrium Y-90 epmtuzumab tetraxetan), IMM1J-114, ImmuTune IMP701 Antagonist Antibody, INCAGN1876, inclactunab, INCSHR1210, indatuximab ravtansine, indusaturnab vedotin, infliximab, inolimomab, inoturtunab ozogamicin, intetumumab, Ipafricept, IPH4102, ipilimumab, iratumumab, isatuximab, Istiratumab, itolizumab, ixekizumab, JNJ-56022473, JNJ-61610588, keliximab, KTN3379, L191L2/L19TNF, Labetuzumab, Labetuzumab Govitecan, LAG525, lambroliztunab, lampalizumab, L-DOS47, lebrikizumab, lemalesomab, lenzilumab, lerdelimumab, Leukotuximab, lexattunumab, libivirumab, lifastuzumab vedotin, ligelizumab, lilotomab satetraxetan, lintuzumab, lirilumab, LICZ145, lodelcizumab, lokivetmab, lorvoturtunab mertansine, lucatttmumab, lulizumab pegol, lumiliximab, lumretuzumab, LY3164530, mapatumumab, margetuximab, maslimomab, matuzumab, mavrilimumab, MB311, MCS-110, MED10562, MEDI-0639, MEDT0680, MEDI-3617, MEDI-551 (inebilizumab), MEDI-565, MED16469, mepolizumab, metelimumab, MGB453, MGD006/S80880, MGD007, MGD009, MGD011, milaturttmab, Milatuzumab-SN-38, minretumomab, mirvetuximab soravtansine, mitumomab, MK-4166, MM-111, MM-151, MM-302, mogamuliztunab, M0R202, MOR208, MORAb-066, morolimumab, motavizumab, moxetumomab pasudotox, muromonab-CD3, nacolomab tafenatox, namilumab, naptumomab estafenatox, narnatumab, natalizumab, nebacumab, necitumumab, nemolizumab, nerelimomab, nesvacumab, nimotuzumab, nivolumab, nofetumomab merpentan, NOV-10, obiltoxaximab, obinutuzumab, ocaratuzumab, ocrelizumab, odulimomab, ofatumumab, olaratttmab, olokizumab, ornalizumab, OMP-131R10, OMP-305B83, onartuzumab, ontuxizttmab, opicinumab, oportuzumab monatox, oregovomab, orticumab, otelixizumab, otlertuzumab, 0X002/MEN1309, oxelumab, ozanezumab, ozoralizumab, pagibaximab, palivizumab, panitumumab, pankomab, PankoMab-GEX, panobacumab, parsatuzumab, pascolizttmab, pasotuxizutnab, pateclizumab, patrittunab, PAT-SC1, PAT-SM6, pembrolizumab, pemtumomab, perakizumab, pertuzumab, pexelizumab, PF-05082566 (utomilumab), PF-06647263, PF-06671008, PF-06801591, pidilizumab, pinatuzumab vedotin, pintumomab, placulumab, polatuzumab vedotin, ponezumab, priliximab, pritoxaximab, prittunumab, PRO 140, PROXINIUM, PSMA ADC, quilizumab, racotumomab, radretumab, rafivirumab, ralpancizumab, ramucirumab, ranibizumab, raxibacumab, refanezumab, regavirumab, REGN1400, REGN2810/5AR439684, resliztunab, RFM-203, RG7356, RG7386, RG7802, RG7813, RG7841, RG7876, RG7888, RG7986, rilottunumab, rinucumab, rituximab, RM-1929, R07009789, robatumumab, roledumab, romosortunab, rontalizumab, rovelizumab, ruplizumab, sacituzumab govitecan, samalizumab, 5AR408701, 5AR566658, sarilumab, SAT 012, satumomab pendetide, SCT200, SCT400, SEA-CD40, secukinumab, seribantumab, setoxaximab, sevirumab, SGN-CD19A, SGN-CD19B, SGN-CD33A, SGN-CD70A, SGN-LIVIA, sibrotuzumab, sifalimumab, siltuximab, simtuzumab, sipliztunab, sirukumab, sofituzumab vedotin, solanezumab, solitomab, sonepcizumab, sontuzumab, stamulumab, sulesomab, suvizumab, 5YD985, SYM004 (futuximab and modotuximab), SYM015, TAB08, tabalumab, tacatuzumab tetraxetan, tadocizumab, talizumab, tanezumab, Tanibirumab, taplitumomab paptox, tarextumab, TB-403, tefibazumab, TELEUKIN, telimomab aritox, tenatumomab, teneliximab, teplizumab, teprotumumab, tesidolumab, tetulomab, TG-1303, TGN1412, Thorium-227-Epratuzumab Conjugate, ticilimumab, tigatuzumab, tildrakizumab, Tisotumab vedotin, TNX-650, tocilizumab, toralizumab, tosatoxumab, tosittunomab, tovetumab, tralokinumab, trastuzumab, trastuzumab emtansine, TRBS07, TRC105, tregalizumab, tremelimumab, trevogrtunab, TRPH 011, TRX518, TSR-042, TTI-200.7, tucotuzumab celmoleukin, tuvirumab, U3-1565, U3-1784, ublituximab, ulocuplumab, urelumab, urtoxazumab, ustekinumab, Vadastuximab Talirine, vandortuzumab vedotin, vantictumab, vanucizumab, vapaliximab, varlilumab, vatelizumab, VB6-845, vedolizumab, veltuzumab, vepalimomab, vesencumab, visilizumab, volociximab, vorseturtunab mafodotin, votumumab, YYB-101, zaluttunumab, zanolimumab, zatuximab, ziralimumab, and zolimomab aritox.

In some embodiments of any of the aspects, the extracellular binding domain comprises an anti-CD19 antibody, e.g., an anti-CD19 scFV. CD19 is also referred to interchangeably as B-Lymphocyte Surface Antigen B4 or T-Cell Surface Antigen Leu-12. Since CD19 is a marker of B cells, the protein has been used to diagnose cancers that arise from this type of cell—notably B cell lymphomas, acute lymphoblastic leukemia (ALL), and chronic lymphocytic leukemia (CLL). The majority of B cell malignancies express normal to high levels of CD19. Non-limiting examples of anti-CD19 antibodies include A3B1, FMC63, FMC63-28Z, SEQ ID NO: 35; see e.g., U.S. Pat. Nos. 10,221,245, 8,906,682, 10,421,810, 10,639,329, the contents of each of which are incorporated herein by reference in their entireties.

In some embodiments of any of the aspects, the extracellular binding domain comprises an anti-CD33 antibody, e.g., an anti-CD33 scFV. CD33 is also referred to interchangeably as Sialic Acid-Binding Ig-Like Lectin 3 (SIGLEC3) or Gp67. CD33 is a myeloid-specific sialic acid-binding receptor expressed on the blasts of approximately 90% of acute myelogenous leukemia (AML) patients and on AML stem cells. Non-limiting examples of anti-CD33 antibodies include SEQ ID NO: 36, gemtuzumab, vadastuximab, 2H12; see e.g., U.S. Pat. Nos. 7,557,189, 9,815,901, 10,556,951, 10,787,514, the contents of each of which are incorporated herein by reference in their entireties.

Depending on the desired antigen to be targeted, the CAR polypeptide described herein can be engineered to include the appropriate extracellular binding domain (e.g., antigen binding moiety) that is specific to the desired antigen target. For example, if CD19 is the desired antigen that is to be targeted, an antibody for CD19 or an extracellular binding domain (e.g., antigen-binding fragment) thereof can be used as the extracellular binding domain for incorporation into a CAR polypeptide as described herein. As another example, if CD33 is the desired antigen that is to be targeted, an antibody for CD33 or an extracellular binding domain (e.g., antigen-binding fragment) thereof can be used as the extracellular binding domain for incorporation into a CAR polypeptide as described herein.

In some embodiments of any of the aspects, the extracellular binding domain of a CAR polypeptide as described herein comprises one of SEQ ID NOs: 35-36, or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of SEQ ID NOs: 35-36, that maintains the same functions as one of SEQ ID NOs: 35-36 (e.g., tumor antigen binding).

In some embodiments of any of the aspects, the extracellular binding domain of a CAR polypeptide as described herein is encoded by a nucleic acid sequence comprising SEQ ID NOs: 33-34 or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 33-34 that maintains the same function or a codon-optimized version of one of SEQ ID NOs: 33-34.

SEQ ID NOs: 33 and 35 provide polynucleotide and amino acid sequences for an exemplary anti-CD19 scFv.

SEQ ID NO: 33
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGC
SEQ ID NO: 35
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSS

SEQ ID NOs: 34 and 36 provide polynucleotide and amino acid sequences for an exemplary anti-CD33 scFv.

SEQ ID NO: 34
GAAGTGCAGCTGGTGCAGAGCGGAGCAGAAGTGAAGAAGCCCGGAAGCAG
CGTGAAGGTGTCTTGCAAGGCCAGCGGCTACACCATCACCGACAGCAACA
TCCATTGGGTCCGGCAGGCTCCAGGACAGTCTCTGGAGTGGATCGGCTAC
ATCTACCCCTACAACGGCGGCACCGACTACAACCAGAAGTTCAAGAACCG
GGCCACCCTGACCGTGGATAACCCCACCAACACCGCCTACATGGAGCTGA
GCAGCCTGAGAAGCGAGGACACCGCCTTCTACTATTGCGTGAACGGCAAC
CCTTGGCTGGCCTATTGGGGACAGGGAACACTGGTGACCGTGTCCTCTGG
CGGCGGAGGCAGCGGCGGCGGAGGCTCCGGAGGCGGAGGATCCGACATCC
AGCTGACCCAGTCTCCTAGCACCCTGAGCGCTAGCGTGGGAGATAGAGTG
ACCATCACTTGCAGAGCCAGCGAGAGCCTGGACAACTACGGCATCCGGTT
CCTGACTTGGTTCCAGCAGAAACCCGGCAAGGCCCCTAAACTGCTGATGT
ACGCCGCCTCTAACCAGGGAAGCGGAGTGCCTAGCAGATTCAGCGGCAGC
GGAAGCGGAACCGAGTTCACCCTGACCATCAGCTCTCTGCAGCCAGACGA
CTTCGCCACCTACTACTGCCAGCAGACCAAGGAGGTGCCTTGGAGCTTCG
GCCAGGGAACCAAGGTGGAAGTGAAGCGGACAGTG
SEQ ID NO: 36
EVQLVQSGAEVKKPGSSVKVSCKASGYTITDSNIHWVRQAPGQSLEWIGY
IYPYNGGTDYNQKFKNRATLTVDNPTNTAYMELSSLRSEDTAFYYCVNGN
PWLAYWGQGTLVTVSSGGGGGGGGSGGGGSDIQLTQSPSTLSASVGDRVT
ITCRASESLDNYGIRFLTWFQQKPGKAPKLLMYAASNQGSGVPSRFSGSG
SGTEFTLTISSLQPDDFATYYCQQTKEVPWSFGQGTKVEVKRTV

In some embodiments of any of the aspects, the extracellular binding domain can target antigens involved in diseases other than cancer (e.g., infectious disease, etc.). As such, in some embodiments of any of the aspects, the extracellular binding domain can target a viral antigen. In some embodiments of any of the aspects, the extracellular binding domain can target a bacterial antigen. In some embodiments of any of the aspects, the viral target antigen is a viral surface antigen, such as a viral glycoprotein. Non-limiting examples of viral glycoproteins include: respiratory syncytial virus (RSV) F protein (e.g., site II of F protein); herpesvirus glycoprotein B (gB); Hepatitis B virus (HBV) surface antigen (HBsAg); human immunodeficiency virus (HIV) glycoprotein 120 (gp120) (e.g., gp120 CD4 binding site (CD4bs); gp120 third variable region (V3)); influenza virus hemagglutinin (HE; e.g., HE stalk; e.g., group 1 (G1) or group 2 (G2) HE stalk); and the like.

In some embodiments of any of the aspects, the bacterial target antigen is a bacterial surface antigen, such as a bacterial surface protein, lipid, or polysaccharide (i.e., exopolysaccharide). Non-limiting examples of bacterial surface antigens include: Staphylococcus species protein A; Staphylococcus species cell wall teichoic acid; lipopolysaccharide (LPS) (e.g., Pseudomonas aeruginosa serotype 011 LPS); alginate (e.g., Pseudomonas aeruginosa extracellular slime alginate); Clostridium difficile cell wall polysaccharide 2 (PsII); and the like. See e.g., Wagner et al. Current Opinion in Chemical Engineering 19 (2018): 131-141. In some embodiments of any of the aspects, the extracellular binding domain can target its antigen using a biotinylated antigen-specific molecule, e.g., instead of an scFv.

Signal Peptides

In some embodiments of any of the aspects, NK CAR polypeptides as described herein comprise a signal peptide, which can also be referred to as a signal, signal sequence, leader sequence, leader peptide, targeting signal, localization signal, localization sequence, or transit peptide. As used herein, the term “signal peptide” refers to an amino-terminal sequence of a polypeptide that designates newly synthesized proteins toward the secretory pathway. The signal peptide typically consists of 13 to 36 rather hydrophobic amino acids. Signal peptides have a common structure: a short, positively charged amino-terminal region (n-region); a central hydrophobic region (h-region); and a more polar carboxy-terminal region (c-region) containing the site that is cleaved by the signal peptidase. On the ER luminal side, the signal peptide is cleaved off by a signal peptidase. After successful folding of the nascent polypeptide by ER resident chaperones and foldases, the protein is further directed to exit the ER. This process may be supported by the presence of an N-terminal pro-sequence. Although most type I membrane-bound proteins have signal peptides, the majority of type II and multi-spanning membrane-bound proteins are targeted to the secretory pathway by their first transmembrane domain, which biochemically resembles a signal sequence except that it is not cleaved.

In some embodiments of any of the aspects, a NK CAR polypeptide as described herein comprises 1, 2, 3, 4, 5, or more signal peptides. In some embodiments of any of the aspects, the CAR polypeptide or system comprises one signal peptide, e.g., at the N-terminus of the polypeptide, preceding all other extracellular domains. In embodiments comprising multiple signal peptides, the multiple signal peptides can be different individual signal peptides or multiple copies of the same signal peptide, or a combination of the foregoing.

In some embodiments of any of the aspects, the signal peptide is a CD8a signal peptide. In some embodiments of any of the aspects, the signal peptide comprises SEQ ID NO: 38 or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of SEQ ID NO: 38 that maintains the same functions as SEQ ID NO: 38 (targeting of a transmembrane protein to the secretory pathway and the cell membrane).

In some embodiments, the signal peptide of a CAR polypeptide as described herein is encoded by a nucleic acid sequence comprising SEQ ID NO: 37 or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 37 that maintains the same function or a codon-optimized version of SEQ ID NO: 37.

SEQ ID NOs: 37 and 38 provide polynucleotide and amino acid sequences for an exemplary CD8 signal peptide.

SEQ ID NO: 37
ATGGCCTTACCAGTGACCGCCTTGCTCCTGCCGCTGGCCTTGCTGCTCCA
CGCCGCCAGGCCG
SEQ ID NO: 38
MALPVTALLLPLALLLHAARP

Detectable Markers

In several aspects, described herein are NK CAR polypeptides comprising at least one detectable marker. As used herein, the term “detectable marker” refers to a moiety that, when attached to the CAR polypeptide, confers detectability upon that polypeptide or another molecule to which the polypeptide binds. In some embodiments of any of the aspects, the CAR polypeptide comprises 1, 2, 3, 4, 5, or more detectable markers. In some embodiments of any of the aspects, the CAR polypeptide or system comprises one detectable marker. In embodiments comprising multiple detectable markers, the multiple detectable markers can be different individual detectable markers or multiple copies of the same detectable markers, or a combination of the foregoing.

In some embodiments of any of the aspects, the detectable marker comprises an affinity tag. Non-limiting examples of affinity tags include Strep-tags, chitin binding proteins (CBP), maltose binding proteins (MBP), glutathione-S-transferase (GST), FLAG-tags, HA-tags, Myc-tags, poly(His)-tags as well as derivatives thereof. In some embodiments of any of the aspects, the detectable marker is 3×FLAG (i.e., the FLAG motif, DYKDDDDK, SEQ ID NO: 131, repeated three times).

In some embodiments of any of the aspects, the detectable marker of a CAR polypeptide as described herein comprises one of SEQ ID NOs: 40, 122, 131, or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of SEQ ID NOs: 40, 122, or 131, that maintains the same functions as one of SEQ ID NOs: 40, 122, or 131 (e.g., detection of the CAR polypeptide).

In some embodiments of any of the aspects, the detectable marker of a CAR polypeptide as described herein is encoded by a nucleic acid sequence comprising one of SEQ ID NOs: 39, 121, or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 39 or 121 that maintains the same function or a codon-optimized version of one of SEQ ID NOs: 39 or 121.

SEQ ID NOs: 39, 40, 121, and 122 provide polynucleotide and amino acid sequences for an exemplary FLAG tag (e.g., 3×Flag).

SEQ ID NO: 39
GATTACAAGGATGACGACGATAAGGACTATAAGGACGACGACGATAAGGA
TTATAAAGACGATGACGACAAA
SEQ ID NO: 40
DYKDDDDKDYKDDDDKDYKDDDDK
SEQ ID NO: 121
GATTACAAGGATGACGACGATAAGGACTATAAGGACGACGACGATAAGGA
TTATAAAGACGATGACGACAAAGGT
SEQ ID NO: 122
DYKDDDDKDYKDDDDKDYKDDDDKG

In some embodiments of any of the aspects, fluorescent moieties can be used as detectable markers, but detectable markers also include, for example, isotopes, fluorescent proteins and peptides, enzymes, components of a specific binding pair, chromophores, affinity tags as described herein or known in the art, antibodies, colloidal metals (i.e. gold) and quantum dots. Detectable markers can be either directly or indirectly detectable. Directly detectable markers do not require additional reagents or substrates in order to generate detectable signal. Examples include isotopes and fluorophores. Indirectly detectable markers require the presence or action of one or more co-factors or substrates. Examples include enzymes such as β-galactosidase which is detectable by generation of colored reaction products upon cleavage of substrates such as the chromogen X-gal (5-bromo-4-chloro-3-indoyl-β-D-galactopyranoside), horseradish peroxidase which is detectable by generation of a colored reaction product in the presence of the substrate diaminobenzidine and alkaline phosphatase which is detectable by generation of colored reaction product in the presence of nitroblue tetrazolium and 5-bromo-4-chloro-3-indolyl phosphate.

In some embodiments of any of the aspects, the detectable marker can be located anywhere within a CAR polypeptide as described herein. In one embodiment, the detectable marker is located between any domain of a CAR polypeptide as described herein, but is not found within a functional domain and does not disrupt the function of a domain. In some embodiments of any of the aspects, the detectable marker is located adjacent to and N-terminal of the extracellular binding domain. Such a marker can be used to detect the expression of the CAR polypeptide, including the cell surface expression. In some embodiments of any of the aspects, the detectable marker is located adjacent to one of the extracellular domains. In some embodiments of any of the aspects, the detectable marker is located adjacent to the transmembrane domain. In some embodiments of any of the aspects, the detectable marker is located adjacent to one of the intracellular domains.

In some embodiments of any of the aspects, CAR polypeptides as described herein, especially those that are administered to a subject or those that are part of a pharmaceutical composition, do not comprise detectable markers that are immunogenic. In some embodiments of any of the aspects, CAR polypeptides as described herein do not comprise GFP, mCherry, HA 1, or any other immunogenic markers.

Linker Domains

In several aspects, described herein are NK CAR polypeptides comprising at least one linker domain. As used herein “linker domain” (used interchangeably with “peptide linker” or simply “linker”) refers to an oligo- or polypeptide region from about 2 to 100 amino acids in length, which links together any of the sequences of the domains as described herein. In some embodiment, linkers can include or be composed of flexible residues such as glycine and serine so that the adjacent protein domains are free to move relative to one another. Longer linkers may be used when it is desirable to ensure that two adjacent domains do not sterically interfere with one another. Linkers can be cleavable (e.g., designed to comprise a substrate for a particular protease enzyme) or non-cleavable.

In some embodiments of any of the aspects, an NK CAR polypeptide as described herein comprises 1, 2, 3, 4, 5, or more linker domains(s). In some embodiments of any of the aspects, the NK CAR polypeptide comprises one linker domain. In embodiments comprising multiple linker domains, the multiple linker domains can be different individual linker domains or multiple copies of the same linker domain, or a combination of the foregoing. In some embodiments of any of the aspects, the linker peptide can be positioned between any two domains as described herein: e.g., between the detectable marker and the spacer domain, and/or between a VH/VL single chain antigen binding domain (e.g., as in an scFv). In some embodiments of any of the aspects, the linker domain is distal to (i.e., farther from the transmembrane domain compared to) the extracellular binding domain. In some embodiments of any of the aspects, the linker domain is proximal to (i.e., closer to the transmembrane domain compared to) the signal peptide and/or detectable marker.

A linker domain can comprise 1 amino acid or more, 5 amino acids or more, 10 amino acids or more, 15 amino acids or more, 20 amino acids or more, 25 amino acids or more, 30 amino acids or more, 35 amino acids or more, 40 amino acids or more, 45 amino acids or more, 50 amino acids or more and beyond. Conversely, a linker domain can comprise less than 50 amino acids, less than 45 amino acids, less than 40 amino acids, less than 35 amino acids, less than 30 amino acids, less than 30 amino acids, less than 25 amino acids, less than 20 amino acids, less than 15 amino acids or less than 10 amino acids.

In some embodiments of the various aspects described herein, the linker domain comprises from about 5 amino acids to about 50 amino acids. For example, the linker domain can comprise from about 5 amino acids to about 45 amino acids, from about 5 amino acids to about 40 amino acids, from about 5 amino acids to about 35 amino acids, from about 10 amino acids to 30 amino acids, or from about 15 amino acids to about 25 amino acids.

Exemplary linker domains include those that consist of glycine and serine residues, the so-called Gly-Ser polypeptide linkers. As used herein, the term “Gly-Ser polypeptide linker” refers to a peptide that consists of glycine and serine residues. In some embodiments of the various aspects described herein, the linker domain comprises the amino acid sequence (GlyₓSer)ₙ, where x is 2, 3, 4 or 5, and n is 1, 2, 3, 4, 5, 6, 7, 8, 9 or 10, e.g., SEQ ID NOs: 42, 120, 139-176 (see e.g., Table 9).

TABLE 9
Exemplary Linker Sequences

| SEQ ID NO | Sequence | SEQ ID NO | Sequence | SEQ ID NO | Sequence | SEQ ID NO | Sequence |
|---|---|---|---|---|---|---|---|
| 139 | (GGS)₁ | 120 | (GGGS)₁ | 42 | (GGGGS)₁ | 167 | (GGGGGS)₁ |
| 140 | (GGS)₂ | 149 | (GGGS)₂ | 158 | (GGGGS)₂ | 168 | (GGGGGS)₂ |
| 141 | (GGS)₃ | 150 | (GGGS)₃ | 159 | (GGGGS)₃ | 169 | (GGGGGS)₃ |
| 142 | (GGS)₄ | 151 | (GGGS)₄ | 160 | (GGGGS)₄ | 170 | (GGGGGS)₄ |
| 143 | (GGS)₅ | 152 | (GGGS)₅ | 161 | (GGGGS)₅ | 171 | (GGGGGS)₅ |
| 144 | (GGS)₆ | 153 | (GGGS)₆ | 162 | (GGGGS)₆ | 172 | (GGGGGS)₆ |
| 145 | (GGS)₇ | 154 | (GGGS)₇ | 163 | (GGGGS)₇ | 173 | (GGGGGS)₇ |
| 146 | (GGS)₈ | 155 | (GGGS)₈ | 164 | (GGGGS)₈ | 174 | (GGGGGS)₈ |
| 147 | (GGS)₉ | 156 | (GGGS)₉ | 165 | (GGGGS)₉ | 175 | (GGGGGS)₉ |
| 148 | (GGS)₁₀ | 157 | (GGGS)₁₀ | 166 | (GGGGS)₁₀ | 176 | (GGGGGS)₁₀ |

In some embodiments of any of the aspects, the linker domain of a CAR polypeptide as described herein comprises one of SEQ ID NOs: 42, 120, 139-176, or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of SEQ ID NOs: 42, 120, 139-176, that maintains the same functions as one of SEQ ID NOs: 42, 120, 139-176 (e.g., flexible linker between domains of the CAR polypeptide).

In some embodiments of any of the aspects, the detectable marker of a CAR polypeptide as described herein is encoded by a nucleic acid sequence comprising one of SEQ ID NOs: 41, 119, or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to one of SEQ ID NOs: 41 or 119 that maintains the same function or a codon-optimized version of one of SEQ ID NOs: 41 or 119.

SEQ ID NOs: 41, 42, 119, and 120 provide polynucleotide and amino acid sequence for an exemplary linker. SEQ ID NO: 41 GGTGGAGGCGGTTCG; SEQ ID NO: 42 GGGGS; SEQ ID NO: 119 GGAGGCGGTTCG; SEQ ID NO: 120 GGGS.

Spacer Domains

In some embodiments of any of the aspects, a NK CAR polypeptide as described herein comprises a spacer domain. In some embodiments of any of the aspects, the spacer domain is located between the extracellular binding domain and the transmembrane domain. In some embodiments of any of the aspects, the spacer domain is C-terminal of the extracellular binding domain and N-terminal of the transmembrane domain. In some embodiments of any of the aspects, the spacer domain is N-terminal of the extracellular binding domain and C-terminal of the transmembrane domain. In some embodiments of any of the aspects, the spacer domain comprises a hinge domain. In some embodiments of any of the aspects, a CAR polypeptide as described herein comprises 1, 2, 3, 4, 5, or more hinge domains. In some embodiments of any of the aspects, the CAR polypeptide or system comprises one hinge domain. In embodiments comprising multiple hinge domains, the multiple hinge domains can be different individual hinge domains or multiple copies of the same hinge domain, or a combination of the foregoing.

In some embodiments of any of the aspects, the hinge domain comprises an immunoglobulin G (IgG)-based hinge or a derivative of the CD8a extracellular domain. Incorporation of a hinge domain has been shown to improve the expansion of chimeric antigen receptor T cells and to increase the antitumor efficacy of CAR T cells (see e.g., Qin et al., Journal of Hematology & Oncology volume 10, Article number: 68 (2017); Stoiber et al., Cells. 2019 May; 8(5): 472). Hinge domains can be derived from IgG subclasses (such as IgGI and IgG4), IgD and CD8 domains, of which IgGI has been most extensively used. A hinge domain preferably provides the following four aspects: (1) reduced binding affinity to the Fcγ receptor, thereby reducing or eliminating off-target activation; (2) enhanced flexibility for the extracellular binding domain (e.g., scFv), thereby relieving the spatial constraints between tumor antigens and CARs, in turn promoting synapse formation between the NK CAR cells and target cells; for example, to overcome steric hindrance in a MUC1-specific CAR, a flexible and elongated hinge of the IgD isotype can be inserted; (3) reduced distance between an extracellular binding domain (e.g., scFv) and the target antigen or epitope, for example, an anti-CD22 CAR needs a hinge domain to exert optimal cytotoxicity; and (4) facilitated detection of CAR expression using anti-Fc reagents. In some embodiments of any of the aspects, the hinge domain promotes CAR dimerization.

In some embodiments of any of the aspects, the hinge domain comprises a CD8 hinge domain. In some embodiments of any of the aspects, the hinge domain comprises a CD8a hinge domain. In some embodiments of any of the aspects, the hinge domain of a CAR polypeptide as described herein comprises SEQ ID NO: 44, or an amino acid sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of SEQ ID NO: 44, that maintains the same functions as SEQ ID NO: 44 (e.g., displaying the extracellular binding region).

In some embodiments of any of the aspects, the hinge domain of a CAR polypeptide as described herein is encoded by a nucleic acid sequence comprising SEQ ID NO: 43 or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to SEQ ID NO: 43 that maintains the same function or a codon-optimized version of SEQ ID NO: 43.

SEQ ID NOs: 43 and 44 provide polynucleotide and amino acid sequences for an exemplary CD8a hinge domain (e.g., residues 138-182 of CD8a; see e.g., SEQ ID NO: 138 for an exemplary full-length CD8a polypeptide sequence).

SEQ ID NO: 43
ACCACAACCCCTGCCCCCAGACCTCCCACACCCGCCCCTACCATCGCTAG
CCAGCCTCTGAGCCTGAGGCCCGAGGCTTGCAGACCTGCTGCTGGAGGCG
CCGTGCACACCAGAGGACTGGATTTCGCCTGCGAC
SEQ ID NO: 44
TTTPAPRPPTPAPTIASQPLSLRPEACRPAAGGAVHTRGLDFACD

### Exemplary Polypeptides

In multiple aspects described herein are NK CAR polypeptides comprising any combination of the domains as described herein. In one aspect, described herein is a chimeric antigen receptor (CAR) polypeptide for expression in natural killer (NK) cells comprising: (a) an extracellular binding domain; (b) a transmembrane domain; and (c) an intracellular domain comprising at least one of the following: (i) an intracellular signaling domain from an NK cell receptor; (ii) an intracellular signaling domain from an NK cell membrane-bound signaling adaptor protein; and/or (iii) an intracellular co-stimulatory domain from a co-stimulatory receptor.

In one aspect, described herein is a chimeric antigen receptor (CAR) polypeptide (e.g., a Group I NK CAR polypeptide) for expression in natural killer (NK) cells comprising: (a) an extracellular binding domain; (b) a transmembrane domain; and (c) an intracellular domain comprising at least one of the following: (i) an intracellular signaling domain from a NK cell membrane-bound signaling adaptor protein; and/or (ii) an intracellular co-stimulatory domain from a co-stimulatory receptor. In one aspect, described herein is a chimeric antigen receptor (CAR) polypeptide (e.g., a Group II-IV NK CAR polypeptide) for expression in natural killer (NK) cells comprising: (a) an extracellular binding domain; (b) a transmembrane domain; and (c) an intracellular domain comprising at least one signaling domain from a NK cell receptor.

In some embodiments of any of the aspects, the polypeptide comprises from N-terminus to C-terminus: (a) the extracellular binding domain; (b) the transmembrane domain; and (c) the intracellular domain; such a polypeptide can also be referred to as a type I transmembrane protein. In some embodiments of any of the aspects, a CAR that is a type I transmembrane protein firther comprises an N-terminal signal peptide. Non-limiting examples of such type I transmembrane protein CARs include CC002, CC003, CC004, CC005, CC007, CC008, CC013, CC016, CC017, CC018, CC024, CC026, CC027, CC029, CC030, CC032, CC033, CC034, CC035, CC037, CC038, CC039, CC103, CC104, CC108, CC118, CC124, CC130, CC135, which correspond to SEQ ID NOs: 80-90, 92-93, 95-96, 98-101, 103-110, and 112-113, respectively.

In some embodiments of any of the aspects, the polypeptide comprises from C-terminus to N-terminus: (a) the extracellular binding domain; (b) the transmembrane domain; and (c) the intracellular domain; such a polypeptide can also be referred to as a type II transmembrane protein. In some embodiments of any of the aspects, a CAR that is a type II transmembrane protein does not necessarily comprise an N-terminal signal peptide. Non-limiting examples of such type II transmembrane protein CARs include CC025, CC028, CC031, CC036, CC125, CC136, which correspond to SEQ ID NOs: 91, 94, 97, 102, 111, 114, respectively.

In some embodiments of any of the aspects, the polypeptide is selected from the group consisting of Group Ic (e.g., CC005, CC007, CC008, or CC108), Group IIa (e.g., CC024, CC025, CC124, CC125), or Group IV (e.g., CC027, CC030, CC034, CC038, or CC130). In some embodiments of any of the aspects, the polypeptide is selected from the group consisting of Group Ic (e.g., CC005, CC007, CC008, or CC108), Group IIa (e.g., CC024, CC025, CC124, CC125), or Group IVd (e.g., CC030 or CC130). In some embodiments of any of the aspects, the polypeptide is selected from the group consisting of Group Ic (e.g., comprising cytokines, including C007, CC008, or CC108), Group IIa (e.g., CC024, CC025, CC124, CC125), or Group IVd (e.g., CC030 or CC130).

In some embodiments of any of the aspects, a CAR polypeptide comprises one of SEQ ID NOs: 80-114 (see e.g., Table 6), or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of 80-114 maintains the same functions (e.g., antigen-binding and intracellular signaling). Table 6 shows exemplary CAR polypeptides and the specific order of their extracellular, transmembrane, and intracellular domains.

TABLE 6
Construct and Domain Amino Acid SEQ ID NOs (“TM” indicates transmembrane domain)

| | | SEQ ID NOs of domains, from 5′ to 3′ | | | | | | | | | | | | |
|---|---|---|---|---|---|---|---|---|---|---|---|---|---|---|
| | Construct | (except where indicated in next column) | | | | | | | | | | | | Domains in 3′ |
| | SEQ ID NO | Extracellular | | | | | | Intracellular | | | | | | to 5′ order |
| Construct | (amino acid) | Domains | | | | | TM | Domains | | | | | | (compare to) |
| CC002 | 80 | 38 | 40 | 42 | 35 | 44 | 32 | 15 | | | | | | |
| CC003 | 81 | 38 | | 42 | 35 | 44 | 32 | 15 | 11 | | | | | |
| CC004 | 82 | 38 | 40 | 42 | 35 | 44 | 32 | 15 | 11 | | | | | |
| CC005 | 83 | 38 | 40 | 42 | 35 | 44 | 32 | 15 | 12 | | | | | |
| CC007 | 84 | 38 | 40 | 42 | 35 | 44 | 32 | 15 | 12 | 19 | 23 | | | |
| CC008 | 85 | 38 | 40 | 42 | 35 | 44 | 32 | 15 | 12 | 19 | 23 | 20 | 24 | |
| CC013 | 86 | 38 | 40 | 42 | 35 | 44 | 32 | 15 | 16 | 5 | | | | |
| CC016 | 87 | 38 | 40 | 42 | 35 | 44 | 32 | 16 | 13 | 5 | | | | |
| CC017 | 88 | 38 | 40 | 42 | 35 | 44 | 32 | 13 | 16 | 12 | | | | |
| CC018 | 89 | 38 | 40 | 42 | 35 | 44 | 32 | 16 | 13 | 12 | | | | |
| CC024 | 90 | 38 | 40 | 42 | 35 | 44 | 29 | 7 | | | | | | |
| CC025 | 91 | | 40 | 42 | 35 | 44 | 29 | 7 | | | | | | 3′ to 5′ (CC024) |
| CC026 | 92 | 38 | 40 | 42 | 35 | 44 | 30 | 9 | | | | | | |
| CC027 | 93 | 38 | 40 | 42 | 35 | 44 | 31 | 7 | | | | | | |
| CC028 | 94 | | 40 | 42 | 35 | 44 | 29 | 10 | | | | | | 3′ to 5′ (CC039) |
| CC029 | 95 | 38 | 40 | 42 | 35 | 44 | 30 | 10 | | | | | | |
| CC030 | 96 | 38 | 40 | 42 | 35 | 44 | 31 | 8 | | | | | | |
| CC031 | 97 | | 40 | 42 | 35 | 44 | 29 | 9 | | | | | | 3′ to 5′ (CC037) |
| CC032 | 98 | 38 | 40 | 42 | 35 | 44 | 30 | 7 | | | | | | |
| CC033 | 99 | 38 | 40 | 42 | 35 | 44 | 30 | 8 | | | | | | |
| CC034 | 100 | 38 | 40 | 42 | 35 | 44 | 31 | 9 | | | | | | |
| CC035 | 101 | 38 | 40 | 42 | 35 | 44 | 29 | 8 | | | | | | |
| CC036 | 102 | | 40 | 42 | 35 | 44 | 29 | 8 | | | | | | 3′ to 5′ (CC035) |
| CC037 | 103 | 38 | 40 | 42 | 35 | 44 | 29 | 9 | | | | | | |
| CC038 | 104 | 38 | 40 | 42 | 35 | 44 | 31 | 10 | | | | | | |
| CC039 | 105 | 38 | 40 | 42 | 35 | 44 | 29 | 10 | | | | | | |
| CC103 | 106 | 38 | | 42 | 36 | 44 | 32 | 15 | 11 | | | | | |
| CC104 | 107 | 38 | 40 | 42 | 36 | 44 | 32 | 15 | 11 | | | | | |
| CC108 | 108 | 38 | 40 | 42 | 36 | 44 | 32 | 15 | 12 | 19 | 23 | 20 | 24 | |
| CC118 | 109 | 38 | 40 | 42 | 36 | 44 | 32 | 16 | 15 | 12 | | | | |
| CC124 | 110 | 38 | 40 | 42 | 36 | 44 | 29 | 7 | | | | | | |
| CC125 | 111 | | 40 | 42 | 36 | 44 | 29 | 7 | | | | | | 3′ to 5′ (CC124) |
| CC130 | 112 | 38 | 40 | 42 | 36 | 44 | 31 | 8 | | | | | | |
| CC135 | 113 | 38 | 40 | 42 | 36 | 44 | 29 | 8 | | | | | | |
| CC136 | 114 | 38 | 40 | 42 | 36 | 44 | 29 | 8 | | | | | | 3′ to 5′ (CC135) |

Group I NK CAR Polypeptides

In one aspect, described herein is a Group I NK CAR polypeptide comprising: (a) an extracellular binding domain; (b) a transmembrane domain; and (c) an intracellular domain comprising at least one of the following: (i) an intracellular signaling domain from an NK cell membrane-bound signaling adaptor protein; and/or (ii) an intracellular co-stimulatory domain from a co-stimulatory receptor. In some embodiments of any of the aspects, the transmembrane domain comprises the transmembrane domain of CD8a.

In one aspect, described herein is a Group Ia NK CAR polypeptide comprising: (a) an extracellular binding domain; (b) a transmembrane domain; and (c) an intracellular domain comprising an intracellular co-stimulatory domain from a co-stimulatory receptor. In some embodiments of any of the aspects, the transmembrane domain comprises the transmembrane domain of CD8a. In some embodiments of any of the aspects, the co-stimulatory receptor is 4-1BB. In some embodiments of any of the aspects, the Group Ia NK CAR polypeptide is used as a control or a reference.

In one aspect, described herein is a Group 1b NK CAR polypeptide comprising: (a) an extracellular binding domain; (b) a transmembrane domain; and (c) an intracellular domain comprising: (i) an intracellular signaling domain from a NK cell membrane-bound signaling adaptor protein; and (ii) an intracellular co-stimulatory domain from a co-stimulatory receptor. In some embodiments of any of the aspects, the transmembrane domain comprises the transmembrane domain of CD8a. In some embodiments of any of the aspects, the co-stimulatory receptor is 4-1BB. In some embodiments of any of the aspects, the adaptor protein is CD3-zeta. In some embodiments of any of the aspects, the Group 1b NK CAR polypeptide is used as a control or a reference.

In one aspect, described herein is a Group Ic NK CAR polypeptide comprising: (a) an extracellular binding domain; (b) a transmembrane domain; and (c) an intracellular domain comprising: (i) an intracellular signaling domain from a NK cell membrane-bound signaling adaptor protein; and (ii) an intracellular co-stimulatory domain from a co-stimulatory receptor. In some embodiments of any of the aspects, the transmembrane domain comprises the transmembrane domain of CD8a. In some embodiments of any of the aspects, the co-stimulatory receptor is 4-1BB. In some embodiments of any of the aspects the adaptor protein is FcεRI. In some embodiments of any of the aspects, the Group Ic NK CAR polypeptide further comprises at least one cytokine (e.g., IL-15 and/or IL-21) and at least one self-cleaving peptide.

In one aspect, described herein is a Group Id NK CAR polypeptide comprising: (a) an extracellular binding domain; (b) a transmembrane domain; and (c) an intracellular domain comprising: (i) an intracellular signaling domain from a NK cell membrane-bound signaling adaptor protein; and (ii) two intracellular co-stimulatory domains from co-stimulatory receptors. In some embodiments of any of the aspects, the transmembrane domain comprises the transmembrane domain of CD8a. In some embodiments of any of the aspects the two co-stimulatory receptors are 4-1BB and IL2RB. In some embodiments of any of the aspects the adaptor protein is CD3-zeta.

In one aspect, described herein is a Group Ie NK CAR polypeptide comprising: (a) an extracellular binding domain; (b) a transmembrane domain; and (c) an intracellular domain comprising: (i) an intracellular signaling domain from a NK cell membrane-bound signaling adaptor protein; and (ii) two intracellular co-stimulatory domains from co-stimulatory receptors. In some embodiments of any of the aspects, the transmembrane domain comprises the transmembrane domain of CD8a. In some embodiments of any of the aspects the two co-stimulatory receptors are 4-1BB and IL2RB. In some embodiments of any of the aspects the adaptor protein is FcεRI.

In some embodiments of any of the aspects, a Group I NK CAR polypeptide comprises one of SEQ ID NOs: 80-89 or 106-109, or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of 80-89 or 106-109 maintains the same functions (e.g., antigen-binding and intracellular signaling).

In some embodiments of any of the aspects, a Group I NK CAR polynucleotide comprises one of SEQ ID NOs: 45-54 or 71-74, or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of SEQ ID NOs: 45-54 or 71-74 that as a polypeptide maintains the same functions, or a codon-optimized version of one of SEQ ID NOs: 45-54 or 71-74.

Anti-CD19 Group I

Non-limiting examples of Group I NK CARs that are specific to CD19 include CC002, CC003, CC004, CC005, CC007, CC008, CC013, CC016, CC017, or CC018, which correspond to polynucleotide SEQ ID NOs: 45-54 and polypeptide SEQ ID NOs: 80-89, respectively. It should be understood that other NK CARs can be generated by switching the CD-19-binding domain for a different tumor antigen- or cell-surface protein-binding domain.

The CC002 construct comprises the following domains from N terminus to C terminus: CD8 signal peptide—3*Flag—linker (e.g., GGGGS, SEQ ID NO: 42)—anti-CD19 scFv—CD8a hinge domain—CD8 transmembrane domain—4-1BB. See e.g., SEQ ID NO: 45 or SEQ ID NO: 80.

SEQ ID NO: 45, CC002
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACATCTACATCTGGGCCCCTCTGGCTGGCACATGTGGCGTG
CTGCTGCTCTCCCTGGTGATCACCCTGTACTGCAAAAGGGGCAGAAAGAA
ACTGCTCTACATCTTCAAGCAGCCCTTCATGAGGCCCGTCCAAACCACCC
AGGAGGAGGACGGATGCAGCTGTAGATTCCCCGAAGAGGAGGAAGGCGGC
TGCGAGCTGTAA
SEQ ID NO: 80, CC002
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDIYIWAPLAGTCGV
LLLSLVITLYC

The CC003 construct comprises the following domains from N terminus to C terminus: CD8 signal peptide—linker (e.g., GGGGS, SEQ ID NO: 42)—anti-CD19 scFv—CD8a hinge domain—CD8 transmembrane domain—4-1BB—CD3z. See e.g., SEQ ID NO: 46 or SEQ ID NO: 81.

SEQ ID NO: 46, CC003
ATGGCCTTACCAGTGACCGCCTTGCTCCTGCCGCTGGCCTTGCTGCTCCA
CGCCGCCAGGCCGGGTGGAGGCGGTTCGGACATCCAGATGACCCAGACCA
CCAGCAGCCTGAGCGCTAGCCTGGGCGATAGAGTGACCATCAGCTGCAGA
GCCAGCCAGGACATCAGCAAGTACCTGAACTGGTACCAGCAGAAGCCCGA
CGGCACAGTGAAACTGCTGATCTACCACACCTCCAGACTGCATAGCGGCG
TGCCTAGCAGGTTTTCCGGCAGCGGCAGCGGAACCGACTACTCCCTGACC
ATCAGCAACCTGGAGCAGGAGGACATCGCTACCTACTTTTGTCAGCAGGG
CAACACCCTGCCTTACACCTTTGGCGGCGGAACAAAGCTGGAGATTACCG
GCGGCGGAGGCAGCGGCGGCGGAGGCTCCGGAGGCGGAGGATCCGAGGTG
AAGCTGCAGGAAAGCGGACCTGGACTGGTCGCCCCTAGCCAGAGCCTGAG
CGTGACCTGTACCGTGAGCGGAGTGAGCCTGCCCGATTACGGCGTGTCCT
GGATCAGGCAGCCCCCCAGGAAGGGACTGGAATGGCTGGGCGTCATCTGG
GGCTCCGAGACAACCTACTACAACAGCGCCCTCAAGTCCAGGCTCACCAT
TATCAAGGACAATAGCAAGAGCCAGGTGTTCCTGAAGATGAACAGCCTGC
AGACCGACGACACAGCCATTTATTACTGCGCCAAGCACTATTATTACGGC
GGCAGCTACGCCATGGACTATTGGGGCCAGGGAACAAGCGTGACCGTCAG
CAGCACCACAACCCCTGCCCCCAGACCTCCCACACCCGCCCCTACCATCG
CTAGCCAGCCTCTGAGCCTGAGGCCCGAGGCTTGCAGACCTGCTGCTGGA
GGCGCCGTGCACACCAGAGGACTGGATTTCGCCTGCGACATCTACATCTG
GGCCCCTCTGGCTGGCACATGTGGCGTGCTGCTGCTCTCCCTGGTGATCA
CCCTGTACTGCAAAAGGGGCAGAAAGAAACTGCTCTACATCTTCAAGCAG
CCCTTCATGAGGCCCGTCCAAACCACCCAGGAGGAGGACGGATGCAGCTG
TAGATTCCCCGAAGAGGAGGAAGGCGGCTGCGAGCTGAGAGTCAAGTTCT
CCAGGTCCGCCGACGCCCCCGCGTACAAGCAGGGCCAGAACCAGCTCTAT
AACGAGCTCAATCTAGGACGAAGAGAGGAGTACGATGTTTTGGACAAGAG
ACGTGGCCGGGACCCTGAGATGGGGGGAAAGCCGAGAAGGAAGAACCCTC
AGGAAGGCCTGTACAATGAACTGCAGAAAGATAAGATGGCGGAGGCCTAC
AGTGAGATTGGGATGAAAGGCGAGCGCCGGAGGGGCAAGGGGCACGATGG
CCTTTACCAGGGTCTCAGTACAGCCACCAAGGACACCTACGACGCCCTTC
ACATGCAGGCCCTGCCCCCTCGCTAA
SEQ ID NO: 81, CC003
MALPVTALLLPLALLLHAARPGGGGSDIQMTQTTSSLSASLGDRVTISCR
ASQDISKYLNWYQQKPDGTVKLLIYHTSRLHSGVPSRFSGSGSGTDYSLT
ISNLEQEDIATYFCQQGNTLPYTFGGGTKLEITGGGGSGGGGSGGGGSEV
KLQESGPGLVAPSQSLSVTCTVSGVSLPDYGVSWIRQPPRKGLEWLGVIW
GSETTYYNSALKSRLTIIKDNSKSQVFLKMNSLQTDDTAIYYCAKHYYYG
GSYAMDYWGQGTSVTVSSTTTPAPRPPTPAPTIASQPLSLRPEACRPAAG
GAVHTRGLDFACDIYIWAPLAGTCGVLLLSLVITLYCKRGRKKLLYIFKQ
PFMRPVQTTQEEDGCSCRFPEEEEGGCELRVKFSRSADAPAYKQGQNQLY
NELNLGRREEYDVLDKRRGRDPEMGGKPRRKNPQEGLYNELQKDKMAEAY
SEIGMKGERRRGKGHDGLYQGLSTATKDTYDALHMQALPPR

The CC004 construct comprises the following domains from N terminus to C terminus: CD8 signal peptide—3*Flag—linker (e.g., GGGGS, SEQ ID NO: 42)—anti-CD19 scFv—CD8a hinge domain—CD8 transmembrane domain—4-1BB—CD3z. See e.g., SEQ ID NO: 47 or SEQ ID NO: 82.

SEQ ID NO: 47, CC004
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACATCTACATCTGGGCCCCTCTGGCTGGCACATGTGGCGTG
CTGCTGCTCTCCCTGGTGATCACCCTGTACTGCAAAAGGGGCAGAAAGAA
ACTGCTCTACATCTTCAAGCAGCCCTTCATGAGGCCCGTCCAAACCACCC
AGGAGGAGGACGGATGCAGCTGTAGATTCCCCGAAGAGGAGGAAGGCGGC
TGCGAGCTGAGAGTCAAGTTCTCCAGGTCCGCCGACGCCCCCGCGTACAA
GCAGGGCCAGAACCAGCTCTATAACGAGCTCAATCTAGGACGAAGAGAGG
AGTACGATGTTTTGGACAAGAGACGTGGCCGGGACCCTGAGATGGGGGGA
AAGCCGAGAAGGAAGAACCCTCAGGAAGGCCTGTACAATGAACTGCAGAA
AGATAAGATGGCGGAGGCCTACAGTGAGATTGGGATGAAAGGCGAGCGCC
GGAGGGGCAAGGGGCACGATGGCCTTTACCAGGGTCTCAGTACAGCCACC
AAGGACACCTACGACGCCCTTCACATGCAGGCCCTGCCCCCTCGCTAA
SEQ ID NO: 82, CC004
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGG
CELRVKFSRSADAPAYKQGQNQLYNELNLGRREEYDVLDKRRGRDPEMGG
KPRRKNPQEGLYNELQKDKMAEAYSEIGMKGERRRGKGHDGLYQGLSTAT
KDTYDALHMQALPPR

The CC005 construct comprises the following domains from N terminus to C terminus: CD8 signal peptide—3*Flag—linker (e.g., GGGGS, SEQ ID NO: 42)—anti-CD19 scFv—CD8a hinge domain—CD8 transmembrane domain—4-1BB—FcεR cytoplasmic domain. See e.g., SEQ ID NO: 48 or SEQ ID NO: 83.

SEQ ID NO: 48, CC005
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACATCTACATCTGGGCCCCTCTGGCTGGCACATGTCTACAT
TGGCGTGCTGCTGCTCTCCCTGGTGATCACCCTGTACTGCAAAAGGGGCA
GAAAGAAACTGCCTTCAAGCAGCCCTTCATGAGGCCCGTCCAAACCACCC
AGGAGGAGGACGGATGCAGCTGTAGATTCCCCGAAGAGGAGGAAGGCGGC
TGCGAGCTGCGTCTGAAGATCCAAGTTAGGAAGGCCGCCATCACCTCCTA
CGAGAAGAGCGACGGCGTGTACACCGGTTTAAGCACAAGGAACCAAGAAA
CCTACGAGACTTTAAAGCACGAGAAGCCCCCCCAGTAA
SEQ ID NO: 83, CC005
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGG
CELRLKIQVRKAAITSYEKSDGVYTGLSTRNQETYETLKHEKPPQ

The CC007 construct comprises the following domains from N terminus to C terminus: CD8 signal peptide—3*Flag—linker (e.g., GGGGS, SEQ ID NO: 42)—anti-CD19 scFv—CD8a hinge domain—CD8 transmembrane domain—4-1BB—FcεR cytoplasmic domain—P2A peptide—IL15 isoform 1. See e.g., SEQ ID NO: 49 or SEQ ID NO: 84.

SEQ ID NO: 49, CC007
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACATCTACATCTGGGCCCCTCTGGCTGGCACATGTGGCGTG
CTGCTGCTCTCCCTGGTGATCACCCTGTACTGCAAAAGGGGCAGAAAGAA
ACTGCTCTACATCTTCAAGCAGCCCTTCATGAGGCCCGTCCAAACCACCC
AGGAGGAGGACGGATGCAGCTGTAGATTCCCCGAAGAGGAGGAAGGCGGC
TGCGAGCTGCGTCTGAAGATCCAAGTTAGGAAGGCCGCCATCACCTCCTA
CGAGAAGAGCGACGGCGTGTACACCGGTTTAAGCACAAGGAACCAAGAAA
CCTACGAGACTTTAAAGCACGAGAAGCCCCCCCAGGGCAGCGGCGCCACC
AACTTCTCTTTACTGAAGCAAGCTGGCGACGTGGAGGAGAATCCCGGTCC
CATGAGGATCAGCAAGCCCCATTTAAGGAGCATCTCCATCCAGTGCTATT
TATGTTTACTGCTGAACAGCCATTTTTTAACCGAGGCCGGCATCCACGTG
TTCATTTTAGGCTGCTTTTCCGCCGGTTTACCCAAGACCGAGGCTAACTG
GGTGAACGTGATCAGCGATTTAAAGAAGATCGAGGATTTAATCCAGAGCA
TGCACATCGATGCCACTTTATATACCGAGAGCGACGTGCACCCCAGCTGC
AAGGTGACCGCCATGAAGTGTTTTTTACTGGAGCTGCAAGTTATCTCTTT
AGAGAGCGGCGACGCCAGCATCCACGACACCGTGGAGAATTTAATCATTT
TAGCCAACAACAGCCTCTCCAGCAACGGCAATGTGACAGAGAGCGGCTGC
AAGGAGTGCGAGGAGCTGGAGGAGAAGAACATCAAGGAGTTTTTACAGAG
CTTCGTGCACATCGTGCAGATGTTCATCAACACCAGCTAA
SEQ ID NO: 84, CC007
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGG
CELRLKIQVRKAAITSYEKSDGVYTGLSTRNQETYETLKHEKPPQGSGAT
NFSLLKQAGDVEENPGPMRISKPHLRSISIQCYLCLLLNSHFLTEAGIHV
FILGCFSAGLPKTEANWVNVISDLKKIEDLIQSMHIDATLYTESDVHPSC
KVTAMKCFLLELQVISLESGDASIHDTVENLIILANNSLSSNGNVTESGC
KECEELEEKNIKEFLQSFVHIVQMFINTS

The CC008 construct comprises the following domains from N terminus to C terminus: CD8 signal peptide—3*Flag—linker (e.g., GGGGS, SEQ ID NO: 42)—anti-CD19 scFv—CD8a hinge domain—CD8 transmembrane domain—4-1BB—FcεR cytoplasmic domain—P2A peptide—IL15 isoform 1—T2A—IL-21 isoform 1. See e.g., SEQ ID NO: 50 or SEQ ID NO: 85.

SEQ ID NO: 50, CC008
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACATCTACATCTGGGCCCCTCTGGCTGGCACATGTGGCGTG
CTGCTGCTCTCCCTGGTGATCACCCTGTACTGCAAAAGGGGCAGAAAGAA
ACTGCTCTACATCTTCAAGCAGCCCTTCATGAGGCCCGTCCAAACCACCC
AGGAGGAGGACGGATGCAGCTGTAGATTCCCCGAAGAGGAGGAAGGCGGC
TGCGAGCTGCGTCTGAAGATCCAAGTTAGGAAGGCCGCCATCACCTCCTA
CGAGAAGAGCGACGGCGTGTACACCGGTTTAAGCACAAGGAACCAAGAAA
CCTACGAGACTTTAAAGCACGAGAAGCCCCCCCAGGGCAGCGGCGCCACC
AACTTCTCTTTACTGAAGCAAGCTGGCGACGTGGAGGAGAATCCCGGTCC
CATGAGGATCAGCAAGCCCCATTTAAGGAGCATCTCCATCCAGTGCTATT
TATGTTTACTGCTGAACAGCCATTTTTTAACCGAGGCCGGCATCCACGTG
TTCATTTTAGGCTGCTTTTCCGCCGGTTTACCCAAGACCGAGGCTAACTG
GGTGAACGTGATCAGCGATTTAAAGAAGATCGAGGATTTAATCCAGAGCA
TGCACATCGATGCCACTTTATATACCGAGAGCGACGTGCACCCCAGCTGC
AAGGTGACCGCCATGAAGTGTTTTTTACTGGAGCTGCAAGTTATCTCTTT
AGAGAGCGGCGACGCCAGCATCCACGACACCGTGGAGAATTTAATCATTT
TAGCCAACAACAGCCTCTCCAGCAACGGCAATGTGACAGAGAGCGGCTGC
AAGGAGTGCGAGGAGCTGGAGGAGAAGAACATCAAGGAGTTTTTACAGAG
CTTCGTGCACATCGTGCAGATGTTCATCAACACCAGCGGCAGCGGAAGAG
CCGAGGGAAGAGGCTCTTTACTGACTTGTGGCGACGTGGAAGAGAATCCC
GGCCCCATGGAGAGAATCGTGATCTGTTTAATGGTGATCTTTTTAGGCAC
TTTAGTGCACAAGTCCAGCTCCCAAGGTCAAGATAGACACATGATTCGTA
TGAGGCAGCTGATCGACATCGTGGACCAGCTGAAGAACTACGTGAACGAT
TTAGTGCCCGAATTTTTACCCGCTCCCGAGGACGTGGAGACCAACTGCGA
GTGGAGCGCCTTCTCTTGTTTCCAGAAGGCTCAGCTGAAGAGCGCCAACA
CCGGCAACAACGAGAGGATTATCAACGTGTCCATCAAGAAGCTGAAGAGG
AAGCCTCCCTCCACCAACGCCGGCAGAAGGCAGAAGCACAGACTGACTTG
TCCCTCTTGTGACAGCTACGAGAAGAAGCCCCCTAAGGAGTTTTTAGAGA
GATTCAAGTCTTTACTGCAGAAGATGATCCACCAGCATTTAAGCTCTCGT
ACCCACGGCAGCGAGGATTCCTAA
SEQ ID NO: 85, CC008
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGG
CELRLKIQVRKAAITSYEKSDGVYTGLSTRNQETYETLKHEKPPQGSGAT
NFSLLKQAGDVEENPGPMRISKPHLRSISIQCYLCLLLNSHFLTEAGIHV
FILGCFSAGLPKTEANWVNVISDLKKIEDLIQSMHIDATLYTESDVHPSC
KVTAMKCFLLELQVISLESGDASIHDTVENLIILANNSLSSNGNVTESGC
KECEELEEKNIKEFLQSFVHIVQMFINTSGSGRAEGRGSLLTCGDVEENP
GPMERIVICLMVIFLGTLVHKSSSQGQDRHMIRMRQLIDIVDQLKNYVND
LVPEFLPAPEDVETNCEWSAFSCFQKAQLKSANTGNNERIINVSIKKLKR
KPPSTNAGRRQKHRLTCPSCDSYEKKPPKEFLERFKSLLQKMIHQHLSSR
THGSEDS

The CC013 construct comprises the following domains from N terminus to C terminus: CD8 signal peptide—3*Flag—linker (e.g., GGGGS, SEQ ID NO: 42)—anti-CD19 scFv—CD8a hinge domain—CD8 transmembrane domain—4-1BB—IL2RB cytoplasmic domain—CD3z. See e.g., SEQ ID NO: 51 or SEQ ID NO: 86.

SEQ ID NO: 51, CC013
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACATCTACATCTGGGCCCCTCTGGCTGGCACATGTGGCGTG
CTGCTGCTCTCCCTGGTGATCACCCTGTACTGCAAAAGGGGCAGAAAGAA
ACTGCTCTACATCTTCAAGCAGCCCTTCATGAGGCCCGTCCAAACCACCC
AGGAGGAGGACGGATGCAGCTGTAGATTCCCCGAAGAGGAGGAAGGCGGC
TGCGAGCTGAACTGCAGGAACACCGGGCCATGGCTGAAGAAGGTCCTGAA
GTGTAACACCCCAGACCCCTCGAAGTTCTTTTCCCAGCTGAGCTCAGAGC
ATGGAGGAGACGTCCAGAAGTGGCTCTCTTCGCCCTTCCCCTCATCGTCC
TTCAGCCCTGGCGGCCTGGCACCTGAGATCTCGCCACTAGAAGTGCTGGA
GAGGGACAAGGTGACGCAGCTGCTCCCCCTGAACACTGATGCCTACTTGT
CCCTCCAAGAACTCCAGGGTCAGGACCCAACTCACTTGGTGAGAGTCAAG
TTCTCCAGGTCCGCCGACGCCCCCGCGTACAAGCAGGGCCAGAACCAGCT
CTATAACGAGCTCAATCTAGGACGAAGAGAGGAGTACGATGTTTTGGACA
AGAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGC
CTACAGTGAGATTGGGATGAAAGGCGAGCGCCGGAGGGGCAAGGGGCACG
ATGGCCTTTACCAGGGTCTCAGTACAGCCACCAAGGACACCTACGACGCC
CTTCACATGCAGGCCCTGCCCCCTCGCTAA
SEQ ID NO: 86, CC013
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGG
CELNCRNTGPWLKKVLKCNTPDPSKFFSQLSSEHGGDVQKWLSSPFPSSS
FSPGGLAPEISPLEVLERDKVTQLLPLNTDAYLSLQELQGQDPTHLVRVK
FSRSADAPAYKQGQNQLYNELNLGRREEYDVLDKRRGRDPEMGGKPRRKN
PQEGLYNELQKDKMAEAYSEIGMKGERRRGKGHDGLYQGLSTATKDTYDA
LHMQALPPR

The CC016 construct comprises the following domains from N terminus to C terminus: CD8 signal peptide—3*Flag—linker (e.g., GGGGS, SEQ ID NO: 42)—anti-CD19 scFv—CD8a hinge domain—CD8 transmembrane domain—IL2RB cytoplasmic domain—4-1BB—CD3z. See e.g., SEQ ID NO: 52 or SEQ ID NO: 87.

SEQ ID NO: 52, CC016
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACATCTACATCTGGGCCCCTCTGGCTGGCACATGTGGCGTG
CTGCTGCTCTCCCTGGTGATCACCCTGTACTGCAACTGCAGGAACACCGG
GCCATGGCTGAAGAAGGTCCTGAAGTGTAACACCCCAGACCCCTCGAAGT
TCTTTTCCCAGCTGAGCTCAGAGCATGGAGGAGACGTCCAGAAGTGGCTC
TCTTCGCCCTTCCCCTCATCGTCCTTCAGCCCTGGCGGCCTGGCACCTGA
GATCTCGCCACTAGAAGTGCTGGAGAGGGACAAGGTGACGCAGCTGCTCC
CCCTGAACACTGATGCCTACTTGTCCCTCCAAGAACTCCAGGGTCAGGAC
CCAACTCACTTGGTGAAAAGGGGCAGAAAGAAACTGCTCTACATCTTCAA
GCAGCCCTTCATGAGGCCCGTCCAAACCACCCAGGAGGAGGACGGATGCA
GCTGTAGATTCCCCGAAGAGGAGGAAGGCGGCTGCGAGCTGAGAGTCAAG
TTCTCCAGGTCCGCCGACGCCCCCGCGTACAAGCAGGGCCAGAACCAGCT
CTATAACGAGCTCAATCTAGGACGAAGAGAGGAGTACGATGTTTTGGACA
AGAGACGTGGCCGGGACCCTGAGATGGGGGGAAAGCCGAGAAGGAAGAAC
CCTCAGGAAGGCCTGTACAATGAACTGCAGAAAGATAAGATGGCGGAGGC
CTACAGTGAGATTGGGATGAAAGGCGAGCGCCGGAGGGGCAAGGGGCACG
ATGGCCTTTACCAGGGTCTCAGTACAGCCACCAAGGACACCTACGACGCC
CTTCACATGCAGGCCCTGCCCCCTCGCTAA
SEQ ID NO: 87, CC016
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDIYIWAPLAGTCGV
LLLSLVITLYCNCRNTGPWLKKVLKCNTPDPSKFFSQLSSEHGGDVQKWL
SSPFPSSSFSPGGLAPEISPLEVLERDKVTQLLPLNTDAYLSLQELQGQD
PTHLVKRGRKKLLYIFKQPFMRPVQTTQEEDGCSCRFPEEEEGGCELRVK
FSRSADAPAYKQGQNQLYNELNLGRREEYDVLDKRRGRDPEMGGKPRRKN
PQEGLYNELQKDKMAEAYSEIGMKGERRRGKGHDGLYQGLSTATKDTYDA
LHMQALPPR

The CC017 construct comprises the following domains from N terminus to C terminus: CD8 signal peptide—3*Flag—linker (e.g., GGGGS, SEQ ID NO: 42)—anti-CD19 scFv—CD8a hinge domain—CD8 transmembrane domain—4-1BB—IL2RB cytoplasmic domain—FcεR cytoplasmic domain. See e.g., SEQ ID NO: 53 or SEQ ID NO: 88.

SEQ ID NO: 53, CC017
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACATCTACATCTGGGCCCCTCTGGCTGGCACATGTGGCGTG
CTGCTGCTCTCCCTGGTGATCACCCTGTACTGCAAAAGGGGCAGAAAGAA
ACTGCTCTACATCTTCAAGCAGCCCTTCATGAGGCCCGTCCAAACCACCC
AGGAGGAGGACGGATGCAGCTGTAGATTCCCCGAAGAGGAGGAAGGCGGC
TGCGAGCTGAACTGCAGGAACACCGGGCCATGGCTGAAGAAGGTCCTGAA
GTGTAACACCCCAGACCCCTCGAAGTTCTTTTCCCAGCTGAGCTCAGAGC
ATGGAGGAGACGTCCAGAAGTGGCTCTCTTCGCCCTTCCCCTCATCGTCC
TTCAGCCCTGGCGGCCTGGCACCTGAGATCTCGCCACTAGAAGTGCTGGA
GAGGGACAAGGTGACGCAGCTGCTCCCCCTGAACACTGATGCCTACTTGT
CCCTCCAAGAACTCCAGGGTCAGGACCCAACTCACTTGGTGCGTCTGAAG
ATCCAAGTTAGGAAGGCCGCCATCACCTCCTACGAGAAGAGCGACGGCGT
GTACACCGGTTTAAGCACAAGGAACCAAGAAACCTACGAGACTTTAAAGC
ACGAGAAGCCCCCCCAGTAA
SEQ ID NO: 88, CC017
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGG
CELNCRNTGPWLKKVLKCNTPDPSKFFSQLSSEHGGDVQKWLSSPFPSSS
FSPGGLAPEISPLEVLERDKVTQLLPLNTDAYLSLQELQGQDPTHLVRLK
IQVRKAAITSYEKSDGVYTGLSTRNQETYETLKHEKPPQ

The CC018 construct comprises the following domains from N terminus to C terminus: CD8 signal peptide—3*Flag—linker (e.g., GGGGS, SEQ ID NO: 42)—anti-CD19 scFv—CD8a hinge domain—CD8 transmembrane domain—IL2RB cytoplasmic domain—4-1BB—FcεR cytoplasmic domain. See e.g., SEQ ID NO: 54 or SEQ ID NO: 89.

SEQ ID NO: 54, CC018
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACATCTACATCTGGGCCCCTCTGGCTGGCACATGTGGCGTG
CTGCTGCTCTCCCTGGTGATCACCCTGTACTGCAACTGCAGGAACACCGG
GCCATGGCTGAAGAAGGTCCTGAAGTGTAACACCCCAGACCCCTCGAAGT
TCTTTTCCCAGCTGAGCTCAGAGCATGGAGGAGACGTCCAGAAGTGGCTC
TCTTCGCCCTTCCCCTCATCGTCCTTCAGCCCTGGCGGCCTGGCACCTGA
GATCTCGCCACTAGAAGTGCTGGAGAGGGACAAGGTGACGCAGCTGCTCC
CCCTGAACACTGATGCCTACTTGTCCCTCCAAGAACTCCAGGGTCAGGAC
CCAACTCACTTGGTGAAAAGGGGCAGAAAGAAACTGCTCTACATCTTCAA
GCAGCCCTTCATGAGGCCCGTCCAAACCACCCAGGAGGAGGACGGATGCA
GCTGTAGATTCCCCGAAGAGGAGGAAGGCGGCTGCGAGCTGCGTCTGAAG
ATCCAAGTTAGGAAGGCCGCCATCACCTCCTACGAGAAGAGCGACGGCGT
GTACACCGGTTTAAGCACAAGGAACCAAGAAACCTACGAGACTTTAAAGC
ACGAGAAGCCCCCCCAGTAA
SEQ ID NO: 89, CC018
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDIYIWAPLAGTCGV
LLLSLVITLYCNCRNTGPWLKKVLKCNTPDPSKFFSQLSSEHGGDVQKWL
SSPFPSSSFSPGGLAPEISPLEVLERDKVTQLLPLNTDAYLSLQELQGQD
PTHLVKRGRKKLLYIFKQPFMRPVQTTQEEDGCSCRFPEEEEGGCELRLK
IQVRKAAITSYEKSDGVYTGLSTRNQETYETLKHEKPPQ

Anti-CD33 Group I

Non-limiting examples of Group I NK CARs that are specific to CD33 include CC103, CC104, CC108, or CC118, which correspond to polynucleotide SEQ ID NOs: 71-74 and polypeptide SEQ ID NOs: 106-109, respectively. It should be understood that other NK CARs can be generated by switching the CD-33-binding domain for a different tumor antigen- or cell-surface protein-binding domain.

The CC103 construct comprises the following domains from N terminus to C terminus: CD8 signal peptide—linker (e.g., GGGGS, SEQ ID NO: 42)—anti-CD33 scFv—CD8a hinge domain—CD8 transmembrane domain—4-1BB—CD3z. See e.g., SEQ ID NO: 71 or SEQ ID NO: 106.

SEQ ID NO: 71, CC103
ATGGCCTTACCAGTGACCGCCTTGCTCCTGCCGCTGGCCTTGCTGCTCCA
CGCCGCCAGGCCGGGTGGAGGCGGTTCGGAAGTGCAGCTGGTGCAGAGCG
GAGCAGAAGTGAAGAAGCCCGGAAGCAGCGTGAAGGTGTCTTGCAAGGCC
AGCGGCTACACCATCACCGACAGCAACATCCATTGGGTCCGGCAGGCTCC
AGGACAGTCTCTGGAGTGGATCGGCTACATCTACCCCTACAACGGCGGCA
CCGACTACAACCAGAAGTTCAAGAACCGGGCCACCCTGACCGTGGATAAC
CCCACCAACACCGCCTACATGGAGCTGAGCAGCCTGAGAAGCGAGGACAC
CGCCTTCTACTATTGCGTGAACGGCAACCCTTGGCTGGCCTATTGGGGAC
AGGGAACACTGGTGACCGTGTCCTCTGGCGGCGGAGGCAGCGGCGGCGGA
GGCTCCGGAGGCGGAGGATCCGACATCCAGCTGACCCAGTCTCCTAGCAC
CCTGAGCGCTAGCGTGGGAGATAGAGTGACCATCACTTGCAGAGCCAGCG
AGAGCCTGGACAACTACGGCATCCGGTTCCTGACTTGGTTCCAGCAGAAA
CCCGGCAAGGCCCCTAAACTGCTGATGTACGCCGCCTCTAACCAGGGAAG
CGGAGTGCCTAGCAGATTCAGCGGCAGCGGAAGCGGAACCGAGTTCACCC
TGACCATCAGCTCTCTGCAGCCAGACGACTTCGCCACCTACTACTGCCAG
CAGACCAAGGAGGTGCCTTGGAGCTTCGGCCAGGGAACCAAGGTGGAAGT
GAAGCGGACAGTGACCACAACCCCTGCCCCCAGACCTCCCACACCCGCCC
CTACCATCGCTAGCCAGCCTCTGAGCCTGAGGCCCGAGGCTTGCAGACCT
GCTGCTGGAGGCGCCGTGCACACCAGAGGACTGGATTTCGCCTGCGACAT
CTACATCTGGGCCCCTCTGGCTGGCACATGTGGCGTGCTGCTGCTCTCCC
TGGTGATCACCCTGTACTGCAAAAGGGGCAGAAAGAAACTGCTCTACATC
TTCAAGCAGCCCTTCATGAGGCCCGTCCAAACCACCCAGGAGGAGGACGG
ATGCAGCTGTAGATTCCCCGAAGAGGAGGAAGGCGGCTGCGAGCTGAGAG
TCAAGTTCTCCAGGTCCGCCGACGCCCCCGCGTACAAGCAGGGCCAGAAC
CAGCTCTATAACGAGCTCAATCTAGGACGAAGAGAGGAGTACGATGTTTT
GGACAAGAGACGTGGCCGGGACCCTGAGATGGGGGGAAAGCCGAGAAGGA
AGAACCCTCAGGAAGGCCTGTACAATGAACTGCAGAAAGATAAGATGGCG
GAGGCCTACAGTGAGATTGGGATGAAAGGCGAGCGCCGGAGGGGCAAGGG
GCACGATGGCCTTTACCAGGGTCTCAGTACAGCCACCAAGGACACCTACG
ACGCCCTTCACATGCAGGCCCTGCCCCCTCGCTAA
SEQ ID NO: 106, CC103
MALPVTALLLPLALLLHAARPGGGGSEVQLVQSGAEVKKPGSSVKVSCKA
SGYTITDSNIHWVRQAPGQSLEWIGYIYPYNGGTDYNQKFKNRATLTVDN
PTNTAYMELSSLRSEDTAFYYCVNGNPWLAYWGQGTLVTVSSGGGGSGGG
GSGGGGSDIQLTQSPSTLSASVGDRVTITCRASESLDNYGIRFLTWFQQK
PGKAPKLLMYAASNQGSGVPSRFSGSGSGTEFTLTISSLQPDDFATYYCQ
QTKEVPWSFGQGTKVEVKRTVTTTPAPRPPTPAPTIASQPLSLRPEACRP
AAGGAVHTRGLDFACDIYIWAPLAGTCGVLLLSLVITLYCKRGRKKLLYI
FKQPFMRPVQTTQEEDGCSCRFPEEEEGGCELRVKFSRSADAPAYKQGQN
QLYNELNLGRREEYDVLDKRRGRDPEMGGKPRRKNPQEGLYNELQKDKMA
EAYSEIGMKGERRRGKGHDGLYQGLSTATKDTYDALHMQALPPR

The CC104 construct comprises the following domains from N terminus to C terminus: CD8 signal peptide—3*Flag—linker (e.g., GGGGS, SEQ ID NO: 42)—anti-CD33 scFv—CD8a hinge domain—CD8 transmembrane domain—4-1BB—CD3z. See e.g., SEQ ID NO: 72 or SEQ ID NO: 107.

SEQ ID NO: 72, CC104
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GAAGTGCAGCTGGTGCAGAGCGGAGCAGAAGTGAAGAAGCCCGGAAGCAG
CGTGAAGGTGTCTTGCAAGGCCAGCGGCTACACCATCACCGACAGCAACA
TCCATTGGGTCCGGCAGGCTCCAGGACAGTCTCTGGAGTGGATCGGCTAC
ATCTACCCCTACAACGGCGGCACCGACTACAACCAGAAGTTCAAGAACCG
GGCCACCCTGACCGTGGATAACCCCACCAACACCGCCTACATGGAGCTGA
GCAGCCTGAGAAGCGAGGACACCGCCTTCTACTATTGCGTGAACGGCAAC
CCTTGGCTGGCCTATTGGGGACAGGGAACACTGGTGACCGTGTCCTCTGG
CGGCGGAGGCAGCGGCGGCGGAGGCTCCGGAGGCGGAGGATCCGACATCC
AGCTGACCCAGTCTCCTAGCACCCTGAGCGCTAGCGTGGGAGATAGAGTG
ACCATCACTTGCAGAGCCAGCGAGAGCCTGGACAACTACGGCATCCGGTT
CCTGACTTGGTTCCAGCAGAAACCCGGCAAGGCCCCTAAACTGCTGATGT
ACGCCGCCTCTAACCAGGGAAGCGGAGTGCCTAGCAGATTCAGCGGCAGC
GGAAGCGGAACCGAGTTCACCCTGACCATCAGCTCTCTGCAGCCAGACGA
CTTCGCCACCTACTACTGCCAGCAGACCAAGGAGGTGCCTTGGAGCTTCG
GCCAGGGAACCAAGGTGGAAGTGAAGCGGACAGTGACCACAACCCCTGCC
CCCAGACCTCCCACACCCGCCCCTACCATCGCTAGCCAGCCTCTGAGCCT
GAGGCCCGAGGCTTGCAGACCTGCTGCTGGAGGCGCCGTGCACACCAGAG
GACTGGATTTCGCCTGCGACATCTACATCTGGGCCCCTCTGGCTGGCACA
TGTGGCGTGCTGCTGCTCTCCCTGGTGATCACCCTGTACTGCAAAAGGGG
CAGAAAGAAACTGCTCTACATCTTCAAGCAGCCCTTCATGAGGCCCGTCC
AAACCACCCAGGAGGAGGACGGATGCAGCTGTAGATTCCCCGAAGAGGAG
GAAGGCGGCTGCGAGCTGAGAGTCAAGTTCTCCAGGTCCGCCGACGCCCC
CGCGTACAAGCAGGGCCAGAACCAGCTCTATAACGAGCTCAATCTAGGAC
GAAGAGAGGAGTACGATGTTTTGGACAAGAGACGTGGCCGGGACCCTGAG
ATGGGGGGAAAGCCGAGAAGGAAGAACCCTCAGGAAGGCCTGTACAATGA
ACTGCAGAAAGATAAGATGGCGGAGGCCTACAGTGAGATTGGGATGAAAG
GCGAGCGCCGGAGGGGCAAGGGGCACGATGGCCTTTACCAGGGTCTCAGT
ACAGCCACCAAGGACACCTACGACGCCCTTCACATGCAGGCCCTGCCCCC
TCGCTAA
SEQ ID NO: 107, CC104
MALPVTALLLPLALLLHAARPDYKDDDDKDYKDDDDKDYKDDDDKGGGGS
EVQLVQSGAEVKKPGSSVKVSCKASGYTITDSNIHWVRQAPGQSLEWIGY
IYPYNGGTDYNQKFKNRATLTVDNPTNTAYMELSSLRSEDTAFYYCVNGN
PWLAYWGQGTLVTVSSGGGGSGGGGSGGGGSDIQLTQSPSTLSASVGDRV
TITCRASESLDNYGIRFLTWFQQKPGKAPKLLMYAASNQGSGVPSRFSGS
GSGTEFTLTISSLQPDDFATYYCQQTKEVPWSFGQGTKVEVKRTVTTTPA
PRPPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEE
EGGCELRVKFSRSADAPAYKQGQNQLYNELNLGRREEYDVLDKRRGRDPE
MGGKPRRKNPQEGLYNELQKDKMAEAYSEIGMKGERRRGKGHDGLYQGLS
TATKDTYDALHMQALPPR

The CC108 construct comprises the following domains from N terminus to C terminus: CD8 signal peptide—3*Flag—linker (e.g., GGGGS, SEQ ID NO: 42)—anti-CD33 scFv—CD8a hinge domain—CD8 transmembrane domain—4-1BB—FcεR cytoplasmic domain—P2A peptide—IL15 isoform 1—T2A—IL-21 isoform 1. See e.g., SEQ ID NO: 73 or SEQ ID NO: 108.

SEQ ID NO: 73, CC108
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GAAGTGCAGCTGGTGCAGAGCGGAGCAGAAGTGAAGAAGCCCGGAAGCAG
CGTGAAGGTGTCTTGCAAGGCCAGCGGCTACACCATCACCGACAGCAACA
TCCATTGGGTCCGGCAGGCTCCAGGACAGTCTCTGGAGTGGATCGGCTAC
ATCTACCCCTACAACGGCGGCACCGACTACAACCAGAAGTTCAAGAACCG
GGCCACCCTGACCGTGGATAACCCCACCAACACCGCCTACATGGAGCTGA
GCAGCCTGAGAAGCGAGGACACCGCCTTCTACTATTGCGTGAACGGCAAC
CCTTGGCTGGCCTATTGGGGACAGGGAACACTGGTGACCGTGTCCTCTGG
CGGCGGAGGCAGCGGCGGCGGAGGCTCCGGAGGCGGAGGATCCGACATCC
AGCTGACCCAGTCTCCTAGCACCCTGAGCGCTAGCGTGGGAGATAGAGTG
ACCATCACTTGCAGAGCCAGCGAGAGCCTGGACAACTACGGCATCCGGTT
CCTGACTTGGTTCCAGCAGAAACCCGGCAAGGCCCCTAAACTGCTGATGT
ACGCCGCCTCTAACCAGGGAAGCGGAGTGCCTAGCAGATTCAGCGGCAGC
GGAAGCGGAACCGAGTTCACCCTGACCATCAGCTCTCTGCAGCCAGACGA
CTTCGCCACCTACTACTGCCAGCAGACCAAGGAGGTGCCTTGGAGCTTCG
GCCAGGGAACCAAGGTGGAAGTGAAGCGGACAGTGACCACAACCCCTGCC
CCCAGACCTCCCACACCCGCCCCTACCATCGCTAGCCAGCCTCTGAGCCT
GAGGCCCGAGGCTTGCAGACCTGCTGCTGGAGGCGCCGTGCACACCAGAG
GACTGGATTTCGCCTGCGACATCTACATCTGGGCCCCTCTGGCTGGCACA
TGTGGCGTGCTGCTGCTCTCCCTGGTGATCACCCTGTACTGCAAAAGGGG
CAGAAAGAAACTGCTCTACATCTTCAAGCAGCCCTTCATGAGGCCCGTCC
AAACCACCCAGGAGGAGGACGGATGCAGCTGTAGATTCCCCGAAGAGGAG
GAAGGCGGCTGCGAGCTGCGTCTGAAGATCCAAGTTAGGAAGGCCGCCAT
CACCTCCTACGAGAAGAGCGACGGCGTGTACACCGGTTTAAGCACAAGGA
ACCAAGAAACCTACGAGACTTTAAAGCACGAGAAGCCCCCCCAGGGCAGC
GGCGCCACCAACTTCTCTTTACTGAAGCAAGCTGGCGACGTGGAGGAGAA
TCCCGGTCCCATGAGGATCAGCAAGCCCCATTTAAGGAGCATCTCCATCC
AGTGCTATTTATGTTTACTGCTGAACAGCCATTTTTTAACCGAGGCCGGC
ATCCACGTGTTCATTTTAGGCTGCTTTTCCGCCGGTTTACCCAAGACCGA
GGCTAACTGGGTGAACGTGATCAGCGATTTAAAGAAGATCGAGGATTTAA
TCCAGAGCATGCACATCGATGCCACTTTATATACCGAGAGCGACGTGCAC
CCCAGCTGCAAGGTGACCGCCATGAAGTGTTTTTTACTGGAGCTGCAAGT
TATCTCTTTAGAGAGCGGCGACGCCAGCATCCACGACACCGTGGAGAATT
TAATCATTTTAGCCAACAACAGCCTCTCCAGCAACGGCAATGTGACAGAG
AGCGGCTGCAAGGAGTGCGAGGAGCTGGAGGAGAAGAACATCAAGGAGTT
TTTACAGAGCTTCGTGCACATCGTGCAGATGTTCATCAACACCAGCGGCA
GCGGAAGAGCCGAGGGAAGAGGCTCTTTACTGACTTGTGGCGACGTGGAA
GAGAATCCCGGCCCCATGGAGAGAATCGTGATCTGTTTAATGGTGATCTT
TTTAGGCACTTTAGTGCACAAGTCCAGCTCCCAAGGTCAAGATAGACACA
TGATTCGTATGAGGCAGCTGATCGACATCGTGGACCAGCTGAAGAACTAC
GTGAACGATTTAGTGCCCGAATTTTTACCCGCTCCCGAGGACGTGGAGAC
CAACTGCGAGTGGAGCGCCTTCTCTTGTTTCCAGAAGGCTCAGCTGAAGA
GCGCCAACACCGGCAACAACGAGAGGATTATCAACGTGTCCATCAAGAAG
CTGAAGAGGAAGCCTCCCTCCACCAACGCCGGCAGAAGGCAGAAGCACAG
ACTGACTTGTCCCTCTTGTGACAGCTACGAGAAGAAGCCCCCTAAGGAGT
TTTTAGAGAGATTCAAGTCTTTACTGCAGAAGATGATCCACCAGCATTTA
AGCTCTCGTACCCACGGCAGCGAGGATTCCTAA
SEQ ID NO: 108, CC108
MALPVTALLLPLALLLHAARPDYKDDDDKDYKDDDDKDYKDDDDKGGGGS
EVQLVQSGAEVKKPGSSVKVSCKASGYTITDSNIHWVRQAPGQSLEWIGY
IYPYNGGTDYNQKFKNRATLTVDNPTNTAYMELSSLRSEDTAFYYCVNGN
PWLAYWGQGTLVTVSSGGGGSGGGGSGGGGSDIQLTQSPSTLSASVGDRV
TITCRASESLDNYGIRFLTWFQQKPGKAPKLLMYAASNQGSGVPSRFSGS
GSGTEFTLTISSLQPDDFATYYCQQTKEVPWSFGQGTKVEVKRTVTTTPA
PRPPTPAPTIASQPLSLRPEACRPAAGGAVHTRGLDFACDIYIWAPLAGT
CGVLLLSLVITLYCKRGRKKLLYIFKQPFMRPVQTTQEEDGCSCRFPEEE
EGGCELRLKIQVRKAAITSYEKSDGVYTGLSTRNQETYETLKHEKPPQGS
GATNFSLLKQAGDVEENPGPMRISKPHLRSISIQCYLCLLLNSHFLTEAG
IHVFILGCFSAGLPKTEANWVNVISDLKKIEDLIQSMHIDATLYTESDVH
PSCKVTAMKCFLLELQVISLESGDASIHDTVENLIILANNSLSSNGNVTE
SGCKECEELEEKNIKEFLQSFVHIVQMFINTSGSGRAEGRGSLLTCGDVE
ENPGPMERIVICLMVIFLGTLVHKSSSQGQDRHMIRMRQLIDIVDQLKNY
VNDLVPEFLPAPEDVETNCEWSAFSCFQKAQLKSANTGNNERIINVSIKK
LKRKPPSTNAGRRQKHRLTCPSCDSYEKKPPKEFLERFKSLLQKMIHQHL
SSRTHGSEDS

The CC118 construct comprises the following domains from N terminus to C terminus: CD8 signal peptide—3*Flag—linker (e.g., GGGGS, SEQ ID NO: 42)—anti-CD33 scFv—CD8a hinge domain—CD8 transmembrane domain—IL2RB cytoplasmic domain—4-1BB—FcεR cytoplasmic domain. See e.g., SEQ ID NO: 74 or SEQ ID NO: 109.

SEQ ID NO: 74, CC118
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GAAGTGCAGCTGGTGCAGAGCGGAGCAGAAGTGAAGAAGCCCGGAAGCAG
CGTGAAGGTGTCTTGCAAGGCCAGCGGCTACACCATCACCGACAGCAACA
TCCATTGGGTCCGGCAGGCTCCAGGACAGTCTCTGGAGTGGATCGGCTAC
ATCTACCCCTACAACGGCGGCACCGACTACAACCAGAAGTTCAAGAACCG
GGCCACCCTGACCGTGGATAACCCCACCAACACCGCCTACATGGAGCTGA
GCAGCCTGAGAAGCGAGGACACCGCCTTCTACTATTGCGTGAACGGCAAC
CCTTGGCTGGCCTATTGGGGACAGGGAACACTGGTGACCGTGTCCTCTGG
CGGCGGAGGCAGCGGCGGCGGAGGCTCCGGAGGCGGAGGATCCGACATCC
AGCTGACCCAGTCTCCTAGCACCCTGAGCGCTAGCGTGGGAGATAGAGTG
ACCATCACTTGCAGAGCCAGCGAGAGCCTGGACAACTACGGCATCCGGTT
CCTGACTTGGTTCCAGCAGAAACCCGGCAAGGCCCCTAAACTGCTGATGT
ACGCCGCCTCTAACCAGGGAAGCGGAGTGCCTAGCAGATTCAGCGGCAGC
GGAAGCGGAACCGAGTTCACCCTGACCATCAGCTCTCTGCAGCCAGACGA
CTTCGCCACCTACTACTGCCAGCAGACCAAGGAGGTGCCTTGGAGCTTCG
GCCAGGGAACCAAGGTGGAAGTGAAGCGGACAGTGACCACAACCCCTGCC
CCCAGACCTCCCACACCCGCCCCTACCATCGCTAGCCAGCCTCTGAGCCT
GAGGCCCGAGGCTTGCAGACCTGCTGCTGGAGGCGCCGTGCACACCAGAG
GACTGGATTTCGCCTGCGACATCTACATCTGGGCCCCTCTGGCTGGCACA
TGTGGCGTGCTGCTGCTCTCCCTGGTGATCACCCTGTACTGCAACTGCAG
GAACACCGGGCCATGGCTGAAGAAGGTCCTGAAGTGTAACACCCCAGACC
CCTCGAAGTTCTTTTCCCAGCTGAGCTCAGAGCATGGAGGAGACGTCCAG
AAGTGGCTCTCTTCGCCCTTCCCCTCATCGTCCTTCAGCCCTGGCGGCCT
GGCACCTGAGATCTCGCCACTAGAAGTGCTGGAGAGGGACAAGGTGACGC
AGCTGCTCCCCCTGAACACTGATGCCTACTTGTCCCTCCAAGAACTCCAG
GGTCAGGACCCAACTCACTTGGTGAAAAGGGGCAGAAAGAAACTGCTCTA
CATCTTCAAGCAGCCCTTCATGAGGCCCGTCCAAACCACCCAGGAGGAGG
ACGGATGCAGCTGTAGATTCCCCGAAGAGGAGGAAGGCGGCTGCGAGCTG
CGTCTGAAGATCCAAGTTAGGAAGGCCGCCATCACCTCCTACGAGAAGAG
CGACGGCGTGTACACCGGTTTAAGCACAAGGAACCAAGAAACCTACGAGA
CTTTAAAGCACGAGAAGCCCCCCCAGTAA
SEQ ID NO: 109, CC118
MALPVTALLLPLALLLHAARPDYKDDDDKDYKDDDDKDYKDDDDKGGGGS
EVQLVQSGAEVKKPGSSVKVSCKASGYTITDSNIHWVRQAPGQSLEWIGY
IYPYNGGTDYNQKFKNRATLTVDNPTNTAYMELSSLRSEDTAFYYCVNGN
PWLAYWGQGTLVTVSSGGGGSGGGGSGGGGSDIQLTQSPSTLSASVGDRV
TITCRASESLDNYGIRFLTWFQQKPGKAPKLLMYAASNQGSGVPSRFSGS
GSGTEFTLTISSLQPDDFATYYCQQTKEVPWSFGQGTKVEVKRTVTTTPA
PRPPTPAPTIASQPLSLRPEACRPAAGGAVHTRGLDFACDIYIWAPLAGT
CGVLLLSLVITLYCNCRNTGPWLKKVLKCNTPDPSKFFSQLSSEHGGDVQ
KWLSSPFPSSSFSPGGLAPEISPLEVLERDKVTQLLPLNTDAYLSLQELQ
GQDPTHLVKRGRKKLLYIFKQPFMRPVQTTQEEDGCSCRFPEEEEGGCEL
RLKIQVRKAAITSYEKSDGVYTGLSTRNQETYETLKHEKPPQ

Group II-IV NK CAR Polypeptides

In one aspect, described herein is Group II-IV NK CAR polypeptide comprising: (a) an extracellular binding domain; (b) a transmembrane domain; and (c) an intracellular domain comprising at least one signaling domain from a NK cell receptor.

In one aspect, described herein is Group II NK CAR polypeptide comprising: (a) an extracellular binding domain; (b) a transmembrane domain; and (c) an intracellular domain comprising at least one signaling domain from a NK cell receptor; wherein the transmembrane domain comprises the transmembrane domain of NKG2D. In some embodiments, the NK cell receptor comprises 2B4 (e.g., Group IIa). In some embodiments, the NK cell receptor comprises NTB-A (e.g., Group IIb). In some embodiments, the NK cell receptor comprises CD2 (e.g., Group IIc). In some embodiments, the NK cell receptor comprises CRACC (e.g., Group IId).

In one aspect, described herein is Group III NK CAR polypeptide comprising: (a) an extracellular binding domain; (b) a transmembrane domain; and (c) an intracellular domain comprising at least one signaling domain from a NK cell receptor; wherein the transmembrane domain comprises the transmembrane domain of NKp46. In some embodiments, the NK CAR further comprises the intracellular signaling domain of NKp46. In some embodiments, the NK cell receptor comprises 2B4 (e.g., Group IIIa). In some embodiments, the NK cell receptor comprises NTB-A (e.g., Group IIIb). In some embodiments, the NK cell receptor comprises CD2 (e.g., Group IIIc). In some embodiments, the NK cell receptor comprises CRACC (e.g., Group IIId).

In one aspect, described herein is Group IV NK CAR polypeptide comprising: (a) an extracellular binding domain; (b) a transmembrane domain; and (c) an intracellular domain comprising at least one signaling domain from a NK cell receptor; wherein the transmembrane domain comprises the transmembrane domain of DNAM1. In some embodiments, the NK CAR further comprises the intracellular signaling domain of DNAM1. In some embodiments, the NK cell receptor comprises 2B4 (e.g., Group IVa). In some embodiments, the NK cell receptor comprises NTB-A (e.g., Group IVb). In some embodiments, the NK cell receptor comprises CD2 (e.g., Group IVc). In some embodiments, the NK cell receptor comprises CRACC (e.g., Group IVd).

In some embodiments of any of the aspects, a Group II-IV NK CAR polypeptide comprises one of SEQ ID NOs: 90-105 or 110-114, or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of 90-105 or 110-114 maintains the same functions (e.g., antigen-binding and intracellular signaling).

In some embodiments of any of the aspects, a Group II-IV NK CAR polynucleotide comprises one of SEQ ID NOs: 55-70 or 75-79, or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of SEQ ID NOs: 55-70 or 75-79 that as a polypeptide maintains the same functions, or a codon-optimized version of one of SEQ ID NOs: 55-70 or 75-79.

Anti-CD19 Groups II-IV

Non-limiting examples of Group II-IV NK CARs that are specific to CD19 include CC024, CC025, CC026, CC027, CC028, CC029, CC030, CC031, CC032, CC033, CC034, CC035, CC036, CC037, CC038, or CC039, which correspond to polynucleotide SEQ ID NOs: 55-70 and polypeptide SEQ ID NOs: 90-105, respectively. It should be understood that other NK CARs can be generated by switching the CD-19-binding domain for a different tumor antigen- or cell-surface protein-binding domain.

The CC024 construct comprises the following domains from N terminus to C terminus: CD8 signal peptide—3*Flag—linker (e.g., GGGGS, SEQ ID NO: 42)—anti-CD19 scFv—CD8a hinge domain—NKG2D transmembrane domain—2B4 cytoplasmic domain. See e.g., SEQ ID NO: 55 or SEQ ID NO: 90.

SEQ ID NO: 55, CC024
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACCCTTTCTTCTTCTGCTGCTTCATCGCCGTGGCCATGGGC
ATTCGTTTCATCATCATGGTGGCCTGGAGGAGGAAAAGGAAGGAGAAGCA
GTCCGAGACATCCCCCAAAGAGTTTTTAACCATTTATGAGGACGTGAAGG
ACCTCAAGACCAGAAGGAACCACGAGCAAGAACAGACCTTTCCCGGCGGC
GGATCCACCATCTACAGCATGATCCAGAGCCAGAGCTCCGCCCCTACAAG
CCAAGAACCCGCTTACACTTTATATTCTTTAATCCAGCCTTCTCGTAAGA
GCGGCTCCCGTAAGAGGAACCACAGCCCCAGCTTTAATAGCACCATTTAC
GAGGTCATCGGCAAGTCCCAGCCTAAGGCCCAAAACCCCGCTCGTCTCTC
TCGTAAGGAGCTGGAGAATTTCGACGTGTACAGCTAA
SEQ ID NO: 90, CC024
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDPFFFCCFIAVAMG
IRFIIMVAWRRKRKEKQSETSPKEFLTIYEDVKDLKTRRNHEQEQTFPGG
GSTIYSMIQSQSSAPTSQEPAYTLYSLIQPSRKSGSRKRNHSPSFNSTIY
EVIGKSQPKAQNPARLSRKELENFDVYS

The CC025 construct (e.g., reverse domain order of CC024, except not including the CD8 signal peptide) comprises the following domains from N terminus to C terminus: 2B4 cytoplasmic domain—NKG2D transmembrane domain—CD8a hinge domain—anti-CD19 scFv—linker (e.g., GGGGS, SEQ ID NO: 42)—3*Flag. See e.g., SEQ ID NO: 56 or SEQ ID NO: 91.

SEQ ID NO: 56, CC025
TGGAGGAGGAAAAGGAAGGAGAAGCAGTCCGAGACATCCCCCAAAGAGTT
TTTAACCATTTATGAGGACGTGAAGGACCTCAAGACCAGAAGGAACCACG
AGCAAGAACAGACCTTTCCCGGCGGCGGATCCACCATCTACAGCATGATC
CAGAGCCAGAGCTCCGCCCCTACAAGCCAAGAACCCGCTTACACTTTATA
TTCTTTAATCCAGCCTTCTCGTAAGAGCGGCTCCCGTAAGAGGAACCACA
GCCCCAGCTTTAATAGCACCATTTACGAGGTCATCGGCAAGTCCCAGCCT
AAGGCCCAAAACCCCGCTCGTCTCTCTCGTAAGGAGCTGGAGAATTTCGA
CGTGTACAGCCCTTTCTTCTTCTGCTGCTTCATCGCCGTGGCCATGGGCA
TTCGTTTCATCATCATGGTGGCCACCACAACCCCTGCCCCCAGACCTCCC
ACACCCGCCCCTACCATCGCTAGCCAGCCTCTGAGCCTGAGGCCCGAGGC
TTGCAGACCTGCTGCTGGAGGCGCCGTGCACACCAGAGGACTGGATTTCG
CCTGCGACGACATCCAGATGACCCAGACCACCAGCAGCCTGAGCGCTAGC
CTGGGCGATAGAGTGACCATCAGCTGCAGAGCCAGCCAGGACATCAGCAA
GTACCTGAACTGGTACCAGCAGAAGCCCGACGGCACAGTGAAACTGCTGA
TCTACCACACCTCCAGACTGCATAGCGGCGTGCCTAGCAGGTTTTCCGGC
AGCGGCAGCGGAACCGACTACTCCCTGACCATCAGCAACCTGGAGCAGGA
GGACATCGCTACCTACTTTTGTCAGCAGGGCAACACCCTGCCTTACACCT
TTGGCGGCGGAACAAAGCTGGAGATTACCGGCGGCGGAGGCAGCGGCGGC
GGAGGCTCCGGAGGCGGAGGATCCGAGGTGAAGCTGCAGGAAAGCGGACC
TGGACTGGTCGCCCCTAGCCAGAGCCTGAGCGTGACCTGTACCGTGAGCG
GAGTGAGCCTGCCCGATTACGGCGTGTCCTGGATCAGGCAGCCCCCCAGG
AAGGGACTGGAATGGCTGGGCGTCATCTGGGGCTCCGAGACAACCTACTA
CAACAGCGCCCTCAAGTCCAGGCTCACCATTATCAAGGACAATAGCAAGA
GCCAGGTGTTCCTGAAGATGAACAGCCTGCAGACCGACGACACAGCCATT
TATTACTGCGCCAAGCACTATTATTACGGCGGCAGCTACGCCATGGACTA
TTGGGGCCAGGGAACAAGCGTGACCGTCAGCAGCGGTGGAGGCGGTTCGG
ATTACAAGGATGACGACGATAAGGACTATAAGGACGACGACGATAAGGAT
TATAAAGACGATGACGACAAATAA
SEQ ID NO: 91, CC025
WRRKRKEKQSETSPKEFLTIYEDVKDLKTRRNHEQEQTFPGGGSTIYSMI
QSQSSAPTSQEPAYTLYSLIQPSRKSGSRKRNHSPSFNSTIYEVIGKSQP
KAQNPARLSRKELENFDVYSPFFFCCFIAVAMGIRFIIMVATTTPAPRPP
TPAPTIASQPLSLRPEACRPAAGGAVHTRGLDFACDDIQMTQTTSSLSAS
LGDRVTISCRASQDISKYLNWYQQKPDGTVKLLIYHTSRLHSGVPSRFSG
SGSGTDYSLTISNLEQEDIATYFCQQGNTLPYTFGGGTKLEITGGGGSGG
GGSGGGGSEVKLQESGPGLVAPSQSLSVTCTVSGVSLPDYGVSWIRQPPR
KGLEWLGVIWGSETTYYNSALKSRLTIIKDNSKSQVFLKMNSLQTDDTAI
YYCAKHYYYGGSYAMDYWGQGTSVTVSSGGGGSDYKDDDDKDYKDDDDKD
YKDDDDK

The CC026 construct comprises the following domains from N terminus to C terminus: CD8 signal peptide—3*Flag—linker (e.g., GGGGS, SEQ ID NO: 42)—anti-CD19 scFv—CD8a hinge domain—NKp46 transmembrane and cytoplasmic domains—CD2 cytoplasmic domain. See e.g., SEQ ID NO: 57 or SEQ ID NO: 92.

SEQ ID NO: 57, CC026
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACATGGGACTGGCCTTTCTGGTGCTGGTGGCTCTGGTGTGG
TTTCTGGTGGAGGACTGGCTGAGCAGAAAGAGGACCAGAGAGAGAGCCAG
CAGAGCCAGCACATGGGAGGGCAGAAGGAGACTGAACACCCAGACCCTGA
AGAGGAAGAAGCAGAGGTCTAGAAGAAACGACGAGGAGCTCGAGACAAGA
GCCCACAGAGTGGCTACCGAAGAGAGAGGCAGAAAGCCCCACCAGATCCC
CGCCAGCACACCCCAGAACCCCGCCACAAGCCAGCATCCCCCCCCTCCTC
CCGGCCACAGATCCCAAGCTCCCAGCCACAGACCTCCTCCCCCCGGCCAT
AGGGTGCAGCATCAGCCCCAGAAAAGACCTCCCGCCCCCAGCGGAACCCA
AGTGCATCAGCAGAAGGGCCCTCCTCTGCCCAGACCTAGGGTGCAACCCA
AGCCCCCTCATGGCGCTGCCGAGAACTCTCTGAGCCCTAGCAGCAACTAA
SEQ ID NO: 92, CC026
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDMGLAFLVLVALVW
FLVEDWLSRKRTRERASRASTWEGRRRLNTQTLKRKKQRSRRNDEELETR
AHRVATEERGRKPHQIPASTPQNPATSQHPPPPPGHRSQAPSHRPPPPGH
RVQHQPQKRPPAPSGTQVHQQKGPPLPRPRVQPKPPHGAAENSLSPSSN

The CC027 construct comprises the following domains from N terminus to C terminus: CD8 signal peptide—3*Flag—linker (e.g., GGGGS, SEQ ID NO: 42)—anti-CD19 scFv—CD8a hinge domain—DNAM1 transmembrane and cytoplasmic domains—2B4 cytoplasmic domain. See e.g., SEQ ID NO: 58 or SEQ ID NO: 93.

SEQ ID NO: 58, CC027
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACGGCGGCACCGTGCTGCTGCTGCTGTTCGTGATCTCCATC
ACCACCATCATCGTCATCTTTCTGAACAGAAGGAGGAGAAGGGAGAGGAG
GGATCTGTTCACCGAGAGCTGGGACACCCAGAAGGCCCCCAACAACTATA
GAAGCCCCATCAGCACCAGCCAGCCCACCAACCAGAGCATGGACGACACC
AGAGAGGACATCTACGTGAACTACCCCACCTTCAGCAGAAGACCCAAGAC
CAGAGTGTGGAGGAGGAAAAGGAAGGAGAAGCAGTCCGAGACATCCCCCA
AAGAGTTTTTAACCATTTATGAGGACGTGAAGGACCTCAAGACCAGAAGG
AACCACGAGCAAGAACAGACCTTTCCCGGCGGCGGATCCACCATCTACAG
CATGATCCAGAGCCAGAGCTCCGCCCCTACAAGCCAAGAACCCGCTTACA
CTTTATATTCTTTAATCCAGCCTTCTCGTAAGAGCGGCTCCCGTAAGAGG
AACCACAGCCCCAGCTTTAATAGCACCATTTACGAGGTCATCGGCAAGTC
CCAGCCTAAGGCCCAAAACCCCGCTCGTCTCTCTCGTAAGGAGCTGGAGA
ATTTCGACGTGTACAGCTAA
SEQ ID NO: 93, CC027
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDGGTVLLLLFVISI
TTIIVIFLNRRRRRERRDLFTESWDTQKAPNNYRSPISTSQPTNQSMDDT
REDIYVNYPTFSRRPKTRVWRRKRKEKQSETSPKEFLTIYEDVKDLKTRR
NHEQEQTFPGGGSTIYSMIQSQSSAPTSQEPAYTLYSLIQPSRKSGSRKR
NHSPSFNSTIYEVIGKSQPKAQNPARLSRKELENFDVYS

The CC028 construct (e.g., reverse domain order of CC039, except not including the CD8 signal peptide) comprises the following domains from N terminus to C terminus: NTB-A cytoplasmic domain—NKG2D transmembrane domain—CD8a hinge domain—anti-CD19 scFv—linker (e.g., GGGGS, SEQ ID NO: 42)—3*Flag. See e.g., SEQ ID NO: 59 or SEQ ID NO: 94.

SEQ ID NO: 59, CC028
CTGAGAAAGAGAAGAGACTCTCTGTCTCTGAGCACCCAAAGAACACAAGG
CCCCGAGAGCGCCAGAAATCTGGAGTACGTGAGCGTGAGCCCCACCAACA
ACACCGTGTACGCCAGCGTGACCCACAGCAACAGAGAGACCGAGATCTGG
ACCCCTAGAGAGAACGACACCATCACCATCTACAGCACCATCAACCACAG
CAAGGAGAGCAAGCCCACATTCTCTAGAGCCACCGCTCTGGATAACGTGG
TGCCTTTCTTCTTCTGCTGCTTCATCGCCGTGGCCATGGGCATTCGTTTC
ATCATCATGGTGGCCACCACAACCCCTGCCCCCAGACCTCCCACACCCGC
CCCTACCATCGCTAGCCAGCCTCTGAGCCTGAGGCCCGAGGCTTGCAGAC
CTGCTGCTGGAGGCGCCGTGCACACCAGAGGACTGGATTTCGCCTGCGAC
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCGGTGGAGGCGGTTCGGATTACAAG
GATGACGACGATAAGGACTATAAGGACGACGACGATAAGGATTATAAAGA
CGATGACGACAAATAA
SEQ ID NO: 94, CC028
LRKRRDSLSLSTQRTQGPESARNLEYVSVSPTNNTVYASVTHSNRETEIW
TPRENDTITIYSTINHSKESKPTFSRATALDNVVPFFFCCFIAVAMGIRF
IIMVATTTPAPRPPTPAPTIASQPLSLRPEACRPAAGGAVHTRGLDFACD
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSGGGGSDYK
DDDDKDYKDDDDKDYKDDDDK

The CC029 construct comprises the following domains from N terminus to C terminus: CD8 signal peptide—3*Flag—linker (e.g., GGGGS, SEQ ID NO: 42)—anti-CD19 scFv—CD8a hinge domain—NKp46 transmembrane and cytoplasmic domains—NTB-A cytoplasmic domain. See e.g., SEQ ID NO: 60 or SEQ ID NO: 95.

SEQ ID NO: 60, CC029
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACATGGGACTGGCCTTTCTGGTGCTGGTGGCTCTGGTGTGG
TTTCTGGTGGAGGACTGGCTGAGCAGAAAGAGGACCAGAGAGAGAGCCAG
CAGAGCCAGCACATGGGAGGGCAGAAGGAGACTGAACACCCAGACCCTGC
TGAGAAAGAGAAGAGACTCTCTGTCTCTGAGCACCCAAAGAACACAAGGC
CCCGAGAGCGCCAGAAATCTGGAGTACGTGAGCGTGAGCCCCACCAACAA
CACCGTGTACGCCAGCGTGACCCACAGCAACAGAGAGACCGAGATCTGGA
CCCCTAGAGAGAACGACACCATCACCATCTACAGCACCATCAACCACAGC
AAGGAGAGCAAGCCCACATTCTCTAGAGCCACCGCTCTGGATAACGTGGT
GTAA
SEQ ID NO: 95, CC029
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDMGLAFLVLVALVW
FLVEDWLSRKRTRERASRASTWEGRRRLNTQTLLRKRRDSLSLSTQRTQG
PESARNLEYVSVSPTNNTVYASVTHSNRETEIWTPRENDTITIYSTINHS
KESKPTFSRATALDNVV

The CC030 construct comprises the following domains from N terminus to C terminus: CD8 signal peptide—3*Flag—linker (e.g., GGGGS, SEQ ID NO: 42)—anti-CD19 scFv—CD8a hinge domain—DNAM1 transmembrane and cytoplasmic domains—CRACC cytoplasmic domain. See e.g., SEQ ID NO: 61 or SEQ ID NO: 96.

CC030
SEQ ID NO: 61
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACGGCGGCACCGTGCTGCTGCTGCTGTTCGTGATCTCCATC
ACCACCATCATCGTCATCTTTCTGAACAGAAGGAGGAGAAGGGAGAGGAG
GGATCTGTTCACCGAGAGCTGGGACACCCAGAAGGCCCCCAACAACTATA
GAAGCCCCATCAGCACCAGCCAGCCCACCAACCAGAGCATGGACGACACC
AGAGAGGACATCTACGTGAACTACCCCACCTTCAGCAGAAGACCCAAGAC
CAGAGTGTGGTTTCTGAAGAGAGAGAGGCAAGAGGAGTACATCGAGGAGA
AGAAGAGGGTGGACATCTGCAGAGAGACCCCCAACATCTGCCCCCATTCC
GGCGAGAACACCGAGTACGACACCATCCCCCACACCAACAGAACCATTCT
GAAGGAGGACCCCGCCAACACCGTGTACAGCACCGTGGAGATCCCCAAGA
AGATGGAGAATCCCCACTCTCTGCTGACCATGCCCGACACCCCCAGACTG
TTCGCCTATGAGAATGTTATCTAA
CC030
SEQ ID NO: 96
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDGGTVLLLLFVISI
TTIIVIFLNRRRRRERRDLFTESWDTQKAPNNYRSPISTSQPTNQSMDDT
REDIYVNYPTFSRRPKTRVWFLKRERQEEYIEEKKRVDICRETPNICPHS
GENTEYDTIPHTNRTILKEDPANTVYSTVEIPKKMENPHSLLTMPDTPRL
FAYENVI,

The CC031 construct (e.g., reverse domain order of CC037, except not including the CD8 signal peptide) comprises the following domains from N terminus to C terminus: CD2 cytoplasmic domain—NKG2D transmembrane domain—CD8a hinge domain—anti-CD19 scFv—linker (e.g., GGGGS, SEQ ID NO: 42)—3*Flag. See e.g., SEQ ID NO: 62 or SEQ ID NO: 97.

CC031
SEQ ID NO: 62
AAGAGGAAGAAGCAGAGGTCTAGAAGAAACGACGAGGAGCTCGAGACAAG
AGCCCACAGAGTGGCTACCGAAGAGAGAGGCAGAAAGCCCCACCAGATCC
CCGCCAGCACACCCCAGAACCCCGCCACAAGCCAGCATCCCCCCCCTCCT
CCCGGCCACAGATCCCAAGCTCCCAGCCACAGACCTCCTCCCCCCGGCCA
TAGGGTGCAGCATCAGCCCCAGAAAAGACCTCCCGCCCCCAGCGGAACCC
AAGTGCATCAGCAGAAGGGCCCTCCTCTGCCCAGACCTAGGGTGCAACCC
AAGCCCCCTCATGGCGCTGCCGAGAACTCTCTGAGCCCTAGCAGCAACCC
TTTCTTCTTCTGCTGCTTCATCGCCGTGGCCATGGGCATTCGTTTCATCA
TCATGGTGGCCACCACAACCCCTGCCCCCAGACCTCCCACACCCGCCCCT
ACCATCGCTAGCCAGCCTCTGAGCCTGAGGCCCGAGGCTTGCAGACCTGC
TGCTGGAGGCGCCGTGCACACCAGAGGACTGGATTTCGCCTGCGACGACA
TCCAGATGACCCAGACCACCAGCAGCCTGAGCGCTAGCCTGGGCGATAGA
GTGACCATCAGCTGCAGAGCCAGCCAGGACATCAGCAAGTACCTGAACTG
GTACCAGCAGAAGCCCGACGGCACAGTGAAACTGCTGATCTACCACACCT
CCAGACTGCATAGCGGCGTGCCTAGCAGGTTTTCCGGCAGCGGCAGCGGA
ACCGACTACTCCCTGACCATCAGCAACCTGGAGCAGGAGGACATCGCTAC
CTACTTTTGTCAGCAGGGCAACACCCTGCCTTACACCTTTGGCGGCGGAA
CAAAGCTGGAGATTACCGGCGGCGGAGGCAGCGGCGGCGGAGGCTCCGGA
GGCGGAGGATCCGAGGTGAAGCTGCAGGAAAGCGGACCTGGACTGGTCGC
CCCTAGCCAGAGCCTGAGCGTGACCTGTACCGTGAGCGGAGTGAGCCTGC
CCGATTACGGCGTGTCCTGGATCAGGCAGCCCCCCAGGAAGGGACTGGAA
TGGCTGGGCGTCATCTGGGGCTCCGAGACAACCTACTACAACAGCGCCCT
CAAGTCCAGGCTCACCATTATCAAGGACAATAGCAAGAGCCAGGTGTTCC
TGAAGATGAACAGCCTGCAGACCGACGACACAGCCATTTATTACTGCGCC
AAGCACTATTATTACGGCGGCAGCTACGCCATGGACTATTGGGGCCAGGG
AACAAGCGTGACCGTCAGCAGCGGTGGAGGCGGTTCGGATTACAAGGATG
ACGACGATAAGGACTATAAGGACGACGACGATAAGGATTATAAAGACGAT
GACGACAAATAA,
CC031
SEQ ID NO: 97
KRKKQRSRRNDEELETRAHRVATEERGRKPHQIPASTPQNPATSQHPPPP
PGHRSQAPSHRPPPPGHRVQHQPQKRPPAPSGTQVHQQKGPPLPRPRVQP
KPPHGAAENSLSPSSNPFFFCCFIAVAMGIRFIIMVATTTPAPRPPTPAP
TIASQPLSLRPEACRPAAGGAVHTRGLDFACDDIQMTQTTSSLSASLGDR
VTISCRASQDISKYLNWYQQKPDGTVKLLIYHTSRLHSGVPSRFSGSGSG
TDYSLTISNLEQEDIATYFCQQGNTLPYTFGGGTKLEITGGGGSGGGGSG
GGGSEVKLQESGPGLVAPSQSLSVTCTVSGVSLPDYGVSWIRQPPRKGLE
WLGVIWGSETTYYNSALKSRLTIIKDNSKSQVFLKMNSLQTDDTAIYYCA
KHYYYGGSYAMDYWGQGTSVTVSSGGGGSDYKDDDDKDYKDDDDKDYKDD
DDK,

The CC032 construct comprises the following domains from N terminus to C terminus: CD8 signal peptide—3*Flag—linker (e.g., GGGGS, SEQ ID NO: 42)—anti-CD19 scFv—CD8a hinge domain—NKp46 transmembrane and cytoplasmic domains—2B4 cytoplasmic domain. See e.g., SEQ ID NO: 63 or SEQ ID NO: 98.

CC032
SEQ ID NO: 63
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACATGGGACTGGCCTTTCTGGTGCTGGTGGCTCTGGTGTGG
TTTCTGGTGGAGGACTGGCTGAGCAGAAAGAGGACCAGAGAGAGAGCCAG
CAGAGCCAGCACATGGGAGGGCAGAAGGAGACTGAACACCCAGACCCTGT
GGAGGAGGAAAAGGAAGGAGAAGCAGTCCGAGACATCCCCCAAAGAGTTT
TTAACCATTTATGAGGACGTGAAGGACCTCAAGACCAGAAGGAACCACGA
GCAAGAACAGACCTTTCCCGGCGGCGGATCCACCATCTACAGCATGATCC
AGAGCCAGAGCTCCGCCCCTACAAGCCAAGAACCCGCTTACACTTTATAT
TCTTTAATCCAGCCTTCTCGTAAGAGCGGCTCCCGTAAGAGGAACCACAG
CCCCAGCTTTAATAGCACCATTTACGAGGTCATCGGCAAGTCCCAGCCTA
AGGCCCAAAACCCCGCTCGTCTCTCTCGTAAGGAGCTGGAGAATTTCGAC
GTGTACAGCTAA,
CC032
SEQ ID NO: 98
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDMGLAFLVLVALVW
FLVEDWLSRKRTRERASRASTWEGRRRLNTQTLWRRKRKEKQSETSPKEF
LTIYEDVKDLKTRRNHEQEQTFPGGGSTIYSMIQSQSSAPTSQEPAYTLY
SLIQPSRKSGSRKRNHSPSFNSTIYEVIGKSQPKAQNPARLSRKELENFD
VYS,

The CC033 construct comprises the following domains from N terminus to C terminus: CD8 signal peptide—3*Flag—linker (e.g., GGGGS, SEQ ID NO: 42)—anti-CD19 scFv—CD8a hinge domain—NKp46 transmembrane and cytoplasmic domains—CRACC cytoplasmic domain. See e.g., SEQ ID NO: 64 or SEQ ID NO: 99.

CC033
SEQ ID NO: 64
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACATGGGACTGGCCTTTCTGGTGCTGGTGGCTCTGGTGTGG
TTTCTGGTGGAGGACTGGCTGAGCAGAAAGAGGACCAGAGAGAGAGCCAG
CAGAGCCAGCACATGGGAGGGCAGAAGGAGACTGAACACCCAGACCCTGT
GGTTTCTGAAGAGAGAGAGGCAAGAGGAGTACATCGAGGAGAAGAAGAGG
GTGGACATCTGCAGAGAGACCCCCAACATCTGCCCCCATTCCGGCGAGAA
CACCGAGTACGACACCATCCCCCACACCAACAGAACCATTCTGAAGGAGG
ACCCCGCCAACACCGTGTACAGCACCGTGGAGATCCCCAAGAAGATGGAG
AATCCCCACTCTCTGCTGACCATGCCCGACACCCCCAGACTGTTCGCCTA
TGAGAATGTTATCTAA,
CC033
SEQ ID NO: 99
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDMGLAFLVLVALVW
FLVEDWLSRKRTRERASRASTWEGRRRLNTQTLWFLKRERQEEYIEEKKR
VDICRETPNICPHSGENTEYDTIPHTNRTILKEDPANTVYSTVEIPKKME
NPHSLLTMPDTPRLFAYENVI,

The CC034 construct comprises the following domains from N terminus to C terminus: CD8 signal peptide—3*Flag—linker (e.g., GGGGS, SEQ ID NO: 42)—anti-CD19 scFv—CD8a hinge domain—DNAM1 transmembrane and cytoplasmic domains—CD2 cytoplasmic domain. See e.g., SEQ ID NO: 65 or SEQ ID NO: 100.

CC034
SEQ ID NO: 65
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACGGCGGCACCGTGCTGCTGCTGCTGTTCGTGATCTCCATC
ACCACCATCATCGTCATCTTTCTGAACAGAAGGAGGAGAAGGGAGAGGAG
GGATCTGTTCACCGAGAGCTGGGACACCCAGAAGGCCCCCAACAACTATA
GAAGCCCCATCAGCACCAGCCAGCCCACCAACCAGAGCATGGACGACACC
AGAGAGGACATCTACGTGAACTACCCCACCTTCAGCAGAAGACCCAAGAC
CAGAGTGAAGAGGAAGAAGCAGAGGTCTAGAAGAAACGACGAGGAGCTCG
AGACAAGAGCCCACAGAGTGGCTACCGAAGAGAGAGGCAGAAAGCCCCAC
CAGATCCCCGCCAGCACACCCCAGAACCCCGCCACAAGCCAGCATCCCCC
CCCTCCTCCCGGCCACAGATCCCAAGCTCCCAGCCACAGACCTCCTCCCC
CCGGCCATAGGGTGCAGCATCAGCCCCAGAAAAGACCTCCCGCCCCCAGC
GGAACCCAAGTGCATCAGCAGAAGGGCCCTCCTCTGCCCAGACCTAGGGT
GCAACCCAAGCCCCCTCATGGCGCTGCCGAGAACTCTCTGAGCCCTAGCA
GCAACTAA,
CC034
SEQ ID NO: 100
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDGGTVLLLLFVISI
TTIIVIFLNRRRRRERRDLFTESWDTQKAPNNYRSPISTSQPTNQSMDDT
REDIYVNYPTFSRRPKTRVKRKKQRSRRNDEELETRAHRVATEERGRKPH
QIPASTPQNPATSQHPPPPPGHRSQAPSHRPPPPGHRVQHQPQKRPPAPS
GTQVHQQKGPPLPRPRVQPKPPHGAAENSLSPSSN,

The CC035 construct comprises the following domains from N terminus to C terminus: CD8 signal peptide—3*Flag—linker (e.g., GGGGS, SEQ ID NO: 42)—anti-CD19 scFv—CD8a hinge domain—NKG2D transmembrane domain—CRACC cytoplasmic domain. See e.g., SEQ ID NO: 66 or SEQ ID NO: 101.

CC035
SEQ ID NO: 66
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACCCTTTCTTCTTCTGCTGCTTCATCGCCGTGGCCATGGGC
ATTCGTTTCATCATCATGGTGGCCTGGTTTCTGAAGAGAGAGAGGCAAGA
GGAGTACATCGAGGAGAAGAAGAGGGTGGACATCTGCAGAGAGACCCCCA
ACATCTGCCCCCATTCCGGCGAGAACACCGAGTACGACACCATCCCCCAC
ACCAACAGAACCATTCTGAAGGAGGACCCCGCCAACACCGTGTACAGCAC
CGTGGAGATCCCCAAGAAGATGGAGAATCCCCACTCTCTGCTGACCATGC
CCGACACCCCCAGACTGTTCGCCTATGAGAATGTTATCTAA,
CC035
SEQ ID NO: 101
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDPFFFCCFIAVAMG
IRFIIMVAWFLKRERQEEYIEEKKRVDICRETPNICPHSGENTEYDTIPH
TNRTILKEDPANTVYSTVEIPKKMENPHSLLTMPDTPRLFAYENVI,

The CC036 (e.g., reverse domain order of CC035, except not including the CD8 signal peptide) construct comprises the following domains from N terminus to C terminus: CRACC cytoplasmic domain—NKG2D transmembrane domain—CD8a hinge domain—anti-CD19 scFv—linker (e.g., GGGGS, SEQ ID NO: 42)—3*Flag. See e.g., SEQ ID NO: 67 or SEQ ID NO: 102.

CC036
SEQ ID NO: 67
TGGTTTCTGAAGAGAGAGAGGCAAGAGGAGTACATCGAGGAGAAGAAGAG
GGTGGACATCTGCAGAGAGACCCCCAACATCTGCCCCCATTCCGGCGAGA
ACACCGAGTACGACACCATCCCCCACACCAACAGAACCATTCTGAAGGAG
GACCCCGCCAACACCGTGTACAGCACCGTGGAGATCCCCAAGAAGATGGA
GAATCCCCACTCTCTGCTGACCATGCCCGACACCCCCAGACTGTTCGCCT
ATGAGAATGTTATCCCTTTCTTCTTCTGCTGCTTCATCGCCGTGGCCATG
GGCATTCGTTTCATCATCATGGTGGCCACCACAACCCCTGCCCCCAGACC
TCCCACACCCGCCCCTACCATCGCTAGCCAGCCTCTGAGCCTGAGGCCCG
AGGCTTGCAGACCTGCTGCTGGAGGCGCCGTGCACACCAGAGGACTGGAT
TTCGCCTGCGACGACATCCAGATGACCCAGACCACCAGCAGCCTGAGCGC
TAGCCTGGGCGATAGAGTGACCATCAGCTGCAGAGCCAGCCAGGACATCA
GCAAGTACCTGAACTGGTACCAGCAGAAGCCCGACGGCACAGTGAAACTG
CTGATCTACCACACCTCCAGACTGCATAGCGGCGTGCCTAGCAGGTTTTC
CGGCAGCGGCAGCGGAACCGACTACTCCCTGACCATCAGCAACCTGGAGC
AGGAGGACATCGCTACCTACTTTTGTCAGCAGGGCAACACCCTGCCTTAC
ACCTTTGGCGGCGGAACAAAGCTGGAGATTACCGGCGGCGGAGGCAGCGG
CGGCGGAGGCTCCGGAGGCGGAGGATCCGAGGTGAAGCTGCAGGAAAGCG
GACCTGGACTGGTCGCCCCTAGCCAGAGCCTGAGCGTGACCTGTACCGTG
AGCGGAGTGAGCCTGCCCGATTACGGCGTGTCCTGGATCAGGCAGCCCCC
CAGGAAGGGACTGGAATGGCTGGGCGTCATCTGGGGCTCCGAGACAACCT
ACTACAACAGCGCCCTCAAGTCCAGGCTCACCATTATCAAGGACAATAGC
AAGAGCCAGGTGTTCCTGAAGATGAACAGCCTGCAGACCGACGACACAGC
CATTTATTACTGCGCCAAGCACTATTATTACGGCGGCAGCTACGCCATGG
ACTATTGGGGCCAGGGAACAAGCGTGACCGTCAGCAGCGGTGGAGGCGGT
TCGGATTACAAGGATGACGACGATAAGGACTATAAGGACGACGACGATAA
GGATTATAAAGACGATGACGACAAATAA,
CC036
SEQ ID NO: 102
WFLKRERQEEYIEEKKRVDICRETPNICPHSGENTEYDTIPHTNRTILKE
DPANTVYSTVEIPKKMENPHSLLTMPDTPRLFAYENVIPFFFCCFIAVAM
GIRFIIMVATTTPAPRPPTPAPTIASQPLSLRPEACRPAAGGAVHTRGLD
FACDDIQMTQTTSSLSASLGDRVTISCRASQDISKYLNWYQQKPDGTVKL
LIYHTSRLHSGVPSRFSGSGSGTDYSLTISNLEQEDIATYFCQQGNTLPY
TFGGGTKLEITGGGGSGGGGSGGGGSEVKLQESGPGLVAPSQSLSVTCTV
SGVSLPDYGVSWIRQPPRKGLEWLGVIWGSETTYYNSALKSRLTIIKDNS
KSQVFLKMNSLQTDDTAIYYCAKHYYYGGSYAMDYWGQGTSVTVSSGGGG
SDYKDDDDKDYKDDDDKDYKDDDDK,

The CC037 construct comprises the following domains from N terminus to C terminus: CD8 signal peptide—3*Flag—linker (e.g., GGGGS, SEQ ID NO: 42)—anti-CD19 scFv—CD8a hinge domain—NKG2D transmembrane domain—CD2 cytoplasmic domain. See e.g., SEQ ID NO: 68 or SEQ ID NO: 103.

CC037
SEQ ID NO: 68
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACCCTTTCTTCTTCTGCTGCTTCATCGCCGTGGCCATGGGC
ATTCGTTTCATCATCATGGTGGCCAAGAGGAAGAAGCAGAGGTCTAGAAG
AAACGACGAGGAGCTCGAGACAAGAGCCCACAGAGTGGCTACCGAAGAGA
GAGGCAGAAAGCCCCACCAGATCCCCGCCAGCACACCCCAGAACCCCGCC
ACAAGCCAGCATCCCCCCCCTCCTCCCGGCCACAGATCCCAAGCTCCCAG
CCACAGACCTCCTCCCCCCGGCCATAGGGTGCAGCATCAGCCCCAGAAAA
GACCTCCCGCCCCCAGCGGAACCCAAGTGCATCAGCAGAAGGGCCCTCCT
CTGCCCAGACCTAGGGTGCAACCCAAGCCCCCTCATGGCGCTGCCGAGAA
CTCTCTGAGCCCTAGCAGCAACTAA,
CC037
SEQ ID NO: 103
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDPFFFCCFIAVAMG
IRFIIMVAKRKKQRSRRNDEELETRAHRVATEERGRKPHQIPASTPQNPA
TSQHPPPPPGHRSQAPSHRPPPPGHRVQHQPQKRPPAPSGTQVHQQKGPP
LPRPRVQPKPPHGAAENSLSPSSN,

The CC038 construct comprises the following domains from N terminus to C terminus: CD8 signal peptide—3*Flag—linker (e.g., GGGGS, SEQ ID NO: 42)—anti-CD19 scFv—CD8a hinge domain—DNAM1 transmembrane and cytoplasmic domains—NTB-A cytoplasmic domain. See e.g., SEQ ID NO: 69 or SEQ ID NO: 104.

CC038
SEQ ID NO: 69
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACGGCGGCACCGTGCTGCTGCTGCTGTTCGTGATCTCCATC
ACCACCATCATCGTCATCTTTCTGAACAGAAGGAGGAGAAGGGAGAGGAG
GGATCTGTTCACCGAGAGCTGGGACACCCAGAAGGCCCCCAACAACTATA
GAAGCCCCATCAGCACCAGCCAGCCCACCAACCAGAGCATGGACGACACC
AGAGAGGACATCTACGTGAACTACCCCACCTTCAGCAGAAGACCCAAGAC
CAGAGTGCTGAGAAAGAGAAGAGACTCTCTGTCTCTGAGCACCCAAAGAA
CACAAGGCCCCGAGAGCGCCAGAAATCTGGAGTACGTGAGCGTGAGCCCC
ACCAACAACACCGTGTACGCCAGCGTGACCCACAGCAACAGAGAGACCGA
GATCTGGACCCCTAGAGAGAACGACACCATCACCATCTACAGCACCATCA
ACCACAGCAAGGAGAGCAAGCCCACATTCTCTAGAGCCACCGCTCTGGAT
AACGTGGTGTAA,
CC038
SEQ ID NO: 104
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDGGTVLLLLFVISI
TTIIVIFLNRRRRRERRDLFTESWDTQKAPNNYRSPISTSQPTNQSMDDT
REDIYVNYPTFSRRPKTRVLRKRRDSLSLSTQRTQGPESARNLEYVSVSP
TNNTVYASVTHSNRETEIWTPRENDTITIYSTINHSKESKPTFSRATALD
NVV,

The CC039 construct comprises the following domains from N terminus to C terminus: CD8 signal peptide—3*Flag—linker (e.g., GGGGS, SEQ ID NO: 42)—anti-CD19 scFv—CD8a hinge domain—NKG2D transmembrane domain—NTB-A cytoplasmic domain. See e.g., SEQ ID NO: 70 or SEQ ID NO: 105.

CC039
SEQ ID NO: 70
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GACATCCAGATGACCCAGACCACCAGCAGCCTGAGCGCTAGCCTGGGCGA
TAGAGTGACCATCAGCTGCAGAGCCAGCCAGGACATCAGCAAGTACCTGA
ACTGGTACCAGCAGAAGCCCGACGGCACAGTGAAACTGCTGATCTACCAC
ACCTCCAGACTGCATAGCGGCGTGCCTAGCAGGTTTTCCGGCAGCGGCAG
CGGAACCGACTACTCCCTGACCATCAGCAACCTGGAGCAGGAGGACATCG
CTACCTACTTTTGTCAGCAGGGCAACACCCTGCCTTACACCTTTGGCGGC
GGAACAAAGCTGGAGATTACCGGCGGCGGAGGCAGCGGCGGCGGAGGCTC
CGGAGGCGGAGGATCCGAGGTGAAGCTGCAGGAAAGCGGACCTGGACTGG
TCGCCCCTAGCCAGAGCCTGAGCGTGACCTGTACCGTGAGCGGAGTGAGC
CTGCCCGATTACGGCGTGTCCTGGATCAGGCAGCCCCCCAGGAAGGGACT
GGAATGGCTGGGCGTCATCTGGGGCTCCGAGACAACCTACTACAACAGCG
CCCTCAAGTCCAGGCTCACCATTATCAAGGACAATAGCAAGAGCCAGGTG
TTCCTGAAGATGAACAGCCTGCAGACCGACGACACAGCCATTTATTACTG
CGCCAAGCACTATTATTACGGCGGCAGCTACGCCATGGACTATTGGGGCC
AGGGAACAAGCGTGACCGTCAGCAGCACCACAACCCCTGCCCCCAGACCT
CCCACACCCGCCCCTACCATCGCTAGCCAGCCTCTGAGCCTGAGGCCCGA
GGCTTGCAGACCTGCTGCTGGAGGCGCCGTGCACACCAGAGGACTGGATT
TCGCCTGCGACCCTTTCTTCTTCTGCTGCTTCATCGCCGTGGCCATGGGC
ATTCGTTTCATCATCATGGTGGCCCTGAGAAAGAGAAGAGACTCTCTGTC
TCTGAGCACCCAAAGAACACAAGGCCCCGAGAGCGCCAGAAATCTGGAGT
ACGTGAGCGTGAGCCCCACCAACAACACCGTGTACGCCAGCGTGACCCAC
AGCAACAGAGAGACCGAGATCTGGACCCCTAGAGAGAACGACACCATCAC
CATCTACAGCACCATCAACCACAGCAAGGAGAGCAAGCCCACATTCTCTA
GAGCCACCGCTCTGGATAACGTGGTGTAA,
CC039
SEQ ID NO: 105
MALPVTALLLPLALLLHAARPDYKDDDDKDYKDDDDKDYKDDDDKGGGGS
DIQMTQTTSSLSASLGDRVTISCRASQDISKYLNWYQQKPDGTVKLLIYH
TSRLHSGVPSRFSGSGSGTDYSLTISNLEQEDIATYFCQQGNTLPYTFGG
GTKLEITGGGGSGGGGSGGGGSEVKLQESGPGLVAPSQSLSVTCTVSGVS
LPDYGVSWIRQPPRKGLEWLGVIWGSETTYYNSALKSRLTIIKDNSKSQV
FLKMNSLQTDDTAIYYCAKHYYYGGSYAMDYWGQGTSVTVSSTTTPAPRP
PTPAPTIASQPLSLRPEACRPAAGGAVHTRGLDFACDPFFFCCFIAVAMG
IRFIIMVALRKRRDSLSLSTQRTQGPESARNLEYVSVSPTNNTVYASVTH
SNRETEIWTPRENDTITIYSTINHSKESKPTFSRATALDNVV,

Anti-CD33 Groups II-IV

Non-limiting examples of Group II-IV NK CARs that are specific to CD33 include CC124, CC125, CC130, CC135, or CC136, which correspond to polynucleotide SEQ ID NOs: 75-79 and polypeptide SEQ ID NOs: 110-114, respectively. It should be understood that other NK CARs can be generated by switching the CD-33-binding domain for a different tumor antigen- or cell-surface protein-binding domain.

The CC124 construct comprises the following domains from N terminus to C terminus: CD8 signal peptide—3*Flag—linker (e.g., GGGGS, SEQ ID NO: 42)—anti-CD33 scFv—CD8a hinge domain—NKG2D transmembrane domain—2B4 cytoplasmic domain. See e.g., SEQ ID NO: 75 or SEQ ID NO: 110.

CC124
SEQ ID NO: 75
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GAAGTGCAGCTGGTGCAGAGCGGAGCAGAAGTGAAGAAGCCCGGAAGCAG
CGTGAAGGTGTCTTGCAAGGCCAGCGGCTACACCATCACCGACAGCAACA
TCCATTGGGTCCGGCAGGCTCCAGGACAGTCTCTGGAGTGGATCGGCTAC
ATCTACCCCTACAACGGCGGCACCGACTACAACCAGAAGTTCAAGAACCG
GGCCACCCTGACCGTGGATAACCCCACCAACACCGCCTACATGGAGCTGA
GCAGCCTGAGAAGCGAGGACACCGCCTTCTACTATTGCGTGAACGGCAAC
CCTTGGCTGGCCTATTGGGGACAGGGAACACTGGTGACCGTGTCCTCTGG
CGGCGGAGGCAGCGGCGGCGGAGGCTCCGGAGGCGGAGGATCCGACATCC
AGCTGACCCAGTCTCCTAGCACCCTGAGCGCTAGCGTGGGAGATAGAGTG
ACCATCACTTGCAGAGCCAGCGAGAGCCTGGACAACTACGGCATCCGGTT
CCTGACTTGGTTCCAGCAGAAACCCGGCAAGGCCCCTAAACTGCTGATGT
ACGCCGCCTCTAACCAGGGAAGCGGAGTGCCTAGCAGATTCAGCGGCAGC
GGAAGCGGAACCGAGTTCACCCTGACCATCAGCTCTCTGCAGCCAGACGA
CTTCGCCACCTACTACTGCCAGCAGACCAAGGAGGTGCCTTGGAGCTTCG
GCCAGGGAACCAAGGTGGAAGTGAAGCGGACAGTGACCACAACCCCTGCC
CCCAGACCTCCCACACCCGCCCCTACCATCGCTAGCCAGCCTCTGAGCCT
GAGGCCCGAGGCTTGCAGACCTGCTGCTGGAGGCGCCGTGCACACCAGAG
GACTGGATTTCGCCTGCGACCCTTTCTTCTTCTGCTGCTTCATCGCCGTG
GCCATGGGCATTCGTTTCATCATCATGGTGGCCTGGAGGAGGAAAAGGAA
GGAGAAGCAGTCCGAGACATCCCCCAAAGAGTTTTTAACCATTTATGAGG
ACGTGAAGGACCTCAAGACCAGAAGGAACCACGAGCAAGAACAGACCTTT
CCCGGCGGCGGATCCACCATCTACAGCATGATCCAGAGCCAGAGCTCCGC
CCCTACAAGCCAAGAACCCGCTTACACTTTATATTCTTTAATCCAGCCTT
CTCGTAAGAGCGGCTCCCGTAAGAGGAACCACAGCCCCAGCTTTAATAGC
ACCATTTACGAGGTCATCGGCAAGTCCCAGCCTAAGGCCCAAAACCCCGC
TCGTCTCTCTCGTAAGGAGCTGGAGAATTTCGACGTGTACAGCTAA,
CC124
SEQ ID NO: 110
MALPVTALLLPLALLLHAARPDYKDDDDKDYKDDDDKDYKDDDDKGGGGS
EVQLVQSGAEVKKPGSSVKVSCKASGYTITDSNIHWVRQAPGQSLEWIGY
IYPYNGGTDYNQKFKNRATLTVDNPTNTAYMELSSLRSEDTAFYYCVNGN
PWLAYWGQGTLVTVSSGGGGSGGGGSGGGGSDIQLTQSPSTLSASVGDRV
TITCRASESLDNYGIRFLTWFQQKPGKAPKLLMYAASNQGSGVPSRFSGS
GSGTEFTLTISSLQPDDFATYYCQQTKEVPWSFGQGTKVEVKRTVTTTPA
PRPPTPAPTIASQPLSLRPEACRPAAGGAVHTRGLDFACDPFFFCCFIAV
AMGIRFIIMVAWRRKRKEKQSETSPKEFLTIYEDVKDLKTRRNHEQEQTF
PGGGSTIYSMIQSQSSAPTSQEPAYTLYSLIQPSRKSGSRKRNHSPSFNS
TIYEVIGKSQPKAQNPARLSRKELENFDVYS,

The CC125 construct (e.g., reverse domain order of CC124, except not including the CD8 signal peptide) comprises the following domains from N terminus to C terminus: 2B4 cytoplasmic domain—NKG2D transmembrane domain—CD8a hinge domain—anti-CD33 scFv—linker (e.g., GGGGS, SEQ ID NO: 42)—3*Flag. See e.g., SEQ ID NO: 76 or SEQ ID NO: 111.

CC125
SEQ ID NO: 76
TGGAGGAGGAAAAGGAAGGAGAAGCAGTCCGAGACATCCCCCAAAGAGTT
TTTAACCATTTATGAGGACGTGAAGGACCTCAAGACCAGAAGGAACCACG
AGCAAGAACAGACCTTTCCCGGCGGCGGATCCACCATCTACAGCATGATC
CAGAGCCAGAGCTCCGCCCCTACAAGCCAAGAACCCGCTTACACTTTATA
TTCTTTAATCCAGCCTTCTCGTAAGAGCGGCTCCCGTAAGAGGAACCACA
GCCCCAGCTTTAATAGCACCATTTACGAGGTCATCGGCAAGTCCCAGCCT
AAGGCCCAAAACCCCGCTCGTCTCTCTCGTAAGGAGCTGGAGAATTTCGA
CGTGTACAGCCCTTTCTTCTTCTGCTGCTTCATCGCCGTGGCCATGGGCA
TTCGTTTCATCATCATGGTGGCCACCACAACCCCTGCCCCCAGACCTCCC
ACACCCGCCCCTACCATCGCTAGCCAGCCTCTGAGCCTGAGGCCCGAGGC
TTGCAGACCTGCTGCTGGAGGCGCCGTGCACACCAGAGGACTGGATTTCG
CCTGCGACGAAGTGCAGCTGGTGCAGAGCGGAGCAGAAGTGAAGAAGCCC
GGAAGCAGCGTGAAGGTGTCTTGCAAGGCCAGCGGCTACACCATCACCGA
CAGCAACATCCATTGGGTCCGGCAGGCTCCAGGACAGTCTCTGGAGTGGA
TCGGCTACATCTACCCCTACAACGGCGGCACCGACTACAACCAGAAGTTC
AAGAACCGGGCCACCCTGACCGTGGATAACCCCACCAACACCGCCTACAT
GGAGCTGAGCAGCCTGAGAAGCGAGGACACCGCCTTCTACTATTGCGTGA
ACGGCAACCCTTGGCTGGCCTATTGGGGACAGGGAACACTGGTGACCGTG
TCCTCTGGCGGCGGAGGCAGCGGCGGCGGAGGCTCCGGAGGCGGAGGATC
CGACATCCAGCTGACCCAGTCTCCTAGCACCCTGAGCGCTAGCGTGGGAG
ATAGAGTGACCATCACTTGCAGAGCCAGCGAGAGCCTGGACAACTACGGC
ATCCGGTTCCTGACTTGGTTCCAGCAGAAACCCGGCAAGGCCCCTAAACT
GCTGATGTACGCCGCCTCTAACCAGGGAAGCGGAGTGCCTAGCAGATTCA
GCGGCAGCGGAAGCGGAACCGAGTTCACCCTGACCATCAGCTCTCTGCAG
CCAGACGACTTCGCCACCTACTACTGCCAGCAGACCAAGGAGGTGCCTTG
GAGCTTCGGCCAGGGAACCAAGGTGGAAGTGAAGCGGACAGTGGGTGGAG
GCGGTTCGGATTACAAGGATGACGACGATAAGGACTATAAGGACGACGAC
GATAAGGATTATAAAGACGATGACGACAAATAA,
CC125
SEQ ID NO: 111
WRRKRKEKQSETSPKEFLTIYEDVKDLKTRRNHEQEQTFPGGGSTIYSMI
QSQSSAPTSQEPAYTLYSLIQPSRKSGSRKRNHSPSFNSTIYEVIGKSQP
KAQNPARLSRKELENFDVYSPFFFCCFIAVAMGIRFIIMVATTTPAPRPP
TPAPTIASQPLSLRPEACRPAAGGAVHTRGLDFACDEVQLVQSGAEVKKP
GSSVKVSCKASGYTITDSNIHWVRQAPGQSLEWIGYIYPYNGGTDYNQKF
KNRATLTVDNPTNTAYMELSSLRSEDTAFYYCVNGNPWLAYWGQGTLVTV
SSGGGGSGGGGSGGGGSDIQLTQSPSTLSASVGDRVTITCRASESLDNYG
IRFLTWFQQKPGKAPKLLMYAASNQGSGVPSRFSGSGSGTEFTLTISSLQ
PDDFATYYCQQTKEVPWSFGQGTKVEVKRTVGGGGSDYKDDDDKDYKDDD
DKDYKDDDDK,

The CC130 construct comprises the following domains from N terminus to C terminus: CD8 signal peptide—3*Flag—linker (e.g., GGGGS, SEQ ID NO: 42)—anti-CD33 scFv—CD8a hinge domain—DNAM1 transmembrane and cytoplasmic domains—CRACC cytoplasmic domain. See e.g., SEQ ID NO: 77 or SEQ ID NO: 112.

CC130
SEQ ID NO: 77
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GAAGTGCAGCTGGTGCAGAGCGGAGCAGAAGTGAAGAAGCCCGGAAGCAG
CGTGAAGGTGTCTTGCAAGGCCAGCGGCTACACCATCACCGACAGCAACA
TCCATTGGGTCCGGCAGGCTCCAGGACAGTCTCTGGAGTGGATCGGCTAC
ATCTACCCCTACAACGGCGGCACCGACTACAACCAGAAGTTCAAGAACCG
GGCCACCCTGACCGTGGATAACCCCACCAACACCGCCTACATGGAGCTGA
GCAGCCTGAGAAGCGAGGACACCGCCTTCTACTATTGCGTGAACGGCAAC
CCTTGGCTGGCCTATTGGGGACAGGGAACACTGGTGACCGTGTCCTCTGG
CGGCGGAGGCAGCGGCGGCGGAGGCTCCGGAGGCGGAGGATCCGACATCC
AGCTGACCCAGTCTCCTAGCACCCTGAGCGCTAGCGTGGGAGATAGAGTG
ACCATCACTTGCAGAGCCAGCGAGAGCCTGGACAACTACGGCATCCGGTT
CCTGACTTGGTTCCAGCAGAAACCCGGCAAGGCCCCTAAACTGCTGATGT
ACGCCGCCTCTAACCAGGGAAGCGGAGTGCCTAGCAGATTCAGCGGCAGC
GGAAGCGGAACCGAGTTCACCCTGACCATCAGCTCTCTGCAGCCAGACGA
CTTCGCCACCTACTACTGCCAGCAGACCAAGGAGGTGCCTTGGAGCTTCG
GCCAGGGAACCAAGGTGGAAGTGAAGCGGACAGTGACCACAACCCCTGCC
CCCAGACCTCCCACACCCGCCCCTACCATCGCTAGCCAGCCTCTGAGCCT
GAGGCCCGAGGCTTGCAGACCTGCTGCTGGAGGCGCCGTGCACACCAGAG
GACTGGATTTCGCCTGCGACGGCGGCACCGTGCTGCTGCTGCTGTTCGTG
ATCTCCATCACCACCATCATCGTCATCTTTCTGAACAGAAGGAGGAGAAG
GGAGAGGAGGGATCTGTTCACCGAGAGCTGGGACACCCAGAAGGCCCCCA
ACAACTATAGAAGCCCCATCAGCACCAGCCAGCCCACCAACCAGAGCATG
GACGACACCAGAGAGGACATCTACGTGAACTACCCCACCTTCAGCAGAAG
ACCCAAGACCAGAGTGTGGTTTCTGAAGAGAGAGAGGCAAGAGGAGTACA
TCGAGGAGAAGAAGAGGGTGGACATCTGCAGAGAGACCCCCAACATCTGC
CCCCATTCCGGCGAGAACACCGAGTACGACACCATCCCCCACACCAACAG
AACCATTCTGAAGGAGGACCCCGCCAACACCGTGTACAGCACCGTGGAGA
TCCCCAAGAAGATGGAGAATCCCCACTCTCTGCTGACCATGCCCGACACC
CCCAGACTGTTCGCCTATGAGAATGTTATCTAA,
CC130
SEQ ID NO: 112
MALPVTALLLPLALLLHAARPDYKDDDDKDYKDDDDKDYKDDDDKGGGGS
EVQLVQSGAEVKKPGSSVKVSCKASGYTITDSNIHWVRQAPGQSLEWIGY
IYPYNGGTDYNQKFKNRATLTVDNPTNTAYMELSSLRSEDTAFYYCVNGN
PWLAYWGQGTLVTVSSGGGGSGGGGSGGGGSDIQLTQSPSTLSASVGDRV
TITCRASESLDNYGIRFLTWFQQKPGKAPKLLMYAASNQGSGVPSRFSGS
GSGTEFTLTISSLQPDDFATYYCQQTKEVPWSFGQGTKVEVKRTVTTTPA
PRPPTPAPTIASQPLSLRPEACRPAAGGAVHTRGLDFACDGGTVLLLLFV
ISITTIIVIFLNRRRRRERRDLFTESWDTQKAPNNYRSPISTSQPTNQSM
DDTREDIYVNYPTFSRRPKTRVWFLKRERQEEYIEEKKRVDICRETPNIC
PHSGENTEYDTIPHTNRTILKEDPANTVYSTVEIPKKMENPHSLLTMPDT
PRLFAYENVI,

The CC135 construct comprises the following domains from N terminus to C terminus: CD8 signal peptide—3*Flag—linker (e.g., GGGGS, SEQ ID NO: 42)—anti-CD33 scFv—CD8a hinge domain—NKG2D transmembrane domain—CRACC cytoplasmic domain. See e.g., SEQ ID NO: 78 or SEQ ID NO: 113.

CC135
SEQ ID NO: 78
ATGGCCTTACCAGTGACCGCCTTGCTCCTGCCGCTGGCCTTGCTGCTCCA
CGCCGCCAGGCCGGATTACAAGGATGACGACGATAAGGACTATAAGGACG
ACGACGATAAGGATTATAAAGACGATGACGACAAAGGTGGAGGCGGTTCG
GAAGTGCAGCTGGTGCAGAGCGGAGCAGAAGTGAAGAAGCCCGGAAGCAG
CGTGAAGGTGTCTTGCAAGGCCAGCGGCTACACCATCACCGACAGCAACA
TCCATTGGGTCCGGCAGGCTCCAGGACAGTCTCTGGAGTGGATCGGCTAC
ATCTACCCCTACAACGGCGGCACCGACTACAACCAGAAGTTCAAGAACCG
GGCCACCCTGACCGTGGATAACCCCACCAACACCGCCTACATGGAGCTGA
GCAGCCTGAGAAGCGAGGACACCGCCTTCTACTATTGCGTGAACGGCAAC
CCTTGGCTGGCCTATTGGGGACAGGGAACACTGGTGACCGTGTCCTCTGG
CGGCGGAGGCAGCGGCGGCGGAGGCTCCGGAGGCGGAGGATCCGACATCC
AGCTGACCCAGTCTCCTAGCACCCTGAGCGCTAGCGTGGGAGATAGAGTG
ACCATCACTTGCAGAGCCAGCGAGAGCCTGGACAACTACGGCATCCGGTT
CCTGACTTGGTTCCAGCAGAAACCCGGCAAGGCCCCTAAACTGCTGATGT
ACGCCGCCTCTAACCAGGGAAGCGGAGTGCCTAGCAGATTCAGCGGCAGC
GGAAGCGGAACCGAGTTCACCCTGACCATCAGCTCTCTGCAGCCAGACGA
CTTCGCCACCTACTACTGCCAGCAGACCAAGGAGGTGCCTTGGAGCTTCG
GCCAGGGAACCAAGGTGGAAGTGAAGCGGACAGTGACCACAACCCCTGCC
CCCAGACCTCCCACACCCGCCCCTACCATCGCTAGCCAGCCTCTGAGCCT
GAGGCCCGAGGCTTGCAGACCTGCTGCTGGAGGCGCCGTGCACACCAGAG
GACTGGATTTCGCCTGCGACCCTTTCTTCTTCTGCTGCTTCATCGCCGTG
GCCATGGGCATTCGTTTCATCATCATGGTGGCCTGGTTTCTGAAGAGAGA
GAGGCAAGAGGAGTACATCGAGGAGAAGAAGAGGGTGGACATCTGCAGAG
AGACCCCCAACATCTGCCCCCATTCCGGCGAGAACACCGAGTACGACACC
ATCCCCCACACCAACAGAACCATTCTGAAGGAGGACCCCGCCAACACCGT
GTACAGCACCGTGGAGATCCCCAAGAAGATGGAGAATCCCCACTCTCTGC
TGACCATGCCCGACACCCCCAGACTGTTCGCCTATGAGAATGTTATCTA
A,
CC135
SEQ ID NO: 113
MALPVTALLLPLALLLHAARPDYKDDDDKDYKDDDDKDYKDDDDKGGGGS
EVQLVQSGAEVKKPGSSVKVSCKASGYTITDSNIHWVRQAPGQSLEWIGY
IYPYNGGTDYNQKFKNRATLTVDNPTNTAYMELSSLRSEDTAFYYCVNGN
PWLAYWGQGTLVTVSSGGGGSGGGGSGGGGSDIQLTQSPSTLSASVGDRV
TITCRASESLDNYGIRFLTWFQQKPGKAPKLLMYAASNQGSGVPSRFSGS
GSGTEFTLTISSLQPDDFATYYCQQTKEVPWSFGQGTKVEVKRTVTTTPA
PRPPTPAPTIASQPLSLRPEACRPAAGGAVHTRGLDFACDPFFFCCFIAV
AMGIRFIIMVAWFLKRERQEEYIEEKKRVDICRETPNICPHSGENTEYDT
IPHTNRTILKEDPANTVYSTVEIPKKMENPHSLLTMPDTPRLFAYENVI,

The CC136 construct (e.g., reverse domain order of CC135, except not including the CD8 signal peptide) comprises the following domains from N terminus to C terminus: CRACC cytoplasmic domain—NKG2D transmembrane domain—CD8a hinge domain—anti-CD33 scFv—linker (e.g., GGGGS, SEQ ID NO: 42)—3*Flag. See e.g., SEQ ID NO: 79 or SEQ ID NO: 114.

CC136
SEQ ID NO: 79
TGGTTTCTGAAGAGAGAGAGGCAAGAGGAGTACATCGAGGAGAAGAAGAG
GGTGGACATCTGCAGAGAGACCCCCAACATCTGCCCCCATTCCGGCGAGA
ACACCGAGTACGACACCATCCCCCACACCAACAGAACCATTCTGAAGGAG
GACCCCGCCAACACCGTGTACAGCACCGTGGAGATCCCCAAGAAGATGGA
GAATCCCCACTCTCTGCTGACCATGCCCGACACCCCCAGACTGTTCGCCT
ATGAGAATGTTATCCCTTTCTTCTTCTGCTGCTTCATCGCCGTGGCCATG
GGCATTCGTTTCATCATCATGGTGGCCACCACAACCCCTGCCCCCAGACC
TCCCACACCCGCCCCTACCATCGCTAGCCAGCCTCTGAGCCTGAGGCCCG
AGGCTTGCAGACCTGCTGCTGGAGGCGCCGTGCACACCAGAGGACTGGAT
TTCGCCTGCGACGAAGTGCAGCTGGTGCAGAGCGGAGCAGAAGTGAAGAA
GCCCGGAAGCAGCGTGAAGGTGTCTTGCAAGGCCAGCGGCTACACCATCA
CCGACAGCAACATCCATTGGGTCCGGCAGGCTCCAGGACAGTCTCTGGAG
TGGATCGGCTACATCTACCCCTACAACGGCGGCACCGACTACAACCAGAA
GTTCAAGAACCGGGCCACCCTGACCGTGGATAACCCCACCAACACCGCCT
ACATGGAGCTGAGCAGCCTGAGAAGCGAGGACACCGCCTTCTACTATTGC
GTGAACGGCAACCCTTGGCTGGCCTATTGGGGACAGGGAACACTGGTGAC
CGTGTCCTCTGGCGGCGGAGGCAGCGGCGGCGGAGGCTCCGGAGGCGGAG
GATCCGACATCCAGCTGACCCAGTCTCCTAGCACCCTGAGCGCTAGCGTG
GGAGATAGAGTGACCATCACTTGCAGAGCCAGCGAGAGCCTGGACAACTA
CGGCATCCGGTTCCTGACTTGGTTCCAGCAGAAACCCGGCAAGGCCCCTA
AACTGCTGATGTACGCCGCCTCTAACCAGGGAAGCGGAGTGCCTAGCAGA
TTCAGCGGCAGCGGAAGCGGAACCGAGTTCACCCTGACCATCAGCTCTCT
GCAGCCAGACGACTTCGCCACCTACTACTGCCAGCAGACCAAGGAGGTGC
CTTGGAGCTTCGGCCAGGGAACCAAGGTGGAAGTGAAGCGGACAGTGGGT
GGAGGCGGTTCGGATTACAAGGATGACGACGATAAGGACTATAAGGACGA
CGACGATAAGGATTATAAAGACGATGACGACAAATAA,
CC136
SEQ ID NO: 114
WFLKRERQEEYIEEKKRVDICRETPNICPHSGENTEYDTIPHTNRTILKE
DPANTVYSTVEIPKKMENPHSLLTMPDTPRLFAYENVIPFFFCCFIAVAM
GIRFIIMVATTTPAPRPPTPAPTIASQPLSLRPEACRPAAGGAVHTRGLD
FACDEVOLVOSGAEVKKPGSSVKVSCKASGYTITDSNIHWVRQAPGQSLE
WIGYIYPYNGGTDYNQKFKNRATLTVDNPTNTAYMELSSLRSEDTAFYYC
VNGNPWLAYWGQGTLVTVSSGGGGSGGGGSGGGGSDIQLTQSPSTLSASV
GDRVTITCRASESLDNYGIRFLTWFQQKPGKAPKLLMYAASNQGSGVPSR
FSGSGSGTEFTLTISSLQPDDFATYYCQQTKEVPWSFGQGTKVEVKRTVG
GGGSDYKDDDDKDYKDDDDKDYKDDDDK,

### Polynucleotides and Vectors

In multiple aspects, described herein are polynucleotides that encode for NK CAR polypeptides. In some embodiments of any of the aspects, a CAR polynucleotide comprises one of SEQ ID NOs: 45-79 (see e.g., Table 5), or a sequence that is at least 70%, at least 75%, at least 80%, at least 85%, at least 90%, at least 91%, at least 92%, at least 93%, at least 94%, at least 95%, at least 96%, at least 97%, at least 98%, or at least 99% identical to the sequence of one of SEQ ID NOs: 45-79, that as a polypeptide maintains the same functions (e.g., antigen-binding and intracellular signaling; see e.g., SEQ ID NOs: 80-114). Table 5 shows exemplary CAR polynucleotides and the specific order of their extracellular, transmembrane, and intracellular domains.

TABLE 5
Construct and Domain Nucleic Acid SEQ ID NOs (“TM” indicates transmembrane domain)

| | | SEQ ID NOs of domains, from 5′ to 3′ | | | | | | | | | | | | |
|---|---|---|---|---|---|---|---|---|---|---|---|---|---|---|
| | Construct | (except where indicated in next column) | | | | | | | | | | | | Domains in 3′ |
| | SEQ ID NO | Extracellular | | | | | | Intracellular | | | | | | to 5′ order |
| Construct | (nucleic acid) | Domains | | | | | TM | Domains | | | | | | (compare to) |
| CC002 | 45 | 37 | 39 | 41 | 33 | 43 | 28 | 13 | | | | | | |
| CC003 | 46 | 37 | | 41 | 33 | 43 | 28 | 13 | 5 | | | | | |
| CC004 | 47 | 37 | 39 | 41 | 33 | 43 | 28 | 13 | 5 | | | | | |
| CC005 | 48 | 37 | 39 | 41 | 33 | 43 | 28 | 13 | 6 | | | | | |
| CC007 | 49 | 37 | 39 | 41 | 33 | 43 | 28 | 13 | 6 | 17 | 21 | | | |
| CC008 | 50 | 37 | 39 | 41 | 33 | 43 | 28 | 13 | 6 | 17 | 21 | 18 | 22 | |
| CC013 | 51 | 37 | 39 | 41 | 33 | 43 | 28 | 13 | 14 | 5 | | | | |
| CC016 | 52 | 37 | 39 | 41 | 33 | 43 | 28 | 14 | 13 | 5 | | | | |
| CC017 | 53 | 37 | 39 | 41 | 33 | 43 | 28 | 13 | 14 | 6 | | | | |
| CC018 | 54 | 37 | 39 | 41 | 33 | 43 | 28 | 14 | 13 | 6 | | | | |
| CC024 | 55 | 37 | 39 | 41 | 33 | 43 | 25 | 1 | | | | | | |
| CC025 | 56 | | 39 | 41 | 33 | 43 | 25 | 1 | | | | | | 3′ to 5′ (CC024) |
| CC026 | 57 | 37 | 39 | 41 | 33 | 43 | 26 | 3 | | | | | | |
| CC027 | 58 | 37 | 39 | 41 | 33 | 43 | 27 | 1 | | | | | | |
| CC028 | 59 | | 39 | 41 | 33 | 43 | 25 | 4 | | | | | | 3′ to 5′ (CC039) |
| CC029 | 60 | 37 | 39 | 41 | 33 | 43 | 26 | 4 | | | | | | |
| CC030 | 61 | 37 | 39 | 41 | 33 | 43 | 27 | 2 | | | | | | |
| CC031 | 62 | | 39 | 41 | 33 | 43 | 25 | 3 | | | | | | 3′ to 5′ (CC037) |
| CC032 | 63 | 37 | 39 | 41 | 33 | 43 | 26 | 1 | | | | | | |
| CC033 | 64 | 37 | 39 | 41 | 33 | 43 | 26 | 2 | | | | | | |
| CC034 | 65 | 37 | 39 | 41 | 33 | 43 | 27 | 3 | | | | | | |
| CC035 | 66 | 37 | 39 | 41 | 33 | 43 | 25 | 2 | | | | | | |
| CC036 | 67 | | 39 | 41 | 33 | 43 | 25 | 2 | | | | | | 3′ to 5′ (CC035) |
| CC037 | 68 | 37 | 39 | 41 | 33 | 43 | 25 | 3 | | | | | | |
| CC038 | 69 | 37 | 39 | 41 | 33 | 43 | 27 | 4 | | | | | | |
| CC039 | 70 | 37 | 39 | 41 | 33 | 43 | 25 | 4 | | | | | | |
| CC103 | 71 | 37 | | 41 | 34 | 43 | 28 | 13 | 5 | | | | | |
| CC104 | 72 | 37 | 39 | 41 | 34 | 43 | 28 | 13 | 5 | | | | | |
| CC108 | 73 | 37 | 39 | 41 | 34 | 43 | 28 | 13 | 6 | 17 | 21 | 18 | 22 | |
| CC118 | 74 | 37 | 39 | 41 | 34 | 43 | 28 | 14 | 13 | 6 | | | | |
| CC124 | 75 | 37 | 39 | 41 | 34 | 43 | 25 | 1 | | | | | | |
| CC125 | 76 | | 39 | 41 | 34 | 43 | 25 | 1 | | | | | | 3′ to 5′ (CC124) |
| CC130 | 77 | 37 | 39 | 41 | 34 | 43 | 27 | 2 | | | | | | |
| CC135 | 78 | 37 | 39 | 41 | 34 | 43 | 25 | 2 | | | | | | |
| CC136 | 79 | | 39 | 41 | 34 | 43 | 25 | 2 | | | | | | 3′ to 5′ (CC135) |

In some embodiments, the CAR polynucleotide is a codon-optimized version of SEQ ID NOs: 145-79. In some embodiments of any of the aspects, the vector or nucleic acid described herein is codon-optimized, e.g., the native or wild-type sequence of the nucleic acid sequence has been altered or engineered to include alternative codons such that altered or engineered nucleic acid encodes the same polypeptide expression product as the native/wild-type sequence, but will be transcribed and/or translated at an improved efficiency in a desired expression system. In some embodiments of any of the aspects, the expression system is an organism other than the source of the native/wild-type sequence (or a cell obtained from such organism). In some embodiments of any of the aspects, the vector and/or nucleic acid sequence described herein is codon-optimized for expression in a mammal or mammalian cell, e.g., a mouse, a murine cell, or a human cell. In some embodiments of any of the aspects, the vector and/or nucleic acid sequence described herein is codon-optimized for expression in a human cell. In some embodiments of any of the aspects, the vector and/or nucleic acid sequence described herein is codon-optimized for expression in a yeast or yeast cell. In some embodiments of any of the aspects, the vector and/or nucleic acid sequence described herein is codon-optimized for expression in a bacterial cell. In some embodiments of any of the aspects, the vector and/or nucleic acid sequence described herein is codon-optimized for expression in an E. coli cell.

In some embodiments, one or more of the genes described herein is expressed in a recombinant expression vector or plasmid. As used herein, the term “vector” refers to a polynucleotide sequence suitable for transferring transgenes into a host cell. A vector can also include a viral particle that carries such a polynucleotide sequence. Vectors include, for example, plasmids, mini-chromosomes, phage, naked DNA and the like. See, for example, U.S. Pat. Nos. 4,980,285; 5,631,150; 5,707,828; 5,759,828; 5,888,783 and, 5,919,670, and, Sambrook et al, Molecular Cloning: A Laboratory Manual, 2nd Ed., Cold Spring Harbor Press (1989). One type of vector is a plasmid, which refers to a circular double stranded DNA loop into which additional DNA segments are ligated. Another type of vector is a viral vector, wherein additional DNA segments are ligated into the viral genome. Certain vectors are capable of autonomous replication in a host cell into which they are introduced (e.g., bacterial vectors having a bacterial origin of replication and episomal mammalian vectors). Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” are used interchangeably as the plasmid is the most commonly used form of vector. However, the technology described herein is intended to include other forms of expression vectors, such as viral vectors (e.g., replication defective retroviruses, adenoviruses and adeno-associated viruses), which serve equivalent functions and facilitate delivery of recombinant constructs to, e.g., NK cells.

A cloning vector is one which is able to replicate in a host cell or becomes integrated into the genome in a host cell, and which is further characterized by one or more endonuclease restriction sites at which the vector may be cut in a determinable fashion and into which a desired DNA sequence can be ligated such that the new recombinant vector retains its ability to replicate in the host cell. In the case of plasmids, replication of the desired sequence can occur many times as the plasmid increases in copy number within the host cell such as a host bacterium or just a single time per host before the host reproduces by mitosis. In the case of phage, replication can occur actively during a lytic phase or passively during a lysogenic phase.

An expression vector is one into which a desired DNA sequence can be inserted by restriction and ligation such that it is operably joined to regulatory sequences and can be expressed as an RNA transcript. Vectors can further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art (e.g., β-galactosidase, luciferase or alkaline phosphatase), and genes which visibly affect the phenotype of transformed or transfected cells, hosts, colonies or plaques (e.g., green fluorescent protein). In certain embodiments, the vectors used herein are capable of autonomous replication and expression of the structural gene products present in the DNA segments to which they are operably joined.

As used herein, a coding sequence and regulatory sequences are said to be “operably” joined when they are covalently linked in such a way as to place the expression or transcription of the coding sequence under the influence or control of the regulatory sequences. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably joined to a coding sequence if the promoter region were capable of effecting transcription of that DNA sequence such that the resulting transcript can be translated into the desired protein or polypeptide.

When the nucleic acid molecule that encodes any of the polypeptides described herein is expressed in a cell, a variety of transcription control sequences (e.g., promoter/enhancer sequences) can be used to direct its expression. The promoter can be a native promoter, i.e., the promoter of the gene in its endogenous context, which provides normal regulation of expression of the gene. In some embodiments the promoter can be constitutive, i.e., the promoter is unregulated allowing for continual transcription of its associated gene. A variety of conditional promoters also can be used, such as promoters controlled by the presence or absence of a molecule.

The precise nature of the regulatory sequences needed for gene expression can vary between species or cell types, but in general can include, as necessary, 5′ non-transcribed and 5′ non-translated sequences involved with the initiation of transcription and translation respectively, such as a TATA box, capping sequence, CAAT sequence, and the like. In particular, such 5′ non-transcribed regulatory sequences will include a promoter region which includes a promoter sequence for transcriptional control of the operably joined gene. Regulatory sequences can also include enhancer sequences or upstream activator sequences as desired. The vectors of the invention may optionally include 5′ leader or signal sequences. The choice and design of an appropriate vector is within the ability and discretion of one of ordinary skill in the art.

In some embodiments of any of the aspects, the promoter is a eukaryotic or human constitutive promoter, including but not limited to EF-1alpha, SFFV, CMV, and the like. In some embodiments of any of the aspects, the vector comprises a human elongation factor-1 alpha (EF-1alpha) promoter, which is a constitutive promoter of human origin that can be used to drive ectopic gene expression in various in vitro and in vivo contexts. In some embodiments of any of the aspects, the vector comprises a silencing-prone spleen focus forming virus (SFFV) promoter, which can result in a higher level of constitutive transgene expression compared with CMV or EF1α promoters. In some embodiments of any of the aspects, the vector comprises a Kozak sequence (e.g., GCCGCCACC), which is a nucleic acid motif that functions as the protein translation initiation site in eukaryotic mRNA transcripts.

Expression vectors containing all the necessary elements for expression are commercially available and known to those skilled in the art. See, e.g., Sambrook et al., Molecular Cloning: A Laboratory Manual, Second Edition, Cold Spring Harbor Laboratory Press, 1989. Cells are genetically engineered by the introduction into the cells of heterologous DNA (RNA). That heterologous DNA (RNA) is placed under operable control of transcriptional elements to permit the expression of the heterologous DNA in the host cell.

In some embodiments of any of the aspects, the vector is a lentiviral vector. The term “lentivirus” refers to a genus of the Retroviridae family. Lentiviruses are unique among the retroviruses in being able to infect non-dividing cells; they can deliver a significant amount of genetic information into the DNA of the host cell, so they provide one of the most efficient methods of a gene delivery to, e.g., mammalian cells. HIV, SIV, and FIV are all examples of lentiviruses. The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

In some embodiments of any of the aspects, the lentiviral vector comprises a central polypurine tract (cPPT). A central polypurine tract/central termination sequence creates a “DNA flap” that increases nuclear importation of the viral genome during target-cell infection. The cPPT/CTS element improves vector integration and transduction efficiency. In some embodiments of any of the aspects, the lentiviral vector comprises a woodchuck hepatitis virus posttranscriptional regulatory element (WPRE), which prevents poly(A) site read-through, promotes RNA processing and maturation, and increases nuclear export of RNA. In genomic transcripts, it enhances vector packaging and increases titer. In transduced target cells, the WPRE boosts transgene expression by facilitating mRNA transcript maturation.

In some embodiments, the vector comprises a selectable marker, e.g., for selectively amplifying the vector in bacteria. Non-limiting examples of selectable marker genes for use in bacteria include antibiotic resistance genes conferring resistance to ampicillin, tetracycline and kanamycin. The tetracycline (tet) and ampicillin (amp) resistance marker genes can be obtained from any of a number of commercially available vectors including pBR322 (available from New England BioLabs, Beverly, Mass., cat. no. 303-3s). The tet coding sequence is contained within nucleotides 86-476; the amp gene is contained within nucleotides 3295-4155. The nucleotide sequence of the kanamycin (kan) gene is available from vector pACYC 177, from New England BioLabs, Cat no. 401-L, GenBank accession No. X06402.

In some embodiments, one or more of the recombinantly expressed genes can be integrated into the genome of the cell.

A nucleic acid molecule that encodes an NK CAR polypeptide as described herein can be introduced into a cell or cells using methods and techniques that are standard in the art. For example, nucleic acid molecules can be introduced by standard protocols such as transformation including chemical transformation and electroporation, transduction, particle bombardment, etc. Expressing the nucleic acid molecule encoding the NK CAR polypeptide as described herein also may be accomplished by integrating the nucleic acid molecule into the genome.

### Cells

In one aspect, described herein is a cell or population thereof comprising at least one NK CAR polypeptide as described herein. In one aspect described herein is a cell (e.g., an NK cell) engineered to express a CAR, wherein the CAR NK cell exhibits an antitumor property. In one aspect a cell is transformed with the CAR and the CAR is expressed on the cell surface. In some embodiments, the cell (e.g., NK cell) is transduced with a viral vector encoding a CAR. In some embodiments, the viral vector is a retroviral vector. In some embodiments, the viral vector is a lentiviral vector. In some such embodiments, the cell may stably express the CAR. In another embodiment, the cell (e.g., NK cell) is transfected with a nucleic acid, e.g., mRNA, cDNA, DNA, encoding a CAR. In some such embodiments, the cell may transiently express the CAR.

In some embodiments of any of the aspects, the cell comprises an immune cell. In some embodiments of any of the aspects, the immune cell comprises a natural killer (NK) cell, a CD4+ T cell, a CD8+ T cell, or a regulatory T cell (Treg). In some embodiments of any of the aspects, the immune cell comprises a natural killer (NK) cell.

In some embodiments of any of the aspects, the cells are isolated from a subject. The term “isolated” as used herein signifies that the cells are placed into conditions other than their natural environment. The term “isolated” does not preclude the later use of these cells thereafter in combinations or mixtures with other cells. In some embodiments of any of the aspects, an immune cell (e.g., NK cell) is: (a) isolated from the subject; (b) genetically modified to express a CAR polypeptide as described herein; and (c) administered to the subject. In some embodiments of any of the aspects, the cells are isolated from a first subject and administered to a second subject. In some embodiments of any of the aspects, the immune cells are first differentiated from a somatic cell sample from the subject and then genetically modified to express a CAR polypeptide as described herein. In other embodiments, the immune cells, e.g., NK cells, are differentiated from induced pluripotent stem cells derived from an individual, e.g., from an individual to whom the CAR-expressing NK cells as described herein will be administered.

In some embodiments, methods of genetically modifying a cell to express a CAR can comprise but are not limited to: transfection or electroporation of a cell with a vector encoding a CAR; transduction with a viral vector (e.g., retrovirus, lentivirus) encoding a CAR; gene editing using zinc finger nucleases (ZFNs), transcription activator-like effector nucleases (TALENs), meganuclease-TALENs, or CRISPR-Cas; or any other methods known in the art of genetically modifying a cell to express a CAR.

### Pharmaceutical Compositions and Administration

In some embodiments, the methods described herein relate to treating a subject having or diagnosed as having a disease or disorder (e.g., cancer, infectious disease, etc.) with a NK CAR polypeptide as described herein. Subjects having such a disease or disorder can be identified by a physician using current methods of diagnosis for cancer or infectious disease. Symptoms and/or complications which characterize these conditions and aid in diagnosis are known in the art. A family history of cancer or infectious disease, or exposure to risk factors for cancer or infectious disease can also aid in determining if a subject is likely to have such a disease or disorder, or in making a diagnosis of cancer or infectious disease.

The compositions described herein can be administered to a subject having or diagnosed as having cancer or infectious disease. In some embodiments, the methods described herein comprise administering an effective amount of a composition as described herein, e.g. a CAR polypeptide as described herein to a subject in order to alleviate a symptom of cancer or infectious disease. As used herein, “alleviating a symptom” is ameliorating any condition or symptom associated with the cancer or infectious disease. As compared with an equivalent untreated control, such reduction is by at least 5%, 10%, 20%, 40%, 50%, 60%, 80%, 90%, 95%, 99% or more as measured by any standard technique.

A variety of means for administering the compositions described herein to subjects are known to those of skill in the art. An agent can be administered intravenously by injection or by gradual infusion over time. Given an appropriate formulation for a given route, for example, agents useful in the methods and compositions described herein can be administered intravenously, intraperitoneally, intramuscularly, subcutaneously, intracavity, intratumorally, and can be delivered by peristaltic means, if desired, or by other means known by those skilled in the art. Local administration, e.g., directly to the site of a tumor or lesion is specifically contemplated.

Therapeutic compositions containing at least one agent can be conventionally administered in a unit dose, for example. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired.

The term “effective amount” as used herein refers to the amount of CAR-expressing NK cells as described herein needed to alleviate at least one or more symptom of the disease or disorder, and relates to a sufficient amount of pharmacological composition to provide the desired effect. The term “therapeutically effective amount” therefore refers to an amount of CAR-expressing NK cells as described herein that is sufficient to provide a particular anti-tumor effect (or anti-infectious disease effect) when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard procedures in cell cultures or experimental animals, e.g., for determining the minimal effective dose and/or maximal tolerated dose. The dosage can vary depending upon the dosage form employed and the route of administration utilized (e.g., systemic vs intratumoral). A therapeutically effective dose can be estimated initially from cell culture assays. Also, a dose can be formulated in animal models to achieve a dosage range between the minimal effective dose and the maximal tolerated dose. The effects of any particular dosage can be monitored by a suitable bioassay, e.g., assay for tumor growth and/or size among others. The dosage can be determined by a physician and adjusted, as necessary, to suit observed effects of the treatment.

It can generally be stated that a pharmaceutical composition comprising the cells, e.g., NK CAR cells, described herein may be administered at a dosage of 10² to 10¹⁰ cells/kg body weight, preferably 10⁵ to 10⁶ cells/kg body weight, including all integer values within those ranges. The number of cells will depend upon the ultimate use for which the composition is intended as will the type of cells included therein. For uses provided herein, the cells are generally in a volume of a liter or less, can be 500 mLs or less, even 250 mLs or 100 mLs or less. Hence the density of the desired cells is typically greater than 10⁶ cells/ml and generally is greater than 10⁷ cells/ml, generally 10⁸ cells/ml or greater. The clinically relevant number of immune cells can be apportioned into multiple infusions that cumulatively equal or exceed 10⁵, 10⁶, 10⁷, 10⁸, 10⁹, 10¹⁰, 10¹¹, or 10¹² cells. In some aspects of the present invention, particularly since all the infused cells will be redirected to a particular target antigen, lower numbers of cells, in the range of 10⁶/kilogram (10⁶-10¹¹ per patient) may be administered. In some embodiments, the dosage can be from about 1×10⁵ cells to about 1×10⁸ cells per kg of body weight. In some embodiments, the dosage can be from about 1×10⁶ cells to about 1×10⁷ cells per kg of body weight. In some embodiments, the dosage can be about 1×10⁶ cells per kg of body weight. CAR expressing cell compositions may be administered multiple times at dosages within these ranges. The cells may be allogeneic, syngeneic, xenogeneic, or autologous to the patient undergoing therapy. If desired, the treatment may also include administration of mitogens (e.g., PHA) or lymphokines, cytokines, and/or chemokines (e.g., IL-10, IL-12, IL-15, IL-18, IL-21, IFN-γ, GM-CSF, TGF-β, TNF-α, and IFN-α etc.) as described herein to enhance induction of the immune response. In some embodiments, one dose of cells can be administered. In some embodiments, the dose of cells can be repeated, e.g., once, twice, or more. In some embodiments, the dose of cells can be administered on, e.g., a daily, weekly, or monthly basis.

In some embodiments, the dose can be administered intravenously. In some embodiments, the intravenous administration can be an infusion occurring over a period of from about 10 minutes to about 3 hours. In some embodiments, the intravenous administration can be an infusion occurring over a period of from about 30 minutes to about 90 minutes.

In some embodiments the dose can be administered about weekly. In some embodiments, the dose can be administered weekly. In some embodiments, the dose can be administered weekly for from about 12 weeks to about 18 weeks. In some embodiments the dose can be administered about every 2 weeks. In some embodiments the dose can be administered about every 3 weeks. In some embodiments, a total of from about 2 to about 10 doses are administered. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, the administration occurs for a total of from about 4 weeks to about 12 weeks. In some embodiments, the administration occurs for a total of about 6 weeks. In some embodiments, the administration occurs for a total of about 8 weeks. In some embodiments, the administration occurs for a total of about 12 weeks. In some embodiments, the initial dose can be from about 1.5 to about 2.5 fold greater than subsequent doses.

Treatment according to the methods described herein can reduce levels of a marker or symptom of a condition, e.g. by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen.

The dosage ranges for the administration of a CAR composition or NK CAR cell composition, according to the methods described herein depend upon, for example, the form of the CAR composition, its potency, and the extent to which symptoms, markers, or indicators of a condition described herein are desired to be reduced, for example the percentage reduction desired for the disease or disorder (e.g., cancer or infectious disease). The dosage should not be so large as to cause adverse side effects, such as autoimmunity. Generally, the dosage will vary with the age, condition, and sex of the patient and can be determined by one of skill in the art. The dosage can also be adjusted by the individual physician in the event of any complication.

The efficacy of a CAR composition in, e.g. the treatment of a condition described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. tumor size. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g., tumor size, or pathogen titer, or antibody titer). It is well within the ability of one skilled in the art to monitor efficacy of administration and/or treatment by measuring any one of such parameters, or any combination of parameters. Efficacy can be assessed in animal models of a condition described herein, for example treatment of cancer. When using an experimental animal model, efficacy of treatment is evidenced when a statistically significant change in a marker is observed, e.g. tumor size, pathogen titer, or antibody titer.

In vitro and animal model assays are provided herein which allow the assessment of a given dose of a CAR composition. The efficacy of a given dosage combination can also be assessed in an animal model, e.g. a specific cancer animal model.

In one aspect, described herein is a pharmaceutical composition comprising the at least one CAR polypeptide, CAR polynucleotide, CAR vector, or CAR-comprising cell as described herein, which are collectively referred to as a “CAR composition”. In some embodiments of any of the aspects, the pharmaceutical composition can comprise any combination of CAR polypeptides described herein.

In some embodiments, the technology described herein relates to a pharmaceutical composition comprising a CAR composition as described herein, and optionally a pharmaceutically acceptable carrier. In some embodiments, the active ingredients of the pharmaceutical composition comprise the CAR polypeptide as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C₂-C₁₂ alcohols, such as ethanol; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent, e.g. the CAR polypeptide as described herein.

In some embodiments, the pharmaceutical composition comprising a CAR composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of CAR compositions as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate.

Pharmaceutical compositions comprising CAR compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

In some embodiments of any of the aspects, the CAR composition described herein is administered as a monotherapy, e.g., another treatment for the disease or disorder (e.g., cancer) is not administered to the subject.

In some embodiments of any of the aspects, the methods described herein can further comprise administering a second agent and/or treatment to the subject, e.g. as part of a combinatorial therapy. Non-limiting examples of a second agent and/or treatment can include radiation therapy, surgery, gemcitabine, cisplatin, paclitaxel, carboplatin, bortezomib, AMG479, vorinostat, rituximab, temozolomide, rapamycin, ABT-737, PI-103; alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylmelamines including altretamine, triethylenemelamine, trietylenephosphoramide, triethylenethiophosphoramide and trimethylol melamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gamma(1)I, calicheamicin theta(1)I, and/or calicheamicin omega(1)I (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomycins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003).

In addition, the methods of treatment can further include the use of radiation or radiation therapy. Further, the methods of treatment can further include the use of surgical treatments.

In some embodiments of any of the aspects, the subject is administered a CAR composition and an antimicrobial agent. As used herein, the term “antimicrobial agent” (also referred to herein as an antimicrobial, antimicrobial therapeutic, and the like) refers to a molecule or composition which destroys microbes (i.e., bacteria, fungi, viruses, parasites and microbial spores) or prevents or inhibits their development, proliferation and/or pathogenic action. The term “antimicrobial” thus comprises antibacterials, antifungals, and antivirals. Exemplary antimicrobial agents include, but are not limited to, small organic or inorganic molecules; peptides; proteins; peptide analogs and derivatives; peptidomimetics; antibodies (polyclonal or monoclonal); antigen binding fragments of antibodies; nucleic acids; nucleic acid analogs and derivatives; an extract made from biological materials such as bacteria, plants, fungi, or animal cells; animal tissues; naturally occurring or synthetic compositions; and any combinations thereof.

In some embodiments of any of the aspects, the antimicrobial agent can be selected from aminoglycosides, ansamycins, beta-lactams, bis-biguanides, carbacephems, carbapenems, cationic polypeptides, cephalosporins, fluoroquinolones, glycopeptides, iron-sequestering glycoproteins, linosamides, lipopeptides, macrolides, monobactams, nitrofurans, oxazolidinones, penicillins, polypeptides, quaternary ammonium compounds, quinolones, silver compounds, sulfonamides, tetracyclines, and any combinations thereof. In some embodiments of any of the aspects, the antimicrobial agent can comprise an antibiotic.

In some embodiments of any of the aspects, the subject is administered a CAR composition and an antibacterial. Some exemplary specific antibacterials include broad penicillins, amoxicillin (e.g., Ampicillin, Bacampicillin, Carbenicillin Indanyl, Mezlocillin, Piperacillin, Ticarcillin), Penicillins and Beta Lactamase Inhibitors (e.g., Amoxicillin-Clavulanic Acid, Ampicillin-Sulbactam, Benzylpenicillin, Cloxacillin, Dicloxacillin, Methicillin, Oxacillin, Penicillin G, Penicillin V, Piperacillin Tazobactam, Ticarcillin Clavulanic Acid, Nafcillin), Cephalosporins (e.g., Cephalosporin I Generation, Cefadroxil, Cefazolin, Cephalexin, Cephalothin, Cephapirin, Cephradine), Cephalosporin II Generation (e.g., Cefaclor, Cefamandole, Cefonicid, Cefotetan, Cefoxitin, Cefprozil, Cefmetazole, Cefuroxime, Loracarbef), Cephalosporin III Generation (e.g., Cefdinir, Ceftibuten, Cefoperazone, Cefixime, Cefotaxime, Cefpodoxime proxetil, Ceftazidime, Ceftizoxime, Ceftriaxone), Cephalosporin IV Generation (e.g., Cefepime), Macrolides and Lincosamides (e.g., Azithromycin, Clarithromycin, Clindamycin, Dirithromycin, Erythromycin, Lincomycin, Troleandomycin), Quinolones and Fluoroquinolones (e.g., Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, Perfloxacin), Carbapenems (e.g., Imipenem-Cilastatin, Meropenem), Monobactams (e.g., Aztreonam), Aminoglycosides (e.g., Amikacin, Gentamicin, Kanamycin, Neomycin, Netilmicin, Streptomycin, Tobramycin, Paromomycin), Glycopeptides (e.g., Teicoplanin, Vancomycin), Tetracyclines (e.g., Demeclocycline, Doxycycline, Methacycline, Minocycline, Oxytetracycline, Tetracycline, Chlortetracycline), Sulfonamides (e.g., Mafenide, Silver Sulfadiazine, Sulfacetamide, Sulfadiazine, Sulfamethoxazole, Sulfasalazine, Sulfisoxazole, Trimethoprim-Sulfamethoxazole, Sulfamethizole), Rifampin (e.g., Rifabutin, Rifampin, Rifapentine), Oxazolidinones (e.g., Linezolid, Streptogramins, Quinupristin Dalfopristin), Bacitracin, Chloramphenicol, Fosfomycin, Isoniazid, Methenamine, Metronidazole, Mupirocin, Nitrofurantoin, Nitrofurazone, Novobiocin, Polymyxin, Spectinomycin, Trimethoprim, Colistin, Cycloserine, Capreomycin, Ethionamide, Pyrazinamide, Para-aminosalicylic acid, Erythromycin ethylsuccinate, and the like.

In some embodiments of any of the aspects, the subject is administered a CAR composition and an antiviral. In some embodiments of any of the aspects, the antiviral is selected from the group consisting of: abacavir, acyclovir, adefovir, amantadine, ampligen, amprenavir, antiretroviral, arbidol, atazanavir, atripla, cidofovir, combivir, darunavir, delavirdine, didanosine, docosanol, dolutegravir, ecoliever, edoxudine, efavirenz, emtricitabine, enfuvirtide, entecavir, famciclovir, favipiravir, fomivirsen, fosamprenavir, foscarnet, fosfonet, fusion inhibitor, ganciclovir, hydroxychloroquine, ibacitabine, idoxuridine, imiquimod, imunovir, indinavir, inosine, integrase inhibitor, interferon, interferon type I, interferon type II, interferon type III, lamivudine, lopinavir, loviride, maraviroc, methisazone, moroxydine, nelfinavir, nevirapine, nexavir, nitazoxanide, norvir, nucleoside analogues, oseltamivir (TAMIFLU), peginterferon alfa-2a, penciclovir, peramivir, pleconaril, podophyllotoxin, protease inhibitor, pyramidine, raltegravir, remdesivir, reverse transcriptase inhibitor, ribavirin, rimantadine, ritonavir, saquinavir, sofosbuvir, stavudine, telaprevir, tenofovir, tenofovir disoproxil, tipranavir, trifluridine, trizivir, tromantadine, truvada, valaciclovir (VALTREX), valganciclovir, vicriviroc, vidarabine, viramidine, zalcitabine, zanamivir (RELENZA), and zidovudine; see e.g., PCT patent applications WO2019018185A1, WO2019079339A1, WO2020055368A2, US Patent application US20200017514A1; US patent U.S. Pat. No. 8,575,195; the contents of each of which are incorporated herein by reference in their entireties. Additional antivirals known in the art can also be used.

### Treatment Methods

The NK CAR compositions described herein can be administered to a subject in need thereof, in particular the treatment of cancer. In some embodiments of any of the aspects, the CAR compositions described herein can be administered for the treatment of cancer or infectious disease. Infectious diseases that can be treated with the CAR compositions described herein include any microorganism with a specific microbial antigen that can be targeted; the infectious diseases can be viral or bacterial infections.

In some embodiments, the method of treatment can comprise first diagnosing a subject or patient who can benefit from treatment by a composition described herein. In some embodiments, such diagnosis comprises detecting or measuring an abnormal level of a marker (e.g., the tumor antigens as described herein or antigens from a virus or bacteria) in a sample from the subject or patient. In some embodiments, the method further comprises administering to the patient a CAR composition as described herein.

In some embodiments, the subject has previously been determined to have an abnormal level of an analyte described herein relative to a reference. In some embodiments, the reference level can be the level in a sample of similar cell type, sample type, sample processing, and/or obtained from a subject of similar age, sex and other demographic parameters as the sample/subject. In some embodiments, the test sample and control reference sample are of the same type, that is, obtained from the same biological source, and comprising the same composition, e.g. the same number and type of cells.

The term “sample” or “test sample” as used herein denotes a sample taken or isolated from a biological organism, e.g., a blood or plasma sample from a subject. In some embodiments of any of the aspects, the technology described herein encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; sperm; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject.

In some embodiments of any of the aspects, the step of determining if the subject has an abnormal level of an analyte described herein can comprise i) obtaining or having obtained a sample from the subject and ii) performing or having performed an assay on the sample obtained from the subject to determine/measure the level of the analyte in the subject. In some embodiments of any of the aspects, the step of determining if the subject has an abnormal level of an analyte described herein can comprise performing or having performed an assay on a sample obtained from the subject to determine/measure the level of analyte in the subject. In some embodiments of any of the aspects, the step of determining if the subject has an abnormal level of an analyte described herein can comprise ordering or requesting an assay on a sample obtained from the subject to determine/measure the level of the analyte in the subject. In some embodiments of any of the aspects, the step of determining if the subject has an abnormal level of an analyte described herein can comprise receiving the results of an assay on a sample obtained from the subject to determine/measure the level of the analyte in the subject. In some embodiments of any of the aspects, the step of determining if the subject has an abnormal level of an analyte described herein can comprise receiving a report, results, or other means of identifying the subject as a subject with a decreased level of the analyte.

In one aspect, described herein is a method of treating a subject in need of a CAR-based therapy comprising administering to the subject a therapeutically effective amount of the CAR-based therapy selected from the group consisting of: a NK CAR polypeptide as described herein, a NK CAR polynucleotide as described herein, a NK CAR vector as described herein, a NK CAR lentivirus as described herein, a NK CAR cell or population thereof as described herein, or a NK CAR pharmaceutical composition as described herein.

In one aspect of any of the embodiments, described herein is a method of treating cancer or an infectious disease in a subject in need thereof, the method comprising: a) determining if the subject has an abnormal level of an analyte (e.g., a tumoral, viral, or bacterial antigen as described herein); and b) instructing or directing that the subject be administered a CAR composition as described herein if the level of the analyte is increased or otherwise abnormal relative to a reference. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results. In some embodiments of any of the aspects, the administration of the CAR-based therapy results in increased specific lysis of cancer cells targeted by the CAR. In some embodiments of any of the aspects, the administration of the CAR-based therapy results in increased specific lysis of infected cells targeted by the CAR.

In one aspect described herein is a method of increasing the activation of an NK cell or population thereof comprising contacting the cell or population thereof with a NK CAR polypeptide as described herein, a NK CAR polynucleotide as described herein, a NK CAR vector as described herein, or a NK CAR lentivirus as described herein.

In some embodiments of any of the aspects, contacting the NK cell or population thereof with the polypeptide, polynucleotide, vector, or lentivirus increases the activity of the NK cell by at least 10%, at least 20%, at least 30%, at least 40%, at least 50%, at least 60%, at least 70%, at least 80%, at least 90%, at least 100%, at least 200%, at least 300%, at least 400%, or at least 500% compared to prior to contacting with the polypeptide, polynucleotide, vector, or lentivirus. In some embodiments of any of the aspects, the increased activation of the NK cell or population thereof comprises increased expression of a cytokine or granzyme selected from the group consisting of TNFα, IFNγ, GM-CSF, and Granzyme B.

In some embodiments of any of the aspects, the increased activation of the NK cell or population thereof results in an increased specific lysis of a target cell. In some embodiments of any of the aspects, the target cell expresses a surface antigen that binds specifically to the extracellular binding domain of the polypeptide. In some embodiments of any of the aspects, the target cell is a cancer cell. In some embodiments of any of the aspects, the target cell is a cell infected by a virus or bacteria.

In another aspect, described herein is a method of making a therapeutic composition, the method comprising introducing a NK CAR polypeptide as described herein, a NK CAR polynucleotide as described herein, a NK CAR vector as described herein, or a NK CAR lentivirus as described herein to an NK cell under conditions permitting expression of the NK CAR polypeptide in the cell.

In some embodiments of any of the aspects, prior to introducing the nucleic acid, polynucleotide, vector, or lentivirus, the NK cell is removed from a subject in need of the therapeutic composition. In some embodiments of any of the aspects, after introducing the nucleic acid, polynucleotide, vector, or lentivirus, the NK cell is returned to the subject.

### Cancer

In some embodiments of the various aspects described herein, CAR polypeptides as described herein can be used to treat cancer. As used herein, the term “cancer” relates generally to a class of diseases or conditions in which abnormal cells divide without control and can invade nearby tissues. Cancer cells can also spread to other parts of the body through the blood and lymph systems. There are several main types of cancer. Carcinoma is a cancer that begins in the skin or in tissues that line or cover internal organs. Sarcoma is a cancer that begins in bone, cartilage, fat, muscle, blood vessels, or other connective or supportive tissue. Leukemia is a cancer that starts in blood-forming tissue such as the bone marrow, and causes large numbers of abnormal blood cells to be produced and enter the blood. Lymphoma and multiple myeloma are cancers that begin in the cells of the immune system. Central nervous system cancers are cancers that begin in the tissues of the brain and spinal cord.

In some embodiments of any of the aspects, the cancer is a primary cancer. In some embodiments of any of the aspects, the cancer is a malignant cancer. As used herein, the term “malignant” refers to a cancer in which a group of tumor cells display one or more of uncontrolled growth (i.e., division beyond normal limits), invasion (i.e., intrusion on and destruction of adjacent tissues), and metastasis (i.e., spread to other locations in the body via lymph or blood). As used herein, the term “metastasize” refers to the spread of cancer from one part of the body to another. A tumor formed by cells that have spread is called a “metastatic tumor” or a “metastasis.” The metastatic tumor contains cells that are like those in the original (primary) tumor. As used herein, the term “benign” or “non-malignant” refers to tumors that may grow larger but do not spread to other parts of the body. Benign tumors are self-limited and typically do not invade or metastasize.

A “cancer cell” or “tumor cell” refers to an individual cell of a cancerous growth or tissue. A tumor refers generally to a swelling or lesion formed by an abnormal growth of cells, which may be benign, pre-malignant, or malignant. Most cancer cells form tumors, but some, e.g., leukemia, do not necessarily form tumors. For those cancer cells that form tumors, the terms cancer (cell) and tumor (cell) are used interchangeably.

As used herein the term “neoplasm” refers to any new and abnormal growth of tissue, e.g., an abnormal mass of tissue, the growth of which exceeds and is uncoordinated with that of the normal tissues. Thus, a neoplasm can be a benign neoplasm, premalignant neoplasm, or a malignant neoplasm.

A subject that has a cancer or a tumor is a subject having objectively measurable cancer cells present in the subject's body. Included in this definition are malignant, actively proliferative cancers, as well as potentially dormant tumors or micrometastases. Cancers which migrate from their original location and seed other vital organs can eventually lead to the death of the subject through the functional deterioration of the affected organs.

Examples of cancer include but are not limited to, carcinoma, lymphoma, blastoma, sarcoma, leukemia, basal cell carcinoma, biliary tract cancer; bladder cancer; bone cancer; brain and CNS cancer; breast cancer; cancer of the peritoneum; cervical cancer; choriocarcinoma; colon and rectum cancer; connective tissue cancer; cancer of the digestive system; endometrial cancer; esophageal cancer; eye cancer; cancer of the head and neck; gastric cancer (including gastrointestinal cancer); glioblastoma (GBM); hepatic carcinoma; hepatoma; intra-epithelial neoplasm; kidney or renal cancer; larynx cancer; leukemia; liver cancer; lung cancer (e.g., small-cell lung cancer, non-small cell lung cancer, adenocarcinoma of the lung, and squamous carcinoma of the lung); lymphoma including Hodgkin's and non-Hodgkin's lymphoma; melanoma; myeloma; neuroblastoma; oral cavity cancer (e.g., lip, tongue, mouth, and pharynx); ovarian cancer; pancreatic cancer; prostate cancer; retinoblastoma; rhabdomyosarcoma; rectal cancer; cancer of the respiratory system; salivary gland carcinoma; sarcoma; skin cancer; squamous cell cancer; stomach cancer; testicular cancer; thyroid cancer; uterine or endometrial cancer; cancer of the urinary system; vulval cancer; as well as other carcinomas and sarcomas; as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In some embodiments of any of the aspects, the subject has or has been diagnosed with adrenal cancer, anal cancer, appendix cancer, bile duct cancer, bladder cancer, bone cancer, brain cancer, breast cancer, cervical cancer, colorectal cancer, gallbladder cancer, gestational trophoblastic disease, head and neck cancer, Hodgkin lymphoma, intestinal cancer, kidney cancer, leukemia, liver cancer, lung cancer, melanoma, Merkel cell carcinoma, mesothelioma, multiple myeloma, neuroendocrine tumors, Non-Hodgkin lymphoma, oral cancer, ovarian cancer, pancreatic cancer, prostate cancer, sinus cancer, skin cancer, a sarcoma, a soft tissue sarcoma, spinal cancer, stomach cancer, testicular cancer, throat cancer, a tumor, thyroid cancer, uterine cancer, vaginal cancer or vulvar cancer.

A “cancer cell” is a cancerous, pre-cancerous, or transformed cell, either in vivo, ex vivo, or in tissue culture, that has spontaneous or induced phenotypic changes that do not necessarily involve the uptake of new genetic material. Although transformation can arise from infection with a transforming virus and incorporation of new genomic nucleic acid, or uptake of exogenous nucleic acid, it can also arise spontaneously or following exposure to a carcinogen, thereby mutating an endogenous gene. Transformation/cancer is associated with, e.g., morphological changes, immortalization of cells, aberrant growth control, foci formation, anchorage independence, malignancy, loss of contact inhibition and density limitation of growth, growth factor or serum independence, tumor specific markers, invasiveness or metastasis, and tumor growth in suitable animal hosts such as nude mice.

### Infectious Diseases

In some embodiments of the various aspects described herein, CAR polypeptides as described herein can be used to treat an infectious disease. In some embodiments of the various aspects described herein, the infectious disease is a bacterial infection. In some embodiments of the various aspects described herein, the infectious disease is a viral infection.

Non-limiting examples of pathogenic bacteria include spirochetes (e.g. Borrelia), actinomycetes (e.g. Actinomyces), mycoplasmas, Rickettsias, Gram negative aerobic rods, Gram negative aerobic cocci, Gram negatively facultatively anaerobic rods (e.g. Erwinia and Yersinia), Gram-negative cocci, Gram negative coccobacilli, Gram positive cocci (e.g. Staphylococcus and Streptococcus), endospore-forming rods, and endospore-forming cocci. Further non-limiting examples of bacterial pathogens include those belonging to the genera Bacillus, Brucella, Burkholderia, Francisella, Yersinia, Streptococcus, Haemophilus, Nisseria, Listeria, Clostridium, Klebsiella, Legionella, Escherichia (e.g., E. coli), Mycobacterium, Staphylococcus, Campylobacter, Vibrio, and Salmonella, as well as drug and multidrug resistant strains and highly virulent strains of these pathogenic bacteria. Non-limiting examples of known food-borne bacterial pathogens include those belonging to the genera Salmonella, Clostridium, Campylobacter spp., Staphylococcus, Salmonella, Escherichia (e.g., E. coli), and Listeria. In some embodiments, non-limiting examples of bacterial pathogens include Bacillus anthracis, Brucella abortus, Brucella melitensis, Brucella suis, Burkholderia mallei, Burkholderia pseudomallei, Francisella tularensis, Yersinia pestis, Streptococcus Group A and B, MRSA, Streptococcus pneumonia, Haemophilus influenza, Neisseria meningitides, Listeria monocytegenes, Clostridium difficile, Klebsiella, highly virulent pathogenic strains of E. coli, Mycobacterium tuberculosis, Staphylococcus aureus, Campylobacter spp, Salmonella spp, and Clostridium perfringens, as well as drug and multidrug resistant strains and highly virulent strains of these pathogenic bacteria. In some embodiments, non-limiting examples of known food-borne bacterial pathogens include Salmonella, non typhoidal Clostridium perfringens, Campylobacter spp., Staphylococcus aureus, Salmonella, nontyphoidal, Campylobacter spp., E. coli (STEC) 0157, and Listeria monocytogenes.

In some embodiments of any of the aspects, the CAR polypeptides described herein are used to treat an RNA virus. As a non-limiting example the RNA virus is a Group III (dsRNA) virus, a Group IV (+ssRNA) virus, a Group V (−ssRNA) virus, or a Group VI (reverse transcriptase) virus.

In some embodiments of any of the aspects, the RNA virus is a Group III (i.e., double stranded RNA (dsRNA)) virus. In some embodiments of any of the aspects, the Group III RNA virus belongs to a viral family selected from the group consisting of: Amalgaviridae, Birnaviridae, Chrysoviridae, Cystoviridae, Endomaviridae, Hypoviridae, Megabirnaviridae, Partitiviridae, Picobirnaviridae, Reoviridae (e.g., Rotavirus), Totiviridae, Quadriviridae. In some embodiments of any of the aspects, the Group III RNA virus belongs to the Genus Botybimavirus. In some embodiments of any of the aspects, the Group III RNA virus is an unassigned species selected from the group consisting of Botrytis porri RNA virus 1, Circulifer tenellus virus 1, Colletotrichum camelliae filamentous virus 1, Cucurbit yellows associated virus, Sclerotinia sclerotiorum debilitation-associated virus, and Spissistilus festinus virus 1.

In some embodiments of any of the aspects, the RNA virus is a Group IV (i.e., positive-sense single stranded (ssRNA)) virus. In some embodiments of any of the aspects, the Group IV RNA virus belongs to a viral order selected from the group consisting of: Nidovirales, Picornavirales, and Tymovirales. In some embodiments of any of the aspects, the Group IV RNA virus belongs to a viral family selected from the group consisting of: Arteriviridae, Coronaviridae (e.g., Coronavirus, SARS-CoV), Mesoniviridae, Roniviridae, Dicistroviridae, Iflaviridae, Marnaviridae, Picornaviridae (e.g., Poliovirus, Rhinovirus (a common cold virus), Hepatitis A virus), Secoviridae (e.g., sub Comovirinae), Alphaflexiviridae, Betaflexiviridae, Gammaflexiviridae, Tymoviridae, Alphatetraviridae, Alvernaviridae, Astroviridae, Barnaviridae, Benyviridae, Bromoviridae, Caliciviridae (e.g., Norwalk virus), Carmotetraviridae, Closteroviridae, Flaviviridae (e.g., Yellow fever virus, West Nile virus, Hepatitis C virus, Dengue fever virus, Zika virus), Fusariviridae, Hepeviridae, Hypoviridae, Leviviridae, Luteoviridae (e.g., Barley yellow dwarf virus), Polycipiviridae, Narnaviridae, Nodaviridae, Permutotetraviridae, Potyviridae, Sarthroviridae, Statovirus, Togaviridae (e.g., Rubella virus, Ross River virus, Sindbis virus, Chikungunya virus), Tombusviridae, and Virgaviridae. In some embodiments of any of the aspects, the Group IV RNA virus belongs to a viral genus selected from the group consisting of: Bacillariornavirus, Dicipivirus, Labyrnavirus, Sequiviridae, Blunervirus, Cilevirus, Higrevirus, Idaeovirus, Negevirus, Ourmiavirus, Polemovirus, Sinaivirus, and Sobemovirus. In some embodiments of any of the aspects, the Group IV RNA virus is an unassigned species selected from the group consisting of: Acyrthosiphon pisum virus, Bastrovirus, Blackford virus, Blueberry necrotic ring blotch virus, Cadicistrovirus, Chara australis virus, Extra small virus, Goji berry chlorosis virus, Hepelivirus, Jingmen tick virus, Le Blanc virus, Nedicistrovirus, Nesidiocoris tenuis virus 1, Niflavirus, Nylanderia fulva virus 1, Orsay virus, Osedax japonicus RNA virus 1, Picalivirus, Plasmopara halstedii virus, Rosellinia necatrix fusarivirus 1, Santeuil virus, Secalivirus, Solenopsis invicta virus 3, Wuhan large pig roundworm virus. In some embodiments of any of the aspects, the Group IV RNA virus is a satellite virus selected from the group consisting of: Family Sarthroviridae, Genus Albetovirus, Genus Aumaivirus, Genus Papanivirus, Genus Virtovirus, and Chronic bee paralysis virus.

In some embodiments of any of the aspects, the RNA virus is a Group V (i.e., negative-sense ssRNA) virus. In some embodiments of any of the aspects, the Group V RNA virus belongs to a viral phylum or subphylum selected from the group consisting of: Negamaviricota, Haploviricotina, and Polyploviricotina. In some embodiments of any of the aspects, the Group V RNA virus belongs to a viral class selected from the group consisting of: Chunqiuviricetes, Ellioviricetes, Insthoviricetes, Milneviricetes, Monjiviricetes, and Yunchangviricetes. In some embodiments of any of the aspects, the Group V RNA virus belongs to a viral order selected from the group consisting of: Articulavirales, Bunyavirales, Goujianvirales, Jingchuvirales, Mononegavirales, Muvirales, and Serpentovirales. In some embodiments of any of the aspects, the Group V RNA virus belongs to a viral family selected from the group consisting of: Amnoonviridae (e.g., Taastrup virus), Arenaviridae (e.g., Lassa virus), Aspiviridae, Bornaviridae (e.g., Boma disease virus), Chuviridae, Cruliviridae, Feraviridae, Filoviridae (e.g., Ebola virus, Marburg virus), Fimoviridae, Hantaviridae, Jonviridae, Mymonaviridae, Nairoviridae, Nyamiviridae, Orthomyxoviridae (e.g., Influenza viruses), Paramyxoviridae (e.g., Measles virus, Mumps virus, Nipah virus, Hendra virus, and NDV), Peribunyaviridae, Phasmaviridae, Phenuiviridae, Pneumoviridae (e.g., RSV and Metapneumovirus), Qinviridae, Rhabdoviridae (e.g., Rabies virus), Sunviridae, Tospoviridae, and Yueviridae. In some embodiments of any of the aspects, the Group V RNA virus belongs to a viral genus selected from the group consisting of: Anphevirus, Arlivirus, Chengtivirus, Crustavirus, Tilapineviridae, Wastrivirus, and Deltavirus (e.g., Hepatitis D virus).

In some embodiments of any of the aspects, the RNA virus is a Group VI RNA virus, which comprise a virally encoded reverse transcriptase. In some embodiments of any of the aspects, the Group VI RNA virus belongs to the viral order Ortervirales. In some embodiments of any of the aspects, the Group VI RNA virus belongs to a viral family or subfamily selected from the group consisting of Belpaoviridae, Caulimoviridae, Metaviridae, Pseudoviridae, Retroviridae (e.g., Retroviruses, e.g. HIV), Orthoretrovirinae, and Spumaretrovirinae. In some embodiments of any of the aspects, the Group VI RNA virus belongs to a viral genus selected from the group consisting of: Alpharetrovirus (e.g., Avian leukosis virus; Rous sarcoma virus), Betaretrovirus (e.g., Mouse mammary tumour virus), Bovispumavirus (e.g., Bovine foamy virus), Deltaretrovirus (e.g., Bovine leukemia virus; Human T-lymphotropic virus), Epsilonretrovirus (e.g., Walleye dermal sarcoma virus), Equispumavirus (e.g., Equine foamy virus), Felispumavirus (e.g., Feline foamy virus), Gammaretrovirus (e.g., Murine leukemia virus; Feline leukemia virus), Lentivirus (e.g., Human immunodeficiency virus 1; Simian immunodeficiency virus; Feline immunodeficiency virus), Prosimiispumavirus (e.g., Brown greater galago prosimian foamy virus), and Simiispumavirus (e.g., Eastern chimpanzee simian foamy virus). In some embodiments of any of the aspects, the virus is an endogenous retrovirus (ERV; e.g., endogenous retrovirus group W envelope member 1 (ERVWE1); HCP5 (HLA Complex P5); Human teratocarcinoma-derived virus), which are endogenous viral elements in the genome that closely resemble and can be derived from retroviruses.

In some embodiments of any of the aspects, the CAR polypeptides described herein are used to treat a DNA virus. As a non-limiting example the DNA virus is a Group I (dsDNA) virus, a Group II (ssDNA) virus, or a Group VII (dsDNA-RT) virus.

In some embodiments of any of the aspects, the DNA virus is a Group I (i.e., dsDNA) virus. In some embodiments of any of the aspects, the Group I dsDNA virus belongs to a viral order selected from the group consisting of: Caudovirales; Herpesvirales; and Ligamenvirales. In some embodiments of any of the aspects, the Group I dsDNA virus belongs to a viral family selected from the group consisting of: Adenoviridae (e.g., adenoviruses), Alloherpesviridae, Ampullaviridae, Ascoviridae, Asfarviridae (e.g., African swine fever virus), Baculoviridae, Bicaudaviridae, Clavaviridae, Corticoviridae, Fuselloviridae, Globuloviridae, Guttaviridae, Herpesviridae (e.g., human herpesviruses, Varicella Zoster virus), Hytrosaviridae, Iridoviridae, Lavidaviridae, Lipothrixviridae, Malacoherpesviridae, Marseilleviridae, Mimiviridae, Myoviridae (e.g., Enterobacteria phage T4), Nimaviridae, Nudiviridae, Pandoraviridae, Papillomaviridae, Phycodnaviridae, Plasmaviridae, Podoviridae (e.g., Enterobacteria phage T7), Polydnaviruses, Polyomaviridae (e.g., Simian virus 40, JC virus, BK virus), Poxviridae (e.g., Cowpox virus, smallpox), Rudiviridae, Siphoviridae (e.g., Enterobacteria phage λ), Sphaerolipoviridae, Tectiviridae, Tristromaviridae, and Turriviridae. In some embodiments of any of the aspects, the Group I dsDNA virus belongs to a viral genus selected from the group consisting of Dinodnavirus, Rhizidiovirus, and Salterprovirus. In some embodiments of any of the aspects, the Group I dsDNA virus belongs to an unassigned viral species selected from the group consisting of: Abalone shriveling syndrome-associated virus, Apis mellifera filamentous virus, Bandicoot papillomatosis carcinomatosis virus, Cedratvirus, Kaumoebavirus, KIs-V, Lentille virus, Leptopilina boulardi filamentous virus, Megavirus, Metallosphaera turreted icosahedral virus, Methanosarcina spherical virus, Mollivirus sibericum virus, Orpheovirus IHUMI-LCC2, Phaeocystis globosa virus, and Pithovirus. In some embodiments of any of the aspects, the Group I dsDNA virus is a virophage selected from the group consisting of: Organic Lake virophage, Ace Lake Mavirus virophage, Dishui Lake virophage 1, Guarani virophage, Phaeocystis globosa virus virophage, Rio Negro virophage, Sputnik virophage 2, Yellowstone Lake virophage 1, Yellowstone Lake virophage 2, Yellowstone Lake virophage 3, Yellowstone Lake virophage 4, Yellowstone Lake virophage 5, Yellowstone Lake virophage 6, Yellowstone Lake virophage 7, and Zamilon virophage 2.

In some embodiments of any of the aspects, the DNA virus is a Group II (i.e., ssDNA) virus. In some embodiments of any of the aspects, the Group II ssDNA virus belongs to a viral family selected from the group consisting of: Anelloviridae, Bacilladnaviridae, Bidnaviridae, Circoviridae, Geminiviridae, Genomoviridae, Inoviridae, Microviridae, Nanoviridae, Parvoviridae, Smacoviridae, and Spiraviridae.

In some embodiments of any of the aspects, the DNA virus is a Group VII (i.e., dsDNA-RT) virus. In some embodiments of any of the aspects, the Group VII dsDNA-RT virus belongs to the Ortervirales order. In some embodiments of any of the aspects, the Group VII dsDNA-RT virus belongs to the Caulimoviridae family or to the Hepadnaviridae family (e.g., Hepatitis B virus). In some embodiments of any of the aspects, the Group VII dsDNA-RT virus belongs to a viral genus selected from the group consisting of: Badnavirus, Caulimovirus, Cavemovirus, Petuvirus, Rosadnavirus, Solendovirus, Soymovirus, Tungrovirus, Avihepadnavirus, and Orthohepadnavirus.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of cancer or infectious disease. A subject can be male or female.

A subject can be one who has been previously diagnosed with or identified as suffering from or having a condition in need of treatment (e.g. cancer or infectious disease) or one or more complications related to such a condition, and optionally, have already undergone treatment for the disease or disorder (e.g., cancer or infectious disease) or the one or more complications related to the disease or disorder (e.g., cancer or infectious disease). Alternatively, a subject can also be one who has not been previously diagnosed as having the disease or disorder (e.g., cancer or infectious disease) or one or more complications related to the disease or disorder (e.g., cancer or infectious disease). For example, a subject can be one who exhibits one or more risk factors for the disease or disorder (e.g., cancer or infectious disease) or one or more complications related to the disease or disorder (e.g., cancer or infectious disease) or a subject who does not exhibit risk factors.

A “subject in need” of treatment for a particular condition can be a subject having that condition, diagnosed as having that condition, or at heightened risk of developing that condition.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

Specific polypeptide domain(s), region(s), or amino acid residue(s) that can likely tolerate variation can be identified by alignment of different intraspecies or interspecies variants of the polypeptide (e.g., an NK cell receptor, NK cell membrane-bound signaling adaptor protein, or a co-stimulatory receptor as described herein) or domains thereof (e.g., intracellular signaling domain, co-stimulatory domain, transmembrane domain, etc.). Domain(s), region(s), or amino acid residue(s) that exhibit minimal intraspecies and/or interspecies variation represent conserved sequences that likely cannot tolerate substantial variation or mutation, or likely can only tolerate conservative substitutions. Domain(s), region(s), or amino acid residue(s) that do exhibit intraspecies and/or interspecies variation represent non-conserved sequences that likely can tolerate variation or mutation (e.g., non-conservative substitutions or conservative substitutions).

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the assays described herein to confirm that a desired activity, e.g. function and specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a peptide which retains at least 50% of the wild-type reference polypeptide's activity according to an assay known in the art or as described herein. A functional fragment can comprise one or more conservative substitutions of the sequences disclosed herein. In some embodiments of any of the aspects, a polypeptide can comprise the first N-terminal amino acid methionine. In embodiments where a polypeptide does not comprise a first N-terminal methionine, it is understood that a variant of the polypeptide does comprise a first N-terminal methionine.

In some embodiments, the polypeptide described herein can be a variant of a sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a variant protein or fragment thereof that retains activity. A wide variety of PCR-based site-specific mutagenesis approaches are known in the art and can be applied by the ordinarily skilled artisan.

A variant amino acid or DNA sequence can be at least 70%, at least 75%, at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as described herein. Accordingly, the percentage of “sequence similarity” is the percentage of amino acids which is either identical or conservatively changed; e.g., “sequence similarity”=(% sequence identity)+(% conservative changes). It should be understood that a sequence that has a specified percent similarity to a reference sequence necessarily encompasses a sequence with the same specified percent identity to that reference sequence. The skilled person will be aware of several different computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelerys Inc., San Diego U.S.A.); the algorithm of E. Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA, cDNA, or vector DNA. Suitable RNA can include, e.g., mRNA.

The term “expression” refers to the cellular processes involved in producing RNA and proteins and as appropriate, secreting proteins, including where applicable, but not limited to, for example, transcription, transcript processing, translation and protein folding, modification and processing. Expression can refer to the transcription and stable accumulation of sense (e.g., mRNA) or antisense RNA derived from a nucleic acid fragment or fragments and/or to the translation of mRNA into a polypeptide.

As used herein, the term “detecting” or “measuring” refers to observing a signal from, e.g. a probe, label, or target molecule to indicate the presence of an analyte in a sample. Any method known in the art for detecting a particular label moiety can be used for detection. Exemplary detection methods include, but are not limited to, spectroscopic, fluorescent, photochemical, biochemical, immunochemical, electrical, optical or chemical methods. In some embodiments of any of the aspects, measuring can be a quantitative observation.

In some embodiments of any of the aspects, a polypeptide, nucleic acid, or cell as described herein can be engineered. As used herein, “engineered” refers to the aspect of having been manipulated by the hand of man. For example, a polypeptide is considered to be “engineered” when at least one aspect of the polypeptide, e.g., its sequence, has been manipulated by the hand of man to differ from the aspect as it exists in nature. As is common practice and is understood by those in the art, progeny of an engineered cell are typically still referred to as “engineered” even though the actual manipulation was performed on a prior entity.

In some embodiments of any of the aspects, a CAR polypeptide described herein is exogenous. In some embodiments of any of the aspects, a CAR polypeptide described herein is ectopic. In some embodiments of any of the aspects, a CAR polypeptide described herein is not endogenous.

The term “exogenous” refers to a substance present in a cell other than its native source. The term “exogenous” when used herein can refer to a nucleic acid (e.g. a nucleic acid encoding a polypeptide) or a polypeptide that has been introduced by a process involving the hand of man into a biological system such as a cell or organism in which it is not normally found and one wishes to introduce the nucleic acid or polypeptide into such a cell or organism. Alternatively, “exogenous” can refer to a nucleic acid or a polypeptide that has been introduced by a process involving the hand of man into a biological system such as a cell or organism in which it is found in relatively low amounts and one wishes to increase the amount of the nucleic acid or polypeptide in the cell or organism, e.g., to create ectopic expression or levels. In contrast, the term “endogenous” refers to a substance that is native to the biological system or cell. As used herein, “ectopic” refers to a substance that is found in an unusual location and/or amount. An ectopic substance can be one that is normally found in a given cell, but at a much lower amount and/or at a different time. Ectopic also includes substance, such as a polypeptide or nucleic acid that is not naturally found or expressed in a given cell in its natural environment.

As described herein, an “antigen” is a molecule that is bound by a binding site on an antibody agent (e.g., extracellular binding domain comprising an antibody-derived structure). Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

In some embodiments, a nucleic acid encoding a polypeptide as described herein (e.g. a CAR polypeptide) is comprised by a vector. In some of the aspects described herein, a nucleic acid sequence encoding a given polypeptide as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

In some embodiments of any of the aspects, the vector is recombinant, e.g., it comprises sequences originating from at least two different sources or is otherwise modified by the hand of man. In some embodiments of any of the aspects, the vector comprises sequences originating from at least two different species. In some embodiments of any of the aspects, the vector comprises sequences originating from at least two different genes, e.g., it comprises a fusion protein or a nucleic acid encoding an expression product which is operably linked to at least one non-native (e.g., heterologous) genetic control element (e.g., a promoter, suppressor, activator, enhancer, response element, or the like).

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification.

As used herein, the term “viral vector” refers to a nucleic acid vector construct that includes at least one element of viral origin and has the capacity to be packaged into a viral vector particle. The viral vector can contain the nucleic acid encoding a polypeptide as described herein in place of non-essential viral genes. The vector and/or particle can be utilized for the purpose of transferring any nucleic acids into cells either in vitro or in vivo. Numerous forms of viral vectors are known in the art. Non-limiting examples of a viral vector useful in connection with the technology disclosed herein include an AAV vector, an adenovirus vector, a lentivirus vector, a retrovirus vector, a herpesvirus vector, an alphavirus vector, a poxvirus vector a baculovirus vector, and a chimeric virus vector.

It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. cancer or infectious disease. The term “treating” includes reducing or alleviating at least one adverse effect or symptom of a condition, disease or disorder (e.g., cancer or infectious disease). Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to an active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be an artificial or engineered carrier, e.g., a carrier that the active ingredient would not be found to occur in or within nature.

As used herein, the term “administering,” refers to the placement of a compound as disclosed herein into a subject by a method or route which results in at least partial delivery of the agent at a desired site. Pharmaceutical compositions comprising the compositions disclosed herein can be administered by any appropriate route which results in an effective treatment in the subject. In some embodiments, administration comprises physical human activity, e.g., an injection, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

As used herein, the term “corresponding to” refers to an amino acid or nucleotide at the enumerated position in a first polypeptide or nucleic acid, or an amino acid or nucleotide that is equivalent to an enumerated amino acid or nucleotide in a second polypeptide or nucleic acid. Equivalent enumerated amino acids or nucleotides can be determined by alignment of candidate sequences using degree of homology programs known in the art, e.g., BLAST.

As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or more times greater than the affinity for the third non-target entity. A reagent specific for a given target is one that exhibits specific binding for that target under the conditions of the assay being utilized.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

In some embodiments of any of the aspects, the disclosure described herein does not concern a process for cloning human beings, processes for modifying the germ line genetic identity of human beings, uses of human embryos for industrial or commercial purposes or processes for modifying the genetic identity of animals which are likely to cause them suffering without any substantial medical benefit to man or animal, and also animals resulting from such processes.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

Some embodiments of the technology described herein can be defined according to any of the following numbered paragraphs:
1. A chimeric antigen receptor (CAR) polypeptide for expression in natural killer (NK) cells comprising:
   a) an extracellular binding domain;
   b) a transmembrane domain; and
   c) an intracellular domain comprising at least one of the following:
      i) an intracellular signaling domain from an NK cell receptor;
      ii) an intracellular signaling domain from an NK cell membrane-bound signaling adaptor protein; and/or
      iii) an intracellular co-stimulatory domain from a co-stimulatory receptor.
2. The polypeptide of paragraph 1, wherein the NK cell receptor:
   a. is selected from the group consisting of:
      i) Natural Killer Cell Receptor 2B4;
      ii) Natural Killer-, T- And B-Cell Antigen (NTB-A);
      iii) CD2-Like Receptor Activating Cytotoxic Cells (CRACC); and
      iv) cluster of differentiation 2 (CD2); or
   b) intracellular signaling domain comprises the amino acid sequence of one of SEQ ID NOs: 7-10 or an amino acid sequence that is at least 80% identical to one of SEQ ID NOs: 7-10.
3. The polypeptide of paragraph 1, wherein the NK cell membrane-bound signaling adaptor protein:
   a) is selected from the group consisting of:
      i) high-affinity IgE receptor (FcεR1); and
      ii) CD3-zeta (CD3ζ); or
   b) intracellular signaling domain comprises the amino acid sequence of one of SEQ ID NOs: 11-12 or an amino acid sequence that is at least 80% identical to one of SEQ ID NOs: 11-12.
4. The polypeptide of paragraph 1, wherein the co-stimulatory receptor:
   a) is 4-1BB and/or IL2 receptor beta (IL2RB); or
   b) intracellular co-stimulatory domain comprises the amino acid sequence of one of SEQ ID NOs: 15-16 or an amino acid sequence that is at least 80% identical to one of SEQ ID NOs: 15-16.
5. The polypeptide of paragraph 1, wherein the intracellular domain further comprises at least one self-cleaving peptide and/or a cytokine.
6. The polypeptide of paragraph 5, wherein the self-cleaving peptide:
   a) is T2A, P2A, E2A, or F2A; or
   b) comprises the amino acid sequence of one of SEQ ID NOs: 19-20 or an amino acid sequence that is at least 80% identical to one of SEQ ID NOs: 19-20.
7. The polypeptide of paragraph 5, wherein the cytokine:
   a) is IL-15 or IL-21;
   b) comprises the amino acid sequence of one of SEQ ID NOs: 23-24 or an amino acid sequence that is at least 80% identical to one of SEQ ID NOs: 23-24; or
   c) is adjacent and distal to the self-cleaving peptide, such that the cytokine is separated from the polypeptide by the self-cleaving peptide.
8. The polypeptide of paragraph 1, wherein the transmembrane domain comprises:
   a) a transmembrane domain of a natural NK cell receptor;
   b) a transmembrane domain of CD8; or
   c) the amino acid sequence of one of SEQ ID NOs: 29-32 or 117-118 or an amino acid sequence that is at least 80% identical to one of SEQ ID NOs: 29-32 or 117-118.
9. The polypeptide of paragraph 8, wherein the transmembrane domain of the natural NK cell receptor is selected from the group consisting of Natural Killer Group 2D (NKG2D); Natural Killer Cell P46-Related Protein (NKp46); and DNAX Accessory Molecule-1 (DNAM1).
10. The polypeptide of paragraph 1, wherein the extracellular binding domain:
   a) is an antibody, an antigen-binding fragment thereof, a F(ab) fragment, a F(ab′) fragment, a single chain variable fragment (scFv), or a single-domain antibody (sdAb);
   b) specifically binds to a tumor-associated antigen; or
   c) comprises the amino acid sequence of one of SEQ ID NOs: 35-36 or an amino acid sequence that is at least 80% identical to one of SEQ ID NOs: 35-36.
11. The polypeptide of paragraph 1, further comprising a signal peptide located N-terminal to the extracellular binding domain.
12. The polypeptide of paragraph 11, wherein the signal peptide:
   a) is a CD8 signal peptide; or
   b) comprises the amino acid sequence of SEQ ID NO: 38 or an amino acid sequence that is at least 80% identical to SEQ ID NO: 38.
13. The polypeptide of paragraph 1, further comprising a detectable marker distal to the extracellular binding domain.
14. The polypeptide of paragraph 11, further comprising a linker domain distal to the extracellular binding domain and/or proximal to the signal peptide.
15. The polypeptide of paragraph 1, further comprising a spacer domain located between the extracellular binding domain and the transmembrane domain.
16. The polypeptide of paragraph 15, wherein the spacer domain comprises
   a) a CD8 hinge domain; or
   b) the amino acid sequence of SEQ ID NO: 44 or an amino acid sequence that is at least 80% identical to SEQ ID NO: 44.
17. The CAR polypeptide of paragraph 1, wherein the polypeptide comprises the amino acid sequence of one of SEQ ID NOs: 80-114 or an amino acid sequence that is at least 80% identical to one of SEQ ID NOs: 80-114.
18. A natural killer (NK) cell or population thereof comprising the polypeptide of paragraph 1.
19. A method of increasing the activation of an NK cell or population thereof comprising contacting the cell or population thereof with the polypeptide of paragraph 1.
20. A method of treating a subject in need of a CAR-based therapy comprising administering to the subject a therapeutically effective amount of the CAR-based therapy comprising the polypeptide of paragraph 1.
21. A chimeric antigen receptor (CAR) polypeptide for expression in natural killer (NK) cells comprising:
   a) an extracellular binding domain;
   b) a transmembrane domain; and
   c) an intracellular domain comprising at least one of the following:
      i) an intracellular signaling domain from an NK cell receptor;
      ii) an intracellular signaling domain from an NK cell membrane-bound signaling adaptor protein; and/or
      iii) an intracellular co-stimulatory domain from a co-stimulatory receptor.
22. The polypeptide of paragraph 21, wherein the polypeptide comprises from N-terminus to C-terminus:
   a) the extracellular binding domain;
   b) the transmembrane domain; and
   c) the intracellular domain.
23. The polypeptide of paragraph 21, wherein the polypeptide comprises from C-terminus to N-terminus:
   a) the extracellular binding domain;
   b) the transmembrane domain; and
   c) the intracellular domain.
24. The polypeptide of any one of paragraphs 21-23, wherein the NK cell receptor or NK cell membrane-bound signaling adaptor protein is selected from the group consisting of:
   a) Natural Killer Cell Receptor 2B4;
   b) Natural Killer-, T- And B-Cell Antigen (NTB-A);
   c) CD2-Like Receptor Activating Cytotoxic Cells (CRACC);
   d) cluster of differentiation 2 (CD2);
   e) high-affinity IgE receptor (FcεR1); and
   f) CD3-zeta (CD3ζ).
25. The polypeptide of any one of paragraphs 21-24, wherein the NK cell receptor is selected from the group consisting of 2B4, NTB-A, CRACC, and CD2.
26. The polypeptide of any one of paragraphs 21-25, wherein the NK cell receptor is 2B4.
27. The polypeptide of any one of paragraphs 21-26, wherein the NK cell receptor is NTB-A.
28. The polypeptide of any one of paragraphs 21-27, wherein the NK cell receptor is CRACC.
29. The polypeptide of any one of paragraphs 21-28, wherein the NK cell receptor is CD2.
30. The polypeptide of any one of paragraphs 21-29, wherein the NK cell membrane-bound signaling adaptor protein is CD3ζ or FcεR1.
31. The polypeptide of any one of paragraphs 21-30, wherein the intracellular signaling domain from the NK cell receptor or the intracellular signaling domain from the NK cell membrane-bound signaling adaptor protein comprises the amino acid sequence of one of SEQ ID NOs: 7-12 or an amino acid sequence that is at least 80% identical to one of SEQ ID NOs: 7-12.
32. The polypeptide of any one of paragraphs 21-31, wherein the co-stimulatory receptor is 4-1BB and/or IL2 receptor beta (IL2RB).
33. The polypeptide of paragraph 32, wherein the intracellular co-stimulatory domain from the co-stimulatory receptor comprises the amino acid sequence of one of SEQ ID NOs: 15-16 or an amino acid sequence that is at least 80% identical to one of SEQ ID NOs: 15-16.
34. The polypeptide of any one of paragraphs 21-33, wherein the intracellular domain further comprises at least one self-cleaving peptide.
35. The polypeptide of paragraph 34, wherein the self-cleaving peptide is T2A, P2A, E2A, or F2A.
36. The polypeptide of paragraph 34 or 35, wherein the self-cleaving peptide comprises the amino acid sequence of one of SEQ ID NOs: 19-20 or an amino acid sequence that is at least 80% identical to one of SEQ ID NOs: 19-20.
37. The polypeptide of any one of paragraphs 21-36, wherein the intracellular domain further comprises a cytokine.
38. The polypeptide of paragraph 37, wherein the cytokine is IL-15 or IL-21.
39. The polypeptide of paragraph 37 or 38, wherein the cytokine comprises the amino acid sequence of one of SEQ ID NOs: 23-24 or an amino acid sequence that is at least 80% identical to one of SEQ ID NOs: 23-24.
40. The polypeptide of any one of paragraphs 21-39, wherein the intracellular domain further comprises at least one self-cleaving peptide and at least one cytokine.
41. The polypeptide of paragraph 40, wherein the cytokine is adjacent and distal to the self-cleaving peptide, such that the cytokine is separated from the polypeptide by the self-cleaving peptide.
42. The polypeptide of any one of paragraphs 21-41, wherein the transmembrane domain comprises a transmembrane domain of a natural NK cell receptor.
43. The polypeptide of any one of paragraphs 21-42, wherein the transmembrane domain of the natural NK cell receptor is selected from the group consisting of Natural Killer Group 2D (NKG2D); Natural Killer Cell P46-Related Protein (NKp46); and DNAX Accessory Molecule-1 (DNAM1).
44. The polypeptide of any one of paragraphs 21-43, wherein the transmembrane domain comprises a transmembrane domain of CD8.
45. The polypeptide of any one of paragraphs 21-44, wherein the transmembrane domain comprises the amino acid sequence of one of SEQ ID NOs: 29-32 or 117-118 or an amino acid sequence that is at least 80% identical to one of SEQ ID NOs: 29-32 or 117-118.
46. The polypeptide of any one of paragraphs 21-45, wherein the extracellular binding domain is an antibody, an antigen-binding fragment thereof, a F(ab) fragment, a F(ab′) fragment, a single chain variable fragment (scFv), or a single-domain antibody (sdAb).
47. The polypeptide of any one of paragraphs 21-46, wherein the extracellular binding domain comprises a scFv.
48. The polypeptide of any one of paragraphs 21-47, wherein the extracellular binding domain specifically binds to a tumor-associated antigen.
49. The polypeptide of any one of paragraphs 21-48, wherein the tumor-associated antigen is CD19.
50. The polypeptide of any one of paragraphs 21-49, wherein the tumor-associated antigen is CD33.
51. The polypeptide of any one of paragraphs 21-50, wherein the extracellular binding domain comprises the amino acid sequence of one of SEQ ID NOs: 35-36 or an amino acid sequence that is at least 80% identical to one of SEQ ID NOs: 35-36.
52. The polypeptide of any one of paragraphs 21-51, further comprising a signal peptide located N-terminal to the extracellular binding domain.
53. The polypeptide of paragraph 52, wherein the signal peptide is a CD8 signal peptide.
54. The polypeptide of paragraph 52 or 53, wherein the signal peptide comprises the amino acid sequence of SEQ ID NO: 38 or an amino acid sequence that is at least 80% identical to SEQ ID NO: 38.
55. The polypeptide of any one of paragraphs 21-54, further comprising a detectable marker distal to the extracellular binding domain.
56. The polypeptide of paragraph 55, wherein the detectable marker is 3×FLAG.
57. The polypeptide of paragraph 55 or 56, wherein the detectable marker comprises the amino acid sequence of SEQ ID NO: 40 or an amino acid sequence that is at least 80% identical to SEQ ID NO: 40.
58. The polypeptide of any one of paragraphs 21-57, further comprising a linker domain distal to the extracellular binding domain and/or proximal to the signal peptide and/or detectable marker.
59. The polypeptide of paragraph 58, wherein the linker comprises the amino acid sequence of SEQ ID NO: 42 or an amino acid sequence that is at least 80% identical to SEQ ID NO: 42.
60. The polypeptide of any one of paragraphs 21-59, further comprising a spacer domain located between the extracellular binding domain and the transmembrane domain.
61. The polypeptide of paragraph 60, wherein the spacer domain comprises a CD8 hinge domain.
62. The polypeptide of paragraph 60 or 61, wherein the spacer comprises the amino acid sequence of SEQ ID NO: 44 or an amino acid sequence that is at least 80% identical to SEQ ID NO: 44.
63. The CAR polypeptide of any one of paragraphs 21-62, wherein the polypeptide comprises the amino acid sequence of one of SEQ ID NOs: 80-114 or an amino acid sequence that is at least 80% identical to one of SEQ ID NOs: 80-114.
64. A chimeric antigen receptor (CAR) polypeptide for expression in natural killer (NK) cells comprising:
   a) an extracellular binding domain;
   b) a transmembrane domain; and
   c) intracellular domain comprising at least one intracellular signaling domain from a NK cell receptor.
65. The polypeptide of paragraph 64, wherein the polypeptide comprises from N-terminus to C-terminus:
   a) the extracellular binding domain;
   b) the transmembrane domain; and
   c) the intracellular domain.
66. The polypeptide of paragraph 64, wherein the polypeptide comprises from C-terminus to N-terminus:
   a) the extracellular binding domain;
   b) the transmembrane domain; and
   c) the intracellular domain.
67. The polypeptide of any one of paragraphs 64-66, wherein the NK cell receptor is selected from the group consisting of 2B4, NTB-A, CRACC, and CD2.
68. The polypeptide of any one of paragraphs 64-67, wherein the NK cell receptor is 2B4.
69. The polypeptide of any one of paragraphs 64-68, wherein the NK cell receptor is NTB-A.
70. The polypeptide of any one of paragraphs 64-69, wherein the NK cell receptor is CRACC.
71. The polypeptide of any one of paragraphs 64-70, wherein the NK cell receptor is CD2.
72. The polypeptide of any one of paragraphs 64-71, wherein the intracellular signaling domain from the NK cell receptor comprises the amino acid sequence of one of SEQ ID NOs: 7-10 or an amino acid sequence that is at least 80% identical to one of SEQ ID NOs: 7-10.
73. The polypeptide of any one of paragraphs 64-72, wherein the transmembrane domain comprises a transmembrane domain of a natural NK cell receptor.
74. The polypeptide of any one of paragraphs 64-73, wherein the transmembrane domain of the natural NK cell receptor is selected from the group consisting of Natural Killer Group 2D (NKG2D); Natural Killer Cell P46-Related Protein (NKp46); and DNAX Accessory Molecule-1 (DNAM1).
75. The polypeptide of any one of paragraphs 64-74, wherein the transmembrane domain comprises the amino acid sequence of one of SEQ ID NOs: 29-31 or 117-118 or an amino acid sequence that is at least 80% identical to one of SEQ ID NOs: 29-31 or 117-118.
76. The polypeptide of any one of paragraphs 64-75, wherein the extracellular binding domain is an antibody, an antigen-binding fragment thereof, a F(ab) fragment, a F(ab′) fragment, a single chain variable fragment (scFv), or a single-domain antibody (sdAb).
77. The polypeptide of any one of paragraphs 64-76, wherein the extracellular binding domain comprises an scFv.
78. The polypeptide of any one of paragraphs 64-77, wherein the extracellular binding domain specifically binds to a tumor-associated antigen.
79. The polypeptide of any one of paragraphs 64-78, wherein the tumor-associated antigen is CD19.
80. The polypeptide of any one of paragraphs 64-79, wherein the tumor-associated antigen is CD33.
81. The polypeptide of any one of paragraphs 64-80, wherein the extracellular binding domain comprises the amino acid sequence of one of SEQ ID NOs: 35-36 or an amino acid sequence that is at least 80% identical to one of SEQ ID NOs: 35-36.
82. The polypeptide of any one of paragraphs 64-81, further comprising a signal peptide located N-terminal to the extracellular binding domain.
83. The polypeptide of paragraph 82, wherein the signal peptide is a CD8 signal peptide.
84. The polypeptide of paragraph 82 or 83, wherein the signal peptide comprises the amino acid sequence of SEQ ID NO: 38 or an amino acid sequence that is at least 80% identical to SEQ ID NO: 38.
85. The polypeptide of any one of paragraphs 64-84, further comprising a detectable marker distal to the extracellular binding domain.
86. The polypeptide of paragraph 85, wherein the detectable marker is 3×FLAG.
87. The polypeptide of paragraph 85 or 86, wherein the detectable marker comprises the amino acid sequence of SEQ ID NO: 40 or an amino acid sequence that is at least 80% identical to SEQ ID NO: 40.
88. The polypeptide of any one of paragraphs 64-87, further comprising a linker domain distal to the extracellular binding domain and/or proximal to the signal peptide and/or detectable marker.
89. The polypeptide of paragraph 88, wherein the linker comprises the amino acid sequence of SEQ ID NO: 42 or an amino acid sequence that is at least 80% identical to SEQ ID NO: 42.
90. The polypeptide of any one of paragraphs 64-89, further comprising a spacer domain located between the extracellular binding domain and the transmembrane domain.
91. The polypeptide of paragraph 90, wherein the spacer domain comprises a CD8 hinge domain.
92. The polypeptide of paragraph 90 or 91, wherein the spacer comprises the amino acid sequence of SEQ ID NO: 44 or an amino acid sequence that is at least 80% identical to SEQ ID NO: 44.
93. The CAR polypeptide of any one of paragraphs 64-92, wherein the polypeptide comprises the amino acid sequence of one of SEQ ID NOs: 90-105 or SEQ ID NOs: 110-114 or an amino acid sequence that is at least 80% identical to one of SEQ ID NOs: 90-105 or SEQ ID NOs: 110-114.
94. A chimeric antigen receptor (CAR) polypeptide for expression in natural killer (NK) cells comprising:
   a) an extracellular binding domain;
   b) a transmembrane domain; and
   c) an intracellular domain comprising at least one of the following:
      i) an intracellular signaling domain from a NK cell membrane-bound signaling adaptor protein; and/or
      ii) an intracellular co-stimulatory domain from a co-stimulatory receptor.
95. The polypeptide of paragraph 94, wherein the polypeptide comprises from N-terminus to C-terminus:
   a) the extracellular binding domain;
   b) the transmembrane domain; and
   c) the intracellular domain.
96. The polypeptide of paragraph 94, wherein the polypeptide comprises from C-terminus to N-terminus:
   a) the extracellular binding domain;
   b) the transmembrane domain; and
   c) the intracellular domain.
97. The polypeptide of any one of paragraphs 94-96, wherein the NK cell membrane-bound signaling adaptor protein is CD3ζ or FcεR1.
98. The polypeptide of any one of paragraphs 94-97, wherein the intracellular signaling domain from the NK cell membrane-bound signaling adaptor protein comprises the amino acid sequence of one of SEQ ID NOs: 11-12 or an amino acid sequence that is at least 80% identical to one of SEQ ID NOs: 11-12.
99. The polypeptide of any one of paragraphs 94-98, wherein the co-stimulatory receptor is 4-1BB and/or IL2 receptor beta (IL2RB).
100. The polypeptide of paragraph 99, wherein the intracellular co-stimulatory domain from the co-stimulatory receptor comprises the amino acid sequence of one of SEQ ID NOs: 15-16 or an amino acid sequence that is at least 80% identical to one of SEQ ID NOs: 15-16.
101. The polypeptide of any one of paragraphs 94-100, wherein the intracellular domain further comprises at least one self-cleaving peptide.
102. The polypeptide of paragraph 101, wherein the self-cleaving peptide is T2A, P2A, E2A, or F2A.
103. The polypeptide of paragraph 101 or 102, wherein the self-cleaving peptide comprises the amino acid sequence of one of SEQ ID NOs: 19-20 or an amino acid sequence that is at least 80% identical to one of SEQ ID NOs: 19-20.
104. The polypeptide of any one of paragraphs 94-103, wherein the intracellular domain further comprises a cytokine.
105. The polypeptide of paragraph 104, wherein the cytokine is IL-15 or IL-21.
106. The polypeptide of paragraph 104 or 105, wherein the cytokine comprises the amino acid sequence of one of SEQ ID NOs: 23-24 or an amino acid sequence that is at least 80% identical to one of SEQ ID NOs: 23-24.
107. The polypeptide of any one of paragraphs 94-106, wherein the intracellular domain further comprises at least one self-cleaving peptide and at least one cytokine.
108. The polypeptide of paragraph 107, wherein the cytokine is adjacent and distal to the self-cleaving peptide, such that the cytokine is separated from the polypeptide by the self-cleaving peptide.
109. The polypeptide of any one of paragraphs 94-108, wherein the transmembrane domain comprises a transmembrane domain of CD8.
110. The polypeptide of any one of paragraphs 94-109, wherein the transmembrane domain comprises the amino acid sequence of SEQ ID NO: 32 or an amino acid sequence that is at least 80% identical to SEQ ID NO: 32.
111. The polypeptide of any one of paragraphs 94-110, wherein the extracellular binding domain is an antibody, an antigen-binding fragment thereof, a F(ab) fragment, a F(ab′) fragment, a single chain variable fragment (scFv), or a single-domain antibody (sdAb).
112. The polypeptide of any one of paragraphs 94-111, wherein the extracellular binding domain comprises a scFv.
113. The polypeptide of any one of paragraphs 94-112, wherein the extracellular binding domain specifically binds to a tumor-associated antigen.
114. The polypeptide of any one of paragraphs 94-113, wherein the tumor-associated antigen is CD19.
115. The polypeptide of any one of paragraphs 94-114, wherein the tumor-associated antigen is CD33.
116. The polypeptide of any one of paragraphs 94-115, wherein the extracellular binding domain comprises the amino acid sequence of one of SEQ ID NOs: 35-36 or an amino acid sequence that is at least 80% identical to one of SEQ ID NOs: 35-36.
117. The polypeptide of any one of paragraphs 94-116, further comprising a signal peptide located N-terminal to the extracellular binding domain.
118. The polypeptide of paragraph 117, wherein the signal peptide is a CD8 signal peptide.
119. The polypeptide of paragraph 117 or 118, wherein the signal peptide comprises the amino acid sequence of SEQ ID NO: 38 or an amino acid sequence that is at least 80% identical to SEQ ID NO: 38.
120. The polypeptide of any one of paragraphs 94-119, further comprising a detectable marker distal to the extracellular binding domain.
121. The polypeptide of paragraph 120, wherein the detectable marker is 3×FLAG.
122. The polypeptide of paragraph 120 or 121, wherein the detectable marker comprises the amino acid sequence of SEQ ID NO: 40 or an amino acid sequence that is at least 80% identical to SEQ ID NO: 40.
123. The polypeptide of any one of paragraphs 94-122, further comprising a linker domain distal to the extracellular binding domain and/or proximal to the signal peptide and/or detectable marker.
124. The polypeptide of paragraph 123, wherein the linker comprises the amino acid sequence of SEQ ID NO: 42 or an amino acid sequence that is at least 80% identical to SEQ ID NO: 42.
125. The polypeptide of any one of paragraphs 74-104, further comprising a spacer domain located between the extracellular binding domain and the transmembrane domain.
126. The polypeptide of paragraph 125, wherein the spacer domain comprises a CD8 hinge domain.
127. The polypeptide of paragraph 125 or 126, wherein the spacer comprises the amino acid sequence of SEQ ID NO: 44 or an amino acid sequence that is at least 80% identical to SEQ ID NO: 44.
128. The polypeptide of any one of paragraphs 94-127, wherein the polypeptide comprises the amino acid sequence of one of SEQ ID NOs: 80-89 or SEQ ID NOs: 106-109 or an amino acid sequence that is at least 80% identical to one of SEQ ID NOs: 80-89 or SEQ ID NOs: 106-109.
129. A polynucleotide encoding the polypeptide of any one of paragraphs 1-17 or 21-128.
130. The polynucleotide of paragraph 129, wherein the polynucleotide comprises one of SEQ ID NOs: 45-79, or a nucleic acid sequence that is at least 80% identical to one of SEQ ID NOs: 45-79.
131. A vector comprising the polynucleotide of paragraph 129 or 130.
132. The vector of paragraph 131, wherein the vector comprises a lentiviral vector.
133. A lentivirus comprising the polypeptide of any one of paragraphs 1-17 or 21-128, the polynucleotide of any one of paragraphs 129-130, or the vector of any one of paragraphs 131-132.
134. A cell or population thereof comprising the polypeptide of any one of paragraphs 1-17 or 21-128, the polynucleotide of any one of paragraphs 129-130, the vector of any one of paragraphs 131-132, or the lentivirus of paragraph 133.
135. The cell of paragraph 134, wherein the cell comprises an immune cell.
136. The cell of paragraph 135, wherein the immune cell comprises a natural killer (NK) cell.
137. A pharmaceutical composition comprising the polypeptide of any one of paragraphs 1-17 or 21-128, the polynucleotide of any one of paragraphs 129-130, the vector of any one of paragraphs 131-132, the lentivirus of paragraph 133, or the cell of any one of paragraphs 18 or 134-136, and a pharmaceutically acceptable carrier.
138. A method of increasing the activation of an NK cell or population thereof comprising contacting the cell or population thereof with the polypeptide of any one of paragraphs 1-17 or 21-128, the polynucleotide of any one of paragraphs 129-130, the vector of any one of paragraphs 131-132, or the lentivirus of paragraph 133.
139. The method of paragraph 138, wherein contacting the NK cell or population thereof with the polypeptide, polynucleotide, vector, or lentivirus increases the activity of the NK cell by at least 10%, at least 20%, at least 30%, at least 40%, at least 50%, at least 60%, at least 70%, at least 80%, at least 90%, at least 100%, at least 200%, at least 300%, at least 400%, or at least 500% compared to prior to contacting with the polypeptide, polynucleotide, vector, or lentivirus.
140. The method of any one of paragraphs 138-139, wherein the increased activation of the NK cell or population thereof comprises increased expression of a cytokine or granzyme selected from the group consisting of TNFα, IFNγ, GM-CSF, and Granzyme B.
141. The method of any one of paragraphs 138-140, wherein the increased activation of the NK cell or population thereof results in an increased specific lysis of a target cell.
142. The method of any one of paragraphs 138-141, wherein the target cell expresses a surface antigen that binds specifically to the extracellular binding domain of the polypeptide.
143. The method of any one of paragraphs 138-142, wherein the target cell is a cancer cell.
144. The method of any one of paragraphs 138-143, wherein the target cell is a cell infected by a virus or bacteria.
145. A method of treating a subject in need of a CAR-based therapy comprising administering to the subject a therapeutically effective amount of the CAR-based therapy selected from the group consisting of: the polypeptide of any one of paragraphs 1-17 or 21-128, the polynucleotide of any one of paragraphs 129-130, the vector of any one of paragraphs 131-132, the lentivirus of paragraph 133, the cell or population thereof of any one of paragraphs 18 or 134-136, and the pharmaceutical composition of paragraph 137.
146. The method of paragraph 145, wherein the subject has cancer or has been diagnosed with cancer.
147. The method of paragraph 145 or 146, wherein the subject has or has been diagnosed with adrenal cancer, anal cancer, appendix cancer, bile duct cancer, bladder cancer, bone cancer, brain cancer, breast cancer, cervical cancer, colorectal cancer, gallbladder cancer, gestational trophoblastic disease, head and neck cancer, Hodgkin lymphoma, intestinal cancer, kidney cancer, leukemia, liver cancer, lung cancer, melanoma, Merkel cell carcinoma, mesothelioma, multiple myeloma, neuroendocrine tumors, Non-Hodgkin lymphoma, oral cancer, ovarian cancer, pancreatic cancer, prostate cancer, sinus cancer, skin cancer, a sarcoma, a soft tissue sarcoma, spinal cancer, stomach cancer, testicular cancer, throat cancer, a tumor, thyroid cancer, uterine cancer, vaginal cancer or vulvar cancer.
148. The method of any one of paragraphs 145-147, wherein the administration of the CAR-based therapy results in increased specific lysis of cancer cells targeted by the CAR.
149. The method of any one of paragraphs 145-148, wherein the subject has an infectious disease or has been diagnosed with an infectious disease.
150. The method of any one of paragraphs 145-149, wherein the infectious disease is a viral or bacterial disease.
151. The method of any one of paragraphs 145-150, wherein the administration of the CAR-based therapy results in increased specific lysis of infected cells targeted by the CAR.
152. A method of making a therapeutic composition, the method comprising introducing a nucleic acid encoding the polypeptide of any one of paragraphs 1-17 or 21-128, the polynucleotide of any one of paragraphs 129-130, the vector of any one of paragraphs 131-132, or the lentivirus of paragraph 133 to an NK cell under conditions permitting expression of the polypeptide in the cell.
153. The method of paragraph 152, wherein prior to introducing the nucleic acid, polynucleotide, vector, or lentivirus, the NK cell is removed from a subject in need of the therapeutic composition.
154. The method of paragraph 152 or 153, wherein after introducing the nucleic acid, polynucleotide, vector, or lentivirus, the NK cell is returned to the subject.

The technology described herein is further illustrated by the following examples which in no way should be construed as being further limiting.

### EXAMPLES

### Example 1

### CD19 CAR-NK Constructs and Embodiments

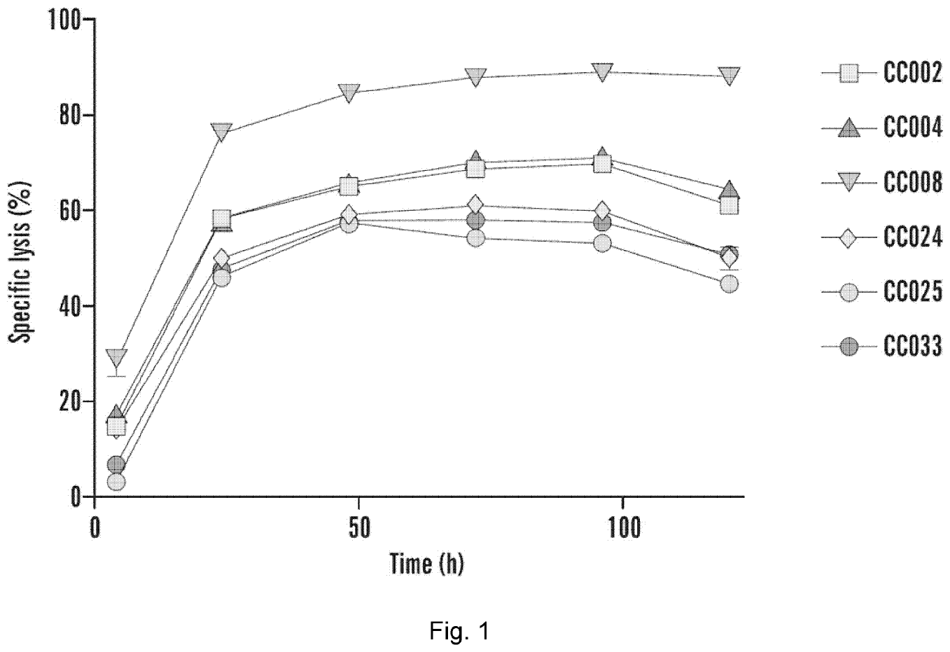

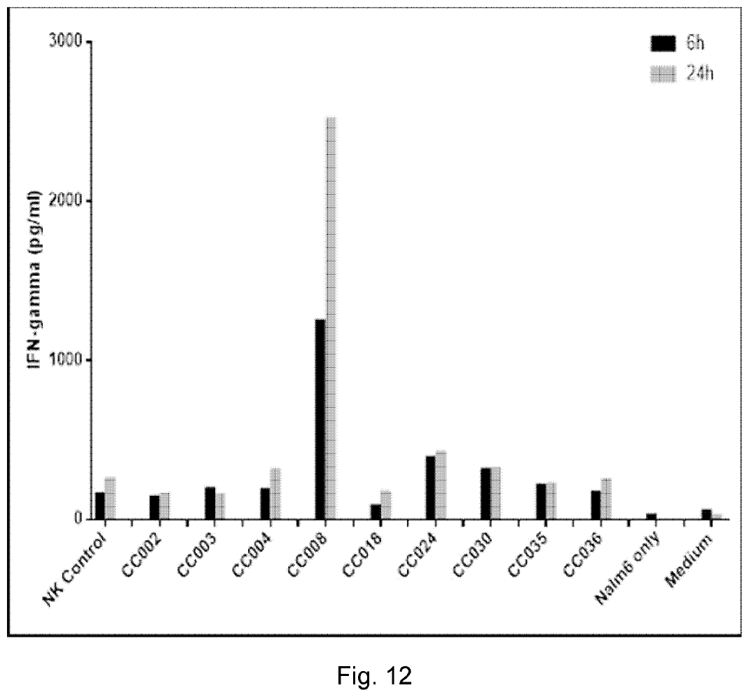

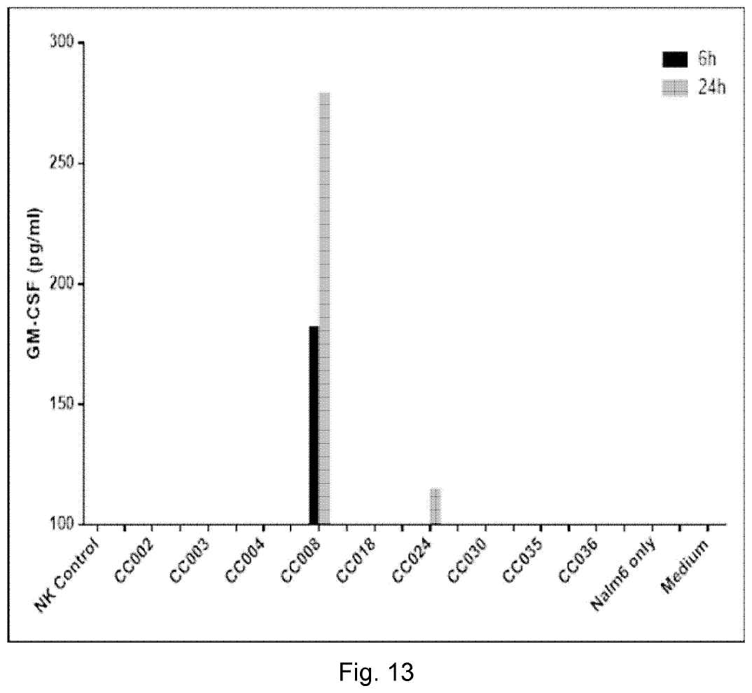

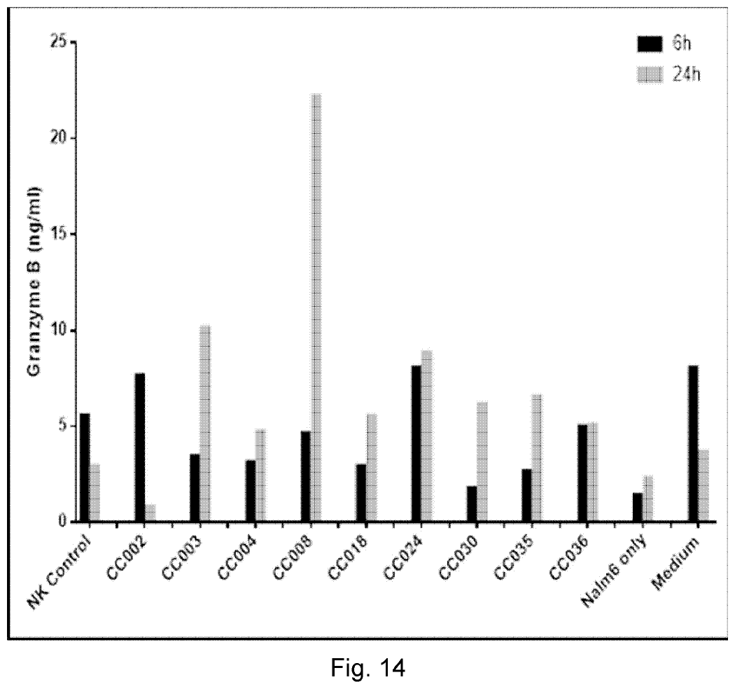

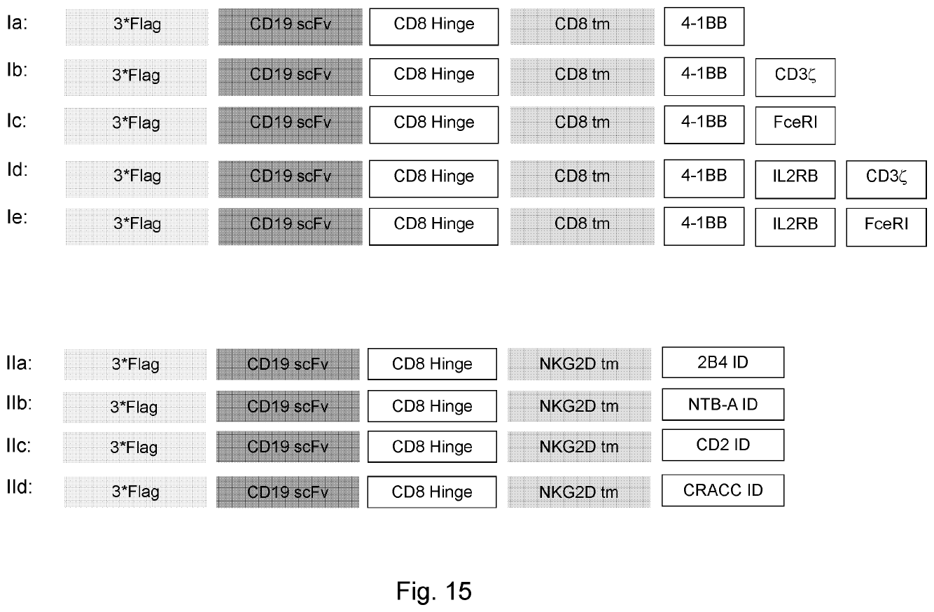

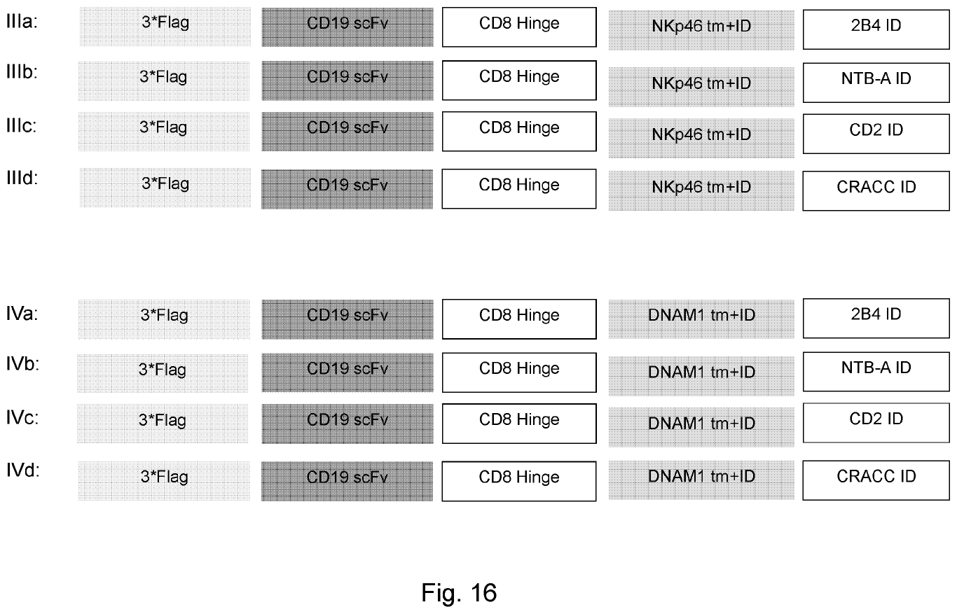

Tables 1 and 2 show exemplary CD19 CAR-NK constructs. For exemplary CD19 CAR-NK data, see e.g., Examples 1-7 or FIGS. 1-17.

TABLE 1
CD19 CAR-NK Constructs with different transmembrane domain (TD) +
costimulatory domain (CD) + signaling domain (SD)

| Construct | Description |
|---|---|
| CC001 | pSIH1-hEF1a-GFP |
| CC002 | pSIH1-hEF1a-CD19-CD8 4-1BB |
| CC003 | CC004 (without 3*Flag) |
| CC004 | pSIH1-hEF1a-CD19FN CD8-4-1BB-CD3z |
| CC005 | pSIH1-hEF1a-CD19FN CD8-4-1BB-FcRIgE |
| CC007 | pSIH1-hEF1a-CD19FN CD8-4-1BB-FcRIgE-P2A-IL15 |
| CC008 | pSIH1-hEF1a-CD19FN CD8-4-1BB-FcRIgE-P2A-IL15-IL21 |
| CC013 | pSIH1-hEF1a-CD19FN CD8-4-1BB-IL2RB-CD3z |
| CC016 | pSIH1-hEF1a-CD19FN CD8-IL2RB-4-1BB-CD3z |
| CC017 | pSIH1-hEF1a-CD19FN CD8-4-1BB-IL2RB-FcR IgE |
| CC018 | pSIH1-hEF1a-CD19FN CD8 IL2RB-4-1BB-FcR IgE |
| CC024 | pSIH1-hEF1a-CD19FNN NKG2D-2B4 |
| CC025 | pSIH1-hEF1a-CD19FCN NKG2D-2B4 |
| CC026 | pSIH1-hEF1a-CD19FNN NKp46-CD2 |
| CC027 | pSIH1-hEF1a-CD19FNN DNAM1-2B4 |
| CC028 | pSIH1-hEF1a-CD19FCN NKG2D-NTB-A |
| CC029 | pSIH1-hEF1a-CD19FNN NKp46-NTB-A |
| CC030 | pSIH1-hEF1a-CD19FNN DNAM1-CRACC |
| CC031 | pSIH1-hEF1a-CD19FCN NKG2D-CD2 |
| CC032 | pSIH1-hEF1a-CD19FNN NKp46-2B4 |
| CC033 | pSIH1-hEF1a-CD19FNN NKp46-CRACC |
| CC034 | pSIH1-hEF1a-CD19FNN DNAM1-CD2 |
| CC035 | pSIH1-hEF1a-CD19FNN NKG2D-CRACC |
| CC036 | pSIH1-hEF1a-CD19FCN NKG2D-CRACC |
| CC037 | pSIH1-hEF1a-CD19FNN NKG2D-CD2 |
| CC038 | pSIH1-hEF1a-CD19FNN DNAM1-NTB-A |
| CC039 | pSIH1-hEF1a-CD19FNN NKG2D-NTB-A |

TABLE 2
CD19 CAR-NK Constructs

| | | | Trans- | Co-stimulatory |
|---|---|---|---|---|
| | | | membrane | Domain (CD) + |
| | | | Domain | Signaling |
| | Construct No. | Hinge | (TD) | Domain (SD) |
| Group | Ia: CC002 | CD8 | CD8 | 4-1BB |
| I: | Ib: CC003; CC004 | CD8 | CD8 | 4-1BB + CD3z |
| | Ic: CC005; CC007; | CD8 | CD8 | 4-1BB + FceRI |
| | CC008 | | | |
| | Id: C0013; C0016 | CD8 | CD8 | 4-1BB/IL2RB + |
| | | | | CD3z |
| | Ie: CC017; CC018 | CD8 | CD8 | 4-1BB/IL2RB + |
| | | | | FceRI |
| Group | IIa: CC024; CC025 | CD8 | NKG2D | 2B4 |
| II | IIb: CC028; CC039 | CD8 | NKG2D | NTB-A |
| | IIc: CC031; CC037 | CD8 | NKG2D | CD2 |
| | IId: CC035; CC036 | CD8 | NKG2D | CRACC |
| Group | IIIa: CC032 | CD8 | NKp46 | NKp46 + 2B4 |
| III | IIIb: CC029 | CD8 | NKp46 | NKp46 + NTB-A |
| | IIIc: CC026 | CD8 | NKp46 | NKp46 + CD2 |
| | IIId: CC033 | CD8 | NKp46 | NKp46 + CRACC |
| Group | IVa: CC027 | CD8 | DNAM1 | DNAM1 + 2B4 |
| IV | IVb: CC038 | CD8 | DNAM1 | DNAM1 + NTB-A |
| | IVc: CC034 | CD8 | DNAM1 | DNAM1 + CD2 |
| | IVd: CC030 | CD8 | DNAM1 | DNAM1 + CRACC |
| Ctrl | CC001 | N/A | N/A | N/A |

This study compares the cytotoxicity of exemplary NK CARs CC008, CC024, CC025, and CC033 with control CARs CC002 and CC004. The CARs were transfected into NK92 cells using the lentiviral based method. For lentiviral production, the CAR constructs were first cloned into the plasmid pSIH1 (SBI Cat #CD510B-1), followed by transfection into the 293FT packaging cells along with helper plasmids, pMDL, VSVG and REV using a CaCl₂ mediated transfection method. Culture supernatants were collected after 48 h, filtered (0.22 μm) and stored at −80° C. The titer of LV particles was determined using a Lenti-X p24 Rapid Titer Kit (Novoprotein™). For NK92 cell transduction, NK92 cells were loaded in 24-well plates followed by the addition of lentivirus at a multiplicity of infection (MOI) of 20 in the presence of 8 μg/ml POLYBRENE and incubated at 37° C. for 48 h. CAR transduction efficiency was determined using Guava Easycyte™ flow cytometry, measuring Flag-tag expression using fluorescent labeled anti-flag tag antibody.

Cytotoxicity of each CAR was evaluated with a real-time cell assay (RTCA) using CD19 expressing A549 cell line as target cell and CAR transfected NK92 cell as effector cell. For measuring of specific lysis (%) of target cell, the CD19-expressing A549 target cells (i.e., tumor cells) were first seeded in the wells of an electronic microtiter plate (E-Plate®) to let cells settle and target cell attachment. On the second day, media was removed from the wells and effector cells were added to the plates and incubated at 37° C. for another 2-5 days. The real-time killing of target cells was detected using the xCELLigence™ RTCA instruments and data was analyzed using xCELLigence™ software.

As shown in FIG. 1, the cytotoxicity of CC008 CAR construct is significantly higher than control CARs CC002 and CC004.

### Example 2

This study compares the cytotoxicity of exemplary NK CARs CC005, CC007, CC008, CC013, CC016, CC017, CC018, and CC024 with NK92 control without CAR transduction. The CARs were transfected into NK92 cells using the lentiviral based method. For lentiviral production, the CAR constructs were first cloned into the plasmid pSIH1 (SBI Cat #CD510B-1), followed by transfection into the 293FT packaging cell along with helper plasmids, pMDL, VSVG and REV using a CaCl₂ mediated transfection method. Culture supernatants were collected after 48 h, filtered (0.22 μm) and stored at −80° C. The titer of LV particles was determined using a Lenti-X p24 Rapid Titer Kit (Novoprotein™). For NK92 cell transduction, NK92 cells were loaded in 24-well plates followed by the addition of lentivirus at an MOI of 20 in the presence of 8 μg/ml Polybrene and incubated at 37° C. for 48 h. CAR transduction efficiency was determined using Guava Easycyte™ flow cytometry, measuring Flag-tag expression using fluorescent labeled anti-flag tag antibody.

Cytotoxicity of each CAR was evaluated with a Calcein-AM assay using Raji cell line as target cell and CAR transfected NK92 cell as effector cell. For measuring of specific lysis (%) of target cell, 1×10⁶ effector cells were stained with 0.15 NM Calcein-AM in NK92 medium for 25 minutes (min) at 37° C. in a volume of 10 mL. Cells were then washed twice in NK92 medium and added to 96-well round bottom plates at a concentration of 5×10⁴ cells/mL in triplicate, and effector cells were added at 5:1 effector-to-target (E:T) ratio. Plates were centrifuged at 300 rpm for 1 min and incubated at 37° C. for 4 h. Cells were centrifuged at 400 g for 5 min and the supernatants were transferred into 96-well flat white plates. Percentage Specific lysis was calculated using fluorescence (Ex 495 nm, Em 520 nm) of 2% TRITON X100-treated cells as maximum cell death and untreated cells as spontaneous cell death and using the formula % specific lysis=100×[(experimental data−spontaneous cell death)/(maximum cell death−spontaneous cell death)].

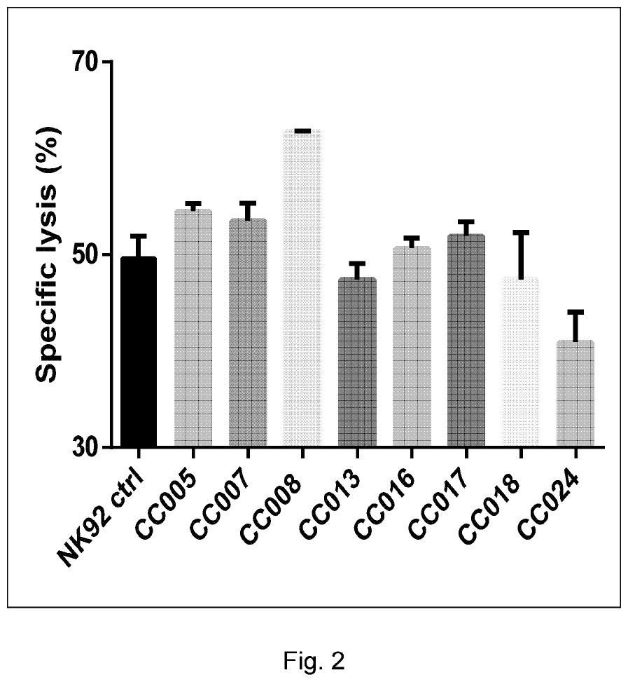

Results as shown in FIG. 2 demonstrate that cytotoxic activity of CC005/CC007/CC008 CAR constructs incorporating FcεRI intracellular domain is higher than the other CAR constructs evaluated.

### Example 3

This study compares the cytotoxicity of exemplary NK CARs CC008, and CC026-CC039 with control CARs CC002 and CC004. The CARs were transfected into NK92 cells using the lentiviral based method. For lentiviral production, the CAR constructs were first cloned into the plasmid pSIH1 (SBI Cat #CD510B-1), followed by transfection into the 293FT packaging cell along with helper plasmids, pMDL, VSVG and REV using a CaCl₂ mediated transfection method. Culture supernatants were collected after 48 h, filtered (0.22 μm) and stored at −80° C. The titer of LV particles was determined using a Lenti-X p24 Rapid Titer Kit (Novoprotein™). For NK92 cell transduction, NK92 cells were loaded in 24-well plates followed by the addition of lentivirus at an MOI of 20 in the presence of 8 μg/ml Polybrene and incubated at 37° C. for 48 h. CAR transduction efficiency was determined using Guava Easycyte™ flow cytometry, measuring Flag-tag expression using fluorescent labeled anti-flag tag antibody.

Cytotoxicity of each CAR was evaluated with a Calcein-AM assay using Raji cell line as target cell and CAR transfected NK92 cell as effector cell. For measuring of specific lysis (%) of target cell, 1×10⁶ effector cells were stained with 0.15 μM Calcein-AM in NK92 medium for 25 min at 37° C. in a volume of 10 mL. Cells were then washed twice in NK92 medium and added to 96-well round bottom plates at a concentration of 5×10⁴ cells/mL in triplicate, and effector cells were added at 5:1 effector-to-target (E:T) ratio. Plates were centrifuged at 300 rpm for 1 min and incubated at 37° C. for 4 h. Cells were centrifuged at 400 g for 5 min and the supernatants were transferred into 96-well flat white plates. Percentage Specific lysis was calculated using fluorescence (Ex 495 nm, Em 520 nm) of 2% TRITON X100-treated cells as maximum cell death and untreated cells as spontaneous cell death and using the formula % specific lysis=100×[(experimental data−spontaneous cell death)/(maximum cell death−spontaneous cell death)].

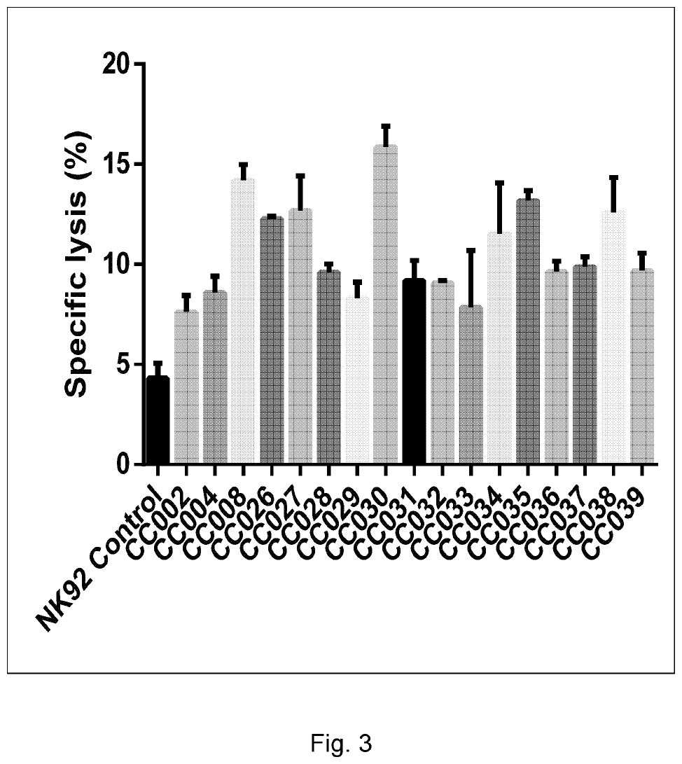

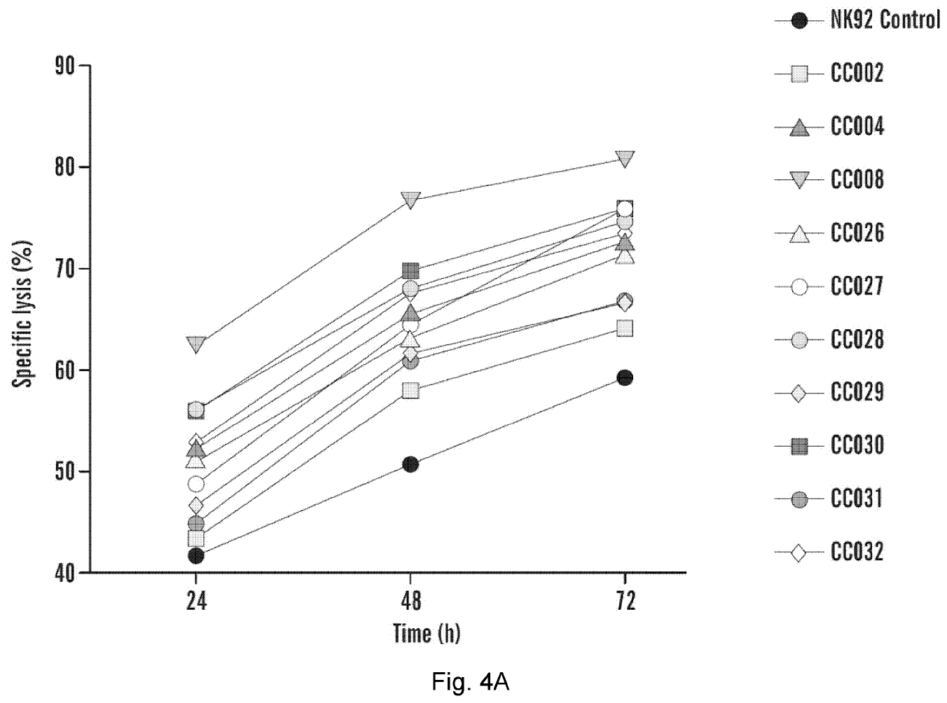

As shown in FIG. 3, CC027/CC030/CC034/CC038 CAR constructs incorporating DNAM1 intracellular domain, CC026 construct incorporating NKp46 and CD2 intracellular domain, and CC028/CC035/CC036 construct incorporating NKG2D and CRACC intracellular domain demonstrate strong cytotoxic activity against Raji tumor cell targets.

Cytotoxicity of each CAR was further evaluated with a bioluminescence assay using Nalm6-GFP-luciferase cell line as target cell and CAR transfected NK92 cell as effector cell. For measuring of specific lysis (%) of target cell, luciferase-expressing tumor cells were placed in 96-well round bottom plates at a concentration of 5×10⁵ cells/mL in triplicate, and effector cells were added at 1:1 effector-to-target (E:T) ratio. Plates were centrifuged at 300 rpm for 1 min and incubated at 37° C. for 24, 48 and 72 hours, respectively. After completion of incubation, D-luciferin (Perkin Elmer™) was added to each well at a final concentration of 0.14 mg/mL and incubated at 37° C. for 10 min. Cells were then mixed and transferred into 96-well flat white plates. Bioluminescence imaging (BLI) was measured for 1 second with a luminometer (Tecan™, Spark™). Percentage specific lysis was calculated using the formula % specific lysis=100×[(spontaneous cell data−experimental data)/(spontaneous cell data−medium control data)].

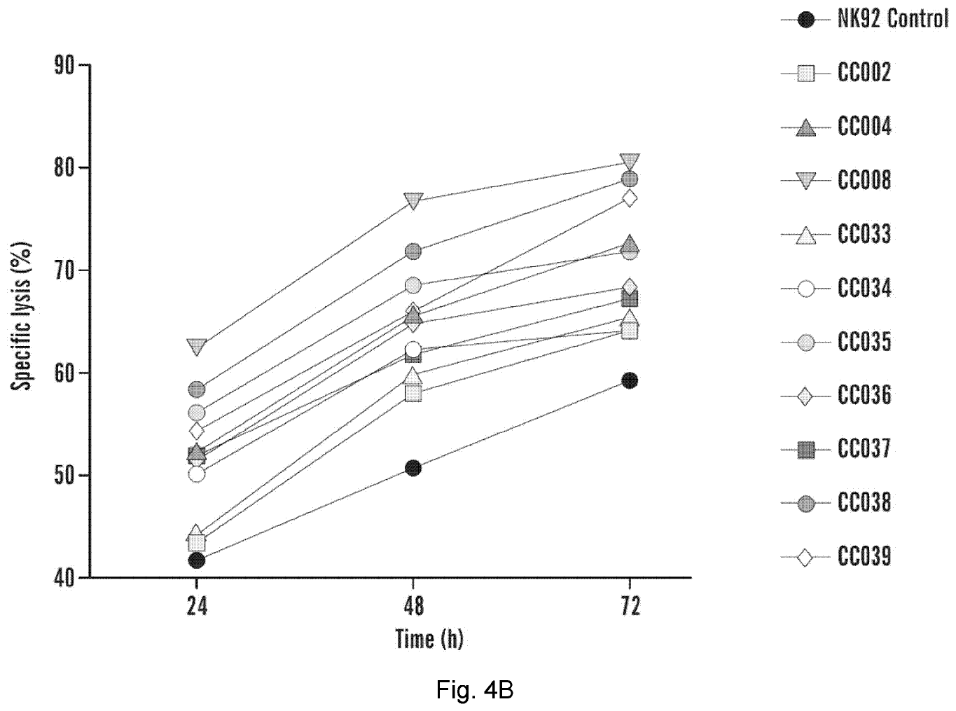

As shown in FIGS. 4A and 4B, CC028/CC039/CC035 CAR constructs incorporating NKG2D plus CRACC or NTB-A intracellular domain, and CC030/CC038 construct incorporating DNAM1 plus CRACC or NTB-A intracellular domain demonstrate strong cytotoxic activity against Nalm6-GFP-luciferase tumor cell targets.

### Example 4

This study compares the cytotoxicity of exemplary NK CARs CC005, CC007, CC008, CC016, CC018, CC024, CC025, CC028, CC030, CC035, CC036, CC038, and CC039 with control CARs CC002 and CC004. The CARs were transfected into NK92 cells using the lentiviral based method. For lentiviral production, the CAR constructs were first cloned into the plasmid pSIH1 (SBI Cat #CD510B-1), followed by transfection into the 293FT packaging cell along with helper plasmids, pMDL, VSVG and REV using a CaCl₂ mediated transfection method. Culture supernatants were collected after 48 h, filtered (0.22 μm) and stored at −80° C. The titer of LV particles was determined using a Lenti-X p24 Rapid Titer Kit (Novoprotein™). For NK92 cell transduction, NK92 cells were loaded in 24-well plates followed by the addition of lentivirus at an MOI of 20 in the presence of 8 μg/ml Polybrene and incubated at 37° C. for 48 h. CAR transduction efficiency was determined using Guava Easycyte™ flow cytometry, measuring Flag-tag expression using fluorescent labeled anti-flag tag antibody.

Cytotoxicity of each CAR was evaluated with a Calcein-AM assay using Raji cell line as target cell and CAR transfected NK92 cell as effector cell. For measuring of specific lysis (%) of target cell, 1×10⁶ effector cells were stained with 0.15 μM Calcein-AM in NK92 medium for 25 min at 37° C. in a volume of 10 mL. Cells were then washed twice in NK92 medium and added to 96-well round bottom plates at a concentration of 5×10⁴ cells/mL in triplicate, and effector cells were added at 5:1 effector-to-target (E:T) ratio. Plates were centrifuged at 300 rpm for 1 min and incubated at 37° C. for 4 h. Cells were centrifuged at 400 g for 5 min and the supernatant were transferred into 96-well flat white plates. Percentage Specific lysis was calculated using fluorescence (Ex 495 nm, Em 520 nm) of 2% TRITON X100-treated cells as maximum cell death and untreated cells as spontaneous cell death and using the formula % specific lysis=100×[(experimental data−spontaneous cell death)/(maximum cell death−spontaneous cell death)].

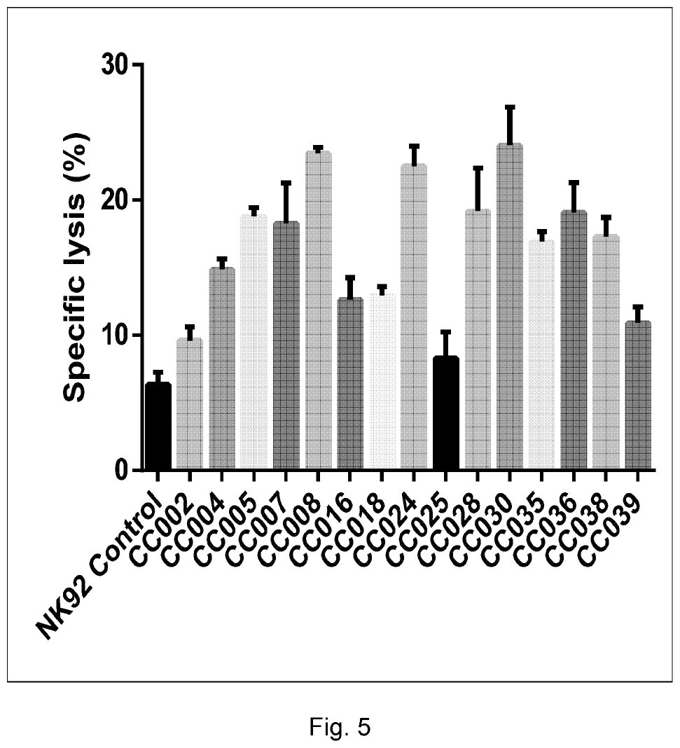

As shown in FIG. 5, CC005/CC007/CC008 CAR constructs incorporating CD8 and FcεRI intracellular domain, CC024/CC028/CC035/CC036 constructs incorporating NKG2D intracellular domain, and CC030/CC038 incorporating DNAM1 intracellular domain demonstrate strong cytotoxic activity against Raji tumor cell targets.

Cytotoxicity of each CAR was further evaluated with a bioluminescence assay using Nalm6-GFP-luciferase cell line as target cell and CAR transfected NK92 cell as effector cell. For measuring of specific lysis (%) of target cell, luciferase-expressing tumor cells were placed in 96-well round bottom plates at a concentration of 5×10⁵ cells/mL in triplicate, and effector cells were added at 1:1 effector-to-target (E:T) ratio. Plates were centrifuged at 300 rpm for 1 min and incubated at 37° C. for 24, 48 and 72 hours, respectively. After completion of incubation, D-luciferin (Perkin Elmer™) was added to each well at a final concentration of 0.14 mg/mL and incubated at 37° C. for 10 mins. Cells were then mixed and transferred into 96-well flat white plates. BLI was measured for 1 second with a luminometer (Tecan™, Spark™). Percentage specific lysis was calculated using the formula % specific lysis=100×[(spontaneous cell data−experimental data)/(spontaneous cell data−medium control data)].

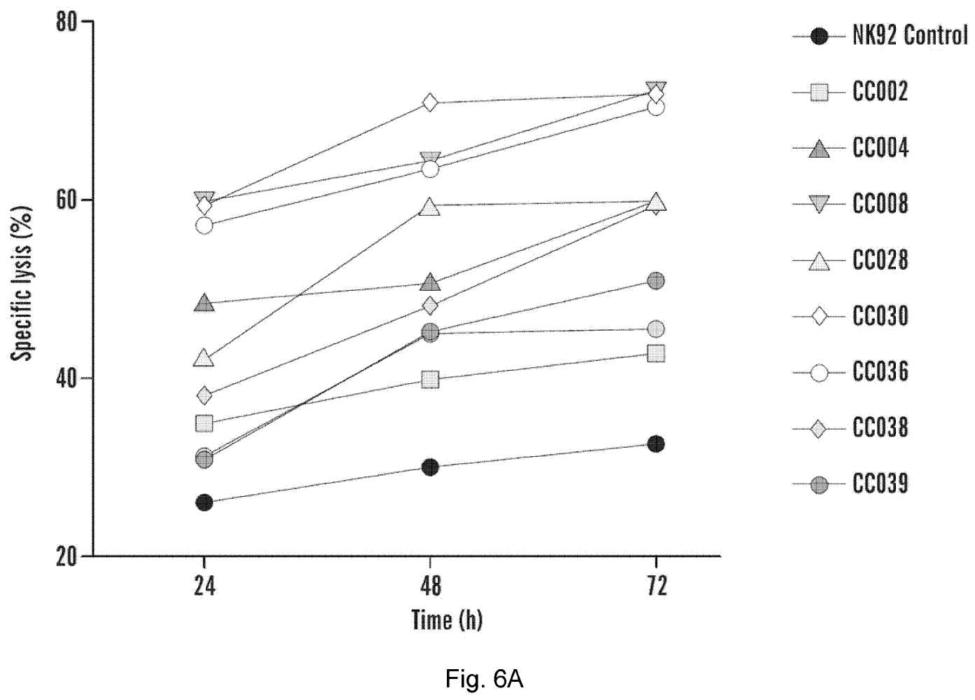

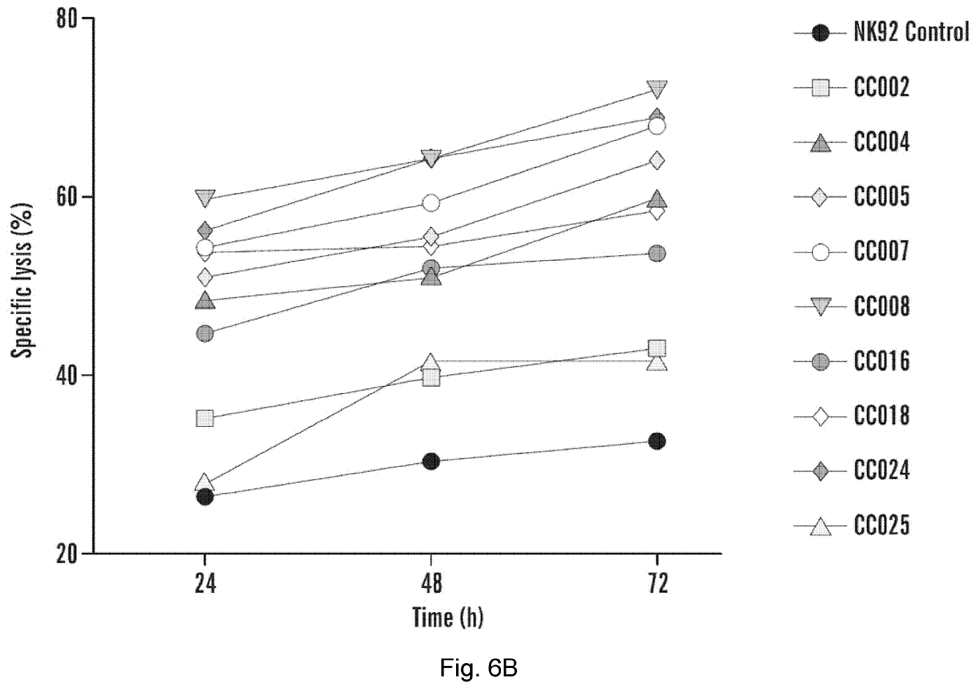

As shown in FIGS. 6A and 6B, CC005/CC007/CC008 CAR constructs incorporating CD8 and FcεRI intracellular domain and CC024/CC036/CC039 incorporating NKG2D intracellular domain demonstrate strong cytotoxic activity against Nalm6-GFP-luciferase tumor cell targets.

### Example 5

This study compares the cytotoxicity of exemplary NK CARs CC005, CC007, CC008, CC024, CC030, CC035, and CC036 with control CARs CC002 and CC004. The CARs were transfected into NK92 cells using the lentiviral based method. For lentiviral production, the CAR constructs were first cloned into the plasmid pSIH1 (SBI Cat #CD510B-1), followed by transfection into the 293FT packaging cell along with helper plasmids, pMDL, VSVG and REV using a CaCl₂ mediated transfection method. Culture supernatants were collected after 48 h, filtered (0.22 μm) and stored at −80° C. The titer of LV particles was determined using a Lenti-X p24 Rapid Titer Kit (Novoprotein™). For NK92 cell transduction, NK92 cells were loaded in 24-well plates followed by the addition of lentivirus at an MOI of 20 in the presence of 8 μg/ml Polybrene and incubated at 37° C. for 48 h. CAR transduction efficiency was determined using Guava Easycyte™ flow cytometry, measuring Flag-tag expression using fluorescent labeled anti-flag tag antibody.

Cytotoxicity of each CAR was evaluated with a Calcein-AM assay using Raji cell line as target cell and CAR transfected NK92 cell as effector cell. For measuring of specific lysis (%) of target cell, 1×10⁶ effector cells were stained with 0.15 μM Calcein-AM in NK92 medium for 25 min at 37° C. in a volume of 10 mL. Cells were then washed twice in NK92 medium and added to 96-well round bottom plates at a concentration of 5×10⁴ cells/mL in triplicate, and effector cells were added at 5:1 effector-to-target (E:T) ratio. Plates were centrifuged at 300 rpm for 1 min and incubated at 37° C. for 4 h. Cells were centrifuged at 400 g for 5 min and the supernatants were transferred into 96-well flat white plates. Percentage Specific lysis was calculated using fluorescence (Ex 495 nm, Em 520 nm) of 2% TRITON X100-treated cells as maximum cell death and untreated cells as spontaneous cell death and using the formula % specific lysis=100×[(experimental data−spontaneous cell death)/(maximum cell death−spontaneous cell death)].

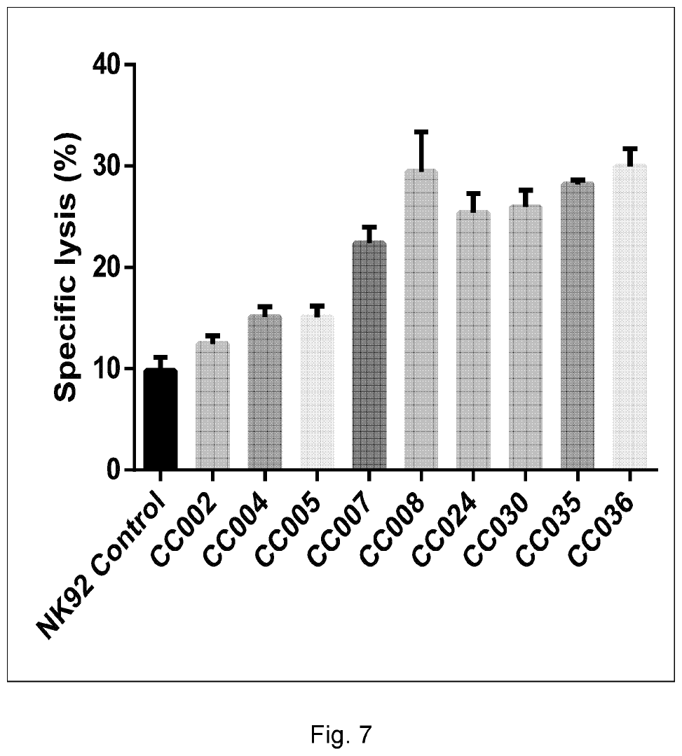

As shown in FIG. 7, CC007/CC008 CAR constructs incorporating CD8 and FcεRI intracellular domain, CC024/CC035/CC036 constructs incorporating NKG2D intracellular domain, and CC030 incorporating DNAM1 intracellular domain demonstrate strong cytotoxic activity against Raji tumor cell targets.

Cytotoxicity of each CAR was further evaluated with a bioluminescence assay using Nalm6-GFP-luciferase cell line as target cell and CAR transfected NK92 cell as effector cell. For measuring of specific lysis (%) of target cell, luciferase-expressing tumor cells were placed in 96-well round bottom plates at a concentration of 5×10⁵ cells/mL in triplicate, and effector cells were added at 1:1 effector-to-target (E:T) ratio. Plates were centrifuged at 300 rpm for 1 min and incubated at 37° C. for 24, 48 and 72 hours, respectively. After completion of incubation, D-luciferin (Perkin Elmer™) was added to each well at a final concentration of 0.14 mg/mL and incubated at 37° C. for 10 min. Cells were then mixed and transferred into 96-well flat white plates. BLI was measured for 1 second with a luminometer (Tecan™, Spark™). Percentage specific lysis was calculated using the formula % specific lysis=100×[(spontaneous cell data−experimental data)/(spontaneous cell data−medium control data)].

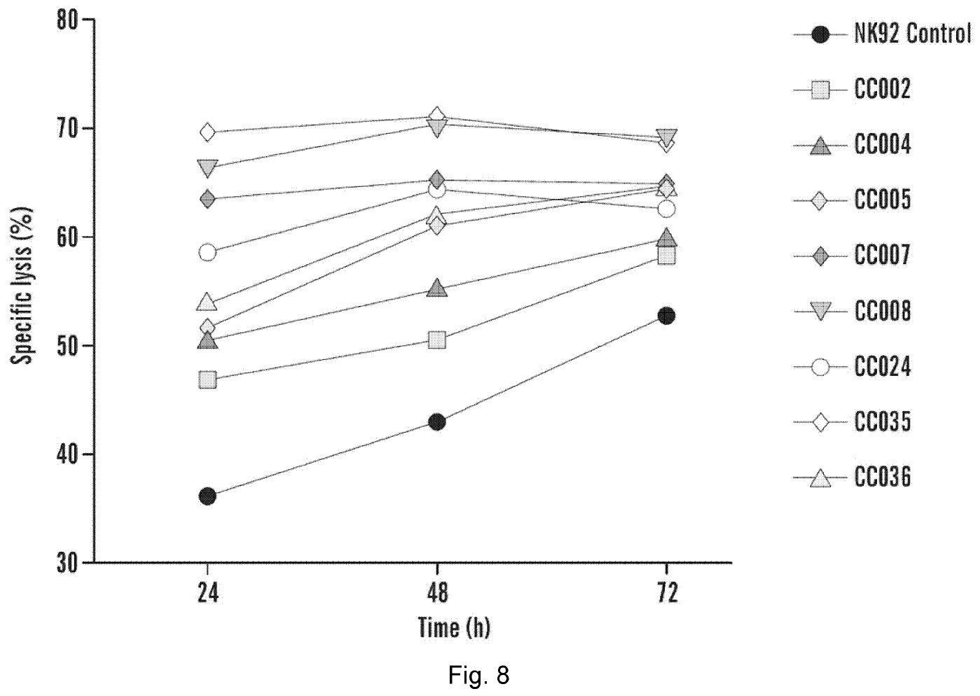

As shown in FIG. 8, CC005/CC007/CC008 CAR constructs incorporating CD8 and FcεRI intracellular domain and CC024/CC035/CC036 incorporating NKG2D intracellular domain demonstrate strong cytotoxic activity against Nalm6-GFP-luciferase tumor cell targets.

### Example 6

This study compares the cytotoxicity of exemplary NK CARs CC005, CC007, CC008, CC018, CC024, CC028, CC030, CC035, and CC036 with control CAR CC004 and NK92 null control. The CARs were transfected into NK92 cells using the lentiviral based method. For lentiviral production, the CAR constructs were first cloned into the plasmid pSIH1 (SBI Cat #CD510B-1), followed by transfection into the 293FT packaging cell along with helper plasmids, pMDL, VSVG and REV using a CaCl₂ mediated transfection method. Culture supernatants were collected after 48 h, filtered (0.22 μm) and stored at −80° C. The titer of LV particles was determined using a Lenti-X p24 Rapid Titer Kit (Novoprotein™). For NK92 cell transduction, NK92 cells were loaded in 24-well plates followed by the addition of lentivirus at an MOI of 20 in the presence of 8 μg/ml Polybrene and incubated at 37° C. for 48 h. CAR transduction efficiency was determined using Guava Easycyte™ flow cytometry, measuring Flag-tag expression using fluorescent labeled anti-flag tag antibody.

Cytotoxicity of each CAR was evaluated with a Calcein-AM assay using Raji cell line as target cell and CAR transfected NK92 cell as effector cell. For measuring of specific lysis (%) of target cell, 1×10⁶ effector cells were stained with 0.15 μM Calcein-AM in NK92 medium for 25 min at 37° C. in a volume of 10 mL. Cells were then washed twice in NK92 medium and added to 96-well round bottom plates at a concentration of 5×10⁴ cells/mL in triplicate, and effector cells were added at 5:1 effector-to-target (E:T) ratio. Plates were centrifuged at 300 rpm for 1 min and incubated at 37° C. for 4 h. Cells were centrifuged at 400 g for 5 min and the supernatant were transferred into 96-well flat white plates. Percentage Specific lysis was calculated using fluorescence (Ex 495 nm, Em 520 nm) of 2% TRITON X100-treated cells as maximum cell death and untreated cells as spontaneous cell death and using the formula % specific lysis=100×[(experimental data−spontaneous cell death)/(maximum cell death−spontaneous cell death)].

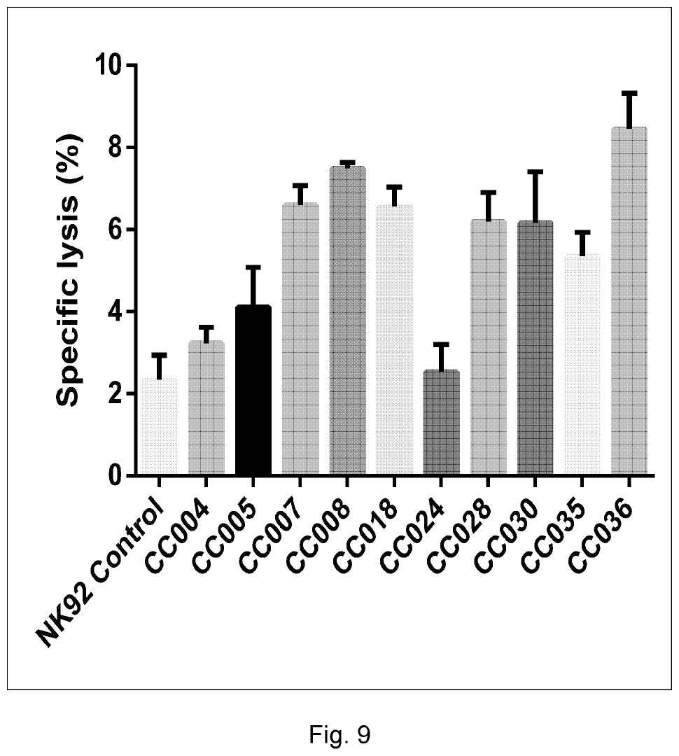

As shown in FIG. 9, CC007/CC008/CC018 CAR constructs incorporating FcεRI intracellular domain, CC028/CC035/CC036 constructs incorporating NKG2D and CRACC or NTB-A intracellular domain demonstrate strong cytotoxic activity against Raji tumor cell targets.

Cytotoxicity of each CAR was further evaluated with a bioluminescence assay using Nalm6-GFP-luciferase cell line as target cell and CAR transfected NK92 cell as effector cell. For measuring of specific lysis (%) of target cell, luciferase-expressing tumor cells were placed in 96-well round bottom plates at a concentration of 5×10⁵ cells/mL in triplicate, and effector cells were added at 1:1 effector-to-target (E:T) ratio. Plates were centrifuged at 300 rpm for 1 min and incubated at 37° C. for 24, 48 and 72 hours, respectively. After completion of incubation, D-luciferin (Perkin Elmer™) was added to each well at a final concentration of 0.14 mg/mL and incubated at 37° C. for 10 min. Cells were then mixed and transferred into 96-well flat white plates. BLI was measured for 1 second with a luminometer (Tecan™, Spark™). Percentage specific lysis was calculated using the formula % specific lysis=100×[(spontaneous cell data−experimental data)/(spontaneous cell data−medium control data)].

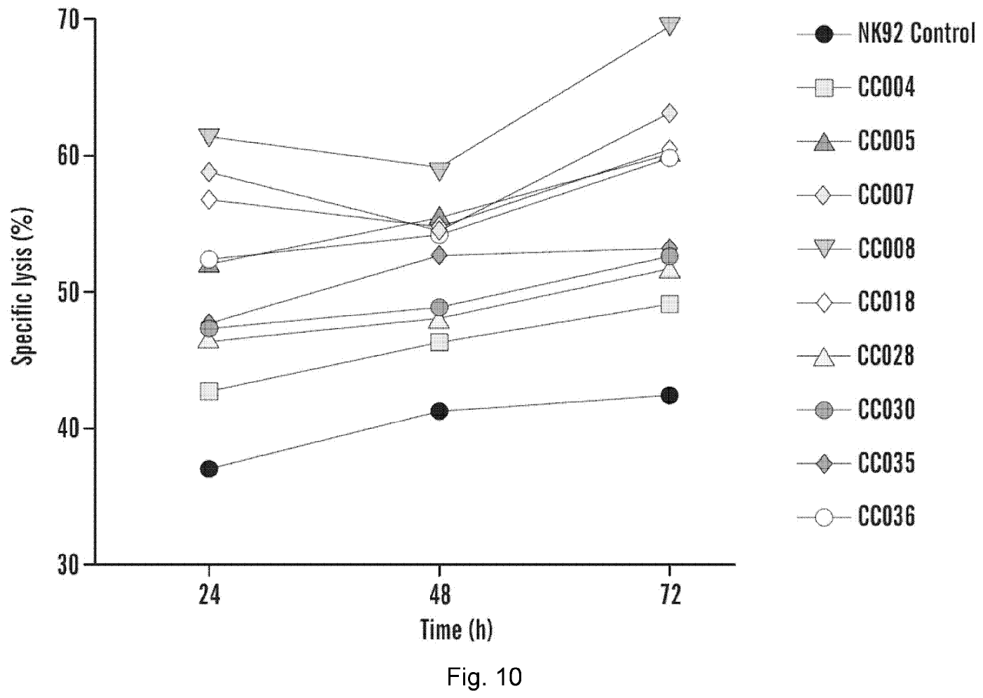

As shown in FIG. 10, CC005/CC007/CC008/CC018 CAR constructs incorporating FcεRI intracellular domain, CC028/CC035/CC036 CAR constructs incorporating NKG2D intracellular domain, and CC030 CAR construct incorporating DNAM1 and CRACC intracellular domain demonstrate strong cytotoxic activity against Nalm6-GFP-luciferase tumor cell targets.

### Example 7

This study compares the cytokine and Granzyme release of exemplary NK CARs CC008, CC018, CC024, CC030, CC035, and CC036 with control CARs CC002-CC004. The release of cytokines TNF-alpha, IFN-gamma, Granzyme B and GM-CSF was analyzed with a Cisbio™ bioassay using HTRF (Homogeneous Time Resolved Fluorescence) technology.

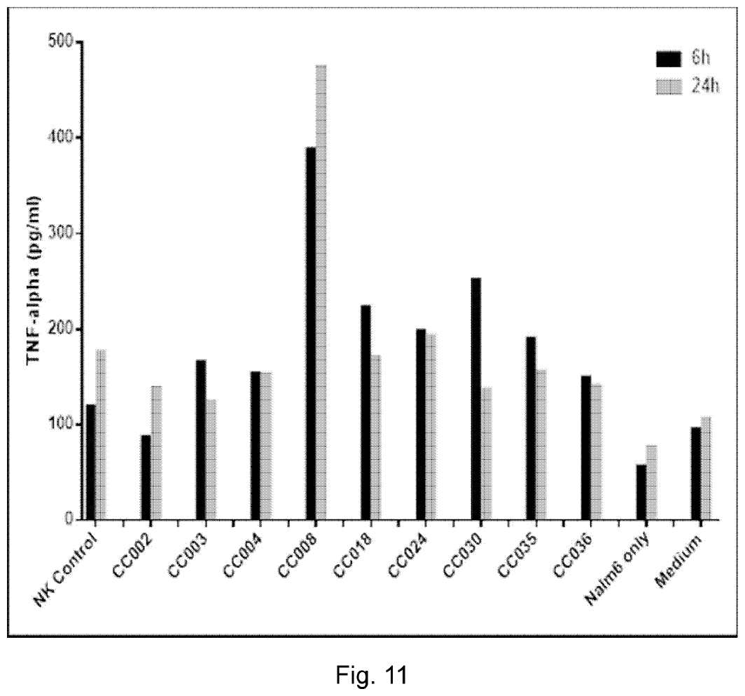

Except CAR construct CC008, all other CARs evaluated showed low levels of cytokine release. See e.g., FIG. 11-14.

### Example 8

### CD33 CAR-NK Constructs and Embodiments

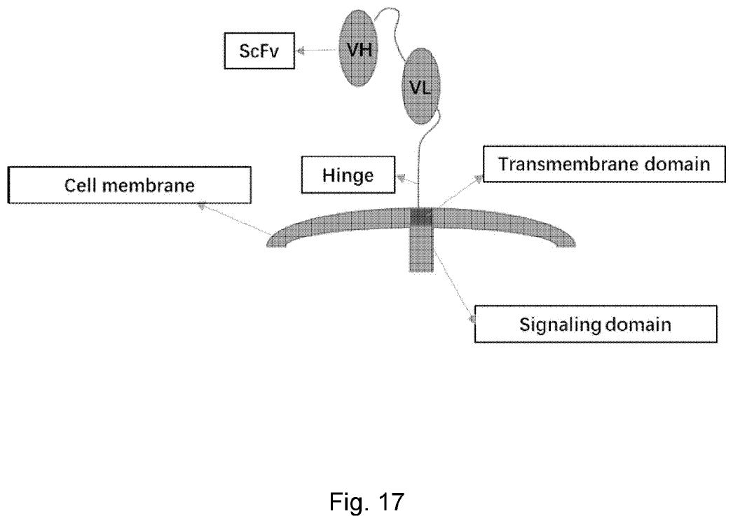

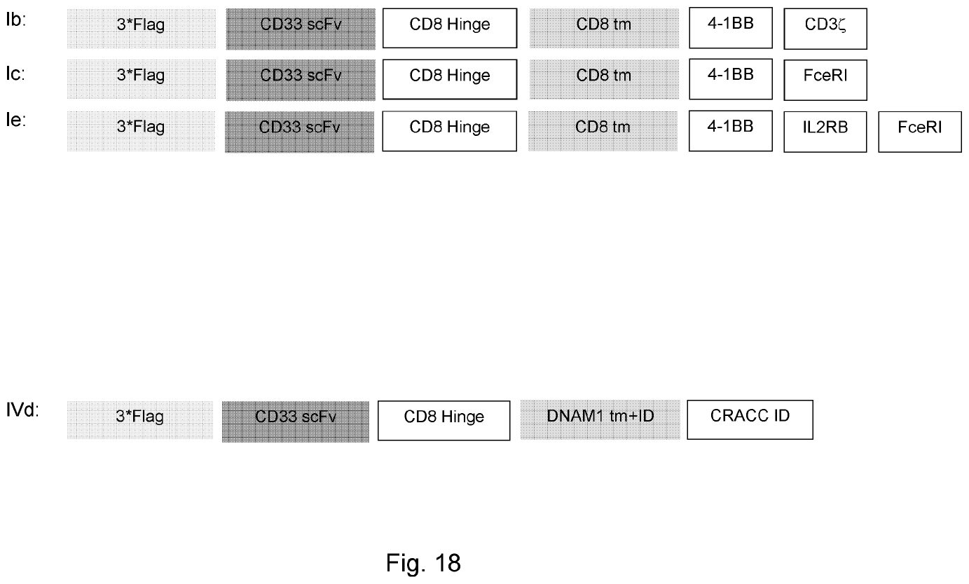

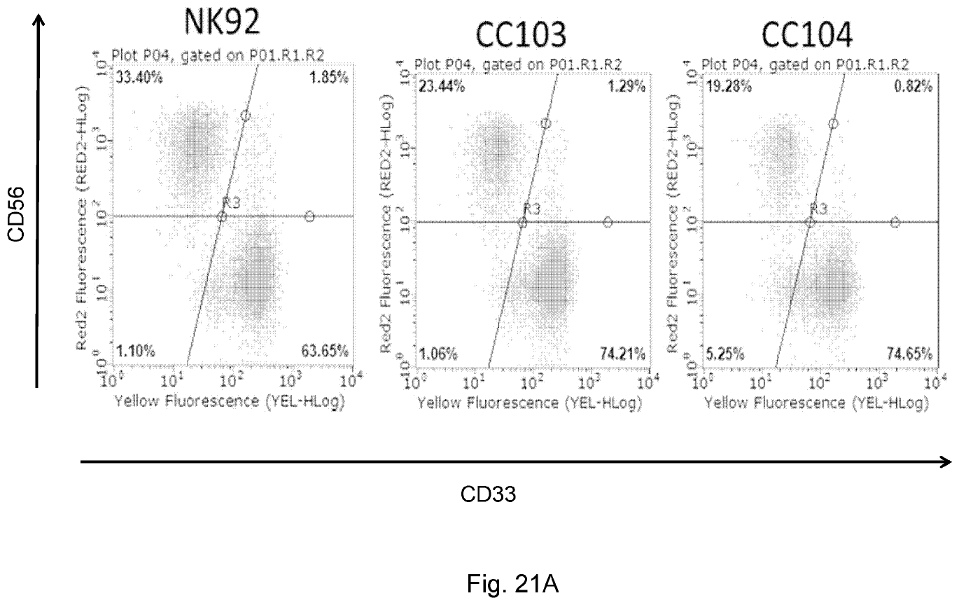

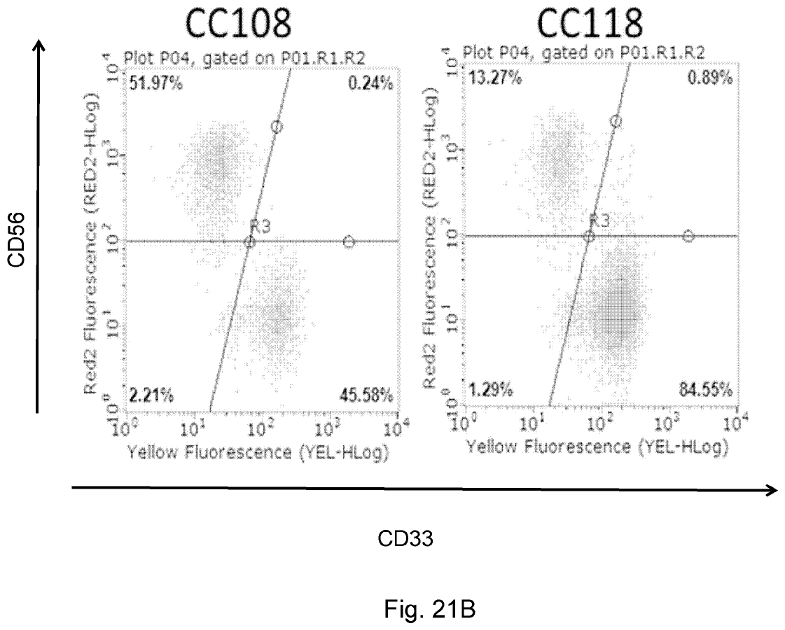

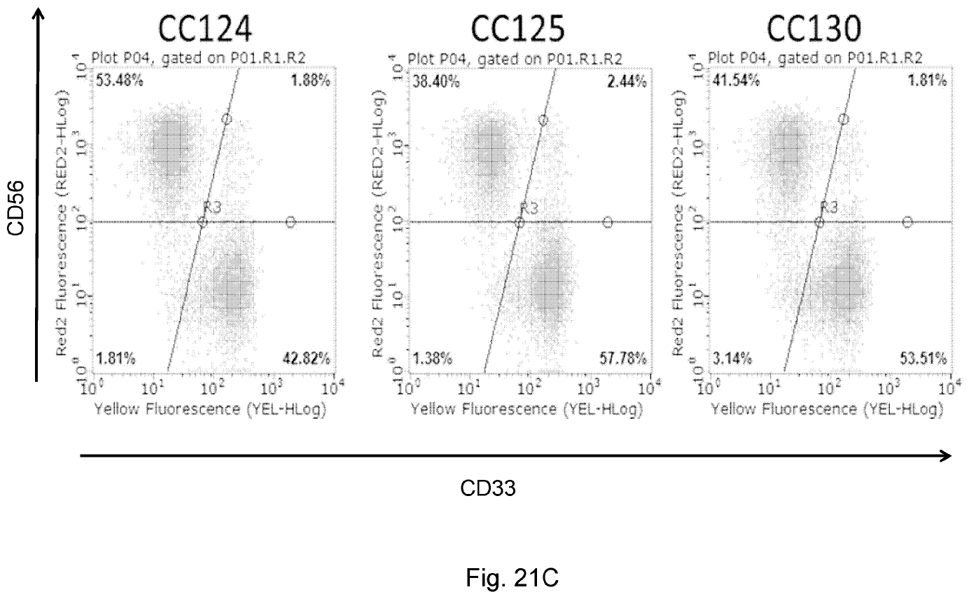

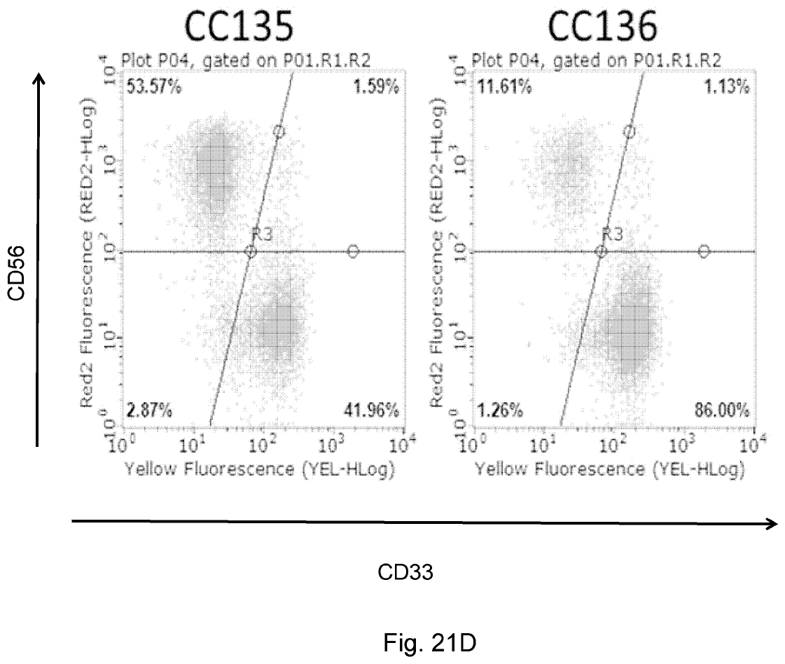

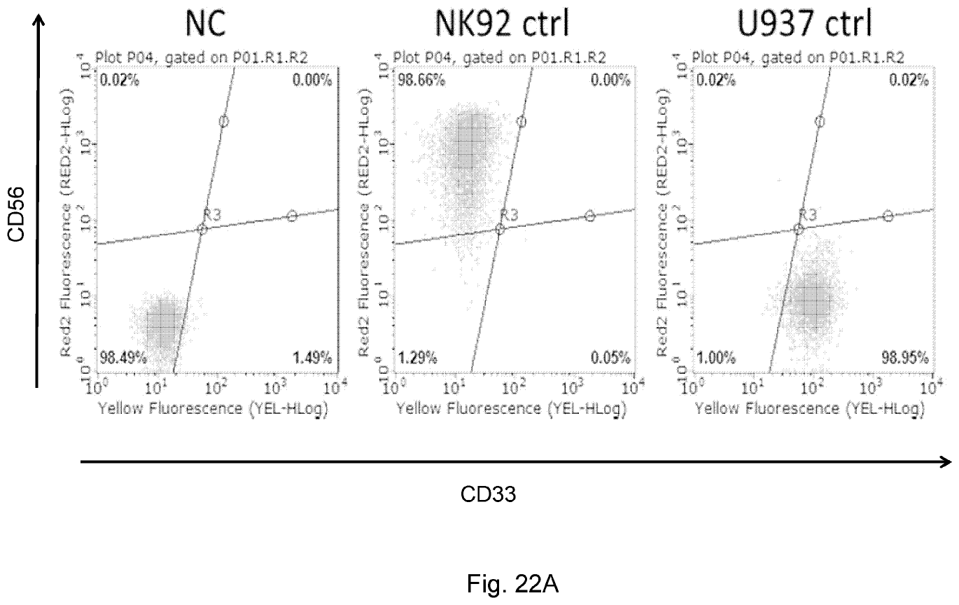

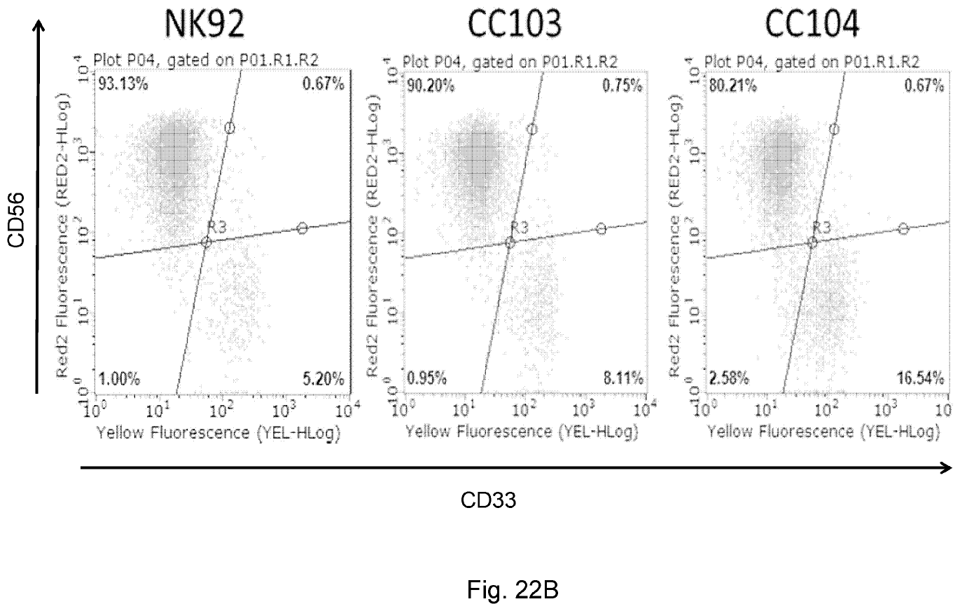

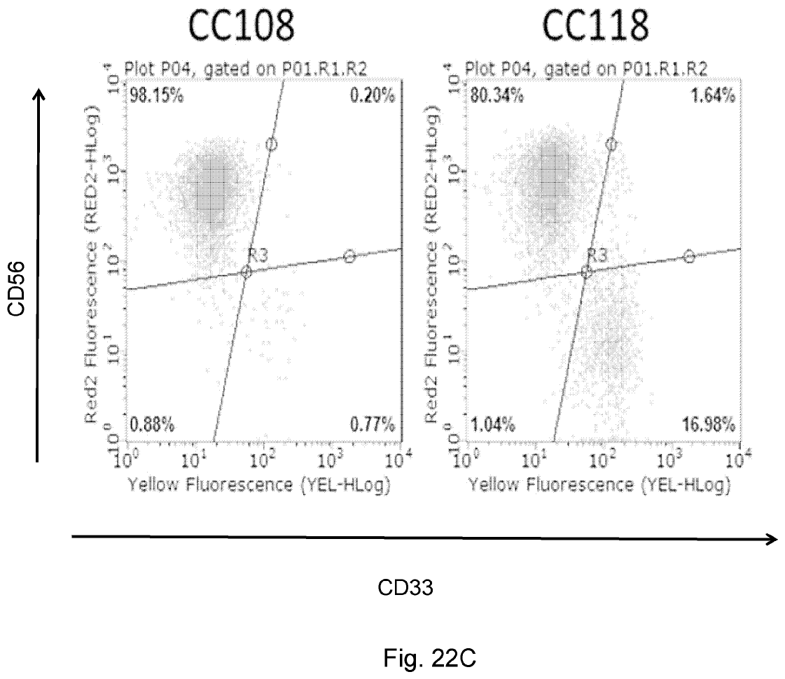

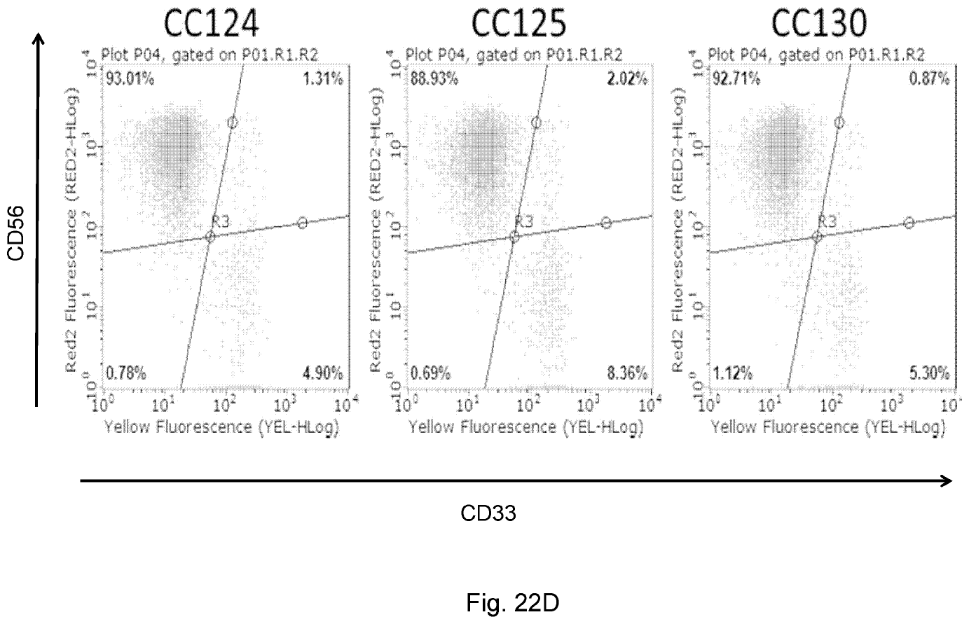

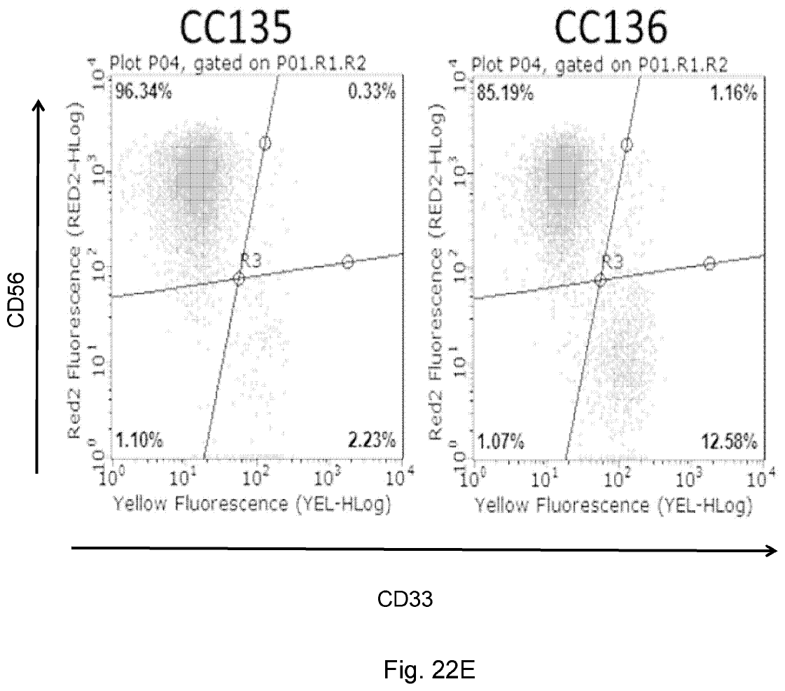

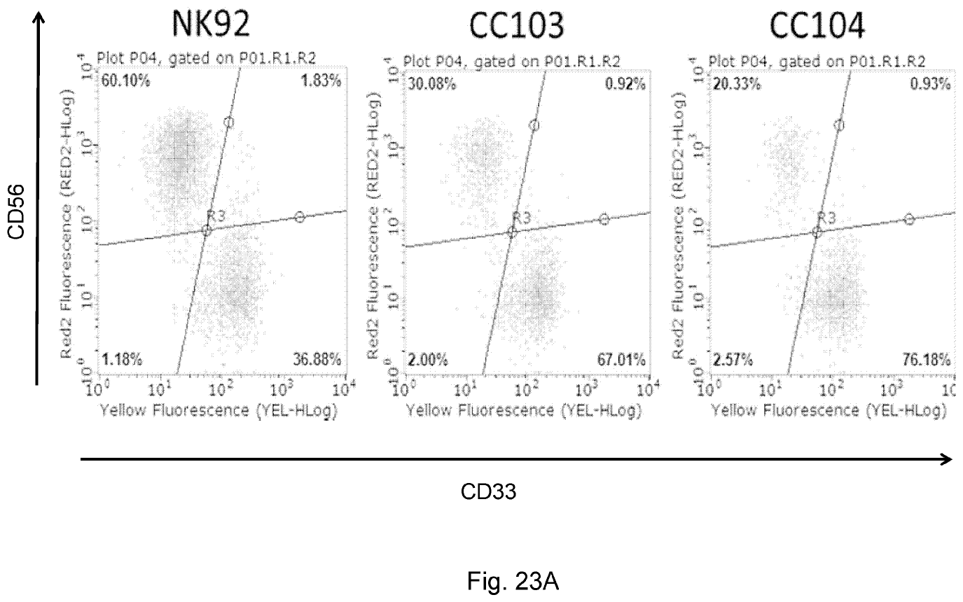

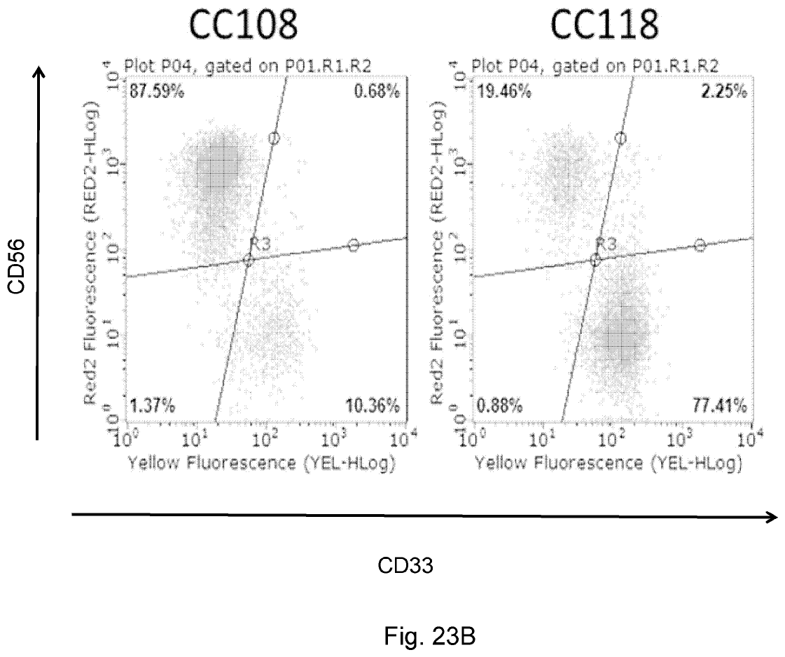

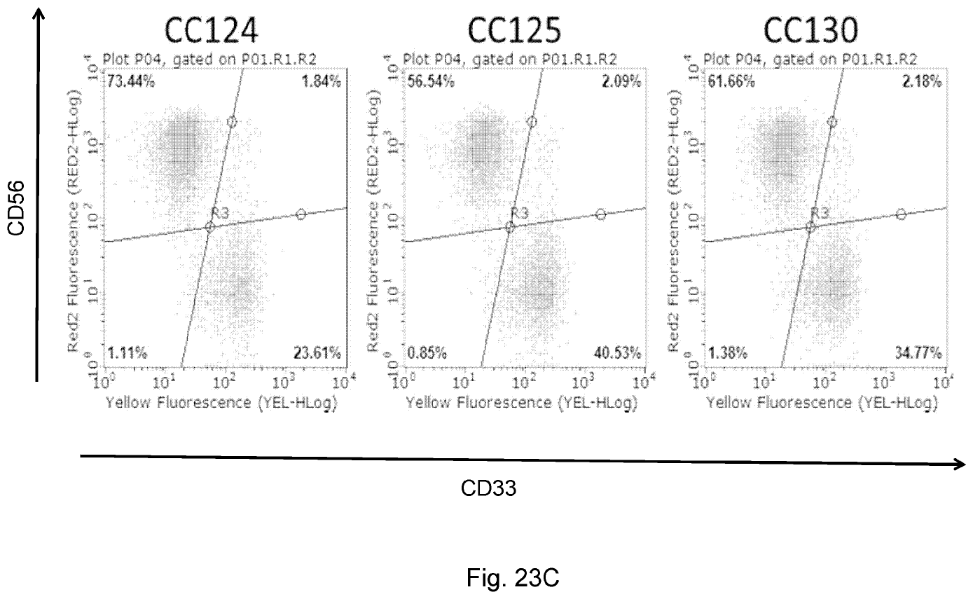

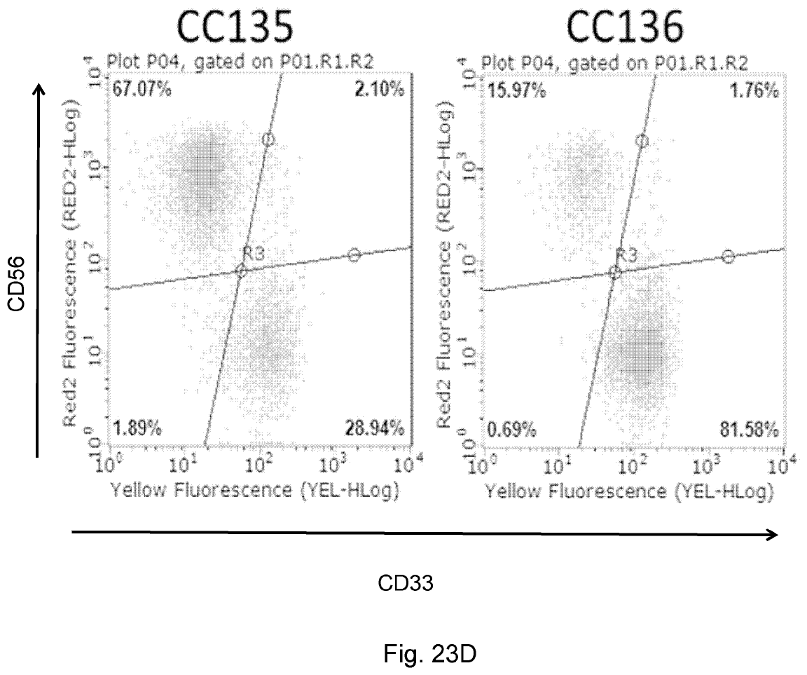

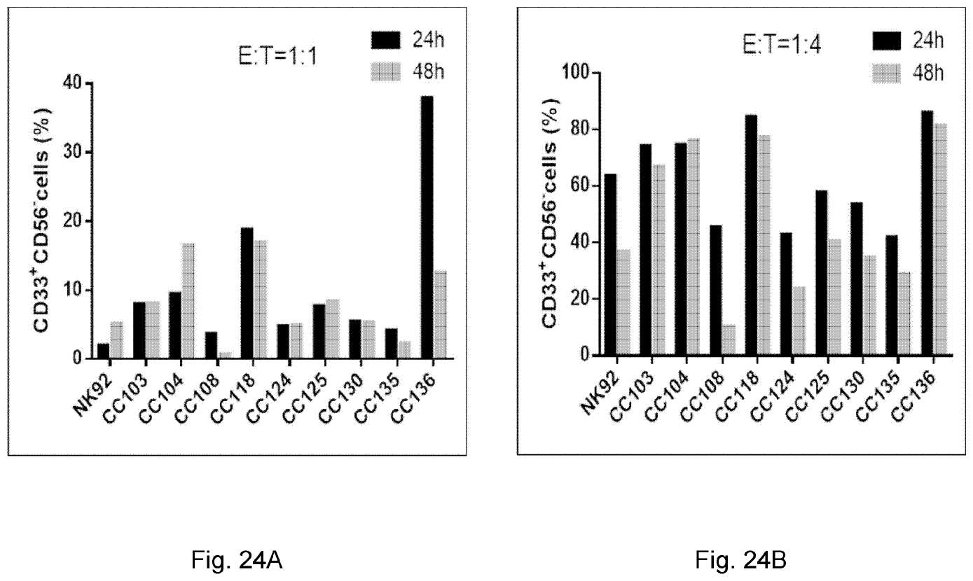

Tables 3 and 4 show exemplary CD33 CAR-NK constructs. For exemplary CD33 CAR-NK data, see e.g., Example 8 or FIGS. 17-24B.

TABLE 3
CD33 CAR-NK Construct with different transmembrane domain (TD) +
costimulatory domain (CD) + signaling domain (SD)

| Construct | Description |
|---|---|
| CC103 | CC104 (without 3*Flag) |
| CC104 | pSIH1-hEF1a-CD33FN CD8-4-1BB-CD3z |
| CC108 | pSIH1-hEF1a-CD33FN CD8-4-1BB-FcRIgE-P2A-IL15-IL21 |
| CC118 | pSIH1-hEF1a-CD33FN CD8 IL2RB-4-1BB-FcR IgE |
| CC124 | pSIH1-hEF1a-CD33FNN NKG2D-2B4 |
| CC125 | pSIH1-hEF1a-CD33FCN NKG2D-2B4 |
| CC130 | pSIH1-hEF1a-CD33FNN DNAM1-CRACC |
| CC135 | pSIH1-hEF1a-CD33FNN NKG2D-CRACC |
| CC136 | pSIH1-hEF1a-CD33FCN NKG2D-CRACC |

TABLE 4
CD33 CAR-NK Constructs

| | | | Trans- | Co-stimulatory |
|---|---|---|---|---|
| | | | membrane | Domain (CD) + |
| | | | Domain | Signaling |
| | Construct No. | Hinge | (TD) | Domain (SD) |
| Group | Ib: CC104 | CD8 | CD8 | 4-1BB + CD3z |
| I | Ic: CC108 | CD8 | CD8 | 4-1BB + FceRI |
| | Ie: CC118 | CD8 | CD8 | 4-1BB/IL2RB + |
| | | | | FceRI |
| Group | IIa: CC124; CC125 | CD8 | NKG2D | 2B4 |
| II | IId: CC135; CC136 | CD8 | NKG2D | CRACC |
| Group | IVd: CC130 | CD8 | DNAM1 | DNAM1 + CRACC |
| IV | | | | |
| Ctrl | CC001 | N/A | N/A | N/A |

This study compares the cytotoxicity of exemplary NK CARs CC104, CC108, CC118, CC124, CC125, CC130, CC135 and CC136 with control CARs CC103 and CC104. The CARs were transfected into NK92 cells using the lentiviral based method. For lentiviral production, the CAR constructs were first cloned into the plasmid pSIH1 (SBI Cat #CD510B-1), followed by transfection into the 293FT packaging cell alongside with helper plasmids, pMDL, VSVG and REV using a CaCl₂ mediated transfection method. Culture supernatants were collected after 48 h, filtered (0.22 μm) and stored at −80° C. The titer of LV particles was determined using a Lenti-X p24 Rapid Titer Kit (Novoprotein™). For NK92 cell transduction, NK92 cells were loaded in 24-well plates followed by the addition of lentivirus at an MOI of 20 in the presence of 8 μg/ml Polybrene and incubated at 37° C. for 48 h. CAR transduction efficiency was determined using Guava Easycyte™ flow cytometry, measuring Flag-tag expression using fluorescent labeled anti-flag tag antibody.

Cytotoxicity of each CAR was evaluated with a Calcein-AM assay using U937 cell line as target cell and CAR transfected NK92 cell as effector cell. For measuring of specific lysis (%) of target cell, 1×10⁶ effector cells were stained with 0.15 μM Calcein-AM in NK92 medium for 25 min at 37° C. in a volume of 10 mL. Cells were then washed twice in NK92 medium and added to 96-well round bottom plates at a concentration of 5×10⁴ cells/mL in triplicate, and effector cells were added at 1:1 or 1:4 effector-to-target (E:T) ratio. Plates were centrifuged at 300 rpm for 1 min and incubated at 37° C. for 24 h and 48 h respectively. Cells were centrifuged at 400 g for 5 min and the supernatant were transferred into 96-well flat white plates. Percentage Specific lysis was calculated using fluorescence (Ex 495 nm, Em 520 nm) of 2% TRITON X100-treated cells as maximum cell death and untreated cells as spontaneous cell death and using the formula % specific lysis=100×[(experimental data−spontaneous cell death)/(maximum cell death−spontaneous cell death)].

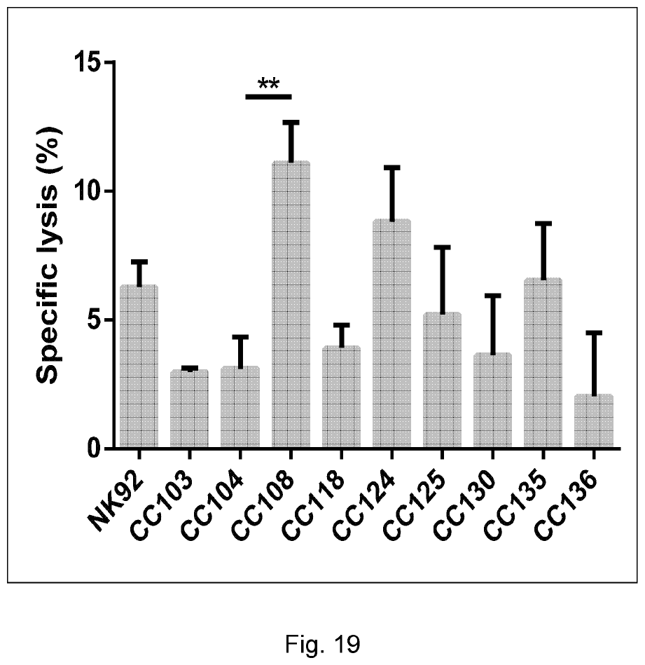

As shown in FIG. 19, the cytotoxicity of CC108 CAR construct is significantly higher than control CARs CC103 and CC104. The CAR constructs CC124 and CC135 also have enhanced cytotoxicity over the control CARs CC103 and CC104.

Cytotoxicity of each CAR was further evaluated with a flow cytometry based method. First, U937 target cells were incubated with effector cells at 1:1 or 1:4 effector to target (E/T) ratio at 37° C. for 24 h and 48 h, respectively. Then PE conjugated anti-CD33 antibody, APC conjugated anti-CD56 antibody, and 7AAD solution were added to each sample and incubated for 30 min at 4° C. before flow cytometric analysis in a Guava Easycyte™ flow cytometer. Dead target cells were identified as 7AAD positive.

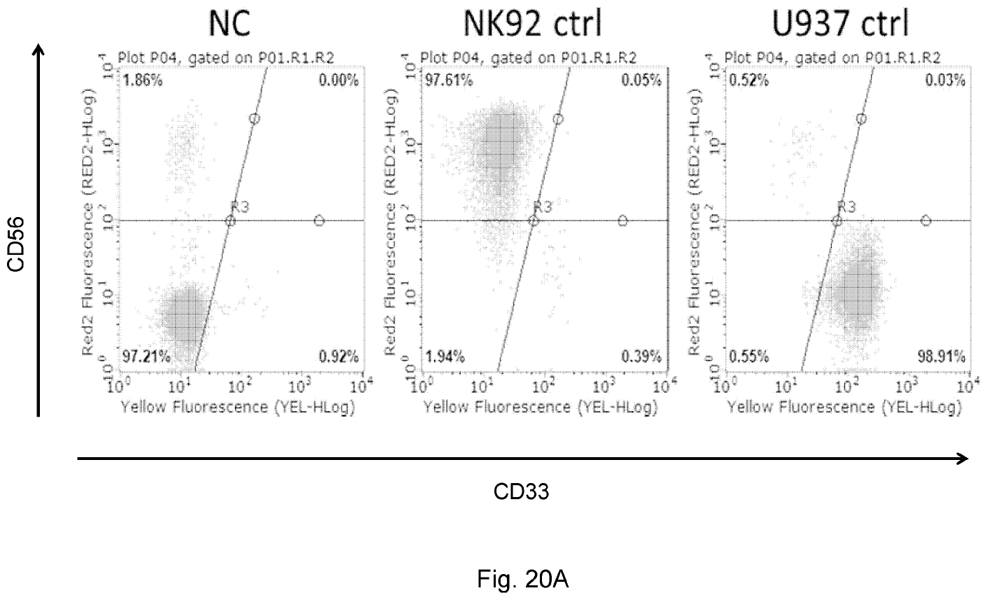

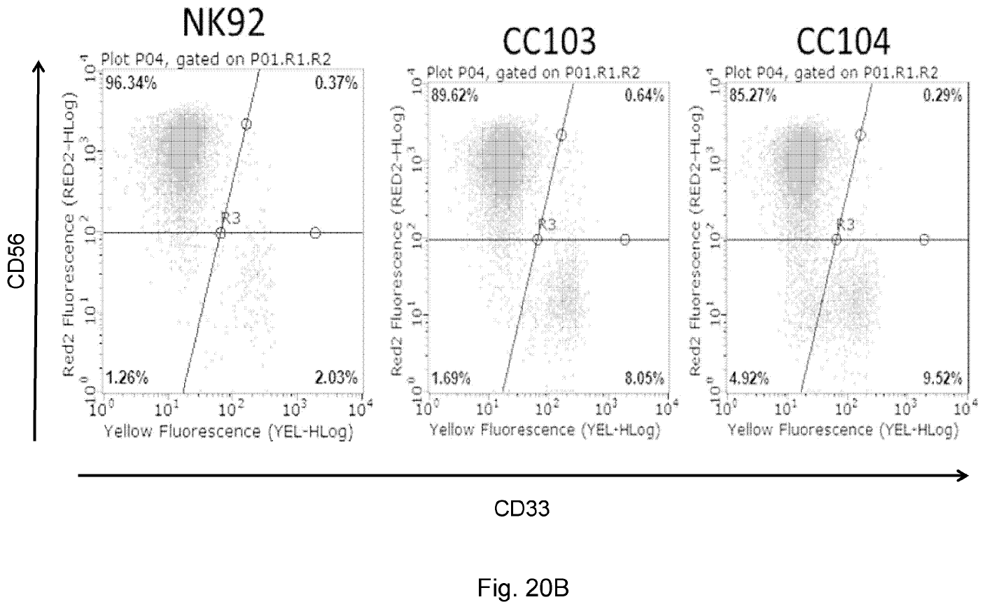

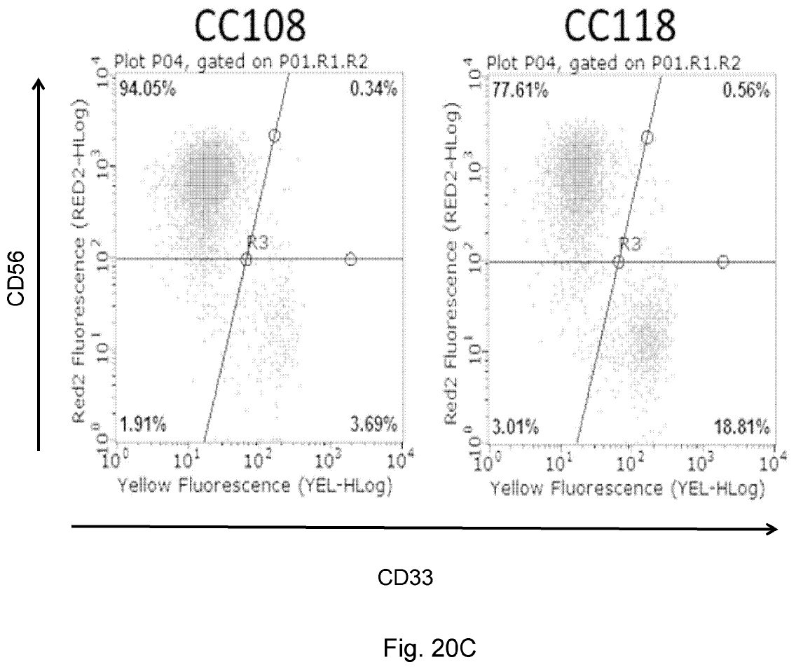

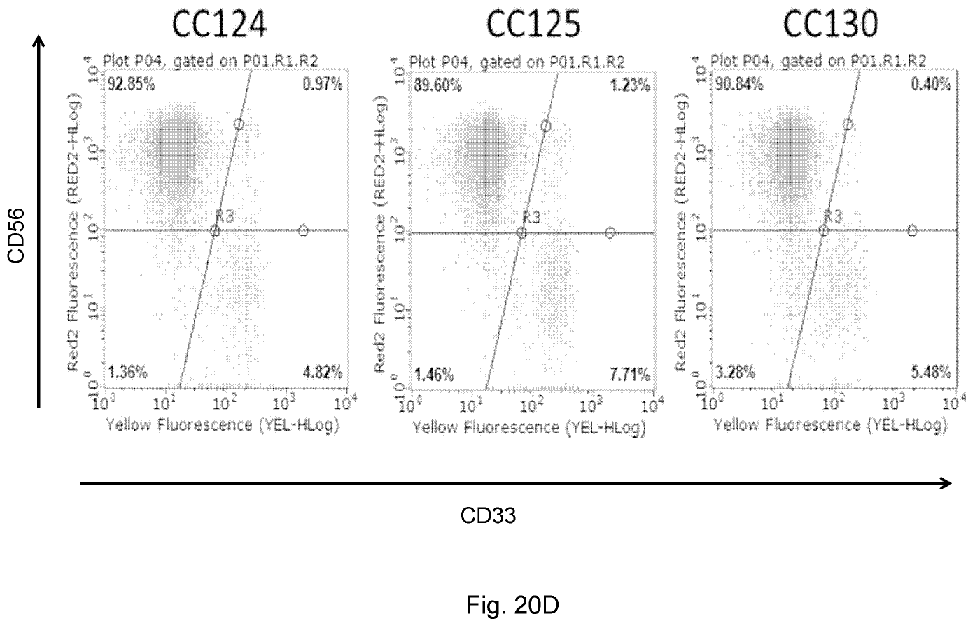

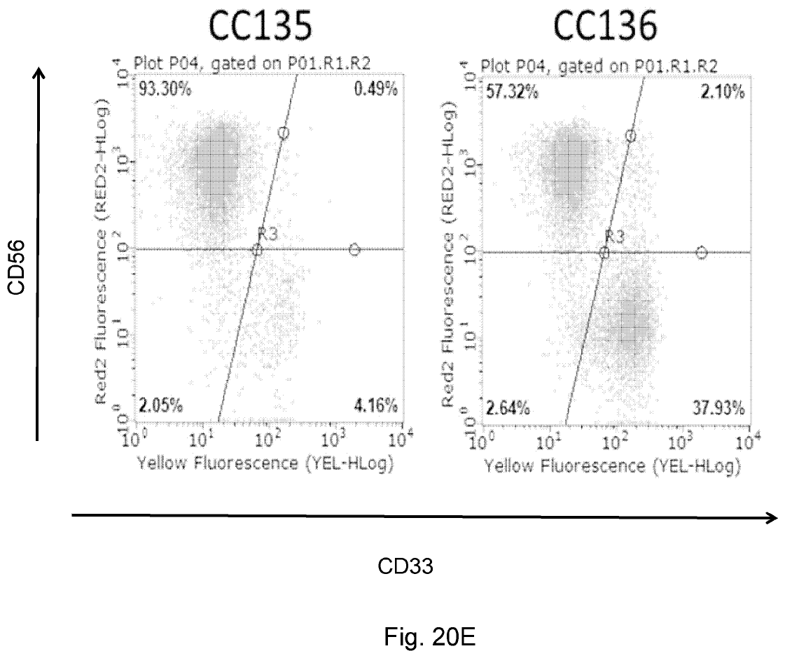

As shown in FIGS. 20A-20E, 21A-21D, 22A-22E, 23A-23D, and 24A-24B, the following CAR constructs demonstrate strong cytotoxic activity against U937 tumor cell targets: CC008 CAR constructs incorporating CD8 and FcεRI intracellular domain together with IL15 and IL21; CC124/CC125 constructs incorporating NKG2D and 2B4 intracellular domain; CC130 constructs incorporating DNAM1 and CRACC intracellular domain; and CC135 construct incorporating NKG2D and CRACC intracellular domain.

### Example 9: Methods

### Cell Culture

NK92 cells were cultured in medium supplemented with 100 IU/mL recombinant human IL-2 (Cytocares™). Raji, U937, Nalm6-GFP-luciferase cells were cultured in 1640 medium (Gibco™) supplemented with 10% fetal bovine serum (FBS) and 1% penicillin/streptomycin. A549-CD19 cells were cultured in DMEM medium (Gibco™) supplemented with 10% fetal bovine serum (FBS) and 1% penicillin/streptomycin. As for peripheral blood natural killer cell (PB-NK) preparation, peripheral blood mononuclear cells (PBMCs) were separated from peripheral blood of healthy donors using Ficoll-Paque PLUS™ (GE Healthcare™) and cultured in stem cell growth medium (SCGM) medium with various cytokines and antibodies for two weeks to obtain primary NK cells with high purity.

### Lentivirus Preparation and NK Cell Transduction

To produce lentiviral particles, the plasmid containing exemplary CARs was transduced into 293FT cells with pMDL, VSVG and REV. 48 hours after transfection, viral supernatant was harvested, filtered and concentrated. The virus was stored at −80° C. and the titer was determined using a Lenti-X p24 Rapid Titer Kit™ (Novoprotein™). NK cells were loaded in 24-well plates followed by the addition of lentivirus at an MOI of 20 in the presence of 8 μg/ml Polybrene and incubated at 37° C. for 48 h. At the endpoint, 7AAD (7-amino-actinomycin D) and flag tag antibody were added 30 min before flow cytometric analysis in a Guava Easycyte™ flow cytometer to detect the CAR transduction efficiency.

### Specific Lysis Using RTCA (Real-Time Cell Analysis):

Adherent target cells (e.g., tumor cells) were first seeded in the wells of an electronic microtiter plate (E-Plate®; “S16 plate” or “S16 wells”) to let cells settle; target cell attachment and proliferation were monitored. On the second day, the S16 plate was removed from the instrument, and the media was removed from wells. Effector cells were then added to the S16 wells and incubated at 37° C. for another 2-5 days. Data was analyzed by xCELLigence™ software.

### Specific Lysis Using Calcein-AM Assay:

1×10⁶ effector cells were stained with 0.15 μM Calcein-AM in NK92 medium for 25 min at 37° C. in a volume of 10 mL. Cells were then washed twice in NK92 medium and added to 96-well round bottom plates at a concentration of 5×10⁴ cells/mL in triplicate, and effector cells were added at 1:1 effector-to-target (E:T) ratio. Plates were centrifuged at 300 rpm for 1 min and incubated at 37° C. for 4 h. Cells were centrifuged at 400 g for 5 min and the supernatants were transferred into 96-well flat white plates. Percentage Specific lysis was calculated using fluorescence (Ex 495 nm, Em 520 nm) of 2% TRITON X100-treated cells as maximum cell death and untreated cells as spontaneous cell death and using the formula % specific lysis=100×[(experimental data−spontaneous cell death)/(maximum cell death−spontaneous cell death)].

### Specific Lysis Using Bioluminescence Assay:

Luciferase-expressing tumor cells were placed in 96-well round bottom plates at a concentration of 5×10⁵ cells/mL in triplicate, and effector cells were added at 1:1 effector-to-target (E:T) ratio. Plates were centrifuged at 300 rpm for 1 min and incubated at 37° C. for 24, 48 and 72 hours, respectively. At the endpoint, D-luciferin (Perkin Elmer™) was added to each well at a final concentration of 0.14 mg/mL and incubated at 37° C. for 10 min. Cells were then mixed and transferred into 96-well flat white plates. BLI was measured for 1 second with a luminometer (Tecan™, Spark™) Percentage specific lysis was calculated using the formula % specific lysis=100×[(spontaneous cell data−experimental data)/(spontaneous cell data−medium control data)].

### Cytokine Release Using Cisbio™:

Cisbio™ bioassay were performed using HTRF Homogeneous Time Resolved Fluorescence) technology followed Cisbio™ protocol. See e.g., Degorce et al., HTRF: A Technology Tailored for Drug Discovery—A Review of Theoretical Aspects and Recent Applications, Curr Chem Genomics. 2009; 3: 22-32; the content of which is incorporated herein by reference in its entirety.

### FACS:

Target cells were incubated with effector cells at different effector to target (E/T) ratios at 37° C. for 24 h and 48 h, respectively. Then various antibody solutions were added to each sample and incubated for 30 min at 4° C. before flow cytometric analysis in a Guava Easycyte™ flow cytometer. Dead target cells were identified as 7AAD positive.

1. A chimeric antigen receptor (CAR) polypeptide for expression in natural killer (NK) cells comprising:
a) an extracellular binding domain;
b) a transmembrane domain; and
c) an intracellular domain comprising at least one of the following:
i) an intracellular signaling domain from an NK cell receptor;
ii) an intracellular signaling domain from an NK cell membrane-bound signaling adaptor protein; and/or
iii) an intracellular co-stimulatory domain from a co-stimulatory receptor.

2. The polypeptide of claim 1, wherein the NK cell receptor:
a) is selected from the group consisting of:
i) Natural Killer Cell Receptor 2B4;
ii) Natural Killer-, T- And B-Cell Antigen (NTB-A);
iii) CD2-Like Receptor Activating Cytotoxic Cells (CRACC); and
iv) cluster of differentiation 2 (CD2); or
b) intracellular signaling domain comprises the amino acid sequence of one of SEQ ID NOs: 7-10 or an amino acid sequence that is at least 80% identical to one of SEQ ID NOs: 7-10.

3. The polypeptide of claim 1, wherein the NK cell membrane-bound signaling adaptor protein:
a) is selected from the group consisting of:
i) high-affinity IgE receptor (FcεR1); and
ii) CD3-zeta (CD3ζ); or
b) intracellular signaling domain comprises the amino acid sequence of one of SEQ ID NOs: 11-12 or an amino acid sequence that is at least 80% identical to one of SEQ ID NOs: 11-12.

4. The polypeptide of claim 1, wherein the co-stimulatory receptor:
a) is 4-1BB and/or IL2 receptor beta (IL2RB); or
b) intracellular co-stimulatory domain comprises the amino acid sequence of one of SEQ ID NOs: 15-16 or an amino acid sequence that is at least 80% identical to one of SEQ ID NOs: 15-16.

5. The polypeptide of claim 1, wherein the intracellular domain further comprises at least one self-cleaving peptide and/or a cytokine.

6. The polypeptide of claim 5, wherein the self-cleaving peptide:
a) is T2A, P2A, E2A, or F2A; or
b) comprises the amino acid sequence of one of SEQ ID NOs: 19-20 or an amino acid sequence that is at least 80% identical to one of SEQ ID NOs: 19-20.

7. The polypeptide of claim 5, wherein the cytokine:
a) is IL-15 or IL-21;
b) comprises the amino acid sequence of one of SEQ ID NOs: 23-24 or an amino acid sequence that is at least 80% identical to one of SEQ ID NOs: 23-24; or
c) is adjacent and distal to the self-cleaving peptide, such that the cytokine is separated from the polypeptide by the self-cleaving peptide.

8. The polypeptide of claim 1, wherein the transmembrane domain comprises:
a) a transmembrane domain of a natural NK cell receptor;
b) a transmembrane domain of CD8; or
c) the amino acid sequence of one of SEQ ID NOs: 29-32 or 117-118 or an amino acid sequence that is at least 80% identical to one of SEQ ID NOs: 29-32 or 117-118.

9. The polypeptide of claim 8, wherein the transmembrane domain of the natural NK cell receptor is selected from the group consisting of Natural Killer Group 2D (NKG2D); Natural Killer Cell P46-Related Protein (NKp46); and DNAX Accessory Molecule-1 (DNAM1).

10. The polypeptide of claim 1, wherein the extracellular binding domain:
a) is an antibody, an antigen-binding fragment thereof, a F(ab) fragment, a F(ab′) fragment, a single chain variable fragment (scFv), or a single-domain antibody (sdAb);
b) specifically binds to a tumor-associated antigen; or
c) comprises the amino acid sequence of one of SEQ ID NOs: 35-36 or an amino acid sequence that is at least 80% identical to one of SEQ ID NOs: 35-36.

11. The polypeptide of claim 1, further comprising a signal peptide located N-terminal to the extracellular binding domain.

12. The polypeptide of claim 11, wherein the signal peptide:
a) is a CD8 signal peptide; or
b) comprises the amino acid sequence of SEQ ID NO: 38 or an amino acid sequence that is at least 80% identical to SEQ ID NO: 38.

13. The polypeptide of claim 1, further comprising a detectable marker distal to the extracellular binding domain.

14. The polypeptide of claim 11, further comprising a linker domain distal to the extracellular binding domain and/or proximal to the signal peptide.

15. The polypeptide of claim 1, further comprising a spacer domain located between the extracellular binding domain and the transmembrane domain.

16. The polypeptide of claim 15, wherein the spacer domain comprises
a) a CD8 hinge domain; or
b) the amino acid sequence of SEQ ID NO: 44 or an amino acid sequence that is at least 80% identical to SEQ ID NO: 44.

17. The CAR polypeptide of claim 1, wherein the polypeptide comprises the amino acid sequence of one of SEQ ID NOs: 80-114 or an amino acid sequence that is at least 80% identical to one of SEQ ID NOs: 80-114.

18. A natural killer (NK) cell or population thereof comprising the polypeptide of claim 1.

19. A method of increasing the activation of an NK cell or population thereof comprising contacting the cell or population thereof with the polypeptide of claim 1.

20. A method of treating a subject in need of a CAR-based therapy comprising administering to the subject a therapeutically effective amount of the CAR-based therapy comprising the polypeptide of claim 1.

21.-154. (canceled)


SEQUENCE LISTING (176 sequences)

SEQ ID NO: 1 - DNA, 360 bp, Artificial Sequence
  synthetic polynucleotide
tggaggaggaaaaggaaggagaagcagtccgagacatcccccaaagagtttttaaccatttatgaggacgtgaaggacctcaagaccagaaggaaccacgagcaagaacagacctttcccggcggcggatccaccatctacagcatgatccagagccagagctccgcccctacaagccaagaacccgcttacactttatattctttaatccagccttctcgtaagagcggctcccgtaagaggaaccacagccccagctttaatagcaccatttacgaggtcatcggcaagtcccagcctaaggcccaaaaccccgctcgtctctctcgtaaggagctggagaatttcgacgtgtacagc

SEQ ID NO: 2 - DNA, 264 bp, Artificial Sequence
  synthetic polynucleotide
tggtttctgaagagagagaggcaagaggagtacatcgaggagaagaagagggtggacatctgcagagagacccccaacatctgcccccattccggcgagaacaccgagtacgacaccatcccccacaccaacagaaccattctgaaggaggaccccgccaacaccgtgtacagcaccgtggagatccccaagaagatggagaatccccactctctgctgaccatgcccgacacccccagactgttcgcctatgagaatgttatc

SEQ ID NO: 3 - DNA, 348 bp, Artificial Sequence
  synthetic polynucleotide
aagaggaagaagcagaggtctagaagaaacgacgaggagctcgagacaagagcccacagagtggctaccgaagagagaggcagaaagccccaccagatccccgccagcacaccccagaaccccgccacaagccagcatcccccccctcctcccggccacagatcccaagctcccagccacagacctcctccccccggccatagggtgcagcatcagccccagaaaagacctcccgcccccagcggaacccaagtgcatcagcagaagggccctcctctgcccagacctagggtgcaacccaagccccctcatggcgctgccgagaactctctgagccctagcagcaac

SEQ ID NO: 4 - DNA, 252 bp, Artificial Sequence
  synthetic polynucleotide
ctgagaaagagaagagactctctgtctctgagcacccaaagaacacaaggccccgagagcgccagaaatctggagtacgtgagcgtgagccccaccaacaacaccgtgtacgccagcgtgacccacagcaacagagagaccgagatctggacccctagagagaacgacaccatcaccatctacagcaccatcaaccacagcaaggagagcaagcccacattctctagagccaccgctctggataacgtggtg

SEQ ID NO: 5 - DNA, 336 bp, Artificial Sequence
  synthetic polynucleotide
agagtcaagttctccaggtccgccgacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 6 - DNA, 126 bp, Artificial Sequence
  synthetic polynucleotide
cgtctgaagatccaagttaggaaggccgccatcacctcctacgagaagagcgacggcgtgtacaccggtttaagcacaaggaaccaagaaacctacgagactttaaagcacgagaagcccccccag

SEQ ID NO: 7 - protein, 120 aa, Artificial Sequence
  synthetic polypeptide
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 8 - protein, 88 aa, Artificial Sequence
  synthetic polypeptide
WFLKRERQEEYIEEKKRVDICRETPNICPHSGENTEYDTIPHTNRTILKEDPANTVYSTVEIPKKMENPHSLLTMPDTPRLFAYENVI

SEQ ID NO: 9 - protein, 116 aa, Artificial Sequence
  synthetic polypeptide
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 10 - protein, 84 aa, Artificial Sequence
  synthetic polypeptide
LRKRRDSLSLSTQRTQGPESARNLEYVSVSPTNNTVYASVTHSNRETEIWTPRENDTITIYSTINHSKESKPTFSRATALDNVV

SEQ ID NO: 11 - protein, 112 aa, Artificial Sequence
  synthetic polypeptide
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 42 aa, Artificial Sequence
  synthetic polypeptide
RLKIQVRKAAITSYEKSDGVYTGLSTRNQETYETLKHEKPPQ

SEQ ID NO: 13 - DNA, 126 bp, Artificial Sequence
  synthetic polynucleotide
aaaaggggcagaaagaaactgctctacatcttcaagcagcccttcatgaggcccgtccaaaccacccaggaggaggacggatgcagctgtagattccccgaagaggaggaaggcggctgcgagctg

SEQ ID NO: 14 - DNA, 282 bp, Artificial Sequence
  synthetic polynucleotide
aactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctccccctgaacactgatgcctacttgtccctccaagaactccagggtcaggacccaactcacttggtg

SEQ ID NO: 15 - protein, 42 aa, Artificial Sequence
  synthetic polypeptide
KRGRKKLLYIFKQPFMRPVQTTQEEDGCSCRFPEEEEGGCEL

SEQ ID NO: 16 - protein, 94 aa, Artificial Sequence
  synthetic polypeptide
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 17 - DNA, 66 bp, Artificial Sequence
  synthetic polynucleotide
ggcagcggcgccaccaacttctctttactgaagcaagctggcgacgtggaggagaatcccggtccc

SEQ ID NO: 18 - DNA, 69 bp, Artificial Sequence
  synthetic polynucleotide
ggcagcggaagagccgagggaagaggctctttactgacttgtggcgacgtggaagagaatcccggcccc

SEQ ID NO: 19 - protein, 22 aa, Artificial Sequence
  synthetic polypeptide
GSGATNFSLLKQAGDVEENPGP

SEQ ID NO: 20 - protein, 23 aa, Artificial Sequence
  synthetic polypeptide
GSGRAEGRGSLLTCGDVEENPGP

SEQ ID NO: 21 - DNA, 486 bp, Homo sapiens
atgaggatcagcaagccccatttaaggagcatctccatccagtgctatttatgtttactgctgaacagccattttttaaccgaggccggcatccacgtgttcattttaggctgcttttccgccggtttacccaagaccgaggctaactgggtgaacgtgatcagcgatttaaagaagatcgaggatttaatccagagcatgcacatcgatgccactttatataccgagagcgacgtgcaccccagctgcaaggtgaccgccatgaagtgttttttactggagctgcaagttatctctttagagagcggcgacgccagcatccacgacaccgtggagaatttaatcattttagccaacaacagcctctccagcaacggcaatgtgacagagagcggctgcaaggagtgcgaggagctggaggagaagaacatcaaggagtttttacagagcttcgtgcacatcgtgcagatgttcatcaacaccagc

SEQ ID NO: 22 - DNA, 465 bp, Homo sapiens
atggagagaatcgtgatctgtttaatggtgatctttttaggcactttagtgcacaagtccagctcccaaggtcaagatagacacatgattcgtatgaggcagctgatcgacatcgtggaccagctgaagaactacgtgaacgatttagtgcccgaatttttacccgctcccgaggacgtggagaccaactgcgagtggagcgccttctcttgtttccagaaggctcagctgaagagcgccaacaccggcaacaacgagaggattatcaacgtgtccatcaagaagctgaagaggaagcctccctccaccaacgccggcagaaggcagaagcacagactgacttgtccctcttgtgacagctacgagaagaagccccctaaggagtttttagagagattcaagtctttactgcagaagatgatccaccagcatttaagctctcgtacccacggcagcgaggattcc

SEQ ID NO: 23 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 24 - protein, 155 aa, Homo sapiens
MERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 25 - DNA, 63 bp, Artificial Sequence
  synthetic polynucleotide
cctttcttcttctgctgcttcatcgccgtggccatgggcattcgtttcatcatcatggtggcc

SEQ ID NO: 26 - DNA, 138 bp, Artificial Sequence
  synthetic polynucleotide
atgggactggcctttctggtgctggtggctctggtgtggtttctggtggaggactggctgagcagaaagaggaccagagagagagccagcagagccagcacatgggagggcagaaggagactgaacacccagaccctg

SEQ ID NO: 27 - DNA, 246 bp, Artificial Sequence
  synthetic polynucleotide
ggcggcaccgtgctgctgctgctgttcgtgatctccatcaccaccatcatcgtcatctttctgaacagaaggaggagaagggagaggagggatctgttcaccgagagctgggacacccagaaggcccccaacaactatagaagccccatcagcaccagccagcccaccaaccagagcatggacgacaccagagaggacatctacgtgaactaccccaccttcagcagaagacccaagaccagagtg

SEQ ID NO: 28 - DNA, 72 bp, Artificial Sequence
  synthetic polynucleotide
atctacatctgggcccctctggctggcacatgtggcgtgctgctgctctccctggtgatcaccctgtactgc

SEQ ID NO: 29 - protein, 21 aa, Artificial Sequence
  synthetic polypeptide
PFFFCCFIAVAMGIRFIIMVA

SEQ ID NO: 30 - protein, 46 aa, Artificial Sequence
  synthetic polypeptide
MGLAFLVLVALVWFLVEDWLSRKRTRERASRASTWEGRRRLNTQTL

SEQ ID NO: 31 - protein, 82 aa, Artificial Sequence
  synthetic polypeptide
GGTVLLLLFVISITTIIVIFLNRRRRRERRDLFTESWDTQKAPNNYRSPISTSQPTNQSMDDTREDIYVNYPTFSRRPKTRV

SEQ ID NO: 32 - protein, 24 aa, Artificial Sequence
  synthetic polypeptide
IYIWAPLAGTCGVLLLSLVITLYC

SEQ ID NO: 33 - DNA, 726 bp, Artificial Sequence
  synthetic polynucleotide
gacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagc

SEQ ID NO: 34 - DNA, 735 bp, Artificial Sequence
  synthetic polynucleotide
gaagtgcagctggtgcagagcggagcagaagtgaagaagcccggaagcagcgtgaaggtgtcttgcaaggccagcggctacaccatcaccgacagcaacatccattgggtccggcaggctccaggacagtctctggagtggatcggctacatctacccctacaacggcggcaccgactacaaccagaagttcaagaaccgggccaccctgaccgtggataaccccaccaacaccgcctacatggagctgagcagcctgagaagcgaggacaccgccttctactattgcgtgaacggcaacccttggctggcctattggggacagggaacactggtgaccgtgtcctctggcggcggaggcagcggcggcggaggctccggaggcggaggatccgacatccagctgacccagtctcctagcaccctgagcgctagcgtgggagatagagtgaccatcacttgcagagccagcgagagcctggacaactacggcatccggttcctgacttggttccagcagaaacccggcaaggcccctaaactgctgatgtacgccgcctctaaccagggaagcggagtgcctagcagattcagcggcagcggaagcggaaccgagttcaccctgaccatcagctctctgcagccagacgacttcgccacctactactgccagcagaccaaggaggtgccttggagcttcggccagggaaccaaggtggaagtgaagcggacagtg

SEQ ID NO: 35 - protein, 242 aa, Artificial Sequence
  synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 36 - protein, 245 aa, Artificial Sequence
  synthetic polypeptide
EVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTV

SEQ ID NO: 37 - DNA, 63 bp, Artificial Sequence
  synthetic polynucleotide
atggccttaccagtgaccgccttgctcctgccgctggccttgctgctccacgccgccaggccg

SEQ ID NO: 38 - protein, 21 aa, Artificial Sequence
  synthetic polypeptide
MALPVTALLLPLALLLHAARP

SEQ ID NO: 39 - DNA, 72 bp, Artificial Sequence
  synthetic polynucleotide
gattacaaggatgacgacgataaggactataaggacgacgacgataaggattataaagacgatgacgacaaa

SEQ ID NO: 40 - protein, 24 aa, Artificial Sequence
  synthetic polypeptide
DYKDDDDKDYKDDDDKDYKDDDDK

SEQ ID NO: 41 - DNA, 15 bp, Artificial Sequence
  synthetic polynucleotide
ggtggaggcggttcg

SEQ ID NO: 42 - protein, 5 aa, Artificial Sequence
  synthetic polypeptide
GGGGS

SEQ ID NO: 43 - DNA, 135 bp, Artificial Sequence
  synthetic polynucleotide
accacaacccctgcccccagacctcccacacccgcccctaccatcgctagccagcctctgagcctgaggcccgaggcttgcagacctgctgctggaggcgccgtgcacaccagaggactggatttcgcctgcgac

SEQ ID NO: 44 - protein, 45 aa, Artificial Sequence
  synthetic polypeptide
TTTPAPRPPTPAPTIASQPLSLRPEACRPAAGGAVHTRGLDFACD

SEQ ID NO: 45 - DNA, 1212 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacatctacatctgggcccctctggctggcacatgtggcgtgctgctgctctccctggtgatcaccctgtactgcaaaaggggcagaaagaaactgctctacatcttcaagcagcccttcatgaggcccgtccaaaccacccaggaggaggacggatgcagctgtagattccccgaagaggaggaaggcggctgcgagctgtaa

SEQ ID NO: 46 - DNA, 1476 bp, Artificial Sequence
  synthetic polynucleotide
atggccttaccagtgaccgccttgctcctgccgctggccttgctgctccacgccgccaggccgggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacatctacatctgggcccctctggctggcacatgtggcgtgctgctgctctccctggtgatcaccctgtactgcaaaaggggcagaaagaaactgctctacatcttcaagcagcccttcatgaggcccgtccaaaccacccaggaggaggacggatgcagctgtagattccccgaagaggaggaaggcggctgcgagctgagagtcaagttctccaggtccgccgacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 47 - DNA, 1548 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacatctacatctgggcccctctggctggcacatgtggcgtgctgctgctctccctggtgatcaccctgtactgcaaaaggggcagaaagaaactgctctacatcttcaagcagcccttcatgaggcccgtccaaaccacccaggaggaggacggatgcagctgtagattccccgaagaggaggaaggcggctgcgagctgagagtcaagttctccaggtccgccgacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 48 - DNA, 1338 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacatctacatctgggcccctctggctggcacatgtggcgtgctgctgctctccctggtgatcaccctgtactgcaaaaggggcagaaagaaactgctctacatcttcaagcagcccttcatgaggcccgtccaaaccacccaggaggaggacggatgcagctgtagattccccgaagaggaggaaggcggctgcgagctgcgtctgaagatccaagttaggaaggccgccatcacctcctacgagaagagcgacggcgtgtacaccggtttaagcacaaggaaccaagaaacctacgagactttaaagcacgagaagcccccccagtaa

SEQ ID NO: 49 - DNA, 1890 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacatctacatctgggcccctctggctggcacatgtggcgtgctgctgctctccctggtgatcaccctgtactgcaaaaggggcagaaagaaactgctctacatcttcaagcagcccttcatgaggcccgtccaaaccacccaggaggaggacggatgcagctgtagattccccgaagaggaggaaggcggctgcgagctgcgtctgaagatccaagttaggaaggccgccatcacctcctacgagaagagcgacggcgtgtacaccggtttaagcacaaggaaccaagaaacctacgagactttaaagcacgagaagcccccccagggcagcggcgccaccaacttctctttactgaagcaagctggcgacgtggaggagaatcccggtcccatgaggatcagcaagccccatttaaggagcatctccatccagtgctatttatgtttactgctgaacagccattttttaaccgaggccggcatccacgtgttcattttaggctgcttttccgccggtttacccaagaccgaggctaactgggtgaacgtgatcagcgatttaaagaagatcgaggatttaatccagagcatgcacatcgatgccactttatataccgagagcgacgtgcaccccagctgcaaggtgaccgccatgaagtgttttttactggagctgcaagttatctctttagagagcggcgacgccagcatccacgacaccgtggagaatttaatcattttagccaacaacagcctctccagcaacggcaatgtgacagagagcggctgcaaggagtgcgaggagctggaggagaagaacatcaaggagtttttacagagcttcgtgcacatcgtgcagatgttcatcaacaccagctaa

SEQ ID NO: 50 - DNA, 2424 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacatctacatctgggcccctctggctggcacatgtggcgtgctgctgctctccctggtgatcaccctgtactgcaaaaggggcagaaagaaactgctctacatcttcaagcagcccttcatgaggcccgtccaaaccacccaggaggaggacggatgcagctgtagattccccgaagaggaggaaggcggctgcgagctgcgtctgaagatccaagttaggaaggccgccatcacctcctacgagaagagcgacggcgtgtacaccggtttaagcacaaggaaccaagaaacctacgagactttaaagcacgagaagcccccccagggcagcggcgccaccaacttctctttactgaagcaagctggcgacgtggaggagaatcccggtcccatgaggatcagcaagccccatttaaggagcatctccatccagtgctatttatgtttactgctgaacagccattttttaaccgaggccggcatccacgtgttcattttaggctgcttttccgccggtttacccaagaccgaggctaactgggtgaacgtgatcagcgatttaaagaagatcgaggatttaatccagagcatgcacatcgatgccactttatataccgagagcgacgtgcaccccagctgcaaggtgaccgccatgaagtgttttttactggagctgcaagttatctctttagagagcggcgacgccagcatccacgacaccgtggagaatttaatcattttagccaacaacagcctctccagcaacggcaatgtgacagagagcggctgcaaggagtgcgaggagctggaggagaagaacatcaaggagtttttacagagcttcgtgcacatcgtgcagatgttcatcaacaccagcggcagcggaagagccgagggaagaggctctttactgacttgtggcgacgtggaagagaatcccggccccatggagagaatcgtgatctgtttaatggtgatctttttaggcactttagtgcacaagtccagctcccaaggtcaagatagacacatgattcgtatgaggcagctgatcgacatcgtggaccagctgaagaactacgtgaacgatttagtgcccgaatttttacccgctcccgaggacgtggagaccaactgcgagtggagcgccttctcttgtttccagaaggctcagctgaagagcgccaacaccggcaacaacgagaggattatcaacgtgtccatcaagaagctgaagaggaagcctccctccaccaacgccggcagaaggcagaagcacagactgacttgtccctcttgtgacagctacgagaagaagccccctaaggagtttttagagagattcaagtctttactgcagaagatgatccaccagcatttaagctctcgtacccacggcagcgaggattcctaa

SEQ ID NO: 51 - DNA, 1830 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacatctacatctgggcccctctggctggcacatgtggcgtgctgctgctctccctggtgatcaccctgtactgcaaaaggggcagaaagaaactgctctacatcttcaagcagcccttcatgaggcccgtccaaaccacccaggaggaggacggatgcagctgtagattccccgaagaggaggaaggcggctgcgagctgaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctccccctgaacactgatgcctacttgtccctccaagaactccagggtcaggacccaactcacttggtgagagtcaagttctccaggtccgccgacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 52 - DNA, 1830 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacatctacatctgggcccctctggctggcacatgtggcgtgctgctgctctccctggtgatcaccctgtactgcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctccccctgaacactgatgcctacttgtccctccaagaactccagggtcaggacccaactcacttggtgaaaaggggcagaaagaaactgctctacatcttcaagcagcccttcatgaggcccgtccaaaccacccaggaggaggacggatgcagctgtagattccccgaagaggaggaaggcggctgcgagctgagagtcaagttctccaggtccgccgacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 53 - DNA, 1620 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacatctacatctgggcccctctggctggcacatgtggcgtgctgctgctctccctggtgatcaccctgtactgcaaaaggggcagaaagaaactgctctacatcttcaagcagcccttcatgaggcccgtccaaaccacccaggaggaggacggatgcagctgtagattccccgaagaggaggaaggcggctgcgagctgaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctccccctgaacactgatgcctacttgtccctccaagaactccagggtcaggacccaactcacttggtgcgtctgaagatccaagttaggaaggccgccatcacctcctacgagaagagcgacggcgtgtacaccggtttaagcacaaggaaccaagaaacctacgagactttaaagcacgagaagcccccccagtaa

SEQ ID NO: 54 - DNA, 1620 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacatctacatctgggcccctctggctggcacatgtggcgtgctgctgctctccctggtgatcaccctgtactgcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctccccctgaacactgatgcctacttgtccctccaagaactccagggtcaggacccaactcacttggtgaaaaggggcagaaagaaactgctctacatcttcaagcagcccttcatgaggcccgtccaaaccacccaggaggaggacggatgcagctgtagattccccgaagaggaggaaggcggctgcgagctgcgtctgaagatccaagttaggaaggccgccatcacctcctacgagaagagcgacggcgtgtacaccggtttaagcacaaggaaccaagaaacctacgagactttaaagcacgagaagcccccccagtaa

SEQ ID NO: 55 - DNA, 1437 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgaccctttcttcttctgctgcttcatcgccgtggccatgggcattcgtttcatcatcatggtggcctggaggaggaaaaggaaggagaagcagtccgagacatcccccaaagagtttttaaccatttatgaggacgtgaaggacctcaagaccagaaggaaccacgagcaagaacagacctttcccggcggcggatccaccatctacagcatgatccagagccagagctccgcccctacaagccaagaacccgcttacactttatattctttaatccagccttctcgtaagagcggctcccgtaagaggaaccacagccccagctttaatagcaccatttacgaggtcatcggcaagtcccagcctaaggcccaaaaccccgctcgtctctctcgtaaggagctggagaatttcgacgtgtacagctaa

SEQ ID NO: 56 - DNA, 1374 bp, Artificial Sequence
  synthetic polynucleotide
tggaggaggaaaaggaaggagaagcagtccgagacatcccccaaagagtttttaaccatttatgaggacgtgaaggacctcaagaccagaaggaaccacgagcaagaacagacctttcccggcggcggatccaccatctacagcatgatccagagccagagctccgcccctacaagccaagaacccgcttacactttatattctttaatccagccttctcgtaagagcggctcccgtaagaggaaccacagccccagctttaatagcaccatttacgaggtcatcggcaagtcccagcctaaggcccaaaaccccgctcgtctctctcgtaaggagctggagaatttcgacgtgtacagccctttcttcttctgctgcttcatcgccgtggccatgggcattcgtttcatcatcatggtggccaccacaacccctgcccccagacctcccacacccgcccctaccatcgctagccagcctctgagcctgaggcccgaggcttgcagacctgctgctggaggcgccgtgcacaccagaggactggatttcgcctgcgacgacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcggtggaggcggttcggattacaaggatgacgacgataaggactataaggacgacgacgataaggattataaagacgatgacgacaaataa

SEQ ID NO: 57 - DNA, 1500 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacatgggactggcctttctggtgctggtggctctggtgtggtttctggtggaggactggctgagcagaaagaggaccagagagagagccagcagagccagcacatgggagggcagaaggagactgaacacccagaccctgaagaggaagaagcagaggtctagaagaaacgacgaggagctcgagacaagagcccacagagtggctaccgaagagagaggcagaaagccccaccagatccccgccagcacaccccagaaccccgccacaagccagcatcccccccctcctcccggccacagatcccaagctcccagccacagacctcctccccccggccatagggtgcagcatcagccccagaaaagacctcccgcccccagcggaacccaagtgcatcagcagaagggccctcctctgcccagacctagggtgcaacccaagccccctcatggcgctgccgagaactctctgagccctagcagcaactaa

SEQ ID NO: 58 - DNA, 1620 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacggcggcaccgtgctgctgctgctgttcgtgatctccatcaccaccatcatcgtcatctttctgaacagaaggaggagaagggagaggagggatctgttcaccgagagctgggacacccagaaggcccccaacaactatagaagccccatcagcaccagccagcccaccaaccagagcatggacgacaccagagaggacatctacgtgaactaccccaccttcagcagaagacccaagaccagagtgtggaggaggaaaaggaaggagaagcagtccgagacatcccccaaagagtttttaaccatttatgaggacgtgaaggacctcaagaccagaaggaaccacgagcaagaacagacctttcccggcggcggatccaccatctacagcatgatccagagccagagctccgcccctacaagccaagaacccgcttacactttatattctttaatccagccttctcgtaagagcggctcccgtaagaggaaccacagccccagctttaatagcaccatttacgaggtcatcggcaagtcccagcctaaggcccaaaaccccgctcgtctctctcgtaaggagctggagaatttcgacgtgtacagctaa

SEQ ID NO: 59 - DNA, 1266 bp, Artificial Sequence
  synthetic polynucleotide
ctgagaaagagaagagactctctgtctctgagcacccaaagaacacaaggccccgagagcgccagaaatctggagtacgtgagcgtgagccccaccaacaacaccgtgtacgccagcgtgacccacagcaacagagagaccgagatctggacccctagagagaacgacaccatcaccatctacagcaccatcaaccacagcaaggagagcaagcccacattctctagagccaccgctctggataacgtggtgcctttcttcttctgctgcttcatcgccgtggccatgggcattcgtttcatcatcatggtggccaccacaacccctgcccccagacctcccacacccgcccctaccatcgctagccagcctctgagcctgaggcccgaggcttgcagacctgctgctggaggcgccgtgcacaccagaggactggatttcgcctgcgacgacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcggtggaggcggttcggattacaaggatgacgacgataaggactataaggacgacgacgataaggattataaagacgatgacgacaaataa

SEQ ID NO: 60 - DNA, 1404 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacatgggactggcctttctggtgctggtggctctggtgtggtttctggtggaggactggctgagcagaaagaggaccagagagagagccagcagagccagcacatgggagggcagaaggagactgaacacccagaccctgctgagaaagagaagagactctctgtctctgagcacccaaagaacacaaggccccgagagcgccagaaatctggagtacgtgagcgtgagccccaccaacaacaccgtgtacgccagcgtgacccacagcaacagagagaccgagatctggacccctagagagaacgacaccatcaccatctacagcaccatcaaccacagcaaggagagcaagcccacattctctagagccaccgctctggataacgtggtgtaa

SEQ ID NO: 61 - DNA, 1524 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacggcggcaccgtgctgctgctgctgttcgtgatctccatcaccaccatcatcgtcatctttctgaacagaaggaggagaagggagaggagggatctgttcaccgagagctgggacacccagaaggcccccaacaactatagaagccccatcagcaccagccagcccaccaaccagagcatggacgacaccagagaggacatctacgtgaactaccccaccttcagcagaagacccaagaccagagtgtggtttctgaagagagagaggcaagaggagtacatcgaggagaagaagagggtggacatctgcagagagacccccaacatctgcccccattccggcgagaacaccgagtacgacaccatcccccacaccaacagaaccattctgaaggaggaccccgccaacaccgtgtacagcaccgtggagatccccaagaagatggagaatccccactctctgctgaccatgcccgacacccccagactgttcgcctatgagaatgttatctaa

SEQ ID NO: 62 - DNA, 1362 bp, Artificial Sequence
  synthetic polynucleotide
aagaggaagaagcagaggtctagaagaaacgacgaggagctcgagacaagagcccacagagtggctaccgaagagagaggcagaaagccccaccagatccccgccagcacaccccagaaccccgccacaagccagcatcccccccctcctcccggccacagatcccaagctcccagccacagacctcctccccccggccatagggtgcagcatcagccccagaaaagacctcccgcccccagcggaacccaagtgcatcagcagaagggccctcctctgcccagacctagggtgcaacccaagccccctcatggcgctgccgagaactctctgagccctagcagcaaccctttcttcttctgctgcttcatcgccgtggccatgggcattcgtttcatcatcatggtggccaccacaacccctgcccccagacctcccacacccgcccctaccatcgctagccagcctctgagcctgaggcccgaggcttgcagacctgctgctggaggcgccgtgcacaccagaggactggatttcgcctgcgacgacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcggtggaggcggttcggattacaaggatgacgacgataaggactataaggacgacgacgataaggattataaagacgatgacgacaaataa

SEQ ID NO: 63 - DNA, 1512 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacatgggactggcctttctggtgctggtggctctggtgtggtttctggtggaggactggctgagcagaaagaggaccagagagagagccagcagagccagcacatgggagggcagaaggagactgaacacccagaccctgtggaggaggaaaaggaaggagaagcagtccgagacatcccccaaagagtttttaaccatttatgaggacgtgaaggacctcaagaccagaaggaaccacgagcaagaacagacctttcccggcggcggatccaccatctacagcatgatccagagccagagctccgcccctacaagccaagaacccgcttacactttatattctttaatccagccttctcgtaagagcggctcccgtaagaggaaccacagccccagctttaatagcaccatttacgaggtcatcggcaagtcccagcctaaggcccaaaaccccgctcgtctctctcgtaaggagctggagaatttcgacgtgtacagctaa

SEQ ID NO: 64 - DNA, 1416 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacatgggactggcctttctggtgctggtggctctggtgtggtttctggtggaggactggctgagcagaaagaggaccagagagagagccagcagagccagcacatgggagggcagaaggagactgaacacccagaccctgtggtttctgaagagagagaggcaagaggagtacatcgaggagaagaagagggtggacatctgcagagagacccccaacatctgcccccattccggcgagaacaccgagtacgacaccatcccccacaccaacagaaccattctgaaggaggaccccgccaacaccgtgtacagcaccgtggagatccccaagaagatggagaatccccactctctgctgaccatgcccgacacccccagactgttcgcctatgagaatgttatctaa

SEQ ID NO: 65 - DNA, 1608 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacggcggcaccgtgctgctgctgctgttcgtgatctccatcaccaccatcatcgtcatctttctgaacagaaggaggagaagggagaggagggatctgttcaccgagagctgggacacccagaaggcccccaacaactatagaagccccatcagcaccagccagcccaccaaccagagcatggacgacaccagagaggacatctacgtgaactaccccaccttcagcagaagacccaagaccagagtgaagaggaagaagcagaggtctagaagaaacgacgaggagctcgagacaagagcccacagagtggctaccgaagagagaggcagaaagccccaccagatccccgccagcacaccccagaaccccgccacaagccagcatcccccccctcctcccggccacagatcccaagctcccagccacagacctcctccccccggccatagggtgcagcatcagccccagaaaagacctcccgcccccagcggaacccaagtgcatcagcagaagggccctcctctgcccagacctagggtgcaacccaagccccctcatggcgctgccgagaactctctgagccctagcagcaactaa

SEQ ID NO: 66 - DNA, 1341 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgaccctttcttcttctgctgcttcatcgccgtggccatgggcattcgtttcatcatcatggtggcctggtttctgaagagagagaggcaagaggagtacatcgaggagaagaagagggtggacatctgcagagagacccccaacatctgcccccattccggcgagaacaccgagtacgacaccatcccccacaccaacagaaccattctgaaggaggaccccgccaacaccgtgtacagcaccgtggagatccccaagaagatggagaatccccactctctgctgaccatgcccgacacccccagactgttcgcctatgagaatgttatctaa

SEQ ID NO: 67 - DNA, 1278 bp, Artificial Sequence
  synthetic polynucleotide
tggtttctgaagagagagaggcaagaggagtacatcgaggagaagaagagggtggacatctgcagagagacccccaacatctgcccccattccggcgagaacaccgagtacgacaccatcccccacaccaacagaaccattctgaaggaggaccccgccaacaccgtgtacagcaccgtggagatccccaagaagatggagaatccccactctctgctgaccatgcccgacacccccagactgttcgcctatgagaatgttatccctttcttcttctgctgcttcatcgccgtggccatgggcattcgtttcatcatcatggtggccaccacaacccctgcccccagacctcccacacccgcccctaccatcgctagccagcctctgagcctgaggcccgaggcttgcagacctgctgctggaggcgccgtgcacaccagaggactggatttcgcctgcgacgacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcggtggaggcggttcggattacaaggatgacgacgataaggactataaggacgacgacgataaggattataaagacgatgacgacaaataa

SEQ ID NO: 68 - DNA, 1425 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgaccctttcttcttctgctgcttcatcgccgtggccatgggcattcgtttcatcatcatggtggccaagaggaagaagcagaggtctagaagaaacgacgaggagctcgagacaagagcccacagagtggctaccgaagagagaggcagaaagccccaccagatccccgccagcacaccccagaaccccgccacaagccagcatcccccccctcctcccggccacagatcccaagctcccagccacagacctcctccccccggccatagggtgcagcatcagccccagaaaagacctcccgcccccagcggaacccaagtgcatcagcagaagggccctcctctgcccagacctagggtgcaacccaagccccctcatggcgctgccgagaactctctgagccctagcagcaactaa

SEQ ID NO: 69 - DNA, 1512 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgacggcggcaccgtgctgctgctgctgttcgtgatctccatcaccaccatcatcgtcatctttctgaacagaaggaggagaagggagaggagggatctgttcaccgagagctgggacacccagaaggcccccaacaactatagaagccccatcagcaccagccagcccaccaaccagagcatggacgacaccagagaggacatctacgtgaactaccccaccttcagcagaagacccaagaccagagtgctgagaaagagaagagactctctgtctctgagcacccaaagaacacaaggccccgagagcgccagaaatctggagtacgtgagcgtgagccccaccaacaacaccgtgtacgccagcgtgacccacagcaacagagagaccgagatctggacccctagagagaacgacaccatcaccatctacagcaccatcaaccacagcaaggagagcaagcccacattctctagagccaccgctctggataacgtggtgtaa

SEQ ID NO: 70 - DNA, 1329 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggacatccagatgacccagaccaccagcagcctgagcgctagcctgggcgatagagtgaccatcagctgcagagccagccaggacatcagcaagtacctgaactggtaccagcagaagcccgacggcacagtgaaactgctgatctaccacacctccagactgcatagcggcgtgcctagcaggttttccggcagcggcagcggaaccgactactccctgaccatcagcaacctggagcaggaggacatcgctacctacttttgtcagcagggcaacaccctgccttacacctttggcggcggaacaaagctggagattaccggcggcggaggcagcggcggcggaggctccggaggcggaggatccgaggtgaagctgcaggaaagcggacctggactggtcgcccctagccagagcctgagcgtgacctgtaccgtgagcggagtgagcctgcccgattacggcgtgtcctggatcaggcagccccccaggaagggactggaatggctgggcgtcatctggggctccgagacaacctactacaacagcgccctcaagtccaggctcaccattatcaaggacaatagcaagagccaggtgttcctgaagatgaacagcctgcagaccgacgacacagccatttattactgcgccaagcactattattacggcggcagctacgccatggactattggggccagggaacaagcgtgaccgtcagcagcaccacaacccctgcccccagacctcccacacccgcccctaccatcgctagccagcctctgagcctgaggcccgaggcttgcagacctgctgctggaggcgccgtgcacaccagaggactggatttcgcctgcgaccctttcttcttctgctgcttcatcgccgtggccatgggcattcgtttcatcatcatggtggccctgagaaagagaagagactctctgtctctgagcacccaaagaacacaaggccccgagagcgccagaaatctggagtacgtgagcgtgagccccaccaacaacaccgtgtacgccagcgtgacccacagcaacagagagaccgagatctggacccctagagagaacgacaccatcaccatctacagcaccatcaaccacagcaaggagagcaagcccacattctctagagccaccgctctggataacgtggtgtaa

SEQ ID NO: 71 - DNA, 1485 bp, Artificial Sequence
  synthetic polynucleotide
atggccttaccagtgaccgccttgctcctgccgctggccttgctgctccacgccgccaggccgggtggaggcggttcggaagtgcagctggtgcagagcggagcagaagtgaagaagcccggaagcagcgtgaaggtgtcttgcaaggccagcggctacaccatcaccgacagcaacatccattgggtccggcaggctccaggacagtctctggagtggatcggctacatctacccctacaacggcggcaccgactacaaccagaagttcaagaaccgggccaccctgaccgtggataaccccaccaacaccgcctacatggagctgagcagcctgagaagcgaggacaccgccttctactattgcgtgaacggcaacccttggctggcctattggggacagggaacactggtgaccgtgtcctctggcggcggaggcagcggcggcggaggctccggaggcggaggatccgacatccagctgacccagtctcctagcaccctgagcgctagcgtgggagatagagtgaccatcacttgcagagccagcgagagcctggacaactacggcatccggttcctgacttggttccagcagaaacccggcaaggcccctaaactgctgatgtacgccgcctctaaccagggaagcggagtgcctagcagattcagcggcagcggaagcggaaccgagttcaccctgaccatcagctctctgcagccagacgacttcgccacctactactgccagcagaccaaggaggtgccttggagcttcggccagggaaccaaggtggaagtgaagcggacagtgaccacaacccctgcccccagacctcccacacccgcccctaccatcgctagccagcctctgagcctgaggcccgaggcttgcagacctgctgctggaggcgccgtgcacaccagaggactggatttcgcctgcgacatctacatctgggcccctctggctggcacatgtggcgtgctgctgctctccctggtgatcaccctgtactgcaaaaggggcagaaagaaactgctctacatcttcaagcagcccttcatgaggcccgtccaaaccacccaggaggaggacggatgcagctgtagattccccgaagaggaggaaggcggctgcgagctgagagtcaagttctccaggtccgccgacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 72 - DNA, 1557 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggaagtgcagctggtgcagagcggagcagaagtgaagaagcccggaagcagcgtgaaggtgtcttgcaaggccagcggctacaccatcaccgacagcaacatccattgggtccggcaggctccaggacagtctctggagtggatcggctacatctacccctacaacggcggcaccgactacaaccagaagttcaagaaccgggccaccctgaccgtggataaccccaccaacaccgcctacatggagctgagcagcctgagaagcgaggacaccgccttctactattgcgtgaacggcaacccttggctggcctattggggacagggaacactggtgaccgtgtcctctggcggcggaggcagcggcggcggaggctccggaggcggaggatccgacatccagctgacccagtctcctagcaccctgagcgctagcgtgggagatagagtgaccatcacttgcagagccagcgagagcctggacaactacggcatccggttcctgacttggttccagcagaaacccggcaaggcccctaaactgctgatgtacgccgcctctaaccagggaagcggagtgcctagcagattcagcggcagcggaagcggaaccgagttcaccctgaccatcagctctctgcagccagacgacttcgccacctactactgccagcagaccaaggaggtgccttggagcttcggccagggaaccaaggtggaagtgaagcggacagtgaccacaacccctgcccccagacctcccacacccgcccctaccatcgctagccagcctctgagcctgaggcccgaggcttgcagacctgctgctggaggcgccgtgcacaccagaggactggatttcgcctgcgacatctacatctgggcccctctggctggcacatgtggcgtgctgctgctctccctggtgatcaccctgtactgcaaaaggggcagaaagaaactgctctacatcttcaagcagcccttcatgaggcccgtccaaaccacccaggaggaggacggatgcagctgtagattccccgaagaggaggaaggcggctgcgagctgagagtcaagttctccaggtccgccgacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaa

SEQ ID NO: 73 - DNA, 2433 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggaagtgcagctggtgcagagcggagcagaagtgaagaagcccggaagcagcgtgaaggtgtcttgcaaggccagcggctacaccatcaccgacagcaacatccattgggtccggcaggctccaggacagtctctggagtggatcggctacatctacccctacaacggcggcaccgactacaaccagaagttcaagaaccgggccaccctgaccgtggataaccccaccaacaccgcctacatggagctgagcagcctgagaagcgaggacaccgccttctactattgcgtgaacggcaacccttggctggcctattggggacagggaacactggtgaccgtgtcctctggcggcggaggcagcggcggcggaggctccggaggcggaggatccgacatccagctgacccagtctcctagcaccctgagcgctagcgtgggagatagagtgaccatcacttgcagagccagcgagagcctggacaactacggcatccggttcctgacttggttccagcagaaacccggcaaggcccctaaactgctgatgtacgccgcctctaaccagggaagcggagtgcctagcagattcagcggcagcggaagcggaaccgagttcaccctgaccatcagctctctgcagccagacgacttcgccacctactactgccagcagaccaaggaggtgccttggagcttcggccagggaaccaaggtggaagtgaagcggacagtgaccacaacccctgcccccagacctcccacacccgcccctaccatcgctagccagcctctgagcctgaggcccgaggcttgcagacctgctgctggaggcgccgtgcacaccagaggactggatttcgcctgcgacatctacatctgggcccctctggctggcacatgtggcgtgctgctgctctccctggtgatcaccctgtactgcaaaaggggcagaaagaaactgctctacatcttcaagcagcccttcatgaggcccgtccaaaccacccaggaggaggacggatgcagctgtagattccccgaagaggaggaaggcggctgcgagctgcgtctgaagatccaagttaggaaggccgccatcacctcctacgagaagagcgacggcgtgtacaccggtttaagcacaaggaaccaagaaacctacgagactttaaagcacgagaagcccccccagggcagcggcgccaccaacttctctttactgaagcaagctggcgacgtggaggagaatcccggtcccatgaggatcagcaagccccatttaaggagcatctccatccagtgctatttatgtttactgctgaacagccattttttaaccgaggccggcatccacgtgttcattttaggctgcttttccgccggtttacccaagaccgaggctaactgggtgaacgtgatcagcgatttaaagaagatcgaggatttaatccagagcatgcacatcgatgccactttatataccgagagcgacgtgcaccccagctgcaaggtgaccgccatgaagtgttttttactggagctgcaagttatctctttagagagcggcgacgccagcatccacgacaccgtggagaatttaatcattttagccaacaacagcctctccagcaacggcaatgtgacagagagcggctgcaaggagtgcgaggagctggaggagaagaacatcaaggagtttttacagagcttcgtgcacatcgtgcagatgttcatcaacaccagcggcagcggaagagccgagggaagaggctctttactgacttgtggcgacgtggaagagaatcccggccccatggagagaatcgtgatctgtttaatggtgatctttttaggcactttagtgcacaagtccagctcccaaggtcaagatagacacatgattcgtatgaggcagctgatcgacatcgtggaccagctgaagaactacgtgaacgatttagtgcccgaatttttacccgctcccgaggacgtggagaccaactgcgagtggagcgccttctcttgtttccagaaggctcagctgaagagcgccaacaccggcaacaacgagaggattatcaacgtgtccatcaagaagctgaagaggaagcctccctccaccaacgccggcagaaggcagaagcacagactgacttgtccctcttgtgacagctacgagaagaagccccctaaggagtttttagagagattcaagtctttactgcagaagatgatccaccagcatttaagctctcgtacccacggcagcgaggattcctaa

SEQ ID NO: 74 - DNA, 1629 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggaagtgcagctggtgcagagcggagcagaagtgaagaagcccggaagcagcgtgaaggtgtcttgcaaggccagcggctacaccatcaccgacagcaacatccattgggtccggcaggctccaggacagtctctggagtggatcggctacatctacccctacaacggcggcaccgactacaaccagaagttcaagaaccgggccaccctgaccgtggataaccccaccaacaccgcctacatggagctgagcagcctgagaagcgaggacaccgccttctactattgcgtgaacggcaacccttggctggcctattggggacagggaacactggtgaccgtgtcctctggcggcggaggcagcggcggcggaggctccggaggcggaggatccgacatccagctgacccagtctcctagcaccctgagcgctagcgtgggagatagagtgaccatcacttgcagagccagcgagagcctggacaactacggcatccggttcctgacttggttccagcagaaacccggcaaggcccctaaactgctgatgtacgccgcctctaaccagggaagcggagtgcctagcagattcagcggcagcggaagcggaaccgagttcaccctgaccatcagctctctgcagccagacgacttcgccacctactactgccagcagaccaaggaggtgccttggagcttcggccagggaaccaaggtggaagtgaagcggacagtgaccacaacccctgcccccagacctcccacacccgcccctaccatcgctagccagcctctgagcctgaggcccgaggcttgcagacctgctgctggaggcgccgtgcacaccagaggactggatttcgcctgcgacatctacatctgggcccctctggctggcacatgtggcgtgctgctgctctccctggtgatcaccctgtactgcaactgcaggaacaccgggccatggctgaagaaggtcctgaagtgtaacaccccagacccctcgaagttcttttcccagctgagctcagagcatggaggagacgtccagaagtggctctcttcgcccttcccctcatcgtccttcagccctggcggcctggcacctgagatctcgccactagaagtgctggagagggacaaggtgacgcagctgctccccctgaacactgatgcctacttgtccctccaagaactccagggtcaggacccaactcacttggtgaaaaggggcagaaagaaactgctctacatcttcaagcagcccttcatgaggcccgtccaaaccacccaggaggaggacggatgcagctgtagattccccgaagaggaggaaggcggctgcgagctgcgtctgaagatccaagttaggaaggccgccatcacctcctacgagaagagcgacggcgtgtacaccggtttaagcacaaggaaccaagaaacctacgagactttaaagcacgagaagcccccccagtaa

SEQ ID NO: 75 - DNA, 1446 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggaagtgcagctggtgcagagcggagcagaagtgaagaagcccggaagcagcgtgaaggtgtcttgcaaggccagcggctacaccatcaccgacagcaacatccattgggtccggcaggctccaggacagtctctggagtggatcggctacatctacccctacaacggcggcaccgactacaaccagaagttcaagaaccgggccaccctgaccgtggataaccccaccaacaccgcctacatggagctgagcagcctgagaagcgaggacaccgccttctactattgcgtgaacggcaacccttggctggcctattggggacagggaacactggtgaccgtgtcctctggcggcggaggcagcggcggcggaggctccggaggcggaggatccgacatccagctgacccagtctcctagcaccctgagcgctagcgtgggagatagagtgaccatcacttgcagagccagcgagagcctggacaactacggcatccggttcctgacttggttccagcagaaacccggcaaggcccctaaactgctgatgtacgccgcctctaaccagggaagcggagtgcctagcagattcagcggcagcggaagcggaaccgagttcaccctgaccatcagctctctgcagccagacgacttcgccacctactactgccagcagaccaaggaggtgccttggagcttcggccagggaaccaaggtggaagtgaagcggacagtgaccacaacccctgcccccagacctcccacacccgcccctaccatcgctagccagcctctgagcctgaggcccgaggcttgcagacctgctgctggaggcgccgtgcacaccagaggactggatttcgcctgcgaccctttcttcttctgctgcttcatcgccgtggccatgggcattcgtttcatcatcatggtggcctggaggaggaaaaggaaggagaagcagtccgagacatcccccaaagagtttttaaccatttatgaggacgtgaaggacctcaagaccagaaggaaccacgagcaagaacagacctttcccggcggcggatccaccatctacagcatgatccagagccagagctccgcccctacaagccaagaacccgcttacactttatattctttaatccagccttctcgtaagagcggctcccgtaagaggaaccacagccccagctttaatagcaccatttacgaggtcatcggcaagtcccagcctaaggcccaaaaccccgctcgtctctctcgtaaggagctggagaatttcgacgtgtacagctaa

SEQ ID NO: 76 - DNA, 1383 bp, Artificial Sequence
  synthetic polynucleotide
tggaggaggaaaaggaaggagaagcagtccgagacatcccccaaagagtttttaaccatttatgaggacgtgaaggacctcaagaccagaaggaaccacgagcaagaacagacctttcccggcggcggatccaccatctacagcatgatccagagccagagctccgcccctacaagccaagaacccgcttacactttatattctttaatccagccttctcgtaagagcggctcccgtaagaggaaccacagccccagctttaatagcaccatttacgaggtcatcggcaagtcccagcctaaggcccaaaaccccgctcgtctctctcgtaaggagctggagaatttcgacgtgtacagccctttcttcttctgctgcttcatcgccgtggccatgggcattcgtttcatcatcatggtggccaccacaacccctgcccccagacctcccacacccgcccctaccatcgctagccagcctctgagcctgaggcccgaggcttgcagacctgctgctggaggcgccgtgcacaccagaggactggatttcgcctgcgacgaagtgcagctggtgcagagcggagcagaagtgaagaagcccggaagcagcgtgaaggtgtcttgcaaggccagcggctacaccatcaccgacagcaacatccattgggtccggcaggctccaggacagtctctggagtggatcggctacatctacccctacaacggcggcaccgactacaaccagaagttcaagaaccgggccaccctgaccgtggataaccccaccaacaccgcctacatggagctgagcagcctgagaagcgaggacaccgccttctactattgcgtgaacggcaacccttggctggcctattggggacagggaacactggtgaccgtgtcctctggcggcggaggcagcggcggcggaggctccggaggcggaggatccgacatccagctgacccagtctcctagcaccctgagcgctagcgtgggagatagagtgaccatcacttgcagagccagcgagagcctggacaactacggcatccggttcctgacttggttccagcagaaacccggcaaggcccctaaactgctgatgtacgccgcctctaaccagggaagcggagtgcctagcagattcagcggcagcggaagcggaaccgagttcaccctgaccatcagctctctgcagccagacgacttcgccacctactactgccagcagaccaaggaggtgccttggagcttcggccagggaaccaaggtggaagtgaagcggacagtgggtggaggcggttcggattacaaggatgacgacgataaggactataaggacgacgacgataaggattataaagacgatgacgacaaataa

SEQ ID NO: 77 - DNA, 1533 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggaagtgcagctggtgcagagcggagcagaagtgaagaagcccggaagcagcgtgaaggtgtcttgcaaggccagcggctacaccatcaccgacagcaacatccattgggtccggcaggctccaggacagtctctggagtggatcggctacatctacccctacaacggcggcaccgactacaaccagaagttcaagaaccgggccaccctgaccgtggataaccccaccaacaccgcctacatggagctgagcagcctgagaagcgaggacaccgccttctactattgcgtgaacggcaacccttggctggcctattggggacagggaacactggtgaccgtgtcctctggcggcggaggcagcggcggcggaggctccggaggcggaggatccgacatccagctgacccagtctcctagcaccctgagcgctagcgtgggagatagagtgaccatcacttgcagagccagcgagagcctggacaactacggcatccggttcctgacttggttccagcagaaacccggcaaggcccctaaactgctgatgtacgccgcctctaaccagggaagcggagtgcctagcagattcagcggcagcggaagcggaaccgagttcaccctgaccatcagctctctgcagccagacgacttcgccacctactactgccagcagaccaaggaggtgccttggagcttcggccagggaaccaaggtggaagtgaagcggacagtgaccacaacccctgcccccagacctcccacacccgcccctaccatcgctagccagcctctgagcctgaggcccgaggcttgcagacctgctgctggaggcgccgtgcacaccagaggactggatttcgcctgcgacggcggcaccgtgctgctgctgctgttcgtgatctccatcaccaccatcatcgtcatctttctgaacagaaggaggagaagggagaggagggatctgttcaccgagagctgggacacccagaaggcccccaacaactatagaagccccatcagcaccagccagcccaccaaccagagcatggacgacaccagagaggacatctacgtgaactaccccaccttcagcagaagacccaagaccagagtgtggtttctgaagagagagaggcaagaggagtacatcgaggagaagaagagggtggacatctgcagagagacccccaacatctgcccccattccggcgagaacaccgagtacgacaccatcccccacaccaacagaaccattctgaaggaggaccccgccaacaccgtgtacagcaccgtggagatccccaagaagatggagaatccccactctctgctgaccatgcccgacacccccagactgttcgcctatgagaatgttatctaa

SEQ ID NO: 78 - DNA, 1350 bp, Artificial Sequence
  synthetic polynucleotide
atggccttaccagtgaccgccttgctcctgccgctggccttgctgctccacgccgccaggccggattacaaggatgacgacgataaggactataaggacgacgacgataaggattataaagacgatgacgacaaaggtggaggcggttcggaagtgcagctggtgcagagcggagcagaagtgaagaagcccggaagcagcgtgaaggtgtcttgcaaggccagcggctacaccatcaccgacagcaacatccattgggtccggcaggctccaggacagtctctggagtggatcggctacatctacccctacaacggcggcaccgactacaaccagaagttcaagaaccgggccaccctgaccgtggataaccccaccaacaccgcctacatggagctgagcagcctgagaagcgaggacaccgccttctactattgcgtgaacggcaacccttggctggcctattggggacagggaacactggtgaccgtgtcctctggcggcggaggcagcggcggcggaggctccggaggcggaggatccgacatccagctgacccagtctcctagcaccctgagcgctagcgtgggagatagagtgaccatcacttgcagagccagcgagagcctggacaactacggcatccggttcctgacttggttccagcagaaacccggcaaggcccctaaactgctgatgtacgccgcctctaaccagggaagcggagtgcctagcagattcagcggcagcggaagcggaaccgagttcaccctgaccatcagctctctgcagccagacgacttcgccacctactactgccagcagaccaaggaggtgccttggagcttcggccagggaaccaaggtggaagtgaagcggacagtgaccacaacccctgcccccagacctcccacacccgcccctaccatcgctagccagcctctgagcctgaggcccgaggcttgcagacctgctgctggaggcgccgtgcacaccagaggactggatttcgcctgcgaccctttcttcttctgctgcttcatcgccgtggccatgggcattcgtttcatcatcatggtggcctggtttctgaagagagagaggcaagaggagtacatcgaggagaagaagagggtggacatctgcagagagacccccaacatctgcccccattccggcgagaacaccgagtacgacaccatcccccacaccaacagaaccattctgaaggaggaccccgccaacaccgtgtacagcaccgtggagatccccaagaagatggagaatccccactctctgctgaccatgcccgacacccccagactgttcgcctatgagaatgttatctaa

SEQ ID NO: 79 - DNA, 1287 bp, Artificial Sequence
  synthetic polynucleotide
tggtttctgaagagagagaggcaagaggagtacatcgaggagaagaagagggtggacatctgcagagagacccccaacatctgcccccattccggcgagaacaccgagtacgacaccatcccccacaccaacagaaccattctgaaggaggaccccgccaacaccgtgtacagcaccgtggagatccccaagaagatggagaatccccactctctgctgaccatgcccgacacccccagactgttcgcctatgagaatgttatccctttcttcttctgctgcttcatcgccgtggccatgggcattcgtttcatcatcatggtggccaccacaacccctgcccccagacctcccacacccgcccctaccatcgctagccagcctctgagcctgaggcccgaggcttgcagacctgctgctggaggcgccgtgcacaccagaggactggatttcgcctgcgacgaagtgcagctggtgcagagcggagcagaagtgaagaagcccggaagcagcgtgaaggtgtcttgcaaggccagcggctacaccatcaccgacagcaacatccattgggtccggcaggctccaggacagtctctggagtggatcggctacatctacccctacaacggcggcaccgactacaaccagaagttcaagaaccgggccaccctgaccgtggataaccccaccaacaccgcctacatggagctgagcagcctgagaagcgaggacaccgccttctactattgcgtgaacggcaacccttggctggcctattggggacagggaacactggtgaccgtgtcctctggcggcggaggcagcggcggcggaggctccggaggcggaggatccgacatccagctgacccagtctcctagcaccctgagcgctagcgtgggagatagagtgaccatcacttgcagagccagcgagagcctggacaactacggcatccggttcctgacttggttccagcagaaacccggcaaggcccctaaactgctgatgtacgccgcctctaaccagggaagcggagtgcctagcagattcagcggcagcggaagcggaaccgagttcaccctgaccatcagctctctgcagccagacgacttcgccacctactactgccagcagaccaaggaggtgccttggagcttcggccagggaaccaaggtggaagtgaagcggacagtgggtggaggcggttcggattacaaggatgacgacgataaggactataaggacgacgacgataaggattataaagacgatgacgacaaataa

SEQ ID NO: 80 - protein, 361 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYC

SEQ ID NO: 81 - protein, 491 aa, Artificial Sequence
  synthetic polypeptide
MALPVTALLLPLALLLHAARPGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 515 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 445 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRLKIQVRKAAITSYEKSDGVYTGLSTRNQETYETLKHEKPPQ

SEQ ID NO: 84 - protein, 629 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRLKIQVRKAAITSYEKSDGVYTGLSTRNQETYETLKHEKPPQGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 85 - protein, 807 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRLKIQVRKAAITSYEKSDGVYTGLSTRNQETYETLKHEKPPQGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGRAEGRGSLLTCGDVEENPGPMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 86 - protein, 609 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 87 - protein, 609 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 539 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVRLKIQVRKAAITSYEKSDGVYTGLSTRNQETYETLKHEKPPQ

SEQ ID NO: 89 - protein, 539 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVKRGRKKLLYIFKQPFMRPVQTTQEEDGCSCRFPEEEEGGCELRLKIQVRKAAITSYEKSDGVYTGLSTRNQETYETLKHEKPPQ

SEQ ID NO: 90 - protein, 478 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDPFFFCCFIAVAMGIRFIIMVAWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 91 - protein, 457 aa, Artificial Sequence
  synthetic polypeptide
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSPFFFCCFIAVAMGIRFIIMVATTTPAPRPPTPAPTIASQPLSLRPEACRPAAGGAVHTRGLDFACDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSDYKDDDDKDYKDDDDKDYKDDDDK

SEQ ID NO: 92 - protein, 499 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDMGLAFLVLVALVWFLVEDWLSRKRTRERASRASTWEGRRRLNTQTLKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 93 - protein, 539 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDGGTVLLLLFVISITTIIVIFLNRRRRRERRDLFTESWDTQKAPNNYRSPISTSQPTNQSMDDTREDIYVNYPTFSRRPKTRVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 94 - protein, 421 aa, Artificial Sequence
  synthetic polypeptide
LRKRRDSLSLSTQRTQGPESARNLEYVSVSPTNNTVYASVTHSNRETEIWTPRENDTITIYSTINHSKESKPTFSRATALDNVVPFFFCCFIAVAMGIRFIIMVATTTPAPRPPTPAPTIASQPLSLRPEACRPAAGGAVHTRGLDFACDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSDYKDDDDKDYKDDDDKDYKDDDDK

SEQ ID NO: 95 - protein, 467 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDMGLAFLVLVALVWFLVEDWLSRKRTRERASRASTWEGRRRLNTQTLLRKRRDSLSLSTQRTQGPESARNLEYVSVSPTNNTVYASVTHSNRETEIWTPRENDTITIYSTINHSKESKPTFSRATALDNVV

SEQ ID NO: 96 - protein, 507 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDGGTVLLLLFVISITTIIVIFLNRRRRRERRDLFTESWDTQKAPNNYRSPISTSQPTNQSMDDTREDIYVNYPTFSRRPKTRVWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 97 - protein, 453 aa, Artificial Sequence
  synthetic polypeptide
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSNPFFFCCFIAVAMGIRFIIMVATTTPAPRPPTPAPTIASQPLSLRPEACRPAAGGAVHTRGLDFACDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSDYKDDDDKDYKDDDDKDYKDDDDK

SEQ ID NO: 98 - protein, 503 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDMGLAFLVLVALVWFLVEDWLSRKRTRERASRASTWEGRRRLNTQTLWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 99 - protein, 471 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDMGLAFLVLVALVWFLVEDWLSRKRTRERASRASTWEGRRRLNTQTLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 100 - protein, 535 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDGGTVLLLLFVISITTIIVIFLNRRRRRERRDLFTESWDTQKAPNNYRSPISTSQPTNQSMDDTREDIYVNYPTFSRRPKTRVKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 101 - protein, 446 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDPFFFCCFIAVAMGIRFIIMVAWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 102 - protein, 425 aa, Artificial Sequence
  synthetic polypeptide
WFLKRERQEEYIEEKKRVDICRETPNICPHSGENTEYDTIPHTNRTILKEDPANTVYSTVEIPKKMENPHSLLTMPDTPRLFAYENVIPFFFCCFIAVAMGIRFIIMVATTTPAPRPPTPAPTIASQPLSLRPEACRPAAGGAVHTRGLDFACDDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGGSDYKDDDDKDYKDDDDKDYKDDDDK

SEQ ID NO: 103 - protein, 474 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDPFFFCCFIAVAMGIRFIIMVAKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 104 - protein, 503 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDGGTVLLLLFVISITTIIVIFLNRRRRRERRDLFTESWDTQKAPNNYRSPISTSQPTNQSMDDTREDIYVNYPTFSRRPKTRVLRKRRDSLSLSTQRTQGPESARNLEYVSVSPTNNTVYASVTHSNRETEIWTPRENDTITIYSTINHSKESKPTFSRATALDNVV

SEQ ID NO: 105 - protein, 442 aa, Artificial Sequence
  synthetic polypeptide
MALPVTALLLPLALLLHAARPDYKDDDDKDYKDDDDKDYKDDDDKGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDPFFFCCFIAVAMGIRFIIMVALRKRRDSLSLSTQRTQGPESARNLEYVSVSPTNNTVYASVTHSNRETEIWTPRENDTITIYSTINHSKESKPTFSRATALDNVV

SEQ ID NO: 106 - protein, 494 aa, Artificial Sequence
  synthetic polypeptide
MALPVTALLLPLALLLHAARPGGGGSEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - protein, 518 aa, Artificial Sequence
  synthetic polypeptide
MALPVTALLLPLALLLHAARPDYKDDDDKDYKDDDDKDYKDDDDKGGGGSEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - protein, 810 aa, Artificial Sequence
  synthetic polypeptide
MALPVTALLLPLALLLHAARPDYKDDDDKDYKDDDDKDYKDDDDKGGGGSEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRLKIQVRKAAITSYEKSDGVYTGLSTRNQETYETLKHEKPPQGSGATNFSLLKQAGDVEENPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGRAEGRGSLLTCGDVEENPGPMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 109 - protein, 542 aa, Artificial Sequence
  synthetic polypeptide
MALPVTALLLPLALLLHAARPDYKDDDDKDYKDDDDKDYKDDDDKGGGGSEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVTTTPAPRPPTPAPTIASQPLSLRPEACRPAAGGAVHTRGLDFACDIYIWAPLAGTCGVLLLSLVITLYCNCRNTGPWLKKVLKCNTPDPSKFFSQLSSEHGGDVQKWLSSPFPSSSFSPGGLAPEISPLEVLERDKVTQLLPLNTDAYLSLQELQGQDPTHLVKRGRKKLLYIFKQPFMRPVQTTQEEDGCSCRFPEEEEGGCELRLKIQVRKAAITSYEKSDGVYTGLSTRNQETYETLKHEKPPQ

SEQ ID NO: 110 - protein, 481 aa, Artificial Sequence
  synthetic polypeptide
MALPVTALLLPLALLLHAARPDYKDDDDKDYKDDDDKDYKDDDDKGGGGSEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVTTTPAPRPPTPAPTIASQPLSLRPEACRPAAGGAVHTRGLDFACDPFFFCCFIAVAMGIRFIIMVAWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 111 - protein, 460 aa, Artificial Sequence
  synthetic polypeptide
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYSPFFFCCFIAVAMGIRFIIMVATTTPAPRPPTPAPTIASQPLSLRPEACRPAAGGAVHTRGLDFACDEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVGGGGSDYKDDDDKDYKDDDDKDYKDDDDK

SEQ ID NO: 112 - protein, 510 aa, Artificial Sequence
  synthetic polypeptide
MALPVTALLLPLALLLHAARPDYKDDDDKDYKDDDDKDYKDDDDKGGGGSEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVTTTPAPRPPTPAPTIASQPLSLRPEACRPAAGGAVHTRGLDFACDGGTVLLLLFVISITTIIVIFLNRRRRRERRDLFTESWDTQKAPNNYRSPISTSQPTNQSMDDTREDIYVNYPTFSRRPKTRVWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 113 - protein, 449 aa, Artificial Sequence
  synthetic polypeptide
MALPVTALLLPLALLLHAARPDYKDDDDKDYKDDDDKDYKDDDDKGGGGSEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVTTTPAPRPPTPAPTIASQPLSLRPEACRPAAGGAVHTRGLDFACDPFFFCCFIAVAMGIRFIIMVAWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 114 - protein, 428 aa, Artificial Sequence
  synthetic polypeptide
WFLKRERQEEYIEEKKRVDICRETPNICPHSGENTEYDTIPHTNRTILKEDPANTVYSTVEIPKKMENPHSLLTMPDTPRLFAYENVIPFFFCCFIAVAMGIRFIIMVATTTPAPRPPTPAPTIASQPLSLRPEACRPAAGGAVHTRGLDFACDEVQLVQSGAEVKKPGSSVKVSCKASGYTITDSNIHWVRQAPGQSLEWIGYIYPYNGGTDYNQKFKNRATLTVDNPTNTAYMELSSLRSEDTAFYYCVNGNPWLAYWGQGTLVTVSSGGGGSGGGGSGGGGSDIQLTQSPSTLSASVGDRVTITCRASESLDNYGIRFLTWFQQKPGKAPKLLMYAASNQGSGVPSRFSGSGSGTEFTLTISSLQPDDFATYYCQQTKEVPWSFGQGTKVEVKRTVGGGGSDYKDDDDKDYKDDDDKDYKDDDDK

SEQ ID NO: 115 - DNA, 63 bp, Artificial Sequence
  synthetic polynucleotide
atgggactggcctttctggtgctggtggctctggtgtggtttctggtggaggactggctgagc

SEQ ID NO: 116 - DNA, 63 bp, Artificial Sequence
  synthetic polynucleotide
ggcggcaccgtgctgctgctgctgttcgtgatctccatcaccaccatcatcgtcatctttctg

SEQ ID NO: 117 - protein, 21 aa, Artificial Sequence
  synthetic polypeptide
MGLAFLVLVALVWFLVEDWLS

SEQ ID NO: 118 - protein, 21 aa, Artificial Sequence
  synthetic polypeptide
GGTVLLLLFVISITTIIVIFL

SEQ ID NO: 119 - DNA, 12 bp, Artificial Sequence
  synthetic polynucleotide
ggaggcggttcg

SEQ ID NO: 120 - protein, 4 aa, Artificial Sequence
  synthetic polypeptide
GGGS

SEQ ID NO: 121 - DNA, 75 bp, Artificial Sequence
  synthetic polynucleotide
gattacaaggatgacgacgataaggactataaggacgacgacgataaggattataaagacgatgacgacaaaggt

SEQ ID NO: 122 - protein, 25 aa, Artificial Sequence
  synthetic polypeptide
DYKDDDDKDYKDDDDKDYKDDDDKG

SEQ ID NO: 123 - protein, 370 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFESLLPDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 124 - protein, 335 aa, Homo sapiens
MAGSPTCLTLIYILWQLTGSAASGPVKELVGSVGGAVTFPLKSKVKQVDSIVWTFNTTPLVTIQPEGGTIIVTQNRNRERVDFPDGGYSLKLSKLKKNDSGIYYVGIYSSSLQQPSTQEYVLHVYEHLSKPKVTMGLQSNKNGTCVTNLTCCMEHGEEDVIYTWKALGQAANESHNGSILPISWRWGESDMTFICVARNPVSRNFSSPILARKLCEGAADDPDSSMVLLCLLLVPLLLSLFVLGLFLWFLKRERQEEYIEEKKRVDICRETPNICPHSGENTEYDTIPHTNRTILKEDPANTVYSTVEIPKKMENPHSLLTMPDTPRLFAYENVI

SEQ ID NO: 125 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 126 - protein, 331 aa, Homo sapiens
MLWLFQSLLFVFCFGPGNVVSQSSLTPLMVNGILGESVTLPLEFPAGEKVNFITWLFNETSLAFIVPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKMILFMVSGICIVFGFIILLLLVLRKRRDSLSLSTQRTQGPESARNLEYVSVSPTNNTVYASVTHSNRETEIWTPRENDTITIYSTINHSKESKPTFSRATALDNVV

SEQ ID NO: 127 - protein, 86 aa, Homo sapiens
MIPAVVLLLLLLVEQAAALGEPQLCYILDAILFLYGIVLTLLYCRLKIQVRKAAITSYEKSDGVYTGLSTRNQETYETLKHEKPPQ

SEQ ID NO: 128 - protein, 163 aa, Homo sapiens
MKWKALFTAAILQAQLPITEAQSFGLLDPKLCYLLDGILFIYGVILTALFLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 130 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 131 - protein, 8 aa, Artificial Sequence
  synthetic polypeptide
DYKDDDDK

SEQ ID NO: 132 - protein, 21 aa, Artificial Sequence
  synthetic polypeptide
GSGEGRGSLLTCGDVEENPGP

SEQ ID NO: 133 - protein, 23 aa, Artificial Sequence
  synthetic polypeptide
GSGQCTNYALLKLAGDVESNPGP

SEQ ID NO: 134 - protein, 25 aa, Artificial Sequence
  synthetic polypeptide
GSGVKQTLNFDLLKLAGDVESNPGP

SEQ ID NO: 135 - protein, 216 aa, Homo sapiens
MGWIRGRRSRHSWEMSEFHNYNLDLKKSDFSTRWQKQRCPVVKSKCRENASPFFFCCFIAVAMGIRFIIMVAIWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 136 - protein, 304 aa, Homo sapiens
MSSTLPALLCVGLCLSQRISAQQQTLPKPFIWAEPHFMVPKEKQVTICCQGNYGAVEYQLHFEGSLFAVDRPKPPERINKVQFYIPDMNSRMAGQYSCIYRVGELWSEPSNLLDLVVTEMYDTPTLSVHPGPEVISGEKVTFYCRLDTATSMFLLLKEGRSSHVQRGYGKVQAEFPLGPVTTAHRGTYRCFGSYNNHAWSFPSEPVKLLVTGDIENTSLAPEDPTFPADTWGTYLLTTETGLQKDHALWDHTAQNLLRMGLAFLVLVALVWFLVEDWLSRKRTRERASRASTWEGRRRLNTQTL

SEQ ID NO: 137 - protein, 336 aa, Homo sapiens
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 138 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 139 - protein, 3 aa, Artificial Sequence
  synthetic polypeptide
GGS

SEQ ID NO: 140 - protein, 6 aa, Artificial Sequence
  synthetic polypeptide
GGSGGS

SEQ ID NO: 141 - protein, 9 aa, Artificial Sequence
  synthetic polypeptide
GGSGGSGGS

SEQ ID NO: 142 - protein, 12 aa, Artificial Sequence
  synthetic polypeptide
GGSGGSGGSGGS

SEQ ID NO: 143 - protein, 15 aa, Artificial Sequence
  synthetic polypeptide
GGSGGSGGSGGSGGS

SEQ ID NO: 144 - protein, 18 aa, Artificial Sequence
  synthetic polypeptide
GGSGGSGGSGGSGGSGGS

SEQ ID NO: 145 - protein, 21 aa, Artificial Sequence
  synthetic polypeptide
GGSGGSGGSGGSGGSGGSGGS

SEQ ID NO: 146 - protein, 24 aa, Artificial Sequence
  synthetic polypeptide
GGSGGSGGSGGSGGSGGSGGSGGS

SEQ ID NO: 147 - protein, 27 aa, Artificial Sequence
  synthetic polypeptide
GGSGGSGGSGGSGGSGGSGGSGGSGGS

SEQ ID NO: 148 - protein, 30 aa, Artificial Sequence
  synthetic polypeptide
GGSGGSGGSGGSGGSGGSGGSGGSGGSGGS

SEQ ID NO: 149 - protein, 8 aa, Artificial Sequence
  synthetic polypeptide
GGGSGGGS

SEQ ID NO: 150 - protein, 12 aa, Artificial Sequence
  synthetic polypeptide
GGGSGGGSGGGS

SEQ ID NO: 151 - protein, 16 aa, Artificial Sequence
  synthetic polypeptide
GGGSGGGSGGGSGGGS

SEQ ID NO: 152 - protein, 20 aa, Artificial Sequence
  synthetic polypeptide
GGGSGGGSGGGSGGGSGGGS

SEQ ID NO: 153 - protein, 24 aa, Artificial Sequence
  synthetic polypeptide
GGGSGGGSGGGSGGGSGGGSGGGS

SEQ ID NO: 154 - protein, 28 aa, Artificial Sequence
  synthetic polypeptide
GGGSGGGSGGGSGGGSGGGSGGGSGGGS

SEQ ID NO: 155 - protein, 32 aa, Artificial Sequence
  synthetic polypeptide
GGGSGGGSGGGSGGGSGGGSGGGSGGGSGGGS

SEQ ID NO: 156 - protein, 36 aa, Artificial Sequence
  synthetic polypeptide
GGGSGGGSGGGSGGGSGGGSGGGSGGGSGGGSGGGS

SEQ ID NO: 157 - protein, 40 aa, Artificial Sequence
  synthetic polypeptide
GGGSGGGSGGGSGGGSGGGSGGGSGGGSGGGSGGGSGGGS

SEQ ID NO: 158 - protein, 10 aa, Artificial Sequence
  synthetic polypeptide
GGGGSGGGGS

SEQ ID NO: 159 - protein, 15 aa, Artificial Sequence
  synthetic polypeptide
GGGGSGGGGSGGGGS

SEQ ID NO: 160 - protein, 20 aa, Artificial Sequence
  synthetic polypeptide
GGGGSGGGGSGGGGSGGGGS

SEQ ID NO: 161 - protein, 25 aa, Artificial Sequence
  synthetic polypeptide
GGGGSGGGGSGGGGSGGGGSGGGGS

SEQ ID NO: 162 - protein, 30 aa, Artificial Sequence
  synthetic polypeptide
GGGGSGGGGSGGGGSGGGGSGGGGSGGGGS

SEQ ID NO: 163 - protein, 35 aa, Artificial Sequence
  synthetic polypeptide
GGGGSGGGGSGGGGSGGGGSGGGGSGGGGSGGGGS

SEQ ID NO: 164 - protein, 40 aa, Artificial Sequence
  synthetic polypeptide
GGGGSGGGGSGGGGSGGGGSGGGGSGGGGSGGGGSGGGGS

SEQ ID NO: 165 - protein, 45 aa, Artificial Sequence
  synthetic polypeptide
GGGGSGGGGSGGGGSGGGGSGGGGSGGGGSGGGGSGGGGSGGGGS

SEQ ID NO: 166 - protein, 50 aa, Artificial Sequence
  synthetic polypeptide
GGGGSGGGGSGGGGSGGGGSGGGGSGGGGSGGGGSGGGGSGGGGSGGGGS

SEQ ID NO: 167 - protein, 6 aa, Artificial Sequence
  synthetic polypeptide
GGGGGS

SEQ ID NO: 168 - protein, 12 aa, Artificial Sequence
  synthetic polypeptide
GGGGGSGGGGGS

SEQ ID NO: 169 - protein, 18 aa, Artificial Sequence
  synthetic polypeptide
GGGGGSGGGGGSGGGGGS

SEQ ID NO: 170 - protein, 24 aa, Artificial Sequence
  synthetic polypeptide
GGGGGSGGGGGSGGGGGSGGGGGS

SEQ ID NO: 171 - protein, 30 aa, Artificial Sequence
  synthetic polypeptide
GGGGGSGGGGGSGGGGGSGGGGGSGGGGGS

SEQ ID NO: 172 - protein, 36 aa, Artificial Sequence
  synthetic polypeptide
GGGGGSGGGGGSGGGGGSGGGGGSGGGGGSGGGGGS

SEQ ID NO: 173 - protein, 42 aa, Artificial Sequence
  synthetic polypeptide
GGGGGSGGGGGSGGGGGSGGGGGSGGGGGSGGGGGSGGGGGS

SEQ ID NO: 174 - protein, 48 aa, Artificial Sequence
  synthetic polypeptide
GGGGGSGGGGGSGGGGGSGGGGGSGGGGGSGGGGGSGGGGGSGGGGGS

SEQ ID NO: 175 - protein, 54 aa, Artificial Sequence
  synthetic polypeptide
GGGGGSGGGGGSGGGGGSGGGGGSGGGGGSGGGGGSGGGGGSGGGGGSGGGGGS

SEQ ID NO: 176 - protein, 60 aa, Artificial Sequence
  synthetic polypeptide
GGGGGSGGGGGSGGGGGSGGGGGSGGGGGSGGGGGSGGGGGSGGGGGSGGGGGSGGGGGS